(19) United States
(12) Patent Application Publication
(10) Pub. No.: US 2024/0324536 A1
(43) Pub. Date: Oct. 3, 2024

(54) METHODS AND COMPOSITIONS FOR REDUCING THORNS OR PRICKLES IN PLANTS

(71) Applicant: Pairwise Plants Services, Inc., Durham, NC (US)

(72) Inventors: Brian Charles Wilding Crawford, Cary, NC (US); Brian Douglas St. Aubin, Cary, NC (US); Thomas J. Poorten, Durham, NC (US); Gina Pham, Durham, NC (US)

(21) Appl. No.: 18/618,141

(22) Filed: Mar. 27, 2024

Related U.S. Application Data
(60) Provisional application No. 63/493,009, filed on Mar. 30, 2023.
(60) Provisional application No. 63/515,159, filed on Jul. 24, 2023.

(51) Int. Cl. A01H 6/74; C07K 14/415; C12N 15/10; C12N 15/82

(52) U.S. Cl. CPC A01H 6/74; C07K 14/415; C12N 15/102; C12N 15/8242

(57) ABSTRACT

This invention relates to compositions and methods for modifying a WUSCHEL (WUS)-Related Homeobox (WOX) functional gene in a Rosaceae plant, optionally to modify the architecture of the thorns and/or spines, to reduce thorn and prickle number and/or size, or to eliminate thorns and/or prickles. The invention further relates to Rosaceae plants having thorns and/or prickles with a modified architecture, a reduced number and/or size thorns and/or prickles, or no thorns and/or prickles produced using the methods and compositions of the invention. The invention additionally relates to methods of identifying and/or breeding plants having thorns and/or prickles with a modified architecture, a reduced number and/or size thorns and/or prickles, or no thorns and/or prickles.

### STATEMENT REGARDING ELECTRONIC FILING OF A SEQUENCE LISTING

A Sequence Listing in XML text format, entitled 1499-127_ST26.xml, 372,120 bytes in size, generated on Mar. 23, 2024, and filed herewith, is hereby incorporated by reference into the specification for its disclosures.

### FIELD OF THE INVENTION

This invention relates to compositions and methods for modifying a functional WUSCHEL (WUS)-Related Homeobox (WOX) gene in a Rosaceae plant, optionally to modify the architecture of the thorns and/or spines, to reduce thorn and prickle number and/or size, or to eliminate thorns and/or prickles. The invention further relates to Rosaceae plants having thorns and/or prickles with a modified architecture, a reduced number and/or size thorns and/or prickles, or no thorns and/or prickles produced using the methods and compositions of the invention. The invention additionally relates to methods of identifying and/or breeding plants having thorns and/or prickles with a modified architecture, a reduced number and/or size thorns and/or prickles, or no thorns and/or prickles.

### BACKGROUND OF THE INVENTION

Structures such as trichomes, thorns, hairs, spines and prickles are found on the surface of plant parts such as stems, leaves and buds of many plant species. These structures have various developmental origins, such as emerging from epidermal cells that form the outer most layers of stem tissues or forming from vasculature cells which are deeper within the plant tissues. Trichomes, hairs, thorns, spines and prickles are considered to have a common function of deterring herbivory of the plant leaves and stems, as well as immature buds. However, in agricultural and horticultural settings, thorns and prickles cause significant problems for those handling the plants. Thorns and prickles interfere with propagation, care and harvesting of such crops, resulting in increased costs. Breeding has been used to develop some plant varieties with reduced thorn/prickle production or thorns/prickles having modified architecture, but this is often associated with other less desirable traits. Thus, improved strategies are needed for the development of plants having thorns and/or prickles that are reduced in number, reduced in size, or having a modified architecture.

### SUMMARY OF THE INVENTION

One aspect of the invention provides a Rosaceae plant or plant part thereof comprising a mutated endogenous WUSCHEL (WUS)-Related Homeobox (WOX) gene, wherein the mutated endogenous WOX gene is provided by and/or includes at least one mutation in at least one (e.g., one or more) endogenous functional WOX gene encoding a WOX transcription factor, optionally wherein the at least one mutation may be a non-natural mutation.

A second aspect of the invention provides a Rosaceae plant cell, comprising an editing system the editing system comprising: (a) a CRISPR-Cas effector protein; and (b) a guide nucleic acid comprising a spacer sequence with complementarity to an endogenous functional target gene encoding a WUSCHEL (WUS)-Related Homeobox (WOX) transcription factor in the Rosaceae plant cell.

A third aspect of the invention provides a Rosaceae plant cell comprising at least one mutation within an endogenous WOX gene, wherein the at least one mutation is a base substitution, a base insertion or a base deletion that is introduced using an editing system that comprises a nucleic acid binding domain that binds to a target site within the endogenous WOX gene, wherein the endogenous WOX gene: (a) comprises a nucleotide sequence having at least 80% sequence identity to any one of SEQ ID NOs: 69, 70, 75, 76, 128, 129 or 162-173; (b) comprises a region having at least 80% sequence identity to a nucleotide sequence of any one of SEQ ID NOs: 78-82, 83-86, 87-90, 91-109, 110-125 or 131-135; (c) encodes an amino acid sequence having at least 80% sequence identity to any one of SEQ ID NOs: 71, 77, 130, or 174-182; and/or (d) encodes a region of an amino acid sequence having at least 80% sequence identity to any one of SEQ ID NO:126, SEQ ID NO: 127, SEQ ID NO:136 or SEQ ID NO: 137, optionally wherein the at least one mutation may be a non-natural mutation.

A fourth aspect of the invention provides a method of producing/breeding a transgene-free edited Rosaceae plant, comprising: crossing a Rosaceae plant of the invention with a transgene-free plant, thereby introducing the at least one mutation into the Rosaceae plant that is transgene-free; and selecting a progeny Rosaceae plant that comprises the at least one mutation and is transgene-free, thereby producing a transgene-free edited Rosaceae plant, optionally wherein the at least one mutation may be a non-natural mutation.

A fifth aspect of the invention provides a method of providing a plurality of Rosaceae plants having phenotype of a reduced number of thorns and/or prickles, thorns and/or prickles that are reduced in size, thorns and/or prickles having a modified architecture, and/or no thorns and/or prickles, the method comprising planting two or more Rosaceae plants of the invention in a growing area, thereby providing the plurality of Rosaceae plants having a phenotype of reduced number of thorns and/or prickles, thorns and/or prickles that are reduced in size, thorns and/or prickles having a modified architecture, and/or no thorns and/or prickles as compared to a plurality of control Rosaceae plants devoid of the at least one mutation, optionally wherein the at least one mutation may be a non-natural mutation.

In a sixth aspect, a method is provided for creating a mutation in an endogenous WUSCHEL (WUS)-Related Homeobox (WOX) gene in a Rosaceae plant, the method comprising: (a) targeting a gene editing system to a portion of the endogenous WOX gene that (i) comprises a sequence having at least 80% sequence identity to any one of SEQ ID NOs: 78-82, 83-86, 87-90, 91-109, 110-125 or 131-135; and/or (ii) encodes a sequence having at least 80% identity to any one of SEQ ID NOs: 126, 127, 136 or 137, and (b) selecting a Rosaceae plant that comprises a modification located in a region of the endogenous WOX gene having at least 80% sequence identity to any one of SEQ ID NOs: 78-82, 83-86, 87-90, 91-109, 110-125 or 131-135.

In a seventh aspect, a method is provided for generating variation in a WUSCHEL (WUS)-Related Homeobox (WOX) transcription factor in a Rosaceae plant cell, comprising: introducing an editing system into a Rosaceae plant cell, wherein the editing system is targeted to a region of a functional WUSCHEL (WUS)-Related Homeobox (WOX) gene; and contacting the region of the functional WOX gene with the editing system, thereby introducing a mutation into the functional WOX gene and generating variation in the WOX transcription factor in the Rosaceae plant cell.

An eighth aspect provides a method for editing a specific site in the genome of a Rosaceae plant cell, the method comprising: cleaving, in a site-specific manner, a target site within an endogenous WUSCHEL (WUS)-Related Homeobox (WOX) gene in the Rosaceae plant cell, the endogenous WOX gene: (a) comprising a nucleotide sequence having at least 80% sequence identity to any one of SEQ ID NOs: 69, 70, 75, 76, 128, 129 or 162-173; (b) comprising a region having at least 80% sequence identity to a nucleotide sequence of any one of SEQ ID NOs: 78-82, 83-86, 87-90, 91-109, 110-125 or 131-135; (c) encoding an amino acid sequence having at least 80% sequence identity to any one of SEQ ID NOs: 71, 77, 130, or 174-182; and/or (d) encoding a region of an amino acid sequence having at least 80% sequence identity to any one of SEQ ID NOs: 126, 127, 136 or 137, thereby generating an edit in the endogenous WOX gene of the Rosaceae plant cell and producing a Rosaceae plant cell comprising the edit in the endogenous WOX gene.

A ninth aspect provides a method for making a Rosaceae plant, the method comprising (a) contacting a population of Rosaceae plant cells comprising an endogenous WUSCHEL (WUS)-Related Homeobox (WOX) gene with a nuclease linked to a nucleic acid binding domain (e.g., editing system) that binds to a sequence (i) having at least 80% sequence identity to a nucleotide sequence of any one of SEQ ID NOs: 69, 70, 75, 76, 128, 129, or 162-173; (ii) comprising a region having at least 80% identity to any one of SEQ ID NOs: 78-82, 83-86, 87-90, 91-109, 110-125 or 131-135; (iii) encoding an amino acid sequence having at least 80% sequence identity to any one of SEQ ID NOs: 71, 77, 130, or 174-182, and/or (iv) encoding a region having at least 80% sequence identity to any one of SEQ ID NOs: 126, 127, 136 or 137;

(b) selecting a Rosaceae plant cell from the population of Rosaceae plant cells in which an endogenous WOX gene has been mutated, thereby producing a Rosaceae plant cell comprising a mutation in the endogenous WOX gene; and (c) growing the selected Rosaceae plant cell into a Rosaceae plant comprising a mutation in the endogenous WOX gene.

A tenth aspect provides a method for reducing the number and/or size of thorns, modifying the architecture of thorns and/or prickles, and/or eliminating thorns and/or prickles (e.g., no thorns and/or prickles) in a Rosaceae plant, comprising (a) contacting a Rosaceae plant cell comprising an endogenous WUSCHEL (WUS)-Related Homeobox (WOX) gene with a nuclease targeting the endogenous WOX gene, wherein the nuclease is linked to a nucleic acid binding domain (e.g., editing system) that binds to a target site within the endogenous WOX gene, wherein the endogenous WOX gene: (i) comprises a nucleotide sequence having at least 80% sequence identity to any one of SEQ ID NOs: 69, 70, 75, 76, 128, 129 or 162-173; (ii) comprises a region having at least 80% sequence identity to a nucleotide sequence of any one of SEQ ID NOs: 78-82, 83-86, 87-90, 91-109, 110-125 or 131-135; (iii) encodes an amino acid sequence having at least 80% sequence identity to any one of SEQ ID NOs: 71, 77, 130, or 174-182; and/or (iv) encodes a region of an amino acid sequence having at least 80% sequence identity to any one of SEQ ID NOs: 126, 127, 136 or 137 to produce a Rosaceae plant cell comprising a mutation in the endogenous WOX gene; and (b) growing the Rosaceae plant cell comprising a mutation in the endogenous WOX gene into a Rosaceae plant comprising the mutation in the endogenous WOX gene thereby producing a Rosaceae plant having a mutated endogenous WOX gene and reduced number of thorns and/or prickles, thorns and/or prickles with a modified architecture, reduced size of thorns and/or prickles, and/or elimination of thorns and/or prickles in the Rosaceae plant.

An eleventh aspect provides a method for producing a Rosaceae plant or part thereof comprising at least one cell having a mutated endogenous WUSCHEL (WUS)-Related Homeobox (WOX) gene, the method comprising contacting a target site within an endogenous WOX gene in the Rosaceae plant or plant part with a nuclease comprising a cleavage domain and a nucleic acid binding domain, wherein the nucleic acid binding domain binds to a target site within the endogenous WOX gene, wherein the endogenous WOX gene (a) comprises a nucleotide sequence having at least 80% sequence identity to any one of SEQ ID NOs: 69, 70, 75, 76, 128, 129 or 162-173; (b) comprises a region having at least 80% sequence identity to a nucleotide sequence of any one of SEQ ID NOs: 78-82, 83-86, 87-90, 91-109, 110-125 or 131-135; (c) encodes an amino acid sequence having at least 80% sequence identity to any one of SEQ ID NOs: 71, 77, 130, or 174-182; and/or (d) encodes a region of an amino acid sequence having at least 80% sequence identity to any one of SEQ ID NOs: 126, 127, 136 or 137, thereby producing the Rosaceae plant or part thereof comprising at least one cell having a mutation in the endogenous WOX gene.

A twelfth aspect of the invention provides a method for producing a Rosaceae plant or part thereof comprising a mutated endogenous WUSCHEL (WUS)-Related Homeobox (WOX) gene and exhibiting a reduced number of thorns and/or prickles, thorns and/or prickles that have a modified architecture, thorns and/or prickles that are reduced in size, and/or no thorns and/or prickles, the method comprising contacting a target site within an endogenous WOX gene in the Rosaceae plant or plant part with a nuclease comprising a cleavage domain and a nucleic acid binding domain, wherein the nucleic acid binding domain binds to a target site within the endogenous WOX gene, wherein the endogenous WOX gene: (a) comprises a nucleotide sequence having at least 80% sequence identity to any one of SEQ ID NOs: 69, 70, 75, 76, 128, 129 or 162-173; (b) comprises a region having at least 80% sequence identity to a nucleotide sequence of any one of SEQ ID NOs: 78-82, 83-86, 87-90, 91-109, 110-125 or 131-135; (c) encodes an amino acid sequence having at least 80% sequence identity to any one of SEQ ID NOs: 71, 77, 130, or 174-182; and/or (d) encodes a region of an amino acid sequence having at least 80% sequence identity to any one of SEQ ID NOs: 126, 127, 136 or 137, thereby producing the Rosaceae plant or part thereof comprising an endogenous WOX gene having a mutation and exhibiting a reduced number of thorns and/or prickles, thorns and/or prickles that have a modified architecture, thorns and/or prickles that are reduced in size, and/or no thorns and/or prickles.

A thirteenth aspect provides a guide nucleic acid that binds to a target site within a WUSCHEL (WUS)-Related Homeobox (WOX) gene, wherein the target site is in a region of the WOX gene having at least 80% sequence identity to any one of SEQ ID NOs: 78-82, 83-86, 87-90, 91-109, 110-125 or 131-135.

In a fourteenth aspect, a system is provided that comprises a guide nucleic acid of the invention and a CRISPR-Cas effector protein that associates with the guide nucleic acid.

A fifteenth aspect provides a gene editing system comprising a CRISPR-Cas effector protein in association with a guide nucleic acid, wherein the guide nucleic acid comprises a spacer sequence that binds to an endogenous functional WUSCHEL (WUS)-Related Homeobox (WOX) gene.

In a sixteenth aspect, a complex comprising a guide nucleic acid and a CRISPR-Cas effector protein comprising a cleavage domain is provided, wherein the guide nucleic acid binds to a target site within endogenous WUSCHEL (WUS)-Related Homeobox (WOX) gene, wherein the endogenous WOX gene: (a) comprises a nucleotide sequence having at least 80% sequence identity to any one of SEQ ID NOs: 69, 70, 75, 76, 128, 129 or 162-173; (b) comprises a region having at least 80% sequence identity to a nucleotide sequence of any one of SEQ ID NOs: 78-82, 83-86, 87-90, 91-109, 110-125 or 131-135; (c) encodes an amino acid sequence having at least 80% sequence identity to any one of SEQ ID NOs: 71, 77, 130, or 174-182; and/or (d) encodes a region of an amino acid sequence having at least 80% sequence identity to any one of SEQ ID NOs: 126, 127, 136 or 137, and the cleavage domain cleaves a target strand in the WOX gene.

In a seventeenth aspect, an expression cassette is provided, the expression cassette comprising (a) a polynucleotide encoding CRISPR-Cas effector protein comprising a cleavage domain and (b) a guide nucleic acid that binds to a target site within an endogenous WUSCHEL (WUS)-Related Homeobox (WOX) gene, wherein the guide nucleic acid comprises a spacer sequence that is complementary to and binds to (i) a portion of a nucleic acid having at least 80% sequence identity to any one of SEQ ID NOs: 69, 70, 75, 76, 128, 129 or 162-173; (ii) a portion of a nucleic acid having at least 80% sequence identity to any one of SEQ ID NOs: 78-82, 83-86, 87-90, 91-109, 110-125 or 131-135; (iii) a portion of a nucleic acid encoding an amino acid sequence having at least 80% sequence identity to any one of SEQ ID NOs: 71, 77, 130, or 174-182; and/or (iv) a portion of a nucleic acid encoding an amino acid sequence having at least 80% identity to any one of SEQ ID NOs: 126, 127, 136 or 137.

In another aspect, Rosaceae plants are provided that comprise in their genome one or more mutated WUSCHEL (WUS)-Related Homeobox (WOX) genes produced by the methods of the invention, optionally wherein the at least one mutation may be a non-natural mutation, optionally wherein the mutated WOX gene comprises a nucleotide sequence having at least 90% sequence identity to any one of the mutated WOX genes of SEQ ID NOs: 143, 144, 145, 147, 149, 151, 153 and/or 155 and/or encodes a WOX transcription factor having at least 90% sequence identity to any one of the mutated WOX transcription factors of SEQ ID NOs: 146, 148, 150, 152, 154 and/or 156.

In a further aspect, a genetic marker for thorns and/or prickles with a modified architecture, a reduced number and/or size thorns and/or prickles, or no thorns and/or prickles in a blackberry plant or part thereof, the genetic marker comprising (a) the nucleic acid sequence of GAACTCTG located between nucleotide position of 2897 and 2898 with reference to the nucleotide position numbering of SEQ ID NO:69 and/or (b) the nucleic acid sequence of SEQ ID NO: 143 in a blackberry plant or part thereof is provided, the genetic marker comprising (a) the nucleic acid sequence of GAACTCTG located between nucleotide position of 2897 and 2898 with reference to the nucleotide position numbering of SEQ ID NO:69 and/or (b) the nucleic acid sequence of SEQ ID NO:143.

Additionally provided is a method of identifying a blackberry plant or part thereof having a phenotype of thorns and/or prickles with a modified architecture, a reduced number and/or size thorns and/or prickles, or no thorns and/or prickles, the method comprising identifying in the genome of a cell of the blackberry plant or part thereof (a) an insertion in an endogenous WUSCHEL (WUS)-Related Homeobox (WOX) gene, the insertion comprising of the nucleic acid sequence of GAACTCTG located between nucleotide position of 2897 and 2898 with reference to the nucleotide position numbering of SEQ ID NO:69 and/or (b) the nucleic acid sequence of SEQ ID NO:143.

Further provided is a method for producing a blackberry plant having thorns and/or prickles with a modified architecture, a reduced number and/or size thorns and/or prickles, or no thorns and/or prickles comprising, crossing a blackberry plant comprising in its genome (a) an insertion in an endogenous WUSCHEL (WUS)-Related Homeobox (WOX) gene, the insertion comprising of the nucleic acid sequence of GAACTCTG located between nucleotide position of 2897 and 2898 with reference to the nucleotide position numbering of SEQ ID NO:69 and/or (b) the nucleic acid sequence of SEQ ID NO:143 with itself or another blackberry plant to produce progeny plants; and selecting one or more progeny plants comprising the insertion to provide selected progeny, thereby producing blackberry plants having thorns and/or prickles with a modified architecture, a reduced number and/or size thorns and/or prickles, or no thorns and/or prickles.

Further provided are polypeptides, polynucleotides, nucleic acid constructs, expression cassettes and vectors for making a Rosaceae plant of this invention.

These and other aspects of the invention are set forth in more detail in the description of the invention below.

### BRIEF DESCRIPTION OF THE SEQUENCES

SEQ ID NOs: 1-17 are exemplary Cas12a amino acid sequences useful with this invention.

SEQ ID NOs: 18-20 are exemplary Cas12a nucleotide sequences useful with this invention.

SEQ ID NO:21-22 are exemplary regulatory sequences encoding a promoter and intron.

SEQ ID NOs: 23-29 are exemplary cytosine deaminase sequences useful with this invention.

SEQ ID NOs: 30-40 are exemplary adenine deaminase amino acid sequences useful with this invention.

SEQ ID NO:41 is an exemplary uracil-DNA glycosylase inhibitor (UGI) sequences useful with this invention.

SEQ ID NOs: 42-44 provide example peptide tags and affinity polypeptides useful with this invention.

SEQ ID NOs: 45-55 provide example RNA recruiting motifs and corresponding affinity polypeptides useful with this invention.

SEQ ID NOs: 56-57 are exemplary Cas9 polypeptide sequences useful with this invention.

SEQ ID NOs: 58-68 are exemplary Cas9 polynucleotide sequences useful with this invention.

SEQ ID NO:69 and SEQ ID NO:72 are example WOX1 genomic sequences from blackberry.

SEQ ID NO:70 and SEQ ID NO:73 are example WOX1 genomic sequences coding sequences from blackberry.

SEQ ID NO:71 and SEQ ID NO:74 are example WOX1 transcription factor sequences from blackberry encoded by SEQ ID NO:69 and SEQ ID NO:70, respectively.

SEQ ID NO:75 is an example WOX23 genomic sequence from black raspberry.

SEQ ID NO:76 is an example WOX23 coding sequence from black raspberry.

SEQ ID NO:77 is an example WOX23 transcription factor sequence from black raspberry, encoded by SEQ ID NO:75 and SEQ ID NO:76.

SEQ ID NOs: 78-90 are example portions or regions from a WOX1 genomic sequence from blackberry (Exon 1).

SEQ ID NOs: 91-109 are example portions or regions from a WOX1 genomic sequence from blackberry (Exon 2).

SEQ ID NOs: 104, 105, 109, and 110-125 are example portions or regions from a WOX23 genomic sequence from black raspberry (Exon 2).

SEQ ID NO:126 is an example portion or region of a WOX1 transcription factor from blackberry.

SEQ ID NO:127 is an example portion or region of a WOX1 transcription factor from blackberry and a WOX23 transcription factor from black raspberry.

SEQ ID NO: 128 is an example WOX1 genomic sequence from red raspberry.

SEQ ID NO: 129 is an example WOX1 coding sequence from red raspberry.

SEQ ID NO: 130 is an example WOX1 polypeptide sequence from red raspberry, encoded by SEQ ID NO: 128 and SEQ ID NO:129.

SEQ ID NOs: 95, 96, 97, 104, 105, 111, 112, 113, 118, 119, 120, 124, 125, and 131-135, are example portions or regions from a WOX1 genomic sequence from red raspberry (Exon 2). SEQ ID NO:136 and SEQ ID NO:136 are example portions or regions of a WOX1 transcription factor from red raspberry.

SEQ ID NO:138-142 are example spacer sequences for nucleic acid guides useful with this invention.

SEQ ID NO:143 is a naturally occurring mutated WOX1 gene.

SEQ ID NOs: 144, 145, 147, 149, 151, 153 and 155 are example mutated WOX1 genomic sequences edited as described herein.

SEQ ID NOs: 146, 148, 150, 152, 154 and 156 are example mutated WOX1 polypeptides, which are encoded by SEQ ID NOs: 145, 147, 149, 151, 153 and 155, respectively.

SEQ ID NO:157-159 are consecutive nucleotides deleted from SEQ ID NO:69.

SEQ ID NO: 160 is the amino acid sequence of the WUS-box motif of the WOX family.

SEQ ID NO:161 is the consensus amino acid sequence of the WUS-box motif of the WOX family.

SEQ ID NO:162-173 are example WOX1 nucleotide sequences from Rosa chinensis.

SEQ ID NO:174-182 are example WOX1 amino acid sequences from Rosa chinensis.

### BRIEF DESCRIPTION OF THE DRAWINGS

FIG. 1 shows a schematic of the domain structure of the WOX1 protein. The homeodomain (HD) is the most prominent and defining feature of the WOX family. The WUS-box motif (oval) of the WOX family is defined in a strict sense, as T-L-[DEQP]-L-F-P-[GITVL]-[GSKNTCV] (SEQ ID NO:160), consensus TLELFPLH (SEQ ID NO:161).

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 5%, 10%, 15%, 20%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 96%, 97%, 98%, 99%, 99.5%, 99.6%, 99.7%, 99.8%, 99.9%, or 100% as compared to a control. In particular embodiments, the reduction can result in no or essentially no (i.e., an insignificant amount, e.g., less than about 10% or even 5%) detectable activity or amount. As an example, a Rosaceae plant comprising a mutation in a WUSCHEL (WUS)-Related Homeobox (WOX) gene as described herein can exhibit a reduction in thorns and/or prickles of at least 50% when compared to a control Rosaceae plant devoid of the at least one mutation (e.g., a reduction in thorns and/or prickles of at least about 50% to about 100%, e.g., about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any range or value therein).

A control Rosaceae plant (e.g., a Rubus spp., Rosa spp., Hulthemia spp., Hesperrhodos spp., a Platyrhodon spp.) is typically the same plant as the edited plant, but the control plant has not been similarly edited and therefore is devoid of the mutation. A control plant maybe an isogenic plant and/or a wild type plant. Thus, a control plant can be the same breeding line, variety, or cultivar as the subject plant into which a mutation as described herein is introgressed, but the control breeding line, variety, or cultivar is free of the mutation. In some embodiments, a comparison between a Rosaceae plant of the invention and a control Rosaceae plant is made under the same growth conditions, e.g., the same environmental conditions (soil, hydration, light, heat, nutrients, and the like).

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, a nucleic acid molecule and/or a nucleotide sequence may express a polypeptide of interest or, for example, a functional untranslated RNA.

A “heterologous” or a “recombinant” nucleotide sequence is a nucleotide sequence not naturally associated with a host cell into which it is introduced, including non-naturally occurring multiple copies of a naturally occurring nucleotide sequence. A “heterologous” nucleotide/polypeptide may originate from a foreign species, or, if from the same species, is substantially modified from its native form in composition and/or genomic locus by deliberate human intervention.

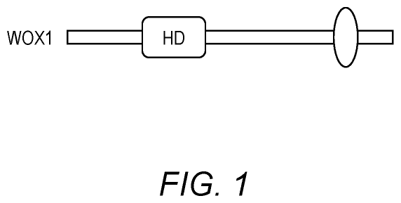

A “native” or “wild type” nucleic acid, nucleotide sequence, polypeptide or amino acid sequence refers to a naturally occurring or endogenous nucleic acid, nucleotide sequence, polypeptide or amino acid sequence. In some contexts, a “wild type” nucleic acid is a nucleic acid that is not edited as described herein and can differ from an “endogenous” gene that may be edited as described herein (e.g., a mutated endogenous gene). In some contexts, a “wild type” nucleic acid (e.g., unedited) may be heterologous to the organism in which the wild type nucleic acid is found (e.g., a transgenic organism). As an example, a “wild type endogenous WUSCHEL (WUS)-Related Homeobox (WOX) gene” is a WOX gene that is naturally occurring in or endogenous to the reference organism, e.g., a Rosaceae plant (e.g., a Rubus spp., Rosa spp., Hulthemia spp., Hesperrhodos spp., a Platyrhodon spp.), and may be subject to modification as described herein, after which, such a modified endogenous gene is no longer wild type. In some embodiments, an endogenous WOX gene may be an endogenous WOX1 gene and/or an endogenous WOX23 gene. In some embodiments, an endogenous WOX gene may be functional or nonfunctional. An endogenous functional WOX gene produces a functional WOX transcription factor and/or expression of the endogenous functional WOX gene provides a plant or plant part that includes thorns and/or prickles. In some embodiments, an endogenous functional WOX gene comprises a nucleotide sequence having at least 80% (e.g., 85%, 90%, 95%, or 100%) sequence identity to one or more of SEQ ID NOs: 69, 70, 75, 76, 78-82, 83-86, 87-90, 91-109, 110-125, 128, 129, 131-135, and 162-173 and/or encodes an amino acid sequence having at least 80% (e.g., 85%, 90%, 95%, or 100%) sequence identity to one or more of SEQ ID NOs: 71, 77, 126, 127, 130, 136, 137 and 174-182. In some embodiments, a functional WOX transcription factor may be a full-length protein (e.g., when compared to a reference WOX transcription factor that is known to be functional) and/or complexes (e.g., dimerizes) to another protein (that may be the same or different) and/or nucleic acid. In some embodiments, a functional WOX transcription factor includes all of its functional domains such as its DNA binding domain, transactivating domain, and homeodomain, can dimerize with itself, bind to proteins other than itself, and/or can bind to DNA. In some embodiments, a functional WOX transcription factor may include a homeodomain and motif as shown in FIG. 1 and/or have a structure, activity, and/or function as described in van der Graaff, et al., Genome Biology 2009, 10:248. A nonfunctional WOX gene (e.g., an endogenous nonfunctional WOX gene) produces a nonfunctional WOX transcription factor (e.g., a truncated protein) or no WOX transcription factor.

As used herein, the term “heterozygous” refers to a genetic status wherein different alleles reside at corresponding loci on homologous chromosomes.

As used herein, the term “homozygous” refers to a genetic status wherein identical alleles reside at corresponding loci on homologous chromosomes.

As used herein, the term “allele” refers to one of two or more different nucleotides or nucleotide sequences that occur at a specific locus.

The term “mutation” refers to point mutations (e.g., missense, or nonsense, or insertions or deletions of single base pairs that result in frame shifts), insertions, deletions, inversions and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue. A truncation can include a truncation at the C-terminal end of a polypeptide or at the N-terminal end of a polypeptide. A truncation of a polypeptide can be the result of a deletion of the corresponding 5′ end or 3′ end of the gene encoding the polypeptide. A frameshift mutation can occur when deletions or insertions of one or more base pairs are introduced into a gene, optionally resulting in an out-of-frame mutation or an in-frame mutation. Frameshift mutations in a gene can result in the production of a polypeptide that is longer, shorter or the same length as the wild type polypeptide depending on when the first stop codon occurs following the mutated region of the gene. As an example, an out-of-frame mutation that produces a premature stop codon can produce a polypeptide that is shorter that the wild type polypeptide, or, in some embodiments, the polypeptide may be absent/undetectable. A DNA inversion is the result of a rotation of a genetic fragment within a region of a chromosome.

A “null allele” is a nonfunctional allele caused by a genetic mutation that results in a complete lack of production of the corresponding protein or produces a protein that is non-functional.

A “knock-out mutation” is a mutation in a gene that produces a nonfunctional protein, which may be detectable, or no production of protein.

A “knock-down mutation” is a mutation in a gene that provides reduced protein production and/or reduced protein activity (e.g., reduced protein function) as compared to the amount of and/or activity of protein produced by the gene in the absence of the mutation.

A “recessive mutation” is a mutation in a gene that produces a phenotype when homozygous but the phenotype is not observable when the locus is heterozygous.

A “dominant mutation” is a mutation in a gene that produces a mutant phenotype in the presence of a non-mutated copy of the gene. A dominant mutation may be a loss or a gain of function mutation, a hypomorphic mutation, a hypermorphic mutation or a weak loss of function or a weak gain of function.

A “dominant negative mutation” is a mutation that produces an altered gene product (e.g., having an aberrant function relative to wild type), which gene product adversely affects the function of the wild-type allele or gene product. For example, a “dominant negative mutation” may block a function of the wild type gene product. A dominant negative mutation may also be referred to as an “antimorphic mutation.”

A “semi-dominant mutation” refers to a mutation in which the penetrance of the phenotype in a heterozygous organism is less than that observed for a homozygous organism.

A “weak loss-of-function mutation” is a mutation that results in a gene product having partial function or reduced function (partially inactivated) as compared to the wildtype gene product.

A “hypomorphic mutation” is a mutation that results in a partial loss of gene function, which may occur through reduced expression (e.g., reduced protein and/or reduced RNA) or reduced functional performance (e.g., reduced activity), but not a complete loss of function/activity. A “hypomorphic” allele is a semi-functional allele caused by a genetic mutation that results in production of the corresponding protein that functions at anywhere between 1% and 99% of normal efficiency.

A “hypermorphic mutation” is a mutation that results in increased expression of the gene product and/or increased activity of the gene product.

As used herein, a “non-natural mutation” refers to a mutation that is generated through human intervention and differs from mutations found in the same gene that have occurred in nature (e.g., occurred naturally and not as a result of a modification made by a human).

A “locus” is a position on a chromosome where a gene or marker or allele is located. In some embodiments, a locus may encompass one or more nucleotides.

As used herein, the terms “desired allele,” “target allele” and/or “allele of interest” are used interchangeably to refer to an allele associated with a desired trait. In some embodiments, a desired allele may be associated with either an increase or a decrease (relative to a control) of or in a given trait, depending on the nature of the desired phenotype.

A marker is “associated with” a trait when said trait is linked to it and when the presence of the marker is an indicator of whether and/or to what extent the desired trait or trait form will occur in a plant/germplasm comprising the marker. Similarly, a marker is “associated with” an allele or chromosome interval when it is linked to it and when the presence of the marker is an indicator of whether the allele or chromosome interval is present in a plant/germplasm comprising the marker.

As used herein, the terms “backcross” and “backcrossing” refer to the process whereby a progeny plant is crossed back to one of its parents one or more times (e.g., 1, 2, 3, 4, 5, 6, 7, 8, etc.). In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-43 (1994). The initial cross gives rise to the FI generation. The term “BC1” refers to the second use of the recurrent parent, “BC2” refers to the third use of the recurrent parent, and so on.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one (e.g., one or more) progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be backcrossed one or more times (e.g., 1, 2, 3, 4, or more times) to a line having a desired genetic background, selecting for the desired allele, with the result being that the desired allele becomes fixed in the desired genetic background. For example, a marker associated with increased yield under non-water stress conditions may be introgressed from a donor into a recurrent parent that does not comprise the marker and does not exhibit increased yield under non-water stress conditions. The resulting offspring could then be backcrossed one or more times and selected until the progeny possess the genetic marker(s) associated with increased yield under non-water stress conditions in the recurrent parent background.

A “genetic map” is a description of genetic linkage relationships among loci on one or more chromosomes within a given species, generally depicted in a diagrammatic or tabular form. For each genetic map, distances between loci are measured by the recombination frequencies between them. Recombination between loci can be detected using a variety of markers. A genetic map is a product of the mapping population, types of markers used, and the polymorphic potential of each marker between different populations. The order and genetic distances between loci can differ from one genetic map to another.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make-up for all the genes in its genome. Genotypes can be indirectly characterized, e.g., using markers and/or directly characterized by nucleic acid sequencing.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell or can be separate from the organism or cell. In general, germplasm provides genetic material with a specific genetic makeup that provides a foundation for some or all of the hereditary qualities of an organism or cell culture. As used herein, germplasm includes cells, seed or tissues from which new plants may be grown, as well as plant parts that can be cultured into a whole plant (e.g., leaves, stems, buds, roots, pollen, cells, etc.).

As used herein, the terms “cultivar” and “variety” refer to a group of similar plants that by structural or genetic features and/or performance can be distinguished from other varieties within the same species.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

As used herein, the term “hybrid” in the context of plant breeding refers to a plant that is the offspring of genetically dissimilar parents produced by crossing plants of different lines or breeds or species, including but not limited to the cross between two inbred lines.

As used herein, the term “inbred” refers to a substantially homozygous plant or variety. The term may refer to a plant or plant variety that is substantially homozygous throughout the entire genome or that is substantially homozygous with respect to a portion of the genome that is of particular interest.

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment.

A Rosaceae plant in which at least one (e.g., one or more, e.g., 1, 2, 3, or 4, or more) endogenous functional WOX gene (e.g., an endogenous functional WOX1 gene and/or an endogenous functional WOX23 gene) is modified as described herein (e.g., comprises a modification as described herein) may have a reduced number of thorns and/or prickles, thorns and/or prickles that have a modified architecture, thorns and/or prickles that are reduced in size, and/or no thorns and/or prickles when compared to a control Rosaceae plant devoid of the at least one mutation.

As used herein, a “prickle” refers to a smaller version of thorn and can be described as a short, slender, sharp-pointed outgrowth projecting from the outer surface, or the epidermis of a plant. As used herein, a “thorn” means a sharp pointed, stiff, straight or curved woody projection. As used herein, a “trichome” refers to a hairlike structure that projects from the surface or the epidermis of a plant. As used herein, a “hair” means a slender, sharp pointed, flexible structure that projects from the surface, or the epidermis of a plant. The terms, thorn, prickle, trichome and hair may all refer to the same type of plant structure and may vary depending upon the stiffness, pointedness and general architecture of the plant structure. It is commonly held that plant hairs are less stiff, less pointed, less sharp than a thorn or a prickle. All of these terms may be used to describe structures that have more than 1 pointed outgrowth and may contain up to as many as 10 pointed structures emerging from a central spot on the plant epidermis.

The terms “thorn” and “prickle” (and grammatical variations thereof) will be used interchangeably herein. Thus, while in blackberry, “thorns” are more accurately referred to as “prickles” or “spines,” they may be referred to herein as “thorns” or “prickles” with the absence of thorns and/or prickles being described as “thornless,” “thornlessness,” “prickleless” or “pricklessness.”

As used herein, “reduced number of thorns and/or prickles” means a reduction in thorns and/or prickles of at least 5% when compared to a control Rosaceae plant devoid of the at least one mutation (e.g., a reduction in number of thorns and/or prickles of at least about 5% to about 100%, e.g., a reduction of about 5, 6, 7, 8, 9, 10, 11, 12, 13, 14, 15, 16, 17, 18, 19, 20, 21, 22, 23, 24, 25, 26, 27, 28, 29, 30, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any range or value therein). In some embodiments, “reduced number of thorns and/or prickles” means a reduction in thorns and/or prickles of at least 50% when compared to a control Rosaceae plant devoid of the at least one mutation (e.g., a reduction in number of thorns and/or prickles of at least about 50% to about 100%, e.g., a reduction of about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any range or value therein). In some embodiments, the reduction in number of thorns and/or prickles” means a reduction in thorns and/or prickles of at least 75% (e.g., 75, 76, 77, 78, 79, 80, 81, 82, 83, 84, 85, 86, 87, 88, 89, 90, 91, 92, 93, 94, 95, 96, 97, 98, 99, or 100%, or any range or value therein)

“Substantially thornless” or “substantially prickleless” as used herein refers to having a reduction in the amount of thorns and/or prickles by about 50%, 51%, 52%, 53%, 54%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5%.

As used herein, “a reduction in size of thorns and/or prickles” means a reduction in size by at least about 10% when compared to the thorns and/or prickles of a control Rosaceae plant devoid of the at least one mutation (e.g., a reduction in size of thorns and/or prickles by at least about 10% to about 100%, e.g., a reduction of about 10, 11, 12, 13, 14, 15, 16, 17, 18, 19, 20, 21, 22, 23, 24, 25, 26, 27, 28, 29, 30, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or any range or value therein), wherein a reduction of 100% means that the prickle or thorn being measured is absent. When all of the prickles and thorns of a plant are reduced by 100%, then the plant may be considered to have “no thorns or prickles.” When all of the prickles and thorns of a specific plant part, such as a stem, or a leaf, are reduced by 100% then the plant may be considered to have “no thorns or prickles”.

In some embodiments, the architecture of a thorn or prickle may be modified. A “modified architecture” as used herein means any change in physical structure, such as a reduction in size, stiffness, number of spines or sharp points on a thorn, sharpness, thickness of a thorn, thickness of sharp point(s) on a thorn, thickness of the region that a thorn/spine attaches to the stem. A modified architecture can include a thorn or prickle that is reduced in strength (e.g., a thorn or prickle that bends like a hair rather than being stiff as compared to thorn or prickle that is not modified). A thorn or prickle comprising a modified architecture may result in the thorn or prickle being less sharp. A thorn or prickle comprising a modified architecture may result in the thorn or prickle being less sharp and hard or stiff.

As used herein a “control plant” means a Rosaceae plant that does not contain an edited WOX gene or genes (e.g., WOX1, WOX23) as described herein that imparts an altered phenotype of reduced number of thorns and/or prickles, thorns and/or prickles that have a modified architecture, thorns and/or prickles that are reduced in size, and/or no thorns and/or prickles. A control Rosaceae plant is used to identify and select a Rosaceae plant edited as described herein and that has an enhanced trait or altered phenotype as compared to the control Rosaceae plant. A suitable control plant can be a plant of the parental line used to generate a plant comprising a mutated WOX gene(s), for example, a wild type plant devoid of an edit in an endogenous WOX gene as described herein. A suitable control plant can also be a plant that contains recombinant nucleic acids that impart other traits, for example, a transgenic plant having enhanced herbicide tolerance. A suitable Rosaceae control plant can in some cases be a progeny of a heterozygous or hemizygous transgenic Rosaceae plant line that is devoid of the mutated WOX gene as described herein, known as a negative segregant, or a negative isogenic line.

Also herein, the term “trait modification” encompasses altering the naturally occurring trait by producing a detectable difference in a characteristic in a Rosaceae plant comprising a mutation in an endogenous functional WOX gene as described herein relative to a Rosaceae plant not comprising the mutation, such as a wild-type plant, or a negative segregant. In some cases, the trait modification can be evaluated quantitatively. For example, the trait modification can entail an increase or decrease in an observed trait characteristic or phenotype as compared to a control plant. It is known that there can be natural variations in a modified trait. Therefore, the trait modification observed can entail a change of the normal distribution and magnitude of the trait characteristics or phenotype in the plants as compared to a control plant.

The present disclosure relates to a Rosaceae plant with improved economically relevant characteristics, more specifically a reduced number of thorns and/or prickles, thorns and/or prickles that have a modified architecture including but not limited to a reduced size, reduced stiffness or hardness, a reduced sharpness and the like, and/or no thorns and/or prickles. More specifically the present disclosure relates to a Rosaceae plant comprising a mutation(s) in a functional WOX gene(s) (e.g., functional WOX1, functional WOX23) as described herein, wherein the Rosaceae plant has a reduced number of thorns and/or prickles, thorns and/or prickles that have a modified architecture, thorns and/or prickles that are reduced in size, and/or no thorns and/or prickles as compared to a control Rosaceae plant devoid of said mutation(s).

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.831-1.835 and the World Intellectual Property Organization (WIPO) Standard ST.26. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein with respect to nucleic acids, the term “fragment” or “portion” refers to a nucleic acid that is reduced in length relative (e.g., reduced by 1, 2, 3, 4, 5, 6, 7, 8, 9, 10, 11, 12, 13, 14, 15, 16, 17, 18, 19, 20, 20, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, or 900 or more nucleotides or any range or value therein) to a reference nucleic acid and that comprises, consists essentially of and/or consists of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. As an example, a repeat sequence of guide nucleic acid of this invention may comprise a “portion” of a wild type CRISPR-Cas repeat sequence (e.g., a wild type CRISPR-Cas repeat; e.g., a repeat from the CRISPR Cas system of, for example, a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or a Cas14c, and the like).

In some embodiments, a nucleic acid fragment may comprise, consist essentially of or consist of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 320, 330, 340, 350, 360, 370, 380, 390, 395, 400, 410, 415, 420, 425, 430, 435, 440, 445, 450, 500, 550, 600, 650, 700, 750, 800, 850, 900, 950, 1000, 1100, 1150, 1200, 1250, 1300, 1350, 1400, 1450, 1500, 1550, 1600, 1650, 1700, 1750, 1800, 1900, 2000, 3000, 4000 or 5000 or more consecutive nucleotides, or any range or value therein, of a nucleic acid encoding a WOX polypeptide, optionally a fragment of a WOX (WOX1, WOX23) gene may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 115, 120, 125, 130, 135, 140, 145, 150 consecutive nucleotides to about 155, 160, 165, 170, 175, 180, 185, 190, 195, 200, 205, 210, 215, 220, 225, 230, 240, 245, 250, 255, 260, 265, 270, 275, 280, 285, 290, 295, 300, 305, 310, 315, 320, 325, 330, 340, 345, 350, 355, 360, 365, 370, 375, 380, 385, 390, 395, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740 or 750 or more consecutive nucleotides in length, or any range or value therein (e.g., a fragment or portion of any one SEQ ID NOs: 69, 70, 75, 76, 128, 129 or 162-173 (e.g., SEQ ID NOs: 78-82, 83-86, 87-90, 91-109, 110-125 or 131-135, optionally any one of SEQ ID NOs: 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 131, 132, 133, 134, and/or 135).

As used herein with respect to polypeptides, the term “fragment” or “portion” may refer to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, a polypeptide fragment may comprise, consist essentially of, or consist of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 260, 270, 280, or 290 or more consecutive amino acids of a reference polypeptide. In some embodiments, a polypeptide fragment may comprise, consist essentially of or consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, or 150, or more consecutive amino acid residues, or any range or value therein, of a WOX transcription factor (e.g., a fragment or a portion of any one of the polypeptides of SEQ ID NOs: 71, 77, 130, or 174-182 (e.g., SEQ ID NOs: 126, 127, 136 or 137)). In some embodiments, a fragment of a WOX transcription factor (WOX1 transcription factor, WOX23 transcription factor) may comprise, consist essentially of or consist of about 20 to about 60 consecutive amino acid residues (e.g., about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 consecutive amino acid residues, optionally about 36 or 42 consecutive amino acid residues, or any range or value therein, of a WOX transcription factor (see, e.g., SEQ ID NOs: 126, 127, 136 or 137). In some embodiments, a fragment of a WOX transcription factor or a WOX polynucleotide maybe the result of a deletion generated in the functional WOX gene resulting in a truncated polypeptide, optionally wherein the amount of the WOX transcription factor is reduced or undetectable. A deletion may result, for example, in an out-of-frame deletion allele. A functional WOX gene may be edited in more than one location, thereby providing a WOX gene comprising more than one mutation (i.e., 2, 3, 4, 5, 6 or more mutations, optionally 2 or 3 mutations).

In some embodiments, a fragment of a WOX transcription factor (WOX1 transcription factor, WOX23 transcription factor) can be a truncated WOX polypeptide resulting from a mutation of the WOX genomic sequence encoding the WOX transcription factor as described herein (e.g., a mutation in Exon 1 or Exon 2 of the functional WOX gene). In some embodiments, a fragment of a WOX transcription factor may be from the C-terminus of the WOX transcription factor. In some embodiments, a fragment of a WOX transcription factor may be comprise at least a portion of the N-terminal portion of the transcription factor in addition to the C-terminus of the transcription factor.

In some embodiments, such a deletion when comprised in a Rosaceae plant can result in the Rosaceae plant exhibiting a reduced number of thorns and/or prickles, thorns and/or prickles having a modified architecture, thorns and/or prickles that are reduced in size, and/or no thorns and/or prickles as compared to a Rosaceae plant not comprising (devoid of) said deletion. A functional WOX gene may be edited in one or more than one location (and using one or more different editing tools), thereby providing a WOX gene comprising one or more than one mutation. In some embodiments, a functional WOX gene mutated as described herein may comprise a deletion that may result in the encoded polypeptide having a deletion of one or more amino acid residues (e.g., a deletion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, or more consecutive amino acid residues, and any range or value therein (e.g., a truncated polypeptide), optionally a deletion of about 100 to about 350 consecutive amino acid residues (e.g., about 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, or 350 consecutive amino acid residues and any range or value therein).

In some embodiments, a “portion” or “region” in reference to a nucleic acid means at least 2, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135,140, 145, 150, 155, 160, 165, 170, 175, 180, 185, 190, 195, 200, 210, 220, 230, 240, 250, 260, 270, 280, 285, 290, 300, 310, 320, 330, 340, 350, 360, 370, 380, 390, 395, 400, 405, 410, 415, 420, 425, 430, 435, 440, 445, 450, 500, 600, 700, 800, 900, 1000, 1100, 1200, 1300, 1400, 1500, 1600, 1700, 1800, 1900, 2000, 2500, 3000, 3500, 4000, 4500, or 5000 or more consecutive nucleotides from a gene (e.g., consecutive nucleotides from an WOX gene), optionally a “portion” or “region” of a WOX gene may be about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 110, 111, 112, 113, 114, 115, 120, 125, 130, 131, 132, 133, 134, 135, 136, 137, 138, 139, 140, 141, 142, 143, 144, 145, 150, 151, 152, 153, 154, 155, 160, 165, 170, 171, 172, 173, 174, 175, 176, 177, 178, 179, 180, 185, 190, 191, 192, 193, 194, 195, 200, 205, 210, 215, 216, 217, 218, 219, 220, 221, 222, 223, 224, 225, 230, 231, 232, 233, 234, 235, 236, 237, 238, 239, 240, 245, 250, 251, 252, 253, 254, 255, 256, 257, 258, 259, 260, 265, 270, 271, 273, 274, 275, 280, 285, 290, 295, 300, 305, 310, 311, 312, 313, 314, 315, 316, 317, 318, 319, 320, 325, 326, 327, 328, 329, 330, 331, 33, 233, 335, 336, 337, 338, 339, 340, 345, 350, 355, 360, 365, 370, 375, 380, 385, 390, 395, 400, 405, 410, 415, 420, 425, 430, 435, 440, 445, or 450, or more consecutive nucleotides in length, or any range or value therein (e.g., a portion or region of any one of SEQ ID NOS: 69, 70, 75, 76, 128, 129, or 162-173 (e.g., SEQ ID NOs: 78-125 or 131-135)).

In some embodiments, a “portion” or “region” of a WOX polypeptide sequence may be about 5 to about 100 or more consecutive amino acid residues in length (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, or more consecutive amino acid residues in length, optionally about 20 to about 50 consecutive amino acid residues (e.g., a portion of any one of SEQ ID NOs: 71, 77, 130, or 174-182, optionally SEQ ID NOs: 126, 127, 136 or 137).

In some embodiments, a “sequence-specific nucleic acid binding domain” may bind to one or more fragments or portions of nucleotide sequences (e.g., DNA, RNA) encoding, for example, a WUSCHEL (WUS)-Related Homeobox (WOX) transcription factor as described herein.

As used herein with respect to nucleic acids, the term “functional fragment” refers to nucleic acid that encodes a functional fragment of a polypeptide. A “functional fragment” with respect to a polypeptide is a fragment of a polypeptide that retains one or more of the activities of the native reference polypeptide.

The term “gene,” as used herein, refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement,” as used herein, can mean 100% complementarity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity) to the comparator nucleotide sequence.

Different nucleic acids or proteins having homology are referred to herein as “homologues.” The term homologue includes homologous sequences from the same and from other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent sequence identity” can refer to the percentage of identical amino acids in an amino acid sequence as compared to a reference polypeptide. In regard to a WOX1 gene, a sequence may have at least 80% sequence identity to the nucleotide sequence of one or more of SEQ ID NOs: 69, 70, 75, 76, 128, 129 and/or 162-173. In some embodiments, a WOX1 gene may have at least 85% sequence identity to the nucleotide sequence of one or more of SEQ ID NOs: 69, 70, 75, 76, 128, 129 and/or 162-173. In some embodiments, a WOX1 gene may have at least 90% sequence identity to the nucleotide sequence of one or more of SEQ ID NOs: 69, 70, 75, 76, 128, 129 and/or 162-173. In some embodiments, a WOX1 gene may have at least 95% sequence identity to the nucleotide sequence of one or more of SEQ ID NOs: 69, 70, 75, 76, 128, 129 and/or 162-173, optionally wherein the WOX1 gene may have 100% sequence identity to the nucleotide sequence of one or more of SEQ ID NOs: 69, 70, 75, 76, 128, 129 and/or 162-173. A WOX1 polypeptide as described herein may have at least 80% sequence identity to the polypeptide sequence of one or more of SEQ ID NOs: 71, 77, 130 and/or 174-182. In some embodiments, a WOX1 polypeptide may have at least 85% sequence identity to the polypeptide sequence of one or more of SEQ ID NOs: 71, 77, 130 and/or 174-182. In some embodiments, a WOX1 polypeptide may have at least 90% sequence identity to the polypeptide sequence of one or more of SEQ ID NOs: 71, 77, 130 and/or 174-182. In some embodiments, a WOX1 polypeptide may have at least 95% sequence identity to the polypeptide sequence of one or more of SEQ ID NOs: 71, 77, 130 and/or 174-182, optionally wherein the WOX1 polypeptide may have 100% sequence identity to the polypeptide sequence of one or more of SEQ ID NOs: 71, 77, 130 and/or 174-182. With regard to regions or portions of a WOX1 gene, the region or portion may have at least 80% sequence identity to the nucleotide sequence of any one of SEQ ID NOs: 78-82, 83-86, 87-90, 91-109, 110-125 and/or 131-135, optionally at least 80% sequence identity to any one of SEQ ID NOs: 81, 82, 84-86, 88-90, 94-97, 99-101, 103-105, 107-109, 113-116, 118-120, 122, 124, 125 and/or 134. In some embodiments, a region or portion of a WOX1 gene may have at least 85% sequence identity to the nucleotide sequence of any one of SEQ ID NOs: 78-82, 83-86, 87-90, 91-109, 110-125 and/or 131-135, optionally at least 85% sequence identity any one of SEQ ID NOs: 81, 82, 84-86, 88-90, 94-97, 99-101, 103-105, 107-109, 113-116, 118-120, 122, 124, 125 and/or 134. In some embodiments, a region or portion of a WOX1 gene may have at least 90% sequence identity to the nucleotide sequence of any one of SEQ ID NOs: 78-82, 83-86, 87-90, 91-109, 110-125 and/or 131-135, optionally at least 90% sequence identity any one of SEQ ID NOs: 81, 82, 84-86, 88-90, 94-97, 99-101, 103-105, 107-109, 113-116, 118-120, 122, 124, 125 and/or 134. In some embodiments, a region or portion of a WOX1 gene may have at least 95% sequence identity to the nucleotide sequence of any one of SEQ ID NOs: 78-82, 83-86, 87-90, 91-109, 110-125 and/or 131-135, optionally at least 95% sequence identity to any one of SEQ ID NOs: 81, 82, 84-86, 88-90, 94-97, 99-101, 103-105, 107-109, 113-116, 118-120, 122, 124, 125 and/or 134. In some embodiments, a region or portion of a WOX1 gene may have 100% sequence identity to the nucleotide sequence of any one of SEQ ID NOs: 78-82, 83-86, 87-90, 91-109, 110-125 and/or 131-135, optionally 100% sequence identity to any one of SEQ ID NOs: 81, 82, 84-86, 88-90, 94-97, 99-101, 103-105, 107-109, 113-116, 118-120, 122, 124, 125 and/or 134. In some embodiments, a mutated WOX1 gene may have at least 90% sequence identity to a mutated WOX1 gene having a nucleotide sequence of any one of SEQ ID NO:143, 144, 145, 147, 149, 151, 153 and/or 155. In some embodiments, a mutated WOX1 gene may have at least 95% sequence identity to a mutated WOX1 gene having a nucleotide sequence of any one of SEQ ID NOs: 143, 144, 145, 147, 149, 151, 153 and/or 155. In some embodiments, a mutated WOX1 gene may have 100% sequence identity to a mutated WOX1 gene having a nucleotide sequence of any one of SEQ ID NOs: 143, 144, 145, 147, 149, 151, 153 and/or 155. In some embodiments, a mutated WOX1 polypeptide may have at least 90% sequence identity to a mutated WOX1 polypeptide having amino acid sequence of any one of SEQ ID NOs: 146, 148, 150, 152, 154 and/or 156. In some embodiments, a mutated WOX1 polypeptide may have at least 95% sequence identity to a mutated WOX1 polypeptide having an amino acid sequence of any one of SEQ ID NOs: 146, 148, 150, 152, 154 and/or 156. In some embodiments, a mutated WOX1 polypeptide may have 100% sequence identity to a mutated WOX1 polypeptide having an amino acid sequence of any one of SEQ ID NOs: 146, 148, 150, 152, 154 and/or 156.

As used herein, the phrase “substantially identical,” or “substantial identity” in the context of two nucleic acid molecules, nucleotide sequences, or polypeptide sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, about 100 nucleotides to about 200 nucleotides, about 100 nucleotides to about 300 nucleotides, about 100 nucleotides to about 400 nucleotides, about 100 nucleotides to about 500 nucleotides, about 100 nucleotides to about 600 nucleotides, about 100 nucleotides to about 800 nucleotides, about 100 nucleotides to about 900 nucleotides, or more in length, or any range therein, up to the full length of the sequence. In some embodiments, nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40, 50, 60, 70, or 80 nucleotides or more).

In some embodiments of the invention, the substantial identity exists over a region of consecutive amino acid residues of a polypeptide of the invention that is about 3 amino acid residues to about 20 amino acid residues, about 5 amino acid residues to about 25 amino acid residues, about 7 amino acid residues to about 30 amino acid residues, about 10 amino acid residues to about 25 amino acid residues, about 15 amino acid residues to about 30 amino acid residues, about 20 amino acid residues to about 40 amino acid residues, about 25 amino acid residues to about 40 amino acid residues, about 25 amino acid residues to about 50 amino acid residues, about 30 amino acid residues to about 50 amino acid residues, about 40 amino acid residues to about 50 amino acid residues, about 40 amino acid residues to about 70 amino acid residues, about 50 amino acid residues to about 70 amino acid residues, about 60 amino acid residues to about 80 amino acid residues, about 70 amino acid residues to about 80 amino acid residues, about 90 amino acid residues to about 100 amino acid residues, or more amino acid residues in length, and any range therein, up to the full length of the sequence. In some embodiments, polypeptide sequences can be substantially identical to one another over at least about 8 consecutive amino acid residues (e.g., about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175, 200, 225, 250, 300, 350 or more amino acids in length or more consecutive amino acid residues). In some embodiments, two or more WOX transcription factors may be identical or substantially identical (e.g., at least 70% to 99.9% identical; e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% identical or any range or value therein) over at least 8 consecutive amino acids to about 350 consecutive amino acids. In some embodiments, two or more WOX transcription factors may be identical or substantially identical over at least 8, 9, 10, 11, 12, 13, 14, or 15 consecutive amino acids to about 20, 21, 22, 23, 24, 25, 26, 27, 28, 29, 30, 31, 32, 33, 34, 35, 36, 37, 38, 39, or 40 consecutive amino acids).

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

A polynucleotide and/or recombinant nucleic acid construct of this invention (e.g., expression cassettes and/or vectors) may be codon optimized for expression. In some embodiments, the polynucleotides, nucleic acid constructs, expression cassettes, and/or vectors of the editing systems of the invention (e.g., comprising/encoding a sequence-specific nucleic acid binding domain (e.g., a sequence-specific nucleic acid binding domain (e.g., DNA binding domain) from a polynucleotide-guided endonuclease, a zinc finger nuclease, a transcription activator-like effector nuclease (TALEN), an Argonaute protein, and/or a CRISPR-Cas endonuclease (e.g., CRISPR-Cas effector protein) (e.g., a Type I CRISPR-Cas effector protein, a Type II CRISPR-Cas effector protein, a Type III CRISPR-Cas effector protein, a Type IV CRISPR-Cas effector protein, a Type V CRISPR-Cas effector protein or a Type VI CRISPR-Cas effector protein)), a nuclease (e.g., an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN)), deaminase proteins/domains (e.g., adenine deaminase, cytosine deaminase), a polynucleotide encoding a reverse transcriptase protein or domain, a polynucleotide encoding a 5′-3′ exonuclease polypeptide, and/or affinity polypeptides, peptide tags, etc.) may be codon optimized for expression in a plant. In some embodiments, the codon optimized nucleic acids, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acids, polynucleotides, expression cassettes, and/or vectors that have not been codon optimized.

In any of the embodiments described herein, a polynucleotide or nucleic acid construct of the invention may be operatively associated with a variety of promoters and/or other regulatory elements for expression in a plant and/or a cell of a plant. Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region” (e.g., Ubi1 promoter and intron) (see, e.g., SEQ ID NO: 21 and SEQ ID NO:22).

By “operably linked” or “operably associated” as used herein in reference to polynucleotides, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” in reference to polypeptides, refers to the attachment of one polypeptide to another. A polypeptide may be linked to another polypeptide (at the N-terminus or the C-terminus) directly (e.g., via a peptide bond) or through a linker.

The term “linker” is art-recognized and refers to a chemical group, or a molecule linking two molecules or moieties, e.g., two domains of a fusion protein, such as, for example, a nucleic acid binding polypeptide or domain and peptide tag and/or a reverse transcriptase and an affinity polypeptide that binds to the peptide tag; or a DNA endonuclease polypeptide or domain and peptide tag and/or a reverse transcriptase and an affinity polypeptide that binds to the peptide tag. A linker may be comprised of a single linking molecule or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide.

In some embodiments, a peptide linker useful with this invention may be about 2 to about 100 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 105, 110, 115, 120, 130, 140 150 or more amino acids in length). In some embodiments, a peptide linker may be a GS linker.

As used herein, the term “linked,” or “fused” in reference to polynucleotides, refers to the attachment of one polynucleotide to another. In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a covalent or non-covenant linkage or binding, including e.g., Watson-Crick base-pairing, or through one or more linking nucleotides. In some embodiments, a polynucleotide motif of a certain structure may be inserted within another polynucleotide sequence (e.g., extension of the hairpin structure in the guide RNA). In some embodiments, the linking nucleotides may be naturally occurring nucleotides. In some embodiments, the linking nucleotides may be non-naturally occurring nucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)). In some embodiments, a promoter useful with this invention is RNA polymerase II (Pol II) promoter. In some embodiments, a U6 promoter or a 7SL promoter from Zea mays may be useful with constructs of this invention. In some embodiments, the U6c promoter and/or 7SL promoter from Zea mays may be useful for driving expression of a guide nucleic acid. In some embodiments, a U6c promoter, U6i promoter and/or 7SL promoter from Glycine max may be useful with constructs of this invention. In some embodiments, the U6c promoter, U6i promoter and/or 7SL promoter from Glycine max may be useful for driving expression of a guide nucleic acid.

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79:87-94), maize (Christensen et al., 1989. Plant Molec. Biol. 12:619-632), and Arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231:150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phascolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP U.S. Pat. No. 255,378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9 (5): 297-306 (2015)), ZmSTK2_USP from maize (Wang et al. Genome 60 (6): 485-495 (2017)), LAT52 and LAT59 from tomato (Twell et al. Development 109 (3): 705-713 (1990)), Zm13 (U.S. Pat. No. 10,421,972), PLA₂-δ promoter from Arabidopsis (U.S. Pat. No. 7,141,424), and/or the ZmC5 promoter from maize (International PCT Publication No. WO1999/042587.

Additional examples of plant tissue-specific/tissue preferred promoters include, but are not limited to, the root hair-specific cis-elements (RHEs) (Kim et al. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37 (8): 1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron (see, e.g., SEQ ID NO:21 and SEQ ID NO:22).

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a one or more polynucleotides of the invention (e.g., a polynucleotide encoding a sequence-specific nucleic acid binding domain, a polynucleotide encoding a deaminase protein or domain, a polynucleotide encoding a reverse transcriptase protein or domain, a polynucleotide encoding a 5′-3′ exonuclease polypeptide or domain, a guide nucleic acid and/or reverse transcriptase (RT) template), wherein polynucleotide(s) is/are operably associated with one or more control sequences (e.g., a promoter, terminator and the like). Thus, in some embodiments, one or more expression cassettes may be provided, which are designed to express, for example, a nucleic acid construct of the invention (e.g., a polynucleotide encoding a sequence-specific nucleic acid binding domain, a polynucleotide encoding a nuclease polypeptide/domain, a polynucleotide encoding a deaminase protein/domain, a polynucleotide encoding a reverse transcriptase protein/domain, a polynucleotide encoding a 5′-3′ exonuclease polypeptide/domain, a polynucleotide encoding a peptide tag, and/or a polynucleotide encoding an affinity polypeptide, and the like, or comprising a guide nucleic acid, an extended guide nucleic acid, and/or RT template, and the like). When an expression cassette of the present invention comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). When two or more separate promoters are used, the promoters may be the same promoter, or they may be different promoters. Thus, a polynucleotide encoding a sequence specific nucleic acid binding domain, a polynucleotide encoding a nuclease protein/domain, a polynucleotide encoding a CRISPR-Cas effector protein/domain, a polynucleotide encoding an deaminase protein/domain, a polynucleotide encoding a reverse transcriptase polypeptide/domain (e.g., RNA-dependent DNA polymerase), and/or a polynucleotide encoding a 5′-3′ exonuclease polypeptide/domain, a guide nucleic acid, an extended guide nucleic acid and/or RT template when comprised in a single expression cassette may each be operably linked to a single promoter, or separate promoters in any combination.

An expression cassette comprising a nucleic acid construct of the invention may be chimeric, meaning that at least one (e.g., one or more) of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to, for example, a gene encoding a sequence-specific nucleic acid binding protein, a gene encoding a nuclease, a gene encoding a reverse transcriptase, a gene encoding a deaminase, and the like, or may be native to a host cell, or may be native to another source (e.g., foreign or heterologous to, for example, to a promoter, to a gene encoding a sequence-specific nucleic acid binding protein, a gene encoding a nuclease, a gene encoding a reverse transcriptase, a gene encoding a deaminase, and the like, or to the host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

In addition to expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct (e.g., expression cassette(s)) comprising the nucleotide sequence(s) to be transferred, delivered or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self-transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g., autonomous replicating plasmid with an origin of replication). Additionally, included are shuttle vectors by which is meant a DNA vehicle capable, naturally or by design, of replication in two different host organisms, which may be selected from actinomycetes and related species, bacteria and eukaryotic (e.g., higher plant, mammalian, yeast or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid or polynucleotide of this invention and/or expression cassettes comprising the same may be comprised in vectors as described herein and as known in the art.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage). As an example, a target nucleic acid may be contacted with a sequence-specific nucleic acid binding protein (e.g., polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein)) and a deaminase or a nucleic acid construct encoding the same, under conditions whereby the sequence-specific nucleic acid binding protein, the reverse transcriptase and/or the deaminase are expressed and the sequence-specific nucleic acid binding protein binds to the target nucleic acid, and the reverse transcriptase and/or deaminase may be fused to either the sequence-specific nucleic acid binding protein or recruited to the sequence-specific nucleic acid binding protein (via, for example, a peptide tag fused to the sequence-specific nucleic acid binding protein and an affinity tag fused to the reverse transcriptase and/or deaminase) and thus, the deaminase and/or reverse transcriptase is positioned in the vicinity of the target nucleic acid, thereby modifying the target nucleic acid. Other methods for recruiting reverse transcriptase and/or deaminase may be used that take advantage of other protein-protein interactions, and also RNA-protein interactions and chemical interactions may be used for protein-protein and protein-nucleic acid recruitment.

As used herein, “modifying” or “modification” in reference to a target nucleic acid includes editing (e.g., mutating), covalent modification, exchanging/substituting nucleic acids/nucleotide bases, deleting, cleaving, nicking, and/or altering transcriptional control of a target nucleic acid. In some embodiments, a modification may include one or more single base changes (SNPs) of any type.

“Introducing,” “introduce,” “introduced” (and grammatical variations thereof) in the context of a polynucleotide of interest means presenting a nucleotide sequence of interest (e.g., polynucleotide, RT template, a nucleic acid construct, and/or a guide nucleic acid) to a plant, plant part thereof, or cell thereof, in such a manner that the nucleotide sequence gains access to the interior of a cell.

The terms “transformation” or transfection” may be used interchangeably and as used herein refer to the introduction of a heterologous nucleic acid into a cell. Transformation of a cell may be stable or transient. Thus, in some embodiments, a host cell or host organism (e.g., a plant) may be stably transformed with a polynucleotide/nucleic acid molecule of the invention. In some embodiments, a host cell or host organism may be transiently transformed with a polynucleotide/nucleic acid molecule of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention (e.g., one or more expression cassettes comprising polynucleotides for editing as described herein) may be transiently introduced into a cell with a guide nucleic acid and as such, no DNA is maintained in the cell.

A nucleic acid construct of the invention may be introduced into a plant cell by any method known to those of skill in the art. Non-limiting examples of transformation methods include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)). General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

In some embodiments of the invention, transformation of a cell may comprise nuclear transformation. In other embodiments, transformation of a cell may comprise plastid transformation (e.g., chloroplast transformation). In still further embodiments, nucleic acids of the invention may be introduced into a cell via conventional breeding techniques. In some embodiments, one or more of the polynucleotides, expression cassettes and/or vectors may be introduced into a plant cell via Agrobacterium transformation.

A polynucleotide therefore can be introduced into a plant, plant part, plant cell in any number of ways that are well known in the art. The methods of the invention do not depend on a particular method for introducing one or more nucleotide sequences into a plant, only that they gain access to the interior the cell. Where more than polynucleotide is to be introduced, they can be assembled as part of a single nucleic acid construct, or as separate nucleic acid constructs, and can be located on the same or different nucleic acid constructs. Accordingly, the polynucleotide can be introduced into the cell of interest in a single transformation event, or in separate transformation events, or, alternatively, a polynucleotide can be incorporated into a plant as part of a breeding protocol.

In agricultural and horticultural settings, thorns and prickles can cause significant problems for those handling the plants. Thorns and prickles interfere with, for example, propagation, care and harvesting of such crops and as a result can lead to increased costs. As an example, the canes of wild Blackberry species and other Rubus species are covered from the bottom to the top with differing densities of thorns. These thorns are not derived from the epidermis rather from the vascular tissue and are more correctly referred to as prickles or spines. In addition to interfering with plant propagation, plant care and fruit harvesting, the presence of thorns can be a liability if a consumer were to inadvertently cat a thorn.

Today, there are four sources of thornlessness available to blackberry plant breeders. One is the recessive gene ‘s’ used by the John Innes program in the United Kingdom to develop ‘Merton Thornless’, which is still the source of thornlessness in modern tetraploid blackberry cultivars. The use of ‘Merton Thornless’ has been very important in advancing thornlessness, particularly in erect and semi-erect genotypes. Gene ‘s’ is a recessive trait. Two advantages of this gene are the complete absence of thorns and the ability to screen seedlings for the absence of glandular trichomes. The disadvantages include the 35:1 segregation ratio in tetraploids and the linkage of this trait to undesirable traits such as lack of winter hardiness, acid fruit and later harvest season.

Another source of thornlessness in blackberry is the dominant gene ‘Sf’ from ‘Austin Thornless’. The advantage of ‘Sf’ is the genetic dominance, which has provided a thornlessness source at the 6× and higher ploidy levels (‘Austin Thornless’ is an octoploid). ‘Sf’ has been important in breeding trailing types of blackberry varieties. However, several detrimental traits are linked to this locus, including a trailing growth habit and susceptibility to downy mildew. Also, plants derived from this source of thornlessness can have thorns on the basal 0.3 m of the cane; these same canes are thornless beyond this point and are commercially thornless since fruit is borne only in the thornless area of the canc.

The dominant ‘Sfte’ locus is another source of thornlessness and is from non-chimeras of ‘Thornless Evergreen’. This locus is not used in breeding as it reverts regularly in the field and also is associated with a lot of undesirable traits. A fourth source of thornlessness available for breeding includes, the semi-dominant ‘Sfl’ gene of ‘Lincoln Logan’. This allele was originally identified as a chimera of ‘Loganberry’. A tissue culture technique in which a ‘Loganberry’-type clone (L654) was used resulted in a spontaneous embryo from callus tissue. ‘Lincoln Logan’ was released from this effort and was then used in New Zealand and USDA-ARS Oregon breeding programs. The first cultivars with the ‘Sfl’ source are not yet use commercially; however, there are extensive fertility problems associated with ‘Sfl’ and also it appears that two separate loci may be required to produce ‘Sfl’ thornlessness.

Blackberry varieties are clonally propagated. Currently, if breeders have a thorny blackberry variety it is not possible to remove the thorns without laborious crossing and backcrossing to introgress one of the four mentioned thornless alleles. Making a blackberry plant thornless in one generation would be a dramatic advance for blackberry breeding. Such an advance would be useful in other Rosaceae plants, including other Rubus spp., as well as Rosa spp., a Hulthemia spp., Hesperrhodos spp. or, Platyrhodon spp.

The present invention is directed to the modification of genes that contribute to thorn and prickle development in order to improve the ability to handle such plants and reduce costs associated with working with these plants as compared to those varieties that comprise thorns and/or prickles. Thus, an editing strategy useful for this invention can include generating a mutation in one or more than one functional WOX gene in a Rosaceae plant. In some embodiments, one or more than one mutation (optionally, a non-natural mutation) may be generated in a functional WOX gene of a plant. Mutations that may be useful for producing Rosaceae plants having reduced number of thorns and/or prickles, thorns and/or prickles that have a modified architecture, thorns and/or prickles that are reduced in size, and/or no thorns and/or prickles include, for example, substitutions, deletions, and/or insertions. In some aspects, a mutation generated by the editing technology can be a point mutation. In some embodiments, the mutation may be an out-of-frame insertion or out-of-frame deletion, optionally wherein the out-of-frame insertion or out-of-frame deletion produces/results in a premature stop codon. In some embodiments, a mutation in one or more than one functional WOX gene as described herein results in production of a modified WOX transcription factor, optionally wherein the modified WOX transcription factor comprises a C-terminal truncation, optionally a truncation of about the last 200-320 consecutive amino acid residues (about 200, 205, 210, 211, 212, 213, 214, 215, 216, 217, 218, 219, 220, 225, 230, 235, 240, 245, 250, 255, 260, 265, 270, 275, 280, 285, 290, 295, 300, 305, 310, 311, 313, 314, 315, 316, 317, 318, 319, or 320 consecutive amino acid residues, or any value or range therein) as compared to a WOX transcription factor produced by an unmodified endogenous functional WOX gene. In some embodiments, a mutation in one or more than one functional WOX gene as described herein results in production of a reduced amount of or no encoded polypeptide or wherein the encoded WOX transcription factor is not detectable.

In some embodiments, a Rosaceae plant or plant part thereof is provided that comprises at least one mutation in at least one (e.g., one or more) endogenous functional WUSCHEL (WUS)-Related Homeobox (WOX) gene encoding a WOX transcription factor, optionally wherein the at least one mutation may be a non-natural mutation. The at least one mutation in the at least one endogenous functional WOX gene provides a mutated WOX gene that can be a nonfunctional WOX gene that may encode a nonfunctional WOX transcription factor. In some embodiments, the encoded WOX transcription factor regulates the production of thorns and/or prickles. In some embodiments, the endogenous functional WOX gene is expressed specifically in tissues that give rise to trichomes, thorns and/or prickles. In some embodiments, the endogenous functional WOX gene is expressed in a tissue specific manner in the vascular tissue, in the epidermal tissue and/or in the cells or tissues which give rise to a thorn or a prickle. In some embodiments, the endogenous functional WOX gene is an endogenous functional WOX1 gene and/or an endogenous functional WOX23 gene and the encoded WOX transcription factor is a WOX1 polypeptide or a WOX23 polypeptide, respectively. In some embodiments, a Rosaceae plant or plant part thereof may comprise at least one mutation in two or more endogenous functional WOX genes.

In some embodiments, a mutation in an endogenous functional WOX gene may be located in the first or second exon of the gene, optionally wherein the mutation is located in the first exon and/or second exon of the WOX1 gene and/or in the second exon of the WOX23 gene. In some embodiments, a mutation may be an out-of-frame insertion or an out-of-frame deletion, optionally wherein the out-of-frame insertion or out-of-frame deletion produces/results in a premature stop codon, optionally resulting in a C-terminal truncation of the encoded transcription factor (e.g., a truncated WOX transcription factor). In some embodiments, a mutation in the endogenous functional WOX gene may result in a mutated WOX gene that produces a truncated WOX transcription factor or produces no detectable WOX transcription factor. In some embodiments, a C-terminal truncation may comprise a deletion of one amino acid residue to about 320 consecutive amino acid residues from the encoded WOX transcription factor. In some embodiments, a mutation of a functional WOX gene in a Rosaceae plant may results in a dominant mutation, a recessive mutation, a semi-dominant mutation or a hypomorphic mutation. In some embodiments, a Rosaceae plant or part thereof comprising at least one mutation in an endogenous functional WOX gene exhibits a phenotype of a reduced number of thorns and/or prickles, thorns and/or prickles having a modified architecture, thorns and/or prickles that are reduced in size, and/or no thorns and/or prickles when compared to a control Rosaceae plant devoid of the at least one mutation.

In some embodiments, a mutated WOX gene that is comprised in a Rosaceae plant cell may have at least 90% sequence identity to any one of the mutated WOX genes (e.g., WOX1, WOX23) described herein.

In some embodiments, a Rosaceae plant cell is provided, the Rosaceae plant cell comprising an editing system, the editing system comprising: (a) a CRISPR-Cas effector protein; and (b) a guide nucleic acid (e.g., gRNA, gDNA, crRNA, crDNA, sgRNA, sgDNA) comprising a spacer sequence with complementarity to an endogenous functional target gene encoding a WUSCHEL (WUS)-Related Homeobox (WOX) transcription factor in the Rosaceae plant cell. The editing system may be used to generate a mutation in the endogenous functional target gene encoding a WOX transcription factor. In some embodiments, the endogenous functional target gene is an endogenous functional WUSCHEL (WUS)-Related Homeobox (WOX) gene (e.g., one or more than one endogenous functional WOX gene(s)), optionally an endogenous functional WOX1 gene and/or an endogenous functional WOX23 gene, and the WOX transcription factor is a WOX1 transcription factor or a WOX23 transcription factor. In some embodiments, the encoded WOX transcription factor regulates the production of thorns and/or prickles. In some embodiments, the endogenous functional WOX gene is expressed specifically in tissues that give rise to trichomes, thorns and/or prickles. In some embodiments, the endogenous functional WOX gene is expressed in a tissue specific manner in the vascular tissue and/or the epidermal tissue. In some embodiments, the endogenous functional target WOX gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 69, 70, 75, 76, 128, 129 or 162-173; (b) comprises a region having at least 80% sequence identity to a nucleotide sequence of any one of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134; (c) encodes an amino acid sequence having at least 80% sequence identity to any one of SEQ ID NOs: 71, 77, 130, or 174-182; and/or (d) encodes a region of an amino acid sequence having at least 80% sequence identity to any one of SEQ ID NO:126, SEQ ID NO: 127, SEQ ID NO:136 or SEQ ID NO: 137, optionally wherein the sequence identity of (a), (b), (c) and/or (d) may be at least 85%, or at least 90%, or it may be at least 95%, optionally the sequence identity may be 100%. In some embodiments, a guide nucleic acid of an editing system may comprise the nucleotide sequence (a spacer sequence, e.g., one or more spacers) of any one of SEQ ID NOs: 138-142, or reverse complement thereof or any combination thereof. In some embodiments, a mutation may be a non-natural mutation.

A mutation in a functional WOX gene of a Rosaceae plant, plant part thereof, or a Rosaceae plant cell useful for this invention may be any type of mutation, including a base substitution, a base deletion, a base insertion and/or an inversion. In some embodiments, the mutation may be a non-natural mutation. In some embodiments, the mutation may comprise a base substitution to an A, a T, a G, or a C. In some embodiments, the mutation may be a deletion or insertion (e.g., of at least one base pair (e.g., 1 base pair to about 100 base pairs; e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 consecutive base pairs, or any range or value therein; optionally 1 to about 50 consecutive base pairs, 1 to about 30 consecutive base pairs, 1 to about 15 consecutive base pairs. In some embodiments, the at least one mutation may be an out-of-frame insertion or an out-of-frame deletion, optionally wherein the mutation is a non-natural mutation. In some embodiments, the deletion or insertion (e.g., out-of-frame insertion or an out-of-frame deletion) may be located in the first and/or second exon of the functional WOX gene, optionally located in the first exon and/or second exon of the functional WOX1 gene and/or in the second exon of the functional WOX23 gene. In some embodiments, a deletion or insertion (e.g., out-of-frame insertion or an out-of-frame deletion) may result in a mutated WOX gene that produces a truncated WOX transcription factor polypeptide or a reduced amount of or no detectable WOX transcription factor polypeptide.

In some embodiments, the out-of-frame deletion or out-of-frame insertion may result in a WOX gene that encodes a truncated polypeptide, optionally a polypeptide having a C-terminal truncation resulting from a premature stop codon generated by the deletion or insertion. In some embodiments, the C-terminal truncation is a deletion of about 200-320 consecutive amino acid residues (about 200, 205, 210, 211, 212, 213, 214, 215, 216, 217, 218, 219, 220, 225, 230, 235, 240, 245, 250, 255, 260, 265, 270, 275, 280, 285, 290, 295, 300, 305, 310, 311, 313, 314, 315, 316, 317, 318, 319, or 320 consecutive amino acid residues, or any value or range therein) as compared to a WOX transcription factor produced by an unmodified endogenous WOX gene.

In some embodiments, a mutation useful with this invention may be a dominant mutation, semi-dominant mutation, a hypomorphic mutation or a recessive mutation, optionally wherein the mutation is a non-natural mutation.

In some embodiments, a mutation in an endogenous functional WOX gene may result in a mutated WOX nucleic acid having at least 90% sequence identity to any one of the mutated WOX genes (e.g., WOX1, WOX23) described herein. The mutated WOX gene may be an endogenous nonfunctional WOX gene and/or may encode a nonfunctional WOX transcription factor.

A Rosaceae plant species that may be modified as described herein may include, but is not limited to Rosa spp., Hulthemia spp., a Hesperrhodos spp., a Platyrhodon spp., or a Rubus spp. In some embodiments, the Rubus spp. may be blackberry or black raspberry, optionally, wherein the Rubus spp. plant is Rubus allegheniensis (Allegheny blackberry), Rubus armeniacus (Himalayan blackberry), Rubus canadensis (Canadian blackberry), Rubus fruticosus agg. (European blackberry), Rubus laciniatus (cutleaf evergreen blackberry), Rubus pensilvanicus (Pennsylvania blackberry), Rubus trifidus (Japanese blackberry), Rubus ursinus (trailing blackberry), Rubus hybrid (e.g., progeny resulting from hybridization of two or more of the above Rubus species), Rubus leucodermis (whitebark raspberry or western black raspberry), or Rubus occidentalis (black raspberry). In some embodiments, the Rubus spp. can be a raspberry (red raspberry) plant including, but not limited to, Rubus crataegifolius (Asian raspberry), Rubus gunnianus (Tasmanian alpine raspberry), Rubus idaeus (red raspberry or European red raspberry), Rubus parvifolius (Australian native raspberry), Rubus phoenicolasius (wine raspberry or wineberry), Rubus rosifolius (Mauritius raspberry), Rubus strigosus (American red raspberry) (syn. R. idaeus var. strigosus), or Rubus ellipticus (yellow Himalayan raspberry). In some embodiments, the Rosa spp. is a rose plant and/or the Rosa spp. is Rosa chinensis.

The types of editing tools that may be used to generate these and other mutations in a Rosaceae functional WOX gene include any base editors or cutters, which are guided to a target site using spacers having at least 80% (e.g., 80, 81, 82, 83, 84, 85, 86, 87, 88, 89, 90, 91, 92, 93, 94, 95, 96, 97, 98, 99, or 100%, or any range or value therein), complementarity to a portion or a region of a functional WOX gene (e.g., one or more than one functional WOX gene, e.g., a functional WOX1 gene and/or a functional WOX23 gene) as described herein.

In some embodiments, a mutation of a WOX gene is within a portion or region of the endogenous WOX gene having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of the nucleotide sequences of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134, optionally wherein the sequence identity may be at least 85%, or at least 90%, or it may be at least 95%, optionally the sequence identity may be 100%.

An endogenous functional WUSCHEL (WUS)-Related Homeobox (WOX) gene useful with this invention (e.g., an endogenous target gene) encodes a functional WOX transcription factor polypeptide and includes an endogenous functional WOX1 gene and an endogenous functional WOX23 gene, which encode an WOX1 transcription factor or a WOX23 transcription factor, respectively. An endogenous WOX gene useful with this invention is expressed specifically in tissues that give rise to trichomes, thorns and/or prickles, e.g., expressed in a tissue specific manner in the vascular tissue, the epidermal tissue and/or cells or tissues that give rise to thorns and/or prickles in a plant or part thereof, and the encoded WOX transcription factor regulates the production of thorns and/or prickles.

In some embodiments, an endogenous WOX gene (e.g., endogenous target gene) (1) may comprise a nucleic acid sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to one or more of SEQ ID NOs: 69, 70, 75, 76, 128, 129 and 162-173; (b) comprises a region having at least 80% sequence identity to a nucleotide sequence of any one of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134; (c) encodes an amino acid sequence having at least 80% sequence identity to one or more of SEQ ID NOs: 71, 77, 130, and 174-182; and/or (d) encodes a region of an amino acid sequence having at least 80% sequence identity to any one of SEQ ID NOs: 126, 127, 136 or 137, optionally wherein the sequence identity of (a), (b), (c) and/or (d) may be at least 85%, or at least 90%, or it may be at least 95%, optionally the sequence identity may be 100%.

Example endogenous functional WOX genes and encoded WOX transcription factor polypeptides useful with this invention, as well as target regions for editing and example edited WOX genes and the respective encoded polypeptides are provided in Table 1.

TABLE 1

| | | Coding and | | |
|---|---|---|---|---|
| | Genomic | polypeptide | | |
| Endogenous | SEQ ID | SEQ ID | Genomic Regions | Polypeptide regions |
| WOX gene | NOs | NOs | SEQ ID NOs | SEQ ID NOs |
| WOX1 | 69 | 70, 71 | Exon 1 78-90 | 126, 127 |
| | | | Exon 2 91-109 | |
| WOX23 | 75 | 76, 77 | Exon 2 104, 105, 109-125 | 127 |
| WOX1 | 128 | 129, 130 | Exon 2 95-97, 104, 105, 111- | 136, 137 |
| | | | 113, 118-120, 124, 125, 131- | |
| | | | 135 | |

In some embodiments, a Rosaceae plant comprising at least one (e.g., one or more, e.g., 1, 2, 3, 4, 5, or more) mutation in an endogenous functional WOX gene (in at least one endogenous functional WOX gene, e.g., in one or more functional WOX genes, e.g., functional WOX1, and/or functional WOX23) exhibits a reduced number of thorns and/or prickles, thorns and/or prickles that have a modified architecture, thorns and/or prickles that are reduced in size, and/or no thorns and/or prickles (is devoid of thorns and/or prickles) when compared to a control Rosaceae plant devoid of the at least one mutation (e.g., an isogenic plant (e.g., wild type unedited plant or a null segregant).

In some embodiments, a Rosaceae plant may be regenerated from a Rosaceae plant part and/or plant cell of the invention comprising a mutation in one or more than one endogenous functional WOX gene (an endogenous functional WOX1 gene and/or an endogenous functional WOX23 gene) as described herein, wherein the regenerated Rosaceae plant comprises the mutation in the one or more than one endogenous functional WOX gene and a phenotype of reduced number of thorns and/or prickles, thorns and/or prickles that have a modified architecture, thorns and/or prickles that are reduced in size, and/or no thorns and/or prickles when compared to a control Rosaceae plant devoid of the at least one mutation.

In some embodiments, a Rosaceae plant cell is provided, the Rosaceae plant cell comprising at least one (e.g., one or more) mutation (optionally a non-natural mutation) within an endogenous functional WOX gene, wherein the at least one mutation is a base substitution, a base insertion or a base deletion that is introduced using an editing system that comprises a nucleic acid binding domain that binds to a target site within the endogenous functional WOX gene, wherein the endogenous functional WOX gene is expressed specifically in tissues that give rise to trichomes, thorns and/or prickles, optionally wherein the endogenous functional WOX gene is expressed in a tissue specific manner in the vascular tissue, the epidermal tissue and/or cells or tissues that give rise to thorns and/or prickles in a plant or part thereof. In some embodiments, the endogenous functional WOX gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 69, 70, 75, 76, 128, 129 or 162-173; (b) comprises a region having at least 80% sequence identity to a nucleotide sequence of any one of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134; (c) encodes an amino acid sequence having at least 80% sequence identity to any one of SEQ ID NOs: 71, 130, or 174-182; and/or (d) encodes a region of an amino acid sequence having at least 80% sequence identity to any one of SEQ ID NO: 126, SEQ ID NO:127, SEQ ID NO: 136 or SEQ ID NO: 137, optionally wherein the sequence identity of (a), (b), (c) and/or (d) may be at least 85%, or at least 90%, or it may be at least 95%, optionally the sequence identity may be 100%.

In some embodiments, the base substitution, base insertion, or base deletion results in, for example, a premature stop codon. In some embodiments, the base substitution, base insertion, or base deletion results in, for example, a truncated WOX transcription factor, optionally a C-terminal truncation. In some embodiments, the at least one mutation is a point mutation. In some embodiments, the at least one mutation within the functional WOX gene is an insertion and/or a deletion, optionally wherein the at least one mutation is an out-of-frame insertion or out-of-frame deletion. In some embodiments, the endogenous functional WOX gene is an endogenous functional WOX1 gene or an endogenous functional WOX23 gene.

In some embodiments, a target site within an WOX gene of a Rosaceae plant cell may be within a region or portion of the endogenous WOX gene, the region having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of the nucleotide sequences SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134, optionally wherein the sequence identity may be at least 85%, or at least 90%, or it may be at least 95%, optionally the sequence identity may be 100%. In some embodiments, the target site within the WOX gene is within a region of the endogenous WOX gene that encodes an amino acid sequence having at least 80% sequence identity to any one of SEQ ID NOs: 126, 127, 136 and/or 137.

In some embodiments, a mutation in an endogenous functional WOX gene may result in a mutated WOX nucleic acid having at least 90% sequence identity to any one of the mutated WOX genes (e.g., WOX1, WOX23) having a nucleotide sequence of SEQ ID NOs: 143, 144, 145, 147, 149, 151, 153 and/or 155 and/or mutated WOX polypeptide comprising an amino acid sequence having at least 90% sequence identity to any one of SEQ ID NOs: 146, 148, 150, 152, 154 and/or 156.

In some embodiments, the editing system further comprises a nuclease, the nucleic acid binding domain binds to a target site within a sequence having least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 69, 70, 75, 76, 128, 129, or 162-173 and/or in a sequence having at least 80% sequence identity to a sequence of any one of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134, optionally wherein the sequence identity may be at least 85%, or at least 90%, or it may be at least 95%, optionally the sequence identity may be 100%, and the at least one mutation within a functional WOX gene is made following cleavage by the nuclease, optionally wherein the at least one mutation is a non-natural mutation. In some embodiments, the at least one mutation may result in a hypomorphic mutation, a dominant mutation, semi-dominant mutation or a recessive mutation.

In some embodiments, the at least one mutation within the functional WOX gene is an insertion and/or a deletion, optionally the at least one mutation is an out-of-frame insertion or an out-of-frame deletion. In some embodiments, the at least one mutation may be an insertion and/or a deletion that results in a premature stop codon, optionally results in an WOX transcription factor with a C-terminal truncation, optionally wherein the C-terminal truncation may be deletion of one amino acid residue to about 320 consecutive amino acid residues, optionally 200-320 consecutive amino acid residues from the encoded WOX transcription factor (e.g., about 200, 205, 210, 211, 212, 213, 214, 215, 216, 217, 218, 219, 220, 225, 230, 235, 240, 245, 250, 255, 260, 265, 270, 275, 280, 285, 290, 295, 300, 305, 310, 311, 313, 314, 315, 316, 317, 318, 319, or 320 consecutive amino acid residues, or any value or range therein). In some embodiments, a mutation within the functional WOX gene may comprise a point mutation. In some embodiments, the mutation results in a mutated WOX gene comprising a nucleotide sequence having at least 90% sequence identity to any one of the mutated WOX genes having a nucleotide sequence of SEQ ID NOs: 143, 144, 145, 147, 149, 151, 153 and/or 155 and/or results in a mutated WOX gene that encodes a WOX transcription factor having at least 90% sequence identity to any one of the mutated WOX transcription factor having an amino acid sequence of SEQ ID NOs: 146, 148, 150, 152, 154 and/or 156, optionally, wherein the at least one mutation is a non-natural mutation.

In some embodiments, the Rosaceae plant cell may be regenerated into a Rosaccae plant that comprises the at least one mutation, optionally wherein the Rosaceae plant regenerated from the Rosaceae plant cell exhibits a phenotype of a reduced number of thorns and/or prickles, thorns and/or prickles that have a modified architecture, thorns and/or prickles that are reduced in size, and/or no thorns and/or prickles when compared to a control Rosaceae plant devoid of the at least one mutation. In some embodiments, a Rosaceae plant comprising the at least one mutation in an endogenous functional WOX gene is not regenerated.

In some embodiments, a method of producing/breeding a transgene-free edited Rosaceae plant is provided, the method comprising: crossing a Rosaceae plant of the present invention (e.g., a Rosaceae plant comprising one or more mutations (optionally, one or more non-natural mutations) in one or more functional WOX genes and having a reduced number of thorns and/or prickles, thorns and/or prickles that have a modified architecture, thorns and/or prickles that are reduced in size, and/or no thorns and/or prickles (e.g., devoid of prickles and/or thorns) with a transgene-free plant, thereby introducing the mutation into the Rosaceae plant that is transgene-free; and selecting a progeny Rosaceae plant that comprises the mutation and is transgene-free, thereby producing a transgene-free edited Rosaceae plant.

Also provided herein is a method of providing a plurality of Rosaceae plants having a reduced number of thorns and/or prickles, thorns and/or prickles that have a modified architecture, thorns and/or prickles that are reduced in size, and/or no thorns and/or prickles, the method comprising planting two or more Rosaceae plants of the invention (e.g., 2, 3, 4, 5, 6, 7, 8, 9, 10, 20, 30, 40, 50, 100, 200, 300, 400, 500, 1000, 2000, 3000, 400, 5000, or 10,000 or more Rosaceae plants comprising one or more mutations (optionally, one or more non-natural mutations) in one or more functional WOX genes and a reduced number of thorns and/or prickles, thorns and/or prickles that have a modified architecture, thorns and/or prickles that are reduced in size, and/or no thorns and/or prickles in a growing area (e.g., a field (e.g., a cultivated field, an agricultural field), a growth chamber, a greenhouse, a recreational area, a lawn, and/or a roadside and the like), thereby providing a plurality of Rosaceae plants having a reduced number of thorns and/or prickles, thorns and/or prickles that have a modified architecture, thorns and/or prickles that are reduced in size, and/or no thorns and/or prickles as compared to a plurality of control Rosaceae plants devoid of the mutation.

In some embodiments, a method of creating a mutation in an endogenous WUSCHEL (WUS)-Related Homeobox (WOX) gene in a Rosaceae plant, comprising: (a) targeting a gene editing system to a portion or a region of the WOX gene that comprises a sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134, optionally wherein the sequence identity may be at least 85%, or at least 90%, or it may be at least 95%, optionally the sequence identity may be 100%; and (b) selecting a Rosaceae plant that comprises a modification located in a region of the gene having at least 80% sequence identity to any one of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134.

In some embodiments, a method of generating variation in a functional WUSCHEL (WUS)-Related Homeobox (WOX) gene is provided, the method comprising: introducing an editing system into a Rosaceae plant cell, wherein the editing system is targeted to a region of a functional WOX gene that encodes a WOX transcription factor and contacting the region of the functional WOX gene with the editing system, thereby introducing a mutation into the WOX gene and generating variation in the WOX gene of the Rosaceae plant cell. In some embodiments, the functional WOX gene is a functional WOX1 gene or a functional WOX23 gene. In some embodiments, the functional WOX gene comprises a nucleotide sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 69, 70, 75, 76, 128, 129, or 162-173, and/or the region of the WOX gene in which variation is generated comprises a nucleotide sequence having at least 80% sequence identity to of any one SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134, and/or the WOX transcription factor comprises an amino acid sequence having at least 80% sequence identity to any one of SEQ ID NOs: 71, 77, 130, or 174-182, and/or the region of the WOX transcription factor in which variation is generated comprises an amino acid sequence having at least 80% sequence identity to any one of SEQ ID NOs: 126, 127, 136 or 137, optionally wherein the sequence identity of the above sequences may be at least 85%, or at least 90%, or it may be at least 95%, optionally the sequence identity may be 100%. In some embodiments, the region of the WOX gene that is targeted comprises at least 80% sequence identity to any one of the nucleotide sequences of SEQ ID NOs: 78-125 or 131-135, optionally to any one of SEQ ID NOs: 81, 82, 84-86, 88-90, 94-97, 99-101, 103-105, 107-109, 113-116, 118-120, 122, 124, 125 or 134.

In some embodiments, contacting the region of the endogenous functional WOX gene in the Rosaceae plant cell with the editing system produces a Rosaceae plant cell comprising in its genome an edited endogenous WOX gene, the method further comprising (a) regenerating a Rosaceae plant from the Rosaceae plant cell; (b) selfing the Rosaceae plant to produce progeny Rosaceae plants (E1); (c) assaying the progeny Rosaceae plants of (b) for a reduced number of thorns and/or prickles, thorns and/or prickles that have a modified architecture, thorns and/or prickles that are reduced in size, and/or no thorns and/or prickles; and (d) selecting the progeny Rosaceae plants exhibiting a reduced number of thorns and/or prickles, thorns and/or prickles that have a modified architecture, thorns and/or prickles that are reduced in size, and/or no thorns and/or prickles to produce selected progeny Rosaceae plants exhibiting reduced number of thorns and/or prickles, thorns and/or prickles that have a modified architecture, thorns and/or prickles that are reduced in size, and/or no thorns and/or prickles as compared to a control Rosaceae plant. In some embodiments, the method may further comprise (e) selfing the selected progeny Rosaceae plants of (d) to produce progeny Rosaceae plants (E2); (f) assaying the progeny Rosaceae plants of (e) for a reduced number of thorns and/or prickles, thorns and/or prickles that have a modified architecture, thorns and/or prickles that are reduced in size, and/or no thorns and/or prickles; and (g) selecting the progeny Rosaceae plants exhibiting a reduced number of thorns and/or prickles, thorns and/or prickles that have a modified architecture, thorns and/or prickles that are reduced in size, and/or no thorns and/or prickles to produce selected progeny Rosaceae plants exhibiting a reduced number of thorns and/or prickles, thorns and/or prickles that have a modified architecture, thorns and/or prickles that are reduced in size, and/or no thorns and/or prickles as compared to a control Rosaceae plant, optionally repeating (c) through (g) one or more additional times. In some embodiments, generating variation in the WOX transcription factor in a Rosaceae plant results in the Rosaceae plant having a reduced number of thorns and/or prickles, thorns and/or prickles that have a modified architecture, thorns and/or prickles that are reduced in size, and/or no thorns and/or prickles.

In some embodiments, a mutation in an endogenous functional WOX gene may result in a mutated WOX nucleic acid having at least 90% sequence identity to any one of the mutated WOX genes (e.g., WOX1, WOX23) having a nucleotide sequence of SEQ ID NOs: 143, 144, 145, 147, 149, 151, 153 and/or a mutated WOX polypeptide comprising an amino acid sequence having at least 90% sequence identity to any one of SEQ ID NOs: 146, 148, 150, 152, 154 and/or 156.

In some embodiments, a method of detecting a mutant WOX gene (a mutation in an endogenous functional WOX gene) in a Rosaceae plant is provided comprising detecting in the genome of the Rosaceae plant a WOX gene having at least one mutation within a region having at least 80% sequence identity to any one of the nucleotide sequences of SEQ ID NOs: 78-82, 83-86, 87-90, 91-109, 110-125 or 131-135, optionally to any one of SEQ ID NOs: 81, 82, 84-86, 88-90, 94-97, 99-101, 103-105, 107-109, 113-116, 118-120, 122, 124, 125 or 134, optionally wherein the sequence identity may be at least 85%, or at least 90%, or it may be at least 95%, optionally the sequence identity may be 100%.

In some embodiments, the mutated WOX gene that is detected comprises a sequence having at least 90% sequence identity to a mutated WOX nucleic acid having a nucleotide sequence of SEQ ID NOs: 143, 144, 145, 147, 149, 151, 153 and/or 155 and/or encoding a mutated WOX polypeptide comprising an amino acid sequence having at least 90% sequence identity to any one of SEQ ID NOs: 146, 148, 150, 152, 154 and/or 156, optionally wherein the mutation that is detected in the WOX gene is a non-natural mutation. In some embodiments, the mutation in mutated WOX gene generated by the methods of this invention may located within Exon 1 or Exon 2 of the WOX gene.

In some embodiments, a method for editing a specific site in the genome of a Rosaceae plant cell is provided, the method comprising: cleaving, in a site-specific manner, a target site within an endogenous WUSCHEL (WUS)-Related Homeobox (WOX) gene in the Rosaceae plant cell, the endogenous WOX gene: (a) comprising a nucleotide sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 69, 70, 75, 76, 128, 129 or 162-173; (b) comprising a region having at least 80% sequence identity to a nucleotide sequence of any one of SEQ ID NOs: 78-82, 83-86, 87-90, 91-109, 110-125 or 131-135, optionally to any one of SEQ ID NOs: 81, 82, 84-86, 88-90, 94-97, 99-101, 103-105, 107-109, 113-116, 118-120, 122, 124, 125 or 134; (c) encoding an amino acid sequence having at least 80% sequence identity to any one of SEQ ID NOs: 71, 77, 130, or 174-182; and/or (d) encoding a region of an amino acid sequence having at least 80% sequence identity to any one of SEQ ID NOs: 126, 127, 136 or 137, optionally wherein the sequence identity of (a), (b), (c) and/or (d) may be at least 85%, or at least 90%, or it may be at least 95%, optionally the sequence identity may be 100%, thereby generating an edit in the endogenous WOX gene of the Rosaceae plant cell and producing a Rosaceae plant cell comprising the edit in the endogenous WOX gene. In some embodiments, the endogenous WOX gene is expressed specifically in tissues that give rise to trichomes, thorns and/or prickles, optionally, wherein the endogenous WOX gene is expressed in a tissue specific manner in the vascular tissue, the epidermal tissue and/or cells or tissues that give rise to thorns and/or prickles in the Rosaceae plant or part thereof. In some embodiments, the endogenous WOX gene is an endogenous WOX1 gene and/or an endogenous WOX23 gene, optionally wherein an edit is generated in two or more endogenous WOX genes.

In some embodiments, an edit in the endogenous functional WOX gene in a Rosaceae plant results in a mutation including, but not limited to, a base deletion, a base substitution, or a base insertion, optionally wherein the at least one mutation is a non-natural mutation. In some embodiments, the at least one mutation may be located in the first exon or second exon of the functional WOX gene. In some embodiments, the edit may result in at least one mutation that is an insertion of at least one base pair (e.g., 1 base pair to about 100 base pairs), optionally wherein the insertion is an out-of-frame insertion. In some embodiments, the edit may result in at least one mutation that is a deletion, optionally wherein the deletion is about 1 to about 100 consecutive base pairs in length, e.g., about 1-50 consecutive base pairs, about 1-30 consecutive base pairs or about 1-15 consecutive base pairs in length, optionally about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30, 31, 32, 33, 34, 35, 36, 37, 38, 39, 40, 41, 42, 43, 44, 45, 46, 47, 48, 49, or 50 consecutive base pairs. A deletion or insertion useful with this invention may be an out-of-frame insertion or an out-of-frame deletion. In some embodiments, an out-of-frame insertion or out-of-frame deletion may result in a premature stop codon and truncated protein. In some embodiments, the edit in a functional WOX gene results in a nonfunctional WOX gene and/or the WOX transcription factor being truncated, optionally a C-terminal truncation, optionally wherein the C-terminal truncation is a deletion of about one to about 320 consecutive amino acid residues, optionally about 200-320 consecutive amino acid residues from the C-terminus of the WOX transcription factor (e.g., about 200, 205, 210, 211, 212, 213, 214, 215, 216, 217, 218, 219, 220, 225, 230, 235, 240, 245, 250, 255, 260, 265, 270, 275, 280, 285, 290, 295, 300, 305, 310, 311, 313, 314, 315, 316, 317, 318, 319, or 320 consecutive amino acid residues, or any value or range therein).

In some embodiments, a Rosaceae plant comprising the edit in its endogenous functional WOX gene exhibits a phenotype of reduced number of thorns and/or prickles, thorns and/or prickles that have a modified architecture, thorns and/or prickles that are reduced in size, and/or no thorns and/or prickles. In some embodiments, the edit results in a mutated WOX gene comprising a nucleotide sequence having at least 90% sequence identity (e.g., at least 90, 91, 92, 93, 94, 95, 96, 97, 98, 99 or 100%, optionally the sequence identity may be at least 95%, optionally the sequence identity may be 100%) to any one of the mutated WOX genes having a nucleotide sequence of SEQ ID NOs: 143, 144, 145, 147, 149, 151, 153 and/or 155 and/or encoding a polypeptide having at least 90% identity to any one of the mutated WOX transcription factors of SEQ ID NOs: 146, 148, 150, 152, 154 and/or 156.

In some embodiments, a method of editing may further comprise regenerating a Rosaceae plant from the Rosaceae plant cell comprising the edit in the endogenous functional WOX gene, thereby producing a Rosaceae plant comprising the edit in its endogenous functional WOX gene (optionally in the first and/or second exon of the functional WOX gene and having a phenotype of reduced number of thorns and/or prickles, thorns and/or prickles that have a modified architecture, thorns and/or prickles that are reduced in size, and/or no thorns and/or prickles when compared to a control Rosaceae plant that is devoid of the edit.

In some embodiments, a method for making a Rosaceae plant is provided, the method comprising (a) contacting a population of Rosaceae plant cells comprising an endogenous WUSCHEL (WUS)-Related Homeobox (WOX) gene with a nuclease linked to a nucleic acid binding domain (e.g., editing system) that binds to a sequence (i) having at least 80% sequence identity (e.g., at least about 80, 81, 82, 83, 84, 85, 86, 87, 88, 89, 90, 91, 92, 93, 94, 95, 96, 97, 99 or 100% sequence identity) to a nucleotide sequence of any one of SEQ ID NOs: 69, 70, 75, 76, 128, 129, or 162-173, (ii) comprising a region having at least 80% identity to any one of SEQ ID NOs: 78-82, 83-86, 87-90, 91-109, 110-125 or 131-135, optionally to any one of SEQ ID NOs: 81, 82, 84-86, 88-90, 94-97, 99-101, 103-105, 107-109, 113-116, 118-120, 122, 124, 125 or 134; (iii) encoding an amino acid sequence having at least 80% sequence identity to any one of SEQ ID NOs: 71, 77, 130, or 174-182, and/or (iv) encoding a region having at least 80% sequence identity to any one of SEQ ID NOs: 126, 127, 136 or 137, optionally wherein the sequence identity of (i), (ii), (iii) and/or (iv) may be at least 85%, or at least 90%, or it may be at least 95%, optionally the sequence identity may be 100%; (b) selecting a Rosaceae plant cell from the population of Rosaceae plant cells in which an endogenous functional WOX gene has been mutated, thereby producing a Rosaceae plant cell comprising a mutation in the endogenous WOX gene; and (c) growing the selected Rosaceae plant cell into a Rosaceae plant comprising a mutation in the endogenous WOX gene.

In some embodiments, a method for reducing the number and/or size of thorns and/or prickles, for modifying the architecture of thorns and/or prickles, and/or eliminating thorns and/or prickles in a Rosaceae plant is provided, the method comprising (a) contacting a Rosaceae plant cell comprising an endogenous WUSCHEL (WUS)-Related Homeobox (WOX) gene with a nuclease targeting the endogenous WOX gene, wherein the nuclease is linked to a nucleic acid binding domain (e.g., editing system) that binds to a target site within the endogenous WOX gene, wherein the endogenous WOX gene: (i) comprises a nucleotide sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 69, 70, 75, 76, 128, 129 or 162-173; (ii) comprises a region having at least 80% sequence identity to a nucleotide sequence of any one of SEQ ID NOs: 78-82, 83-86, 87-90, 91-109, 110-125 or 131-135, optionally to any one of SEQ ID NOs: 81, 82, 84-86, 88-90, 94-97, 99-101, 103-105, 107-109, 113-116, 118-120, 122, 124, 125 or 134; (iii) encodes an amino acid sequence having at least 80% sequence identity to any one of SEQ ID NOs: 71, 77, 130, or 174-182; and/or (iv) encodes a region of an amino acid sequence having at least 80% sequence identity to any one of SEQ ID NOs: 126, 127, 136 or 137, optionally wherein the sequence identity of (i), (ii), (iii) and/or (iv) may be at least 85%, or at least 90%, or it may be at least 95%, optionally the sequence identity may be 100% to produce a Rosaceae plant cell comprising a mutation in the endogenous WOX gene; and (b) growing the Rosaceae plant cell comprising a mutation in the endogenous WOX gene into a Rosaceae plant comprising the mutation in the endogenous WOX gene thereby producing a Rosaceae plant having a mutated endogenous WOX gene and reduced number of thorns and/or prickles, thorns and/or prickles with a modified architecture, reduced size of thorns and/or prickles and/or elimination of thorns and/or prickles in the Rosaceae plant.

In some embodiments, a method for producing a Rosaceae plant or part thereof comprising at least one cell having a mutated endogenous WUSCHEL (WUS)-Related Homeobox (WOX) gene (e.g., one or more mutated endogenous WOX gene), the method comprising contacting a target site within an endogenous WOX gene in the Rosaceae plant or plant part with a nuclease comprising a cleavage domain and a nucleic acid binding domain, wherein the nucleic acid binding domain binds to a target site within the endogenous WOX gene, wherein the endogenous WOX gene (a) comprises a nucleotide sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 69, 70, 75, 76, 128, 129 or 162-173; (b) comprises a region having at least 80% sequence identity to a nucleotide sequence of any one of SEQ ID NOs: 78-82, 83-86, 87-90, 91-109, 110-125 or 131-135, optionally to any one of SEQ ID NOs: 81, 82, 84-86, 88-90, 94-97, 99-101, 103-105, 107-109, 113-116, 118-120, 122, 124, 125 or 134; (c) encodes an amino acid sequence having at least 80% sequence identity to any one of SEQ ID NOs: 71, 77, 130, or 174-182; and/or (d) encodes a region of an amino acid sequence having at least 80% sequence identity to any one of SEQ ID NOs: 126, 127, 136 or 137, optionally wherein the sequence identity of (i), (ii), (iii) and/or (iv) may be at least 85%, or at least 90%, or it may be at least 95%, optionally the sequence identity may be 100%, thereby producing the Rosaceae plant or part thereof comprising at least one cell having a mutation in the endogenous WOX gene (e.g., a mutation in one or more endogenous WOX genes).

Also provided herein is a method for producing a Rosaceae plant or part thereof comprising a mutated endogenous WUSCHEL (WUS)-Related Homeobox (WOX) gene and exhibiting a reduced number of thorns and/or prickles, thorns and/or prickles that have a modified architecture, thorns and/or prickles that are reduced in size, and/or no thorns and/or prickles, the method comprising contacting a target site within an endogenous WOX gene in the Rosaceae plant or plant part with a nuclease comprising a cleavage domain and a nucleic acid binding domain, wherein the nucleic acid binding domain binds to a target site within the endogenous WOX gene, wherein the endogenous WOX gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 69, 70, 75, 76, 128, 129 or 162-173; (b) comprises a region having at least 80% sequence identity to a nucleotide sequence of any one of SEQ ID NOs: 78-82, 83-86, 87-90, 91-109, 110-125 or 131-135, optionally to any one of SEQ ID NOs: 81, 82, 84-86, 88-90, 94-97, 99-101, 103-105, 107-109, 113-116, 118-120, 122, 124, 125 or 134; (c) encodes an amino acid sequence having at least 80% sequence identity to any one of SEQ ID NOs: 71, 77, 130, or 174-182; and/or (d) encodes a region of an amino acid sequence having at least 80% sequence identity to any one of SEQ ID NOs: 126, 127, 136 or 137, optionally wherein the sequence identity of (i), (ii), (iii) and/or (iv) may be at least 85%, or at least 90%, or it may be at least 95%, optionally the sequence identity may be 100%, thereby producing the Rosaccae plant or part thereof comprising an endogenous WOX gene having a mutation and exhibiting a reduced number of thorns and/or prickles, thorns and/or prickles that have a modified architecture, thorns and/or prickles that are reduced in size, and/or no thorns and/or prickles.

In some embodiments, a target site may be a region or within a region of the WOX gene (a) having at least 80% sequence identity (e.g., at least about 80, 81, 82, 83, 84, 85, 86, 87, 88, 89, 90, 91, 92, 93, 94, 95, 96, 97, 99 or 100% sequence identity, optionally the sequence identity may be at least 85%, or may be at least 90% or it may be at least 95%, optionally the sequence identity may be 100%) to a nucleotide sequence of any one of SEQ ID NOs: 78-82, 83-86, 87-90, 91-109, 110-125 or 131-135, optionally to any one of SEQ ID NOs: 81, 82, 84-86, 88-90, 94-97, 99-101, 103-105, 107-109, 113-116, 118-120, 122, 124, 125 or 134 or (b) having at least 80% sequence identity to a nucleotide sequence encoding any one of the amino acid sequences of SEQ ID NOs: 126, 127, 136 or 137, optionally wherein the sequence identity of (a) and/or (b) may be at least 85%, or at least 90%, or it may be at least 95%, optionally the sequence identity may be 100%.

In some embodiments, a mutation may be a base substitution, a base deletion, a base insertion and/or an inversion, optionally a non-natural mutation. In some embodiments, a mutation is a base substitution to an A, a T, a G, or a C. In some embodiments, the mutation provided in the functional WOX gene is a deletion or an insertion, optionally an out-of-frame deletion or out-of-frame insertion. In some embodiments, a mutation provided in an endogenous functional WOX gene may be a dominant mutation, a semi-dominant mutation, a recessive mutation, or a hypomorphic mutation. In some embodiments, a mutation may be a deletion (e.g., out-of-frame deletion) of about 1 base pair to about 320 consecutive base pairs, optionally, 1 to about 50 consecutive base pairs, 1 to about 30 consecutive base pairs, 1 to about 15 consecutive base pairs. In some embodiments, a mutation may be an insertion (e.g., an out-of-frame insertion) of at least one base pair (e.g., 1 base pair to about 100 consecutive base pairs). In some embodiments, a mutation may be located in the first exon and/or the second exon of the WOX gene. In some embodiments, a mutation provided in a functional WOX gene is an out-of-frame deletion or an out-of-frame insertion may result in a premature stop codon and a truncated WOX transcription factor. In some embodiments, a mutation provided in an endogenous functional WOX gene may result in the encoded WOX transcription factor having a truncation, optionally a C-terminal truncation. In some embodiments, a mutation provided in an endogenous functional WOX gene may result in a reduced amount of the encoded WOX transcription factor or no detectable encoded WOX transcription factor.

In some embodiments, a plant or part thereof that is produced by the methods of this invention comprises a mutated endogenous WOX gene as described herein and exhibits a reduced number of thorns and/or prickles, thorns and/or prickles that have a modified architecture, thorns and/or prickles that are reduced in size, and/or no thorns and/or prickles as compared to a control Rosaceae plant that is devoid of the mutation in the endogenous functional WOX gene, e.g., the plant or plant part has not had a target site within its endogenous functional WOX gene contacted with an editing system or a nuclease comprising a cleavage domain and a nucleic acid binding domain (e.g., a DNA binding domain).

In some embodiments, a mutation in an endogenous functional WOX gene may result in a mutated WOX nucleic acid having at least 90% sequence identity to any one of the mutated WOX genes (e.g., WOX1, WOX23) having a nucleotide sequence of SEQ ID NOs: 143, 144, 145, 147, 149, 151, 153 and/or 155 and/or encode a mutated WOX polypeptide comprising an amino acid sequence having at least 90% sequence identity to any one of SEQ ID NOs: 146, 148, 150, 152, 154 and/or 156.

A nuclease contacting a plant cell, a population of plant cells and/or a target site cleaves an endogenous functional WOX gene, thereby introducing a mutation into the endogenous functional WOX gene. A nuclease useful with the invention may be any nuclease that can be utilized to edit/modify a target nucleic acid. Such nucleases include, but are not limited to a zinc finger nuclease, transcription activator-like effector nucleases (TALEN), endonuclease (e.g., Fok1) and/or a CRISPR-Cas effector protein. Likewise, any nucleic acid binding domain useful with the invention may be any DNA binding domain or RNA binding domain that can be utilized to edit/modify a target nucleic acid. Such nucleic acid binding domains include, but are not limited to, a zinc finger, transcription activator-like DNA binding domain (TAL), an argonaute and/or a CRISPR-Cas effector DNA binding domain.

In some embodiments, a nucleic acid binding domain (e.g., DNA binding domain) is comprised in a nucleic acid binding polypeptide. A “nucleic acid binding protein” or “nucleic acid binding polypeptide” as used herein refers to a polypeptide that binds and/or is capable of binding a nucleic acid in a site- and/or sequence-specific manner. In some embodiments, a nucleic acid binding polypeptide may be a sequence-specific nucleic acid binding polypeptide (e.g., a sequence-specific DNA binding domain) such as, but not limited to, a sequence-specific binding polypeptide and/or domain from, for example, a polynucleotide-guided endonuclease, a CRISPR-Cas effector protein (e.g., a CRISPR-Cas endonuclease), a zinc finger nuclease, a transcription activator-like effector nuclease (TALEN) and/or an Argonaute protein. In some embodiments, a nucleic acid binding polypeptide comprises a cleavage polypeptide (e.g., a nuclease polypeptide and/or domain) such as, but not limited to, an endonuclease (e.g., Fok1), a polynucleotide-guided endonuclease, a CRISPR-Cas endonuclease, a zinc finger nuclease, and/or a transcription activator-like effector nuclease (TALEN). In some embodiments, the nucleic acid binding polypeptide associates with and/or is capable of associating with (e.g., forms a complex with) one or more nucleic acid molecule(s) (e.g., forms a complex with a guide nucleic acid as described herein) that can direct or guide the nucleic acid binding polypeptide to a specific target nucleotide sequence (e.g., a gene locus of a genome) that is complementary to the one or more nucleic acid molecule(s) (or a portion or region thereof), thereby causing the nucleic acid binding polypeptide to bind to the nucleotide sequence at the specific target site. In some embodiments, the nucleic acid binding polypeptide is a CRISPR-Cas effector protein as described herein. In some embodiments, reference is made to specifically to a CRISPR-Cas effector protein for simplicity, but a nucleic acid binding polypeptide as described herein may be used. In some embodiments, a polynucleotide and/or a nucleic acid construct of the invention can be an “expression cassette” or can be comprised within an expression cassette.

In some embodiments, a method of editing an endogenous WUSCHEL (WUS)-Related Homeobox (WOX) gene (e.g., WOX1 and/or WOX23) in a Rosaceae plant or plant part is provided, the method comprising contacting a target site within an endogenous WOX gene in the Rosaceae plant or plant part with a cytosine base editing system comprising a cytosine deaminase and a nucleic acid binding domain that binds to a target site within the endogenous WOX gene, wherein the endogenous WOX gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 69, 70, 75, 76, 128, 129 or 162-173; (b) comprises a region having at least 80% sequence identity to a nucleotide sequence of any one of SEQ ID NOs: 78-82, 83-86, 87-90, 91-109, 110-125 or 131-135, optionally to any one of SEQ ID NOs: 81, 82, 84-86, 88-90, 94-97, 99-101, 103-105, 107-109, 113-116, 118-120, 122, 124, 125 or 134; (c) encodes an amino acid sequence having at least 80% sequence identity to any one of SEQ ID NOs: 71, 77, 130, or 174-182; and/or (d) encodes a region of an amino acid sequence having at least 80% sequence identity to any one of SEQ ID NOs: 126, 127, 136 or 137, optionally wherein the sequence identity of (a), (b), (c) and/or (d) may be at least 85%, or at least 90%, or it may be at least 95%, optionally the sequence identity may be 100%, thereby editing the endogenous WOX gene in the Rosaceae plant or part thereof and producing a Rosaceae plant or part thereof comprising at least one cell having a mutation in the endogenous WOX gene. In some embodiments, the nucleic acid that is detected may comprise a non-natural mutation.

In some embodiments, a method of editing an endogenous WUSCHEL (WUS)-Related Homeobox (WOX) gene (e.g., WOX1 and/or WOX23) in a Rosaceae plant or plant part is provided, the method comprising contacting a target site within an WOX gene in the Rosaceae plant or plant part with an adenosine base editing system comprising an adenosine deaminase and a nucleic acid binding domain that binds to a target site within the WOX gene, wherein the WOX gene (a) comprises a nucleotide sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 69, 70, 75, 76, 128, 129 or 162-173; (b) comprises a region having at least 80% sequence identity to a nucleotide sequence of any one of SEQ ID NOs: 78-82, 83-86, 87-90, 91-109, 110-125 or 131-135, optionally to any one of SEQ ID NOs: 81, 82, 84-86, 88-90, 94-97, 99-101, 103-105, 107-109, 113-116, 118-120, 122, 124, 125 or 134; (c) encodes an amino acid sequence having at least 80% sequence identity to any one of SEQ ID NOs: 71, 77, 130, or 174-182; and/or (d) encodes a region of an amino acid sequence having at least 80% sequence identity to any one of SEQ ID NOs: 126, 127, 136 or 137, optionally wherein the sequence identity of (a), (b), (c) and/or (d) may be at least 85%, or at least 90%, or it may be at least 95%, optionally the sequence identity may be 100%, thereby editing the endogenous WOX gene in the Rosaceae plant or part thereof and producing a Rosaceae plant or part thereof comprising at least one cell having a mutation in the endogenous WOX gene.

In some embodiments, the present invention provides a method of producing a Rosaceae plant comprising a mutation in an endogenous WUSCHEL (WUS)-Related Homeobox (WOX) gene (e.g., WOX1 and/or WOX23) and at least one polynucleotide of interest, the method comprising crossing a Rosaceae plant of the invention comprising at least one mutation in an endogenous functional WOX gene (a first Rosaceae plant) with a second Rosaceae plant that comprises the at least one polynucleotide of interest to produce progeny Rosaceae plants; and selecting progeny Rosaceae plants comprising at least one mutation in the WOX gene and the at least one polynucleotide of interest, thereby producing the Rosaceae plant comprising a mutation in an endogenous functional WOX gene and at least one polynucleotide of interest.

The present invention further provides a method of producing a Rosaceae plant comprising a mutation in an endogenous WUSCHEL (WUS)-Related Homeobox (WOX) gene (e.g., WOX1 and/or WOX23) and at least one polynucleotide of interest, the method comprising introducing at least one polynucleotide of interest into a Rosaceae plant of the present invention comprising at least one mutation in a functional WOX gene, thereby producing a Rosaceae plant comprising at least one mutation in a WOX gene and at least one polynucleotide of interest.

In some embodiments, also provided is a method of producing a Rosaceae plant comprising a mutation in an endogenous WUSCHEL (WUS)-Related Homeobox (WOX) gene (e.g., WOX1 and/or WOX23) and exhibiting a phenotype of a reduced number of thorns and/or prickles, thorns and/or prickles that have a modified architecture, thorns and/or prickles that are reduced in size, and/or no thorns and/or prickles, the method comprising crossing a first Rosaceae plant, which is a Rosaceae plant of the present invention comprising at least one mutation in a functional WOX gene, with a second Rosaceae plant that exhibits a phenotype of reduced number of thorns and/or prickles, thorns and/or prickles that have a modified architecture, thorns and/or prickles that are reduced in size, and/or no thorns and/or prickles; and selecting progeny Rosaceae plants comprising the mutation in the WOX gene and a phenotype of a reduced number of thorns and/or prickles, thorns and/or prickles that have a modified architecture, thorns and/or prickles that are reduced in size, and/or no thorns and/or prickles, thereby producing the Rosaceae plant comprising a mutation in an endogenous WOX gene and exhibiting a phenotype of a reduced number of thorns and/or prickles, thorns and/or prickles that have a modified architecture, thorns and/or prickles that are reduced in size, and/or no thorns and/or prickles as compared to a control plant.

Further provided is a method of controlling weeds in a container (e.g., pot, or seed tray and the like), a growth chamber, a greenhouse, a field, a recreational area, a lawn, or on a roadside, the method comprising applying an herbicide to one or more (a plurality) Rosaceae plants of the invention (e.g., a Rosaceae plant comprising at least one mutation in a functional WOX gene as described herein) growing in a container, a growth chamber, a greenhouse, a field, a recreational area, a lawn, or on a roadside, thereby controlling the weeds in the container, the growth chamber, the greenhouse, the field, the recreational area, the lawn, or on the roadside in which the one or more Rosaceae plants are growing.

In some embodiments, a method of reducing insect predation on a Rosaceae plant is provided, the method comprising applying an insecticide to one or more Rosaceae plants of the invention, optionally, wherein the one or more Rosaceae plants are growing in a container, a growth chamber, a greenhouse, a field, a recreational area, a lawn, or on a roadside, thereby reducing insect predation on the one or more Rosaceae plants.

In some embodiments, a method of reducing fungal disease on a Rosaceae plant is provided, the method comprising applying a fungicide to one or more Rosaceae plants of the invention, optionally, wherein the one or more Rosaceae plants are growing in a container, a growth chamber, a greenhouse, a field, a recreational area, a lawn, or on a roadside, thereby reducing fungal disease on the one or more Rosaceae plants.

A polynucleotide of interest may be any polynucleotide that can confer a desirable phenotype or otherwise modify the phenotype or genotype of a plant. In some embodiments, a polynucleotide of interest may be polynucleotide that confers herbicide tolerance, insect resistance, nematode resistance, disease resistance, increased yield, increased nutrient use efficiency or abiotic stress resistance.

A WUSCHEL (WUS)-Related Homeobox (WOX) functional gene useful with this invention includes any Rosaceae WOX gene in which a mutation as described herein can confer a reduced number of thorns and/or prickles, a modified thorn and/or prickle architecture, are reduced size of thorns and/or prickles, and/or no thorns and/or prickles in a Rosaceae plant or part thereof comprising the mutation. An endogenous functional WOX gene useful with this invention is expressed specifically in tissues that give rise to trichomes, thorns and/or prickles, optionally the WOX gene is expressed in a tissue specific manner in the vascular tissue, the epidermal tissue and/or cells or tissues that give rise to thorns and/or prickles in a Rosaceae plant or part thereof. In some embodiments, the endogenous functional WOX gene is an endogenous functional WOX1 gene and/or an endogenous functional WOX23 gene and the encoded WOX transcription factor is a WOX1 polypeptide or a WOX23 polypeptide, respectively. In some embodiments, an endogenous functional WOX gene (a) comprises a nucleotide sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 69, 70, 75, 76, 128, 129 or 162-173; (b) comprises a region having at least 80% sequence identity to a nucleotide sequence of any one of SEQ ID NOs: 78-82, 83-86, 87-90, 91-109, 110-125 or 131-135, optionally to any one of SEQ ID NOs: 81, 82, 84-86, 88-90, 94-97, 99-101, 103-105, 107-109, 113-116, 118-120, 122, 124, 125 or 134; (c) encodes an amino acid sequence having at least 80% sequence identity to any one of SEQ ID NOs: 71, 77, 130, or 174-182; and/or (d) encodes a region of an amino acid sequence having at least 80% sequence identity to any one of SEQ ID NOs: 126, 127, 136 or 137, optionally wherein the sequence identity of (a), (b), (c) and/or (d) may be at least 85%, or at least 90%, or it may be at least 95%, optionally the sequence identity may be 100%.

In some embodiments, the at least one mutation in an endogenous functional WOX gene in a Rosaceae plant may be a base substitution, a base deletion, a base insertion and/or an inversion, optionally wherein the at least one mutation may be a non-natural mutation. In some embodiments, the at least one mutation in an endogenous functional WOX gene in a Rosaceae plant may result in a Rosaceae plant having the phenotype of a reduced number of thorns and/or prickles, thorns and/or prickles that have a modified architecture, thorns and/or prickles that are reduced in size, and/or no thorns and/or prickles as compared to a control plant devoid of the edit/mutation.

In some embodiments, a mutation in an endogenous functional WOX gene may be a base substitution, a base deletion and/or a base insertion of at least 1 base pair. In some embodiments, a base deletion may be 1 nucleotide to about 100 nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 base pairs, or any range or value therein, e.g., 1 to about 50 base pairs, 1 to about 30 base pairs, 1 to about 15 base pairs, or any range or value therein), optionally where the mutation is at about 2 to about 100 consecutive nucleotides (e.g., 1 to about 50 consecutive base pairs, 1 to about 30 consecutive base pairs, 1 to about 15 consecutive base pairs). In some embodiments, a mutation in an endogenous functional WOX gene may be a base insertion of 1 to about 100 consecutive nucleotides of the WOX nucleic acid. In some embodiments, a mutation in an endogenous functional WOX gene may be an out-of-frame insertion or an out-of-frame deletion that results in an WOX transcription having a C-terminal truncation. In some embodiments, a mutation in an endogenous functional WOX gene may be an out-of-frame insertion or an out-of-frame deletion that results in a reduced amount of the encoded WOX transcription factor or wherein the encoded WOX transcription factor is not detectable. In some embodiments, a mutation may be a base substitution, optionally a substitution to an A, a T, a G, or a C. A mutation may be a point mutation. In some embodiments, a mutation may be a non-natural mutation.

In some embodiments, a mutation in an endogenous WOX gene may be made following cleavage by an editing system that comprises a nuclease and a nucleic acid binding domain that binds to a target site within a functional target nucleic acid (e.g., an endogenous functional WOX gene, e.g., functional WOX1, and/or functional WOX23), the target nucleic acid comprising a sequence having at least 80% sequence identity to any one of the nucleotide sequences of SEQ ID NOs: 69, 70, 75, 76, 128, 129, or 162-173, and/or encoding an amino acid sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 71, 77, 130, or 174-182, optionally wherein the target site is located in a region of the WOX gene: the region comprising a sequence having at least 80% identity to any one of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134, and/or encoding a sequence having at least 80% sequence identity to an amino acid sequence of any one of SEQ ID NOs: 126, 127, 136 or 137, optionally wherein the sequence identity of target nucleic acid, the region and/or the encoded polypeptide may be at least 85%, or at least 90%, or it may be at least 95%, optionally the sequence identity may be 100%.

Further provided are guide nucleic acids (e.g., gRNA, gDNA, crRNA, crDNA) that bind to a target site within an endogenous WUSCHEL (WUS)-Related Homeobox (WOX) gene (e.g., WOX1 and/or WOX23), the endogenous gene (a) comprising a sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of the nucleotide sequences of SEQ ID NOs: 69, 70, 75, 76, 128, 129 or 162-173; (b) comprising a region having at least 80% sequence identity to a nucleotide sequence of any one of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134; (c) encoding an amino acid sequence having at least 80% sequence identity to any one of SEQ ID NOs: 71, 77, 130, or 174-182; and/or (d) encoding a region of an amino acid sequence having at least 80% sequence identity to any one of SEQ ID NOs: 126, 127, 136 or 137, optionally wherein the sequence identity of (a), (b), (c) and/or (d) may be at least 85%, or at least 90%, or it may be at least 95%, optionally the sequence identity may be 100%. In some embodiments, the target site is within a region of the WOX gene having at least 80% sequence identity to of any one of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134 (e.g., may comprise a portion of consecutive nucleotides of any one or more of the nucleotide sequences of any one of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134, or may comprise a portion of consecutive nucleotides of a nucleic acid encoding the amino acid sequence of any one of SEQ ID NOs: 126, 127, 136 or 137), optionally wherein the sequence identity of may be at least 85%, or at least 90%, or it may be at least 95%, optionally the sequence identity may be 100%. In some embodiments, a guide nucleic acid may comprise a spacer comprising any one of the nucleotide sequences of SEQ ID NOs: 138-142.

Spacer sequences useful with a guide of this invention comprise complementarity to a fragment or portion of a nucleotide sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of the nucleotide sequences of SEQ ID NOs: 69, 70, 75, 76, 128, 129, or 162-173 and/or SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134, optionally wherein the sequence identity may be at least 85%, or at least 90%, or it may be at least 95%, optionally the sequence identity may be 100%; or a fragment or portion of a nucleotide sequence encoding a polypeptide comprising a sequence having at least 80% sequence identity to the amino acid sequence of any one of SEQ ID NOs: 71, 77, 130, or 174-182 and/or SEQ ID NOs: 126, 127, 136 or 137, optionally wherein the sequence identity may be at least 85%, or at least 90%, or it may be at least 95%, optionally the sequence identity may be 100%. In some embodiments, a guide nucleic acid may comprise a spacer having a nucleotide sequence having at least 80% sequence identity of any one of SEQ ID NOs: 138-142, or any combination thereof, optionally wherein the sequence identity may be at least 85%, or at least 90%, or it may be at least 95%, optionally the sequence identity may be 100%.

In some embodiments, a Rosaceae plant or plant part thereof is provided that comprises at least one mutation in at least one endogenous functional WUSCHEL (WUS)-Related Homeobox (WOX) gene (e.g., WOX1 and/or WOX23). In some embodiments, the at least one mutation may be a non-natural mutation. In some embodiments, the mutation results in a mutated WOX gene comprising a sequence having at least 90% identity to any one of SEQ ID NOs: 143, 144, 145, 147, 149, 151, 153 and/or 155, optionally wherein the percent identity to SEQ ID NOs: 143, 144, 145, 147, 149, 151, 153 and/or 155 may be at least 95% or it may be 100%.

In some embodiments, a system is provided comprising a guide nucleic acid that binds to a target site within a WOX gene, wherein the target site is in a region of the WOX gene having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134, optionally wherein the sequence identity may be at least 85%, or at least 90%, or it may be at least 95%, optionally the sequence identity may be 100%, and/or comprising a spacer (e.g., one or more spacers) having the nucleotide sequence of any one of SEQ ID NOs: 138-142, and a CRISPR-Cas effector protein that associates with the guide nucleic acid. In some embodiments, the system may further comprise a tracr nucleic acid that associates with the guide nucleic acid and a CRISPR-Cas effector protein, optionally wherein the tracr nucleic acid and the guide nucleic acid are covalently linked.

As used herein, “a CRISPR-Cas effector protein in association with a guide nucleic acid” refers to the complex that is formed between a CRISPR-Cas effector protein and a guide nucleic acid in order to direct the CRISPR-Cas effector protein to a target site within a gene. The invention further provides a gene editing system comprising a CRISPR-Cas effector protein in association with a guide nucleic acid and the guide nucleic acid comprises a spacer sequence that binds to a functional WUSCHEL (WUS)-Related Homeobox (WOX) gene, optionally wherein the WOX gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 69, 70, 75, 76, 128, 129 or 162-173; (b) comprises a region having at least 80% sequence identity to a nucleotide sequence of any one of SEQ ID NOs: 78-82, 83-86, 87-90, 91-109, 110-125 or 131-135, optionally to any one of SEQ ID NOs: 81, 82, 84-86, 88-90, 94-97, 99-101, 103-105, 107-109, 113-116, 118-120, 122, 124, 125 or 134; (c) encodes an amino acid sequence having at least 80% sequence identity to any one of SEQ ID NOs: 71, 77, 130, or 174-182; and/or (d) encodes a region of an amino acid sequence having at least 80% sequence identity to any one of SEQ ID NOs: 126, 127, 136 or 137, optionally wherein the sequence identity of (a), (b), (c) and/or (d) may be at least 85%, or at least 90%, or it may be at least 95%, optionally the sequence identity may be 100%. In some embodiments, a spacer sequence of the guide nucleic acid may comprise the nucleotide sequence of any of SEQ ID NOs: 138-142. In some embodiments, the gene editing system may further comprise a tracr nucleic acid that associates with the guide nucleic acid and a CRISPR-Cas effector protein, optionally wherein the tracr nucleic acid and the guide nucleic acid are covalently linked.

The present invention further provides a complex comprising a CRISPR-Cas effector protein comprising a cleavage domain and a guide nucleic acid, wherein the guide nucleic acid binds to a target site within an endogenous WUSCHEL (WUS)-Related Homeobox (WOX) gene, wherein the endogenous WOX gene: (a) comprises a nucleotide sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 69, 70, 75, 76, 128, 129 or 162-173; (b) comprises a region having at least 80% sequence identity to a nucleotide sequence of any one of SEQ ID NOs: 78-82, 83-86, 87-90, 91-109, 110-125 or 131-135, optionally to any one of SEQ ID NOs: 81, 82, 84-86, 88-90, 94-97, 99-101, 103-105, 107-109, 113-116, 118-120, 122, 124, 125 or 134; (c) encodes an amino acid sequence having at least 80% sequence identity to any one of SEQ ID NOs: 71, 77, 130, or 174-182; and/or (d) encodes a region of an amino acid sequence having at least 80% sequence identity to any one of SEQ ID NOs: 126, 127, 136 or 137, optionally wherein the sequence identity of (a), (b), (c) and/or (d) may be at least 85%, or at least 90%, or it may be at least 95%, optionally the sequence identity may be 100%, and the cleavage domain cleaves a target strand in the WOX gene.

In some embodiments, an expression cassette(s) is/are provided that comprise (a) a polynucleotide encoding CRISPR-Cas effector protein comprising a cleavage domain and (b) a guide nucleic acid that binds to a target site within an endogenous WUSCHEL (WUS)-Related Homeobox (WOX) gene in Rosaceae, wherein the guide nucleic acid comprises a spacer sequence that is complementary to and binds to (i) a portion of a nucleic acid having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 69, 70, 75, 76, 128, 129 or 162-173; (ii) a portion of a nucleic acid having at least 80% sequence identity to any one of SEQ ID NOs: 78-82, 83-86, 87-90, 91-109, 110-125 or 131-135, optionally to any one of SEQ ID NOs: 81, 82, 84-86, 88-90, 94-97, 99-101, 103-105, 107-109, 113-116, 118-120, 122, 124, 125 or 134; (iii) a portion of a nucleic acid encoding an amino acid sequence having at least 80% sequence identity to any one of SEQ ID NOs: 71, 77, 130, or 174-182; and/or (iv) a portion of a nucleic acid encoding an amino acid sequence having at least 80% identity to any one of SEQ ID NOs: 126, 127, 136 or 137, optionally wherein the sequence identity of (i), (ii), (iii) and/or (iv) may be at least 85%, or at least 90%, or it may be at least 95%, optionally the sequence identity may be 100%.

Also provided are nucleic acids encoding a WUSCHEL (WUS)-Related Homeobox (WOX) transcription factor (e.g., WOX1, WOX23), optionally wherein when present in a Rosaceae plant or plant part the mutated WOX gene results in the Rosaceae plant comprising a phenotype of reduced number of thorns and/or prickles, thorns and/or prickles that have a modified architecture, thorns and/or prickles that are reduced in size, and/or no thorns and/or prickles as compared to a control Rosaceae plant or plant part devoid of the mutation. In some embodiments, a mutated WOX gene has at least 90% sequence identity (e.g., at least about 90, 91, 92, 93, 94, 95, 96, 97, 99 or 100% sequence identity, optionally the sequence identity may be at least 95%, optionally the sequence identity may be 100%) to any one of the mutated WOX genes (e.g., WOX1, WOX23) of SEQ ID NOs: 143, 144, 145, 147, 149, 151, 153 and/or 155 and/or encodes a mutated WOX polypeptide comprising an amino acid sequence having at least 90% sequence identity to any one of SEQ ID NOs: 146, 148, 150, 152, 154 and/or 156.

Further provided are genetic markers for use in a blackberry plant or part thereof for identifying plants having thorns and/or prickles with a modified architecture, a reduced number and/or size thorns and/or prickles, or no thorns and/or prickles, the genetic marker comprising (a) the nucleic acid sequence of GAACTCTG located between nucleotide position of 2897 and 2898 with reference to the nucleotide position numbering of SEQ ID NO:69 and/or (b) the nucleic acid sequence of SEQ ID NO:143. In some embodiments, the blackberry plant may be Rubus allegheniensis (Allegheny blackberry), Rubus armeniacus (Himalayan blackberry), Rubus canadensis (Canadian blackberry), Rubus fruticosus agg. (European blackberry), Rubus laciniatus (cutleaf evergreen blackberry), Rubus pensilvanicus (Pennsylvania blackberry), Rubus trifidus (Japanese blackberry), Rubus ursinus (trailing blackberry). In some embodiments, a genetic marker as described herein may be used to identify a cell of a blackberry plant or part thereof that carries the genetic marker and therefore is capable of being regenerated into a plant having the phenotype of having thorns and/or prickles with a modified architecture, a reduced number and/or size thorns and/or prickles, or no thorns and/or prickles. In some embodiments, plants or plant parts are provided that comprise the genetic markers of the invention.

In some embodiments, a method is provided for identifying a blackberry plant or part thereof having a phenotype of thorns and/or prickles with a modified architecture, a reduced number and/or size thorns and/or prickles, or no thorns and/or prickles, the method comprising identifying in the genome of a cell of the blackberry plant or part thereof (a) an insertion in an endogenous WUSCHEL (WUS)-Related Homeobox (WOX) gene, the insertion comprising of the nucleic acid sequence of GAACTCTG located between nucleotide position of 2897 and 2898 with reference to the nucleotide position numbering of SEQ ID NO:69 and/or (b) the nucleic acid sequence of SEQ ID NO:143. In some embodiments, plants or plant parts are provided that are identified using the genetic markers of the invention.

In some embodiments, the present invention further provides a method for breeding a blackberry plant having thorns and/or prickles with a modified architecture, a reduced number and/or size thorns and/or prickles, or no thorns and/or prickles, the method comprising (a) crossing a blackberry plant comprising in its genome (i) an insertion in an endogenous WUSCHEL (WUS)-Related Homeobox (WOX) gene, the insertion comprising of the nucleic acid sequence of GAACTCTG located between nucleotide position of 2897 and 2898 with reference to the nucleotide position numbering of SEQ ID NO:69 and/or (ii) the nucleic acid sequence of SEQ ID NO:143 with itself or another blackberry plant to produce progeny plants; and (b) selecting one or more progeny plants comprising the insertion and having thorns and/or prickles with a modified architecture, a reduced number and/or size thorns and/or prickles, or no thorns and/or prickles, thereby producing blackberry plants having thorns and/or prickles with a modified architecture, a reduced number and/or size thorns and/or prickles, or no thorns and/or prickles. In some embodiments a method for producing a blackberry plant having thorns and/or prickles with a modified architecture, a reduced number and/or size thorns and/or prickles, or no thorns and/or prickles may further comprise (c) crossing a selected progeny plant of (b) with itself or another blackberry plant one or more additional times and selecting progeny plants comprising the insertion and having thorns and/or prickles with a modified architecture, a reduced number and/or size thorns and/or prickles, or no thorns and/or prickles, thereby producing blackberry plants having thorns and/or prickles with a modified architecture, having a reduced number and/or size thorns and/or prickles, or having no thorns and/or prickles. In some embodiments, the blackberry plant is Rubus allegheniensis (Allegheny blackberry), Rubus armeniacus (Himalayan blackberry), Rubus canadensis (Canadian blackberry), Rubus fruticosus agg. (European blackberry), Rubus laciniatus (cutleaf evergreen blackberry), Rubus pensilvanicus (Pennsylvania blackberry), Rubus trifidus (Japanese blackberry), Rubus ursinus (trailing blackberry).

In some embodiments, a blackberry plant or part thereof is provided produced by the methods of identifying and breeding as described herein and exhibiting a phenotype of thorns and/or prickles with a modified architecture, a reduced number and/or size thorns and/or prickles, or no thorns and/or prickles as compared to a control blackberry plant devoid of the insertion.

Nucleic acid constructs of the invention (e.g., a construct comprising a sequence specific nucleic acid binding domain (e.g., sequence specific DNA binding domain), a CRISPR-Cas effector domain, a deaminase domain, reverse transcriptase (RT), RT template and/or a guide nucleic acid, etc.) and expression cassettes/vectors comprising the same may be used as an editing system of this invention for modifying functional target nucleic acids (e.g., functional endogenous WOX genes, e.g., endogenous functional WOX1 gene, endogenous functional WOX23 gene) and/or their expression.

Any Rosaceae plant comprising an endogenous WOX gene that is capable of conferring a reduced number of thorns and/or prickles, thorns and/or prickles that have a modified architecture, thorns and/or prickles that are reduced in size, and/or no thorns and/or prickles when a functional WOX gene is modified as described herein, may be modified (e.g., mutated, e.g., base edited, cleaved, nicked, etc.) as described herein (e.g., using the polypeptides, polynucleotides, RNPs, nucleic acid constructs, expression cassettes, and/or vectors of the invention) to modify the architecture of thorns and/or prickles, to reduce the number of size of thorns and/or prickles, and/or to eliminate thorns and/or prickles in the Rosaceae plant.

An editing system useful with this invention can be any site-specific (sequence-specific) genome editing system now known or later developed, which system can introduce mutations in a target specific manner. For example, an editing system (e.g., site- or sequence-specific editing system) can include, but is not limited to, a CRISPR-Cas editing system, a meganuclease editing system, a zinc finger nuclease (ZFN) editing system, a transcription activator-like effector nuclease (TALEN) editing system, a base editing system and/or a prime editing system, each of which can comprise one or more polypeptides and/or one or more polynucleotides that when expressed as a system in a cell can modify (mutate) a target nucleic acid in a sequence specific manner. In some embodiments, an editing system (e.g., site- or sequence-specific editing system) can comprise one or more polynucleotides and/or one or more polypeptides, including but not limited to a nucleic acid binding domain (DNA binding domain), a nuclease, and/or other polypeptide, and/or a polynucleotide.

In some embodiments, an editing system can comprise one or more sequence-specific nucleic acid binding domains (DNA binding domains) that can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, an editing system can comprise one or more cleavage domains (e.g., nucleases) including, but not limited to, an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN). In some embodiments, an editing system can comprise one or more polypeptides that include, but are not limited to, a deaminase (e.g., a cytosine deaminase, an adenine deaminase), a reverse transcriptase, a Dna2 polypeptide, and/or a 5′ flap endonuclease (FEN). In some embodiments, an editing system can comprise one or more polynucleotides, including, but is not limited to, a CRISPR array (CRISPR guide) nucleic acid, extended guide nucleic acid, and/or a reverse transcriptase template.

In some embodiments, a method of modifying or editing a functional WUSCHEL (WUS)-Related Homeobox (WOX) gene may comprise contacting a target nucleic acid (e.g., a nucleic acid encoding WOX transcription factor, e.g., a WOX1 polypeptide, a WOX23 polypeptide) with a base-editing fusion protein (e.g., a sequence specific DNA binding protein (e.g., a CRISPR-Cas effector protein or domain) fused to a deaminase domain (e.g., an adenine deaminase and/or a cytosine deaminase) and a guide nucleic acid, wherein the guide nucleic acid is capable of guiding/targeting the base editing fusion protein to the target nucleic acid, thereby editing a locus within the target nucleic acid. In some embodiments, a base editing fusion protein and guide nucleic acid may be comprised in one or more expression cassettes. In some embodiments, the target nucleic acid may be contacted with a base editing fusion protein and an expression cassette comprising a guide nucleic acid. In some embodiments, the sequence-specific nucleic acid binding fusion proteins and guides may be provided as ribonucleoproteins (RNPs). In some embodiments, a cell may be contacted with more than one base-editing fusion protein and/or one or more guide nucleic acids that may target one or more target nucleic acids in the cell.

In some embodiments, a method of modifying or editing a functional WUSCHEL (WUS)-Related Homeobox (WOX) gene may comprise contacting a target nucleic acid (e.g., a nucleic acid encoding a WOX polypeptide) with a sequence-specific nucleic acid binding fusion protein (e.g., a sequence-specific DNA binding protein (e.g., a CRISPR-Cas effector protein or domain) fused to a peptide tag, a deaminase fusion protein comprising a deaminase domain (e.g., an adenine deaminase and/or a cytosine deaminase) fused to an affinity polypeptide that is capable of binding to the peptide tag, and a guide nucleic acid, wherein the guide nucleic acid is capable of guiding/targeting the sequence-specific nucleic acid binding fusion protein to the target nucleic acid and the sequence-specific nucleic acid binding fusion protein is capable of recruiting the deaminase fusion protein to the target nucleic acid via the peptide tag-affinity polypeptide interaction, thereby editing a locus within the target nucleic acid. In some embodiments, the sequence-specific nucleic acid binding fusion protein may be fused to the affinity polypeptide that binds the peptide tag and the deaminase may be fused to the peptide tag, thereby recruiting the deaminase to the sequence-specific nucleic acid binding fusion protein and to the target nucleic acid. In some embodiments, the sequence-specific binding fusion protein, deaminase fusion protein, and guide nucleic acid may be comprised in one or more expression cassettes. In some embodiments, the target nucleic acid may be contacted with a sequence-specific binding fusion protein, deaminase fusion protein, and an expression cassette comprising a guide nucleic acid. In some embodiments, the sequence-specific nucleic acid binding fusion proteins, deaminase fusion proteins and guides may be provided as ribonucleoproteins (RNPs).

In some embodiments, methods such as prime editing may be used to generate a mutation in an endogenous functional WOX gene in a Rosaceae plant or part thereof. In prime editing, RNA-dependent DNA polymerase (reverse transcriptase, RT) and reverse transcriptase templates (RT template) are used in combination with sequence specific nucleic acid binding domains that confer the ability to recognize and bind the target in a sequence-specific manner, and which can also cause a nick of the PAM-containing strand within the target. The nucleic acid binding domain may be a CRISPR-Cas effector protein and in this case, the CRISPR array or guide RNA may be an extended guide that comprises an extended portion comprising a primer binding site (PSB) and the edit to be incorporated into the genome (the template). Similar to base editing, prime editing can take advantages of the various methods of recruiting proteins for use in the editing to the target site, such methods including both non-covalent and covalent interactions between the proteins and nucleic acids used in the selected process of genome editing.

As used herein, a “CRISPR-Cas effector protein” is a protein or polypeptide or domain thereof that cleaves or cuts a nucleic acid, binds a nucleic acid (e.g., a target nucleic acid and/or a guide nucleic acid), and/or that identifies, recognizes, or binds a guide nucleic acid as defined herein. In some embodiments, a CRISPR-Cas effector protein may be an enzyme (e.g., a nuclease, endonuclease, nickase, etc.) or portion thereof and/or may function as an enzyme. In some embodiments, a CRISPR-Cas effector protein refers to a CRISPR-Cas nuclease polypeptide or domain thereof that comprises nuclease activity or in which the nuclease activity has been reduced or eliminated, and/or comprises nickase activity or in which the nickase has been reduced or eliminated, and/or comprises single stranded DNA cleavage activity (ss DNAse activity) or in which the ss DNAse activity has been reduced or eliminated, and/or comprises self-processing RNAse activity or in which the self-processing RNAse activity has been reduced or eliminated. A CRISPR-Cas effector protein may bind to a target nucleic acid.

In some embodiments, a sequence-specific nucleic acid binding domain may be a CRISPR-Cas effector protein. In some embodiments, a CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein of the invention may be from a Type II CRISPR-Cas system or a Type V CRISPR-Cas system. In some embodiments, a CRISPR-Cas effector protein may be Type II CRISPR-Cas effector protein, for example, a Cas9 effector protein. In some embodiments, a CRISPR-Cas effector protein may be Type V CRISPR-Cas effector protein, for example, a Cas12 effector protein.

In some embodiments, a CRISPR-Cas effector protein may include, but is not limited to, a Cas9, C2c1, C2c3, Cas12a (also referred to as Cpf1), Cas12b, Cas12c, Cas12d, Cas12c, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Csc2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12c (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site (e.g., RuvC, HNH, e.g., RuvC site of a Cas12a nuclease domain; e.g., RuvC site and/or HNH site of a Cas9 nuclease domain). A CRISPR-Cas effector protein having a mutation in its nuclease active site, and therefore, no longer comprising nuclease activity, is commonly referred to as “dead,” e.g., dCas. In some embodiments, a CRISPR-Cas effector protein domain or polypeptide having a mutation in its nuclease active site may have impaired activity or reduced activity as compared to the same CRISPR-Cas effector protein without the mutation, e.g., a nickase, e.g., Cas9 nickase, Cas12a nickase.

A CRISPR Cas9 effector protein or CRISPR Cas9 effector domain useful with this invention may be any known or later identified Cas9 nuclease. In some embodiments, a CRISPR Cas9 polypeptide can be a Cas9 polypeptide from, for example, Streptococcus spp. (e.g., S. pyogenes, S. thermophilus), Lactobacillus spp., Bifidobacterium spp., Kandleria spp., Leuconostoc spp., Oenococcus spp., Pediococcus spp., Weissella spp., and/or Olsenella spp. Example Cas9 sequences include, but are not limited to, the amino acid sequences of SEQ ID NO: 56 and SEQ ID NO:57 or the nucleotide sequences of SEQ ID NOs: 58-68.

In some embodiments, the CRISPR-Cas effector protein may be a Cas9 polypeptide derived from Streptococcus pyogenes and recognizes the PAM sequence motif NGG, NAG, NGA (Mali et al, Science 2013; 339 (6121): 823-826). In some embodiments, the CRISPR-Cas effector protein may be a Cas9 polypeptide derived from Streptococcus thermophiles and recognizes the PAM sequence motif NGGNG and/or NNAGAAW (W=A or T) (See, e.g., Horvath et al, Science, 2010; 327 (5962): 167-170, and Deveau et al, J Bacteriol 2008; 190 (4): 1390-1400). In some embodiments, the CRISPR-Cas effector protein may be a Cas9 polypeptide derived from Streptococcus mutans and recognizes the PAM sequence motif NGG and/or NAAR (R=A or G) (See, e.g., Deveau et al, J BACTERIOL 2008; 190 (4): 1390-1400). In some embodiments, the CRISPR-Cas effector protein may be a Cas9 polypeptide derived from Streptococcus aureus and recognizes the PAM sequence motif NNGRR (R=A or G). In some embodiments, the CRISPR-Cas effector protein may be a Cas9 protein derived from S. aureus, which recognizes the PAM sequence motif NGRRT (R=A or G). In some embodiments, the CRISPR-Cas effector protein may be a Cas9 polypeptide derived from S. aureus, which recognizes the PAM sequence motif NGRRV (R=A or G). In some embodiments, the CRISPR-Cas effector protein may be a Cas9 polypeptide that is derived from Neisseria meningitidis and recognizes the PAM sequence motif NGATT or NGCTT (R=A or G, V=A, G or C) (See, e.g., Hou et ah, PNAS 2013, 1-6). In the aforementioned embodiments, N can be any nucleotide residue, e.g., any of A, G, C or T. In some embodiments, the CRISPR-Cas effector protein may be a Cas13a protein derived from Leptotrichia shahii, which recognizes a protospacer flanking sequence (PFS) (or RNA PAM (rPAM)) sequence motif of a single 3′ A, U, or C, which may be located within the target nucleic acid.

In some embodiments, the CRISPR-Cas effector protein may be derived from Cas12a, which is a Type V Clustered Regularly Interspaced Short Palindromic Repeats (CRISPR)-Cas nuclease, see, e.g., amino acid sequences of SEQ ID NOs: 1-17, nucleic acid sequences of SEQ ID NOs: 18-20. Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a effector protein/domain useful with this invention may be any known or later identified Cas12a polypeptide (previously known as Cpf1) (see, e.g., U.S. Pat. No. 9,790,490, which is incorporated by reference for its disclosures of Cpf1 (Cas12a) sequences). The term “Cas12a”, “Cas12a polypeptide” or “Cas12a domain” refers to an RNA-guided nuclease comprising a Cas12a polypeptide, or a fragment thereof, which comprises the guide nucleic acid binding domain of Cas12a and/or an active, inactive, or partially active DNA cleavage domain of Cas12a. In some embodiments, a Cas12a useful with the invention may comprise a mutation in the nuclease active site (e.g., RuvC site of the Cas12a domain). A Cas12a domain or Cas12a polypeptide having a mutation in its nuclease active site, and therefore, no longer comprising nuclease activity, is commonly referred to as deadCas12a (e.g., dCas12a). In some embodiments, a Cas12a domain or Cas12a polypeptide having a mutation in its nuclease active site may have impaired activity, e.g., may have nickase activity.

Any deaminase domain/polypeptide useful for base editing may be used with this invention. In some embodiments, the deaminase domain may be a cytosine deaminase domain or an adenine deaminase domain. A cytosine deaminase (or cytidine deaminase) useful with this invention may be any known or later identified cytosine deaminase from any organism (see, e.g., U.S. Pat. No. 10,167,457 and Thuronyi et al. Nat. Biotechnol. 37:1070-1079 (2019), each of which is incorporated by reference herein for its disclosure of cytosine deaminases). Cytosine deaminases can catalyze the hydrolytic deamination of cytidine or deoxycytidine to uridine or deoxyuridine, respectively. Thus, in some embodiments, a deaminase or deaminase domain useful with this invention may be a cytidine deaminase domain, catalyzing the hydrolytic deamination of cytosine to uracil. In some embodiments, a cytosine deaminase may be a variant of a naturally occurring cytosine deaminase, including but not limited to a primate (e.g., a human, monkey, chimpanzee, gorilla), a dog, a cow, a rat or a mouse. Thus, in some embodiments, a cytosine deaminase useful with the invention may be about 70% to about 100% identical to a wild type cytosine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring cytosine deaminase).

In some embodiments, a cytosine deaminase useful with the invention may be an apolipoprotein B mRNA-editing complex (APOBEC) family deaminase. In some embodiments, the cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, a human activation induced deaminase (hAID), an rAPOBEC1, FERNY, and/or a CDA1, optionally a pmCDA1, an atCDA1 (e.g., At2g19570), and evolved versions of the same (e.g., SEQ ID NO:27, SEQ ID NO:28 or SEQ ID NO: 29). In some embodiments, the cytosine deaminase may be an APOBEC1 deaminase having the amino acid sequence of SEQ ID NO:23. In some embodiments, the cytosine deaminase may be an APOBEC3A deaminase having the amino acid sequence of SEQ ID NO: 24. In some embodiments, the cytosine deaminase may be an CDA1 deaminase, optionally a CDA1 having the amino acid sequence of SEQ ID NO:25. In some embodiments, the cytosine deaminase may be a FERNY deaminase, optionally a FERNY having the amino acid sequence of SEQ ID NO:26. In some embodiments, a cytosine deaminase useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical) to the amino acid sequence of a naturally occurring cytosine deaminase (e.g., an evolved deaminase). In some embodiments, a cytosine deaminase useful with the invention may be about 70% to about 99.5% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical) to the amino acid sequence of SEQ ID NO:23, SEQ ID NO:24, SEQ ID NO: 25 or SEQ ID NO:26 (e.g., at least 80%, at least 85%, at least 90%, at least 92%, at least 95%, at least 96%, at least 97%, at least 98%, at least 99%, or at least 99.5% identical to the amino acid sequence of SEQ ID NO:23, SEQ ID NO:24, SEQ ID NO:25, SEQ ID NO:26, SEQ ID NO:27, SEQ ID NO:28 or SEQ ID NO:29). In some embodiments, a polynucleotide encoding a cytosine deaminase may be codon optimized for expression in a plant and the codon optimized polypeptide may be about 70% to 99.5% identical to the reference polynucleotide.

In some embodiments, a nucleic acid construct of this invention may further encode a uracil glycosylase inhibitor (UGI) (e.g., uracil-DNA glycosylase inhibitor) polypeptide/domain. Thus, in some embodiments, a nucleic acid construct encoding a CRISPR-Cas effector protein and a cytosine deaminase domain (e.g., encoding a fusion protein comprising a CRISPR-Cas effector protein domain fused to a cytosine deaminase domain, and/or a CRISPR-Cas effector protein domain fused to a peptide tag or to an affinity polypeptide capable of binding a peptide tag and/or a deaminase protein domain fused to a peptide tag or to an affinity polypeptide capable of binding a peptide tag) may further encode a uracil-DNA glycosylase inhibitor (UGI), optionally wherein the UGI may be codon optimized for expression in a plant. In some embodiments, the invention provides fusion proteins comprising a CRISPR-Cas effector polypeptide, a deaminase domain, and a UGI and/or one or more polynucleotides encoding the same, optionally wherein the one or more polynucleotides may be codon optimized for expression in a plant. In some embodiments, the invention provides fusion proteins, wherein a CRISPR-Cas effector polypeptide, a deaminase domain, and a UGI may be fused to any combination of peptide tags and affinity polypeptides as described herein, thereby recruiting the deaminase domain and UGI to the CRISPR-Cas effector polypeptide and a target nucleic acid. In some embodiments, a guide nucleic acid may be linked to a recruiting RNA motif and one or more of the deaminase domain and/or UGI may be fused to an affinity polypeptide that is capable of interacting with the recruiting RNA motif, thereby recruiting the deaminase domain and UGI to a target nucleic acid.

A “uracil glycosylase inhibitor” useful with the invention may be any protein that is capable of inhibiting a uracil-DNA glycosylase base-excision repair enzyme. In some embodiments, a UGI domain comprises a wild type UGI or a fragment thereof. In some embodiments, a UGI domain useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical and any range or value therein) to the amino acid sequence of a naturally occurring UGI domain. In some embodiments, a UGI domain may comprise the amino acid sequence of SEQ ID NO:41 or a polypeptide having about 70% to about 99.5% sequence identity to the amino acid sequence of SEQ ID NO:41 (e.g., at least 80%, at least 85%, at least 90%, at least 92%, at least 95%, at least 96%, at least 97%, at least 98%, at least 99%, or at least 99.5% identical to the amino acid sequence of SEQ ID NO:41). For example, in some embodiments, a UGI domain may comprise a fragment of the amino acid sequence of SEQ ID NO:41 that is 100% identical to a portion of consecutive nucleotides (e.g., 10, 15, 20, 25, 30, 35, 40, 45, 50, 55, 60, 65, 70, 75, 80 consecutive nucleotides; e.g., about 10, 15, 20, 25, 30, 35, 40, 45, to about 50, 55, 60, 65, 70, 75, 80 consecutive nucleotides) of the amino acid sequence of SEQ ID NO:41. In some embodiments, a UGI domain may be a variant of a known UGI (e.g., SEQ ID NO:41) having about 70% to about 99.5% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% sequence identity, and any range or value therein) to the known UGI. In some embodiments, a polynucleotide encoding a UGI may be codon optimized for expression in a plant (e.g., a plant) and the codon optimized polypeptide may be about 70% to about 99.5% identical to the reference polynucleotide.

An adenine deaminase (or adenosine deaminase) useful with this invention may be any known or later identified adenine deaminase from any organism (see, e.g., U.S. Pat. No. 10,113,163, which is incorporated by reference herein for its disclosure of adenine deaminases). An adenine deaminase can catalyze the hydrolytic deamination of adenine or adenosine. In some embodiments, the adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A→G conversion in the sense (e.g., “+”; template) strand of the target nucleic acid or a T→C conversion in the antisense (e.g., “−”, complementary) strand of the target nucleic acid.

In some embodiments, an adenosine deaminase may be a variant of a naturally occurring adenine deaminase. Thus, in some embodiments, an adenosine deaminase may be about 70% to 100% identical to a wild type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, the deaminase or deaminase does not occur in nature and may be referred to as an engineered, mutated or evolved adenosine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus, and the like). In some embodiments, a polynucleotide encoding an adenine deaminase polypeptide/domain may be codon optimized for expression in a plant.

In some embodiments, an adenine deaminase domain may be a wild type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coli. In some embodiments, the TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA. In some embodiments, a TadA polypeptide or TadA domain does not comprise an N-terminal methionine. In some embodiments, a wild type E. coli TadA comprises the amino acid sequence of SEQ ID NO:30. In some embodiments, a mutated/evolved E. coli TadA* comprises the amino acid sequence of SEQ ID NOs: 31-40 (e.g., SEQ ID NOs: 31, 32, 33, 34, 35, 36, 37, 38, 39 or 40). In some embodiments, a polynucleotide encoding a TadA/TadA* may be codon optimized for expression in a plant.

A cytosine deaminase catalyzes cytosine deamination and results in a thymidine (through a uracil intermediate), causing a C to T conversion, or a G to A conversion in the complementary strand in the genome. Thus, in some embodiments, the cytosine deaminase encoded by the polynucleotide of the invention generates a C→T conversion in the sense (e.g., “+”; template) strand of the target nucleic acid or a G→A conversion in antisense (e.g., “−”, complementary) strand of the target nucleic acid.

In some embodiments, the adenine deaminase encoded by the nucleic acid construct of the invention generates an A→G conversion in the sense (e.g., “+”; template) strand of the target nucleic acid or a T→C conversion in the antisense (e.g., “−”, complementary) strand of the target nucleic acid.

The nucleic acid constructs of the invention encoding a base editor comprising a sequence-specific nucleic acid binding protein and a cytosine deaminase polypeptide, and nucleic acid constructs/expression cassettes/vectors encoding the same, may be used in combination with guide nucleic acids for modifying target nucleic acid including, but not limited to, generation of C→T or G→A mutations in a target nucleic acid including, but not limited to, a plasmid sequence; generation of C→T or G→A mutations in a coding sequence to alter an amino acid identity; generation of C→T or G→A mutations in a coding sequence to generate a stop codon; generation of C→T or G→A mutations in a coding sequence to disrupt a start codon; generation of point mutations in genomic DNA to disrupt function; and/or generation of point mutations in genomic DNA to disrupt splice junctions.

The nucleic acid constructs of the invention encoding a base editor comprising a sequence-specific nucleic acid binding protein and an adenine deaminase polypeptide, and expression cassettes and/or vectors encoding the same may be used in combination with guide nucleic acids for modifying a target nucleic acid including, but not limited to, generation of A→G or T→C mutations in a target nucleic acid including, but not limited to, a plasmid sequence; generation of A→G or T→C mutations in a coding sequence to alter an amino acid identity; generation of A→G or T→C mutations in a coding sequence to generate a stop codon; generation of A→G or T→C mutations in a coding sequence to disrupt a start codon; generation of point mutations in genomic DNA to disrupt function; and/or generation of point mutations in genomic DNA to disrupt splice junctions.

The nucleic acid constructs of the invention comprising a CRISPR-Cas effector protein or a fusion protein thereof may be used in combination with a guide RNA (gRNA, CRISPR array, CRISPR RNA, crRNA), designed to function with the encoded CRISPR-Cas effector protein or domain, to modify a target nucleic acid. A guide nucleic acid useful with this invention comprises at least one spacer sequence and at least one repeat sequence. The guide nucleic acid is capable of forming a complex with the CRISPR-Cas nuclease domain encoded and expressed by a nucleic acid construct of the invention and the spacer sequence is capable of hybridizing to a target nucleic acid, thereby guiding the complex (e.g., a CRISPR-Cas effector fusion protein (e.g., CRISPR-Cas effector domain fused to a deaminase domain and/or a CRISPR-Cas effector domain fused to a peptide tag or an affinity polypeptide to recruit a deaminase domain and optionally, a UGI) to the target nucleic acid, wherein the target nucleic acid may be modified (e.g., cleaved or edited) or modulated (e.g., modulating transcription) by the deaminase domain.

As an example, a nucleic acid construct encoding a Cas9 domain linked to a cytosine deaminase domain (e.g., fusion protein) may be used in combination with a Cas9 guide nucleic acid to modify a target nucleic acid, wherein the cytosine deaminase domain of the fusion protein deaminates a cytosine base in the target nucleic acid, thereby editing the target nucleic acid. In a further example, a nucleic acid construct encoding a Cas9 domain linked to an adenine deaminase domain (e.g., fusion protein) may be used in combination with a Cas9 guide nucleic acid to modify a target nucleic acid, wherein the adenine deaminase domain of the fusion protein deaminates an adenosine base in the target nucleic acid, thereby editing the target nucleic acid.

Likewise, a nucleic acid construct encoding a Cas12a domain (or other selected CRISPR-Cas nuclease, e.g., C2c1, C2c3, Cas12b, Cas12c, Cas12d, Cas12c, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Csc2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5) linked to a cytosine deaminase domain or adenine deaminase domain (e.g., fusion protein) may be used in combination with a Cas12a guide nucleic acid (or the guide nucleic acid for the other selected CRISPR-Cas nuclease) to modify a target nucleic acid, wherein the cytosine deaminase domain or adenine deaminase domain of the fusion protein deaminates a cytosine base in the target nucleic acid, thereby editing the target nucleic acid.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target DNA (e.g., protospacer), and at least one repeat sequence (e.g., a repeat of a Type V Cas12a CRISPR-Cas system, or a fragment or portion thereof; a repeat of a Type II Cas9 CRISPR-Cas system, or fragment thereof; a repeat of a Type V C2c1 CRISPR Cas system, or a fragment thereof; a repeat of a CRISPR-Cas system of, for example, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5, or a fragment thereof), wherein the repeat sequence may be linked to the 5′ end and/or the 3′ end of the spacer sequence. The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made, and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35 (Web Server issue): W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more (e.g., 99.1, 99.2, 99.3, 99.4, 99.5, 99.6, 99.7, 99.8, 99.9, or 100%)) to the same region (e.g., 5′ end) of a wild type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g., protospacer) (e.g., a portion of consecutive nucleotides of a sequence that (a) comprises a nucleotide sequence having at least 80% sequence identity (e.g., at least about 80, 81, 82, 83, 84, 85, 86, 87, 88, 89, 90, 91, 92, 93, 94, 95, 96, 97, 99 or 100% sequence identity) to any one of SEQ ID NOs: 69, 70, 75, 76, 128, 129 or 162-173; (b) comprises a region having at least 80% sequence identity to a nucleotide sequence of any one of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134; (c) encodes an amino acid sequence having at least 80% sequence identity to any one of SEQ ID NOs: 71, 77, 130, or 174-182; and/or (d) encodes a region of an amino acid sequence having at least 80% sequence identity to any one of SEQ ID NOs: 126, 127, 136 or 137, optionally wherein the sequence identity of (a), (b), (c) and/or (d) may be at least 85%, or at least 90%, or it may be at least 95%, optionally the sequence identity may be 100%. In some embodiments, a spacer sequence (e.g., one or more spacers) may include, but is not limited to, the nucleotide sequence of any one of SEQ ID NOs: 138-142, or any combination thereof. The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more (e.g., 99.1, 99.2, 99.3, 99.4, 99.5, 99.6, 99.7, 99.8, 99.9, or 100%)) to a target nucleic acid. Thus, in some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 21, 22, or 23 nucleotides in length. In some embodiments, a spacer sequence may comprise the sequence of SEQ ID NOs: 138-142, or the reverse complement thereof.

In some embodiments, the 5′ region of a spacer sequence of a guide nucleic acid may be identical to a target DNA, while the 3′ region of the spacer may be substantially complementary to the target DNA (see, for example, a spacer sequence of a Type V CRISPR-Cas system), or the 3′ region of a spacer sequence of a guide nucleic acid may be identical to a target DNA, while the 5′ region of the spacer may be substantially complementary to the target DNA (see, for example, a spacer sequence of a Type II CRISPR-Cas system), and therefore, the overall complementarity of the spacer sequence to the target DNA may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target DNA, while the remaining nucleotides in the 3′ region of the spacer sequence are substantially complementary (e.g., at least about 70% complementary) to the target DNA. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target DNA, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA.

As a further example, in a guide for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target DNA, while the remaining nucleotides in the 5′ region of the spacer sequence are substantially complementary (e.g., at least about 70% complementary) to the target DNA. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target DNA, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target DNA.

In some embodiments, a seed region of a spacer may be about 8 to about 10 nucleotides in length, about 5 to about 6 nucleotides in length, or about 6 nucleotides in length.

As used herein, a “target nucleic acid”, “target DNA,” “target nucleotide sequence,” “target region,” or a “target region in the genome” refers to a region of a plant's genome that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid of this invention. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” refers to the target double stranded DNA and specifically to the portion of the target DNA (e.g., or target region in the genome) that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand (see below, as an example).

```
      5′-NNNNNNNNNNNNNNNNNNN-3′ RNA Spacer
              ||||||||||||||||||
3′-AAANNNNNNNNNNNNNNNNNNN-5′ Target strand
      ||||
5′-TTTNNNNNNNNNNNNNNNNNNN-3′ Non-target strand
```

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems (Nature Reviews Microbiology 13:722-736 (2015)). Guide structures and PAMs are described in by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

In some embodiments, the present invention provides expression cassettes and/or vectors comprising the nucleic acid constructs of the invention (e.g., one or more components of an editing system of the invention). In some embodiments, expression cassettes and/or vectors comprising the nucleic acid constructs of the invention and/or one or more guide nucleic acids may be provided. In some embodiments, a nucleic acid construct of the invention encoding a base editor (e.g., a construct comprising a CRISPR-Cas effector protein and a deaminase domain (e.g., a fusion protein)) or the components for base editing (e.g., a CRISPR-Cas effector protein fused to a peptide tag or an affinity polypeptide, a deaminase domain fused to a peptide tag or an affinity polypeptide, and/or a UGI fused to a peptide tag or an affinity polypeptide), may be comprised on the same or on a separate expression cassette or vector from that comprising the one or more guide nucleic acids. When the nucleic acid construct encoding a base editor or the components for base editing is/are comprised on separate expression cassette(s) or vector(s) from that comprising the guide nucleic acid, a target nucleic acid may be contacted with (e.g., provided with) the expression cassette(s) or vector(s) encoding the base editor or components for base editing in any order from one another and the guide nucleic acid, e.g., prior to, concurrently with, or after the expression cassette comprising the guide nucleic acid is provided (e.g., contacted with the target nucleic acid).

Fusion proteins of the invention may comprise sequence-specific nucleic acid binding domains (e.g., sequence-specific DNA binding domains), CRISPR-Cas polypeptides, and/or deaminase domains fused to peptide tags or affinity polypeptides that interact with the peptide tags, as known in the art, for use in recruiting the deaminase to the target nucleic acid. Methods of recruiting may also comprise guide nucleic acids linked to RNA recruiting motifs and deaminases fused to affinity polypeptides capable of interacting with RNA recruiting motifs, thereby recruiting the deaminase to the target nucleic acid. Alternatively, chemical interactions may be used to recruit polypeptides (e.g., deaminases) to a target nucleic acid.

A peptide tag (e.g., epitope) useful with this invention may include, but is not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. Any epitope that may be linked to a polypeptide and for which there is a corresponding affinity polypeptide that may be linked to another polypeptide may be used with this invention as a peptide tag. In some embodiments, a peptide tag may comprise 1 or 2 or more copies of a peptide tag (e.g., repeat unit, multimerized epitope (e.g., tandem repeats)) (e.g., 1, 2, 3, 4, 5, 6, 7, 8, 9, 10, 11, 12, 13, 14, 15, 16, 17, 18, 19, 20, 21, 22, 23, 24, 25 or more repeat units. In some embodiments, an affinity polypeptide that interacts with/binds to a peptide tag may be an antibody. In some embodiments, the antibody may be a scFv antibody. In some embodiments, an affinity polypeptide that binds to a peptide tag may be synthetic (e.g., evolved for affinity interaction) including, but not limited to, an affibody, an anticalin, a monobody and/or a DARPin (see, e.g., Sha et al., Protein Sci. 26 (5): 910-924 (2017)); Gilbreth (Curr Opin Struc Biol 22 (4): 413-420 (2013)), U.S. Pat. No. 9,982,053, each of which are incorporated by reference in their entireties for the teachings relevant to affibodies, anticalins, monobodies and/or DARPins. Example peptide tag sequences and their affinity polypeptides include, but are not limited to, the amino acid sequences of SEQ ID NOs: 42-44.

In some embodiments, a guide nucleic acid may be linked to an RNA recruiting motif, and a polypeptide to be recruited (e.g., a deaminase) may be fused to an affinity polypeptide that binds to the RNA recruiting motif, wherein the guide binds to the target nucleic acid and the RNA recruiting motif binds to the affinity polypeptide, thereby recruiting the polypeptide to the guide and contacting the target nucleic acid with the polypeptide (e.g., deaminase). In some embodiments, two or more polypeptides may be recruited to a guide nucleic acid, thereby contacting the target nucleic acid with two or more polypeptides (e.g., deaminases). Example RNA recruiting motifs and their affinity polypeptides include, but are not limited to, the sequences of SEQ ID NOs: 45-55.

In some embodiments, a polypeptide fused to an affinity polypeptide may be a reverse transcriptase and the guide nucleic acid may be an extended guide nucleic acid linked to an RNA recruiting motif. In some embodiments, an RNA recruiting motif may be located on the 3′ end of the extended portion of an extended guide nucleic acid (e.g., 5′-3′, repeat-spacer-extended portion (RT template-primer binding site)-RNA recruiting motif). In some embodiments, an RNA recruiting motif may be embedded in the extended portion.

In some embodiments of the invention, an extended guide RNA and/or guide RNA may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif and corresponding affinity polypeptide may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Com RNA binding protein, a PUF binding site (PBS) and the affinity polypeptide Pumilio/fem-3 mRNA binding factor (PUF), and/or a synthetic RNA-aptamer and the aptamer ligand as the corresponding affinity polypeptide. In some embodiments, the RNA recruiting motif and corresponding affinity polypeptide may be an MS2 phage operator stem-loop and the affinity polypeptide MS2 Coat Protein (MCP). In some embodiments, the RNA recruiting motif and corresponding affinity polypeptide may be a PUF binding site (PBS) and the affinity polypeptide Pumilio/fem-3 mRNA binding factor (PUF).

In some embodiments, the components for recruiting polypeptides and nucleic acids may those that function through chemical interactions that may include, but are not limited to, rapamycin-inducible dimerization of FRB-FKBP; Biotin-streptavidin; SNAP tag; Halo tag; CLIP tag; DmrA-DmrC heterodimer induced by a compound; bifunctional ligand (e.g., fusion of two protein-binding chemicals together, e.g., dihyrofolate reductase (DHFR).

In some embodiments, the nucleic acid constructs, expression cassettes or vectors of the invention that are optimized for expression in a plant may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleic acid constructs, expression cassettes or vectors comprising the same polynucleotide(s) but which have not been codon optimized for expression in a plant.

Further provided herein are cells comprising one or more polynucleotides, guide nucleic acids, nucleic acid constructs, expression cassettes or vectors of the invention.

The nucleic acid constructs of the invention (e.g., a construct comprising a sequence specific DNA binding domain, a CRISPR-Cas effector domain, a deaminase domain, reverse transcriptase (RT), RT template and/or a guide nucleic acid, etc.) and expression cassettes/vectors comprising the same may be used as an editing system of this invention for modifying target nucleic acids and/or their expression.

A functional target WOX nucleic acid (e.g., that is expressed specifically in tissues that give rise to trichomes, thorns and/or prickles; e.g., that is expressed in a tissue specific manner in the vascular tissue and/or the epidermal tissue) present in any Rosaceae plant or plant part may be modified (e.g., mutated, e.g., base edited, cleaved, nicked, etc.) as described herein to modify the architecture of thorns and/or prickles, to reduce the number and/or size of thorns and/or prickles, and/or eliminate thorns and/or prickles using the polypeptides, polynucleotides, ribonucleoproteins (RNPs), nucleic acid constructs, expression cassettes, and/or vectors of the invention. Non-limiting examples of Rosaceae plant species that may be modified as described herein may include, but are not limited to Rosa spp., Hulthemia spp., a Hesperrhodos spp., a Platyrhodon spp., or a Rubus spp. In some embodiments, the Rubus plant can be a blackberry plant, a black raspberry plant, or a red raspberry plant. In some embodiments, the Rubus spp. may be blackberry or black raspberry, optionally, wherein the Rubus spp. plant is Rubus allegheniensis (Allegheny blackberry), Rubus armeniacus (Himalayan blackberry), Rubus canadensis (Canadian blackberry), Rubus fruticosus agg. (European blackberry), Rubus laciniatus (cutleaf evergreen blackberry), Rubus pensilvanicus (Pennsylvania blackberry), Rubus trifidus (Japanese blackberry), Rubus ursinus (trailing blackberry), Rubus hybr (e.g., progeny resulting from hybridization of two or more of the above Rubus species), Rubus leucodermis (whitebark raspberry or western black raspberry), or Rubus occidentalis (black raspberry). In some embodiments, the Rubus plant can be a raspberry (red raspberry) plant including, but not limited to, Rubus crataegifolius (Asian raspberry), Rubus gunnianus (Tasmanian alpine raspberry), Rubus idaeus (red raspberry or European red raspberry), Rubus parvifolius (Australian native raspberry), Rubus phoenicolasius (wine raspberry or wineberry), Rubus rosifolius (Mauritius raspberry), Rubus strigosus (American red raspberry) (syn. R. idaeus var. strigosus), or Rubus ellipticus (yellow Himalayan raspberry). In some embodiments, the Rosa spp. is a rose plant and/or the Rosa spp. is Rosa chinensis.

The term “plant part,” as used herein, includes but is not limited to reproductive tissues (e.g., petals, sepals, stamens, pistils, receptacles, anthers, pollen, flowers, fruits, flower bud, ovules, seeds, embryos, nuts, kernels, cars, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ. In some embodiments, a plant cell can be an algal cell. A “protoplast” is an isolated plant cell without a cell wall or with only parts of the cell wall. Thus, in some embodiments of the invention, a transgenic cell comprising a nucleic acid molecule and/or nucleotide sequence of the invention is a cell of any plant or plant part including, but not limited to, a root cell, a leaf cell, a tissue culture cell, a seed cell, a flower cell, a fruit cell, a pollen cell, and the like. In some aspects of the invention, the plant part can be a plant germplasm. In some aspects, a plant cell can be non-propagating plant cell that does not regenerate into a plant.

“Plant cell culture” means cultures of plant units such as, for example, protoplasts, cell culture cells, cells in plant tissues, pollen, pollen tubes, ovules, embryo sacs, zygotes and embryos at various stages of development.

As used herein, a “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

In some embodiments of the invention, a transgenic tissue culture or transgenic plant cell culture is provided, wherein the transgenic tissue or cell culture comprises a nucleic acid molecule/nucleotide sequence of the invention. In some embodiments, transgenes may be eliminated from a plant developed from the transgenic tissue or cell by breeding of the transgenic plant with a non-transgenic plant and selecting among the progeny for the plants comprising the desired gene edit and not the transgenes used in producing the edit.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1. Editing Strategy

A strategy to generate knock-out or knock-down edits in the blackberry WOX1 gene (SEQ ID NO:69) was developed to alter the formation of thorns on any of the plant parts including stem and leaves. The blackberry variety employed in this example is tetraploid and contains two WOX1 genes, which were found to be homozygous at one genetic locus and heterozygous at the second locus. One of the four copies of the WOX1 gene comprises the nucleic acid sequence of SEQ ID NO:69 and the remaining three copies of the WOX1 gene comprises the nucleic acid sequence of SEQ ID NO:72. The WOX1 gene copies having the nucleic acid sequence of SEQ ID NO:72 were found to contain a polymorphism that leads to a non-functional protein. Based upon this analysis, only the WOX1 gene of SEQ ID NO:69 was considered a functional copy of the WOX1 gene in the blackberry variety that was selected for editing as described in this example. To generate a range of alleles of SEQ ID NO:69, multiple CRISPR guide nucleic acids comprising spacers (SEQ ID NOs: 141-142) having complementarity to the 3′ end of exon 1 within the WOX1 gene of SEQ ID NO:69 were designed and placed into the construct pWISE8806.

Lines carrying edits in the WOX1 gene were screened and those that showed about 10% of the sequencing reads having edits in the targeted gene were advanced to the next generation.

### Example 2: Edited Alleles of WOX1

Blackberry plants with edited alleles of the WOX1 gene were generated as described in Example 1 and are further described in Table 2.

Plants generated as described in Example 1 were transferred from tissue culture to plugging media and eventually transferred to a greenhouse environment. Plants were inspected for the development of prickles and glandular trichomes one month after transfer from tissue culture. At this stage in their growth, plants were sufficiently developed such that on unedited plants (or plants including a functional copy of the WOX1 gene), prickles were apparent on the cane as well as on leaf petioles and midveins.

The edited alleles of WOX1 generated in Example 1 were clustered at the 3′ end of exon 1 and all of the edits were deletions. In some of the lines, the deletion is predicted to affect the exon 1: intron 1 splice site. While not wishing to be limited by any theory, it is expected that in those alleles where the intron 1 splice site has been altered, intron 1 is deleted using a secondary splice site. It is noted that out-of-frame deletions rendered the functional copy of WOX1 non-functional. Plants comprising these out-of-frame edits displayed a thornless phenotype. In-frame deletions (including those that affect the intron 1 splice site) did not alter WOX1 activity, i.e., the functional copy remained functional and thus thorns were present on these plants.

TABLE 2
Edited blackberry plants generated with pWISE8806

| Plant Name | Phenotype | Description of Edit | Comments |
|---|---|---|---|
| CE196774 | Thornless | 10 bp deletion | Out of frame mutation that |
| | | (CAGCATCAGG; SEQ ID | also removes splice site at |
| | | NO: 157) starting at position | 3′ end of exon 1 and leads |
| | | 2626 of SEQ ID NO: 69 | to an early stop codon. |
| | | giving rise to the genomic | |
| | | sequence of SEQ ID NO: 143 | |
| CE196763 | Thornless | 6 bp deletion (CAGGGT) | Out of frame mutation that |
| | | starting at position 2632 of | also removes splice site at |
| | | SEQ ID NO: 69 giving rise to | 3′ end of exon 1 and leads |
| | | the genomic sequence of | to an early stop codon. |
| | | SEQ ID NO: 144 | |
| CE196749 | Thorny | 3 bp deletion (CAG) starting | In frame deletion |
| | | at position 2632 of SEQ ID | |
| | | NO: 69 | |
| CE196760 | Thorny | 6 bp deletion (CATCAG) | In frame deletion |
| | | starting at position 2629 of | |
| | | SEQ ID NO: 69 | |
| CE196780 | Thorny | 10 bp deletion | In frame deletion that also |
| | | (GCATCAGGGT; SEQ ID | removes splice site at 3′ |
| | | NO: 158) starting at position | end of exon 1. |
| | | 2628 of SEQ ID NO: 69 | |
| CE196762 | Thorny | 9 bp deletion | In frame deletion |
| | | (CCAGCATCA) starting at | |
| | | position 2625 of SEQ ID | |
| | | NO: 69 | |
| CE196764 | Thorny | 3 bp deletion (TCA) starting | In frame deletion |
| | | at position 2631 of SEQ ID | |
| | | NO: 69 | |

### Example 3: Knock-Out or Knock-Down Editing Strategy

A strategy to generate knock-out or knock-down edits in the blackberry WOX1 gene (SEQ ID NO:69) was developed to alter the formation of thorns on any of the plant parts including stem and leaves. The blackberry variety employed in this example is tetraploid and contains two WOX1 genes, which were found to be homozygous at one genetic locus and heterozygous at the second locus. One of the four copies of the WOX1 gene comprises the nucleic acid sequence of SEQ ID NO:69 and the remaining three copies of the WOX1 gene comprise the nucleic acid sequence of SEQ ID NO:72. The WOX1 gene copies having the nucleic acid sequence of SEQ ID NO:72 were found to contain a polymorphism that leads to a non-functional protein. To generate a range of alleles of SEQ ID NO:69, multiple CRISPR guide nucleic acids comprising spacers (SEQ ID NOs: 138-140) having complementarity to the homeodomain within the WOX1 gene of SEQ ID NO:69 were designed and placed into the construct pWISE8807.

Lines carrying edits in the WOX1 gene were screened and those that show about 10% of the sequencing reads having edits in the targeted gene were advanced to the next generation.

### Example 4: Edited Alleles of WOX1

Blackberry plants with edited alleles of the WOX1 gene were generated as described in Example 3 and are further described in Table 3.

Plants generated as described in Example 3 were transferred from tissue culture to plugging media and eventually transferred to a greenhouse environment. Plants were inspected for the development of prickles and glandular trichomes one month after transfer from tissue culture. At this stage in their growth, plants were sufficiently developed such that on unedited plants (or plants including a functional copy of the WOX1 gene), prickles were apparent on the cane as well as on leaf petioles and midveins.

The edited alleles of WOX1 generated in Example 3 were clustered in exon 2 in the homeodomain region of the WOX1 protein.

TABLE 3
Edited blackberry plants generated with pWISE8807

| Plant Name | Phenotype | Description of Edit | Comments |
|---|---|---|---|
| CE197153 | Thornless | 9 bp deletion | Out of frame mutation leading |
| | | (CATCAAGAC) starting at | to an early stop codon and a |
| | | position 2918 of SEQ ID | protein having the amino acid |
| | | NO: 69; and 1 bp deletion (C) | sequence of SEQ ID NO: 146 |
| | | at position 2961 of SEQ ID | |
| | | NO: 69 giving rise to the | |
| | | genomic sequence of SEQ ID | |
| | | NO: 145 | |
| CE197154 | Thornless | 6 bp deletion (ATCAAG) | Out of frame mutation leading |
| | | starting at position 2919 of | to an early stop codon and a |
| | | SEQ ID NO: 69; and 9 bp | protein having the amino acid |
| | | deletion (CGGCGCTGC) | sequence of SEQ ID NO: 148 |
| | | starting at position 2956 of | |
| | | SEQ ID NO: 69; and 7 bp | |
| | | deletion (GGTTTCA) starting | |
| | | at position 3007 of SEQ ID | |
| | | NO: 69 giving rise to the | |
| | | genomic sequence of SEQ ID | |
| | | NO: 147 | |
| CE197157 | Thornless | 2 bp (GC) deletion starting at | Out of frame mutation leading |
| | | position 2958 of SEQ ID | to an early stop codon and a |
| | | NO: 69; and 11 bp | protein having the amino acid |
| | | (GTTTCAGAACC; SEQ ID | sequence of SEQ ID NO: 150 |
| | | NO: 159) deletion starting at | |
| | | position 3008 of SEQ ID | |
| | | NO: 69 giving rise to the | |
| | | genomic sequence of SEQ ID | |
| | | NO: 149 | |
| CE197158 | Thornless | 5 bp deletion (AAGAC) | Out of frame mutation leading |
| | | starting at position 2922 of | to an early stop codon and a |
| | | SEQ ID NO: 69; and 5 bp | protein having the amino acid |
| | | deletion (GCGCT) starting at | sequence of SEQ ID NO: 152 |
| | | position 2958 of SEQ ID | |
| | | NO: 69; giving rise to the | |
| | | genomic sequence of SEQ ID | |
| | | NO: 151 | |
| CE197159 | Thornless | 3 bp insertion (CGG) after | Out of frame mutation leading |
| | | position 2955 of SEQ ID | to an early stop codon and a |
| | | NO: 69; and 1 bp deletion (T) | protein having the amino acid |
| | | at position 3010 of SEQ ID | sequence of SEQ ID NO: 154 |
| | | NO: 69; giving rise to the | |
| | | genomic sequence of SEQ ID | |
| | | NO: 153 | |
| CE196211 | Thornless | 8 bp deletion (CGGCGCTG) | Out of frame mutation leading |
| | | starting at position 2956 of | to an early stop codon and a |
| | | SEQ ID NO: 69 giving rise to | protein having the amino acid |
| | | the genomic sequence of SEQ | sequence of SEQ ID NO: 156 |
| | | ID NO: 155 | |

The foregoing is illustrative of the present invention and is not to be construed as limiting thereof. The invention is defined by the following claims, with equivalents of the claims to be included therein.

1. A Rosaceae plant or plant part thereof comprising at least one mutation in at least one (e.g., one or more) endogenous functional WUSCHEL (WUS)-Related Homeobox (WOX) gene encoding a WOX transcription factor, wherein the endogenous functional WOX gene: (a) comprises a nucleotide sequence having at least 80% sequence identity to any one of SEQ ID NOs: 69, 70, 75, 76, 128, 129 or 162-173; (b) comprises a region having at least 80% sequence identity to a nucleotide sequence of any one of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134; (c) encodes an amino acid sequence having at least 80% sequence identity to any one of SEQ ID NOs: 71, 77, 130, or 174-182; and/or (d) encodes a region of an amino acid sequence having at least 80% sequence identity to any one of SEQ ID NO:126, SEQ ID NO: 127, SEQ ID NO:136 or SEQ ID NO: 137, optionally wherein the at least one mutation is located in a region of the endogenous functional WOX gene, the region having at least 80% sequence identity to any one of the nucleotide sequences of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134.

2. (canceled)

3. The Rosaceae plant or part thereof of claim 1, wherein the endogenous functional WOX gene is expressed specifically in tissues that give rise to trichomes, thorns and/or prickles.

4. The Rosaceae plant or part thereof of claim 1, wherein the endogenous functional WOX gene is expressed in a tissue specific manner in the vascular tissue, the epidermal tissue and/or cells or tissues that give rise to thorns and/or prickles in the Rosaceae plant or part thereof.

5-6. (canceled)

7. The Rosaceae plant or part thereof of claim 1, wherein the at least one mutation is a base substitution, a base deletion, a base insertion and/or an inversion.

8. (canceled)

9. The Rosaceae plant or part thereof of claim 1, wherein the at least one mutation is a deletion of at least one base pair, optionally one base pair to about 100 consecutive base pairs.

10-11. (canceled)

12. The Rosaceae plant or part thereof of claim 1, wherein the at least one mutation is an out-of-frame insertion, in-frame deletion or an out-of-frame deletion.

13-15. (canceled)

16. The Rosaceae plant or part thereof of claim 1, wherein the Rosaceae plant or plant part thereof comprises at least one mutation in two or more endogenous functional WOX genes.

17-18. (canceled)

19. The Rosaceae plant or part thereof of claim 1, wherein the Rosaceae plant comprising the at least one mutation exhibits a reduced number of thorns and/or prickles, thorns and/or prickles that have a modified architecture, thorns and/or prickles that are reduced in size, and/or no thorns and/or prickles when compared to a control Rosaceae plant devoid of the at least one mutation.

20-22. (canceled)

23. The Rosaceae plant or part thereof of claim 1, wherein the at least one mutation results in a mutated WOX gene comprising a nucleotide sequence having at least 90% sequence identity to any one of SEQ ID NOs: 143, 144, 145, 147, 149, 151, 153 and/or 155 or encoding a WOX transcription factor having at least 90% sequence identity to any one of SEQ ID NOs: 146, 148, 150, 152, 154 and/or 156.

24-31. (canceled)

32. A Rosaceae plant regenerated from the plant part of claim 1 and exhibiting a phenotype of a reduced number of thorns and/or prickles, thorns and/or prickles that have a modified architecture, thorns and/or prickles that are reduced in size, and/or no thorns and/or prickles when compared to a control Rosaceae plant devoid of the at least one mutation.

33. The Rosaceae plant of claim 32, wherein the Rosaceae plant comprises a mutated WOX gene comprising a nucleotide sequence having at least 90% sequence identity to any one of SEQ ID NOs: 143, 144, 145, 147, 149, 151, 153 and/or 155 and/or encodes a WOX1 transcription factor having at least 90% sequence identity to any one of SEQ ID NOs: 146, 148, 150, 152, 154 and/or 156.

34-77. (canceled)

78. A method for producing a Rosaceae plant or part thereof comprising at least one cell having a mutated endogenous WUSCHEL (WUS)-Related Homeobox (WOX) gene, the method comprising
contacting a target site within an endogenous functional WOX gene in the Rosaceae plant or plant part with a nuclease comprising a cleavage domain and a nucleic acid binding domain, wherein the nucleic acid binding domain binds to a target site within the endogenous functional WOX gene, wherein the endogenous functional WOX gene:
(a) comprises a nucleotide sequence having at least 80% sequence identity to any one of SEQ ID NOs: 69, 70, 75, 76, 128, 129 or 162-173;
(b) comprises a region having at least 80% sequence identity to a nucleotide sequence of any one of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134;
(c) encodes an amino acid sequence having at least 80% sequence identity to any one of SEQ ID NOs: 71, 77, 130, or 174-182; and/or
(d) encodes a region of an amino acid sequence having at least 80% sequence identity to any one of SEQ ID NOs: 126, 127, 136 or 137, thereby producing the Rosaceae plant or part thereof comprising at least one cell having a mutation in the endogenous WOX gene.

79-80. (canceled)

81. The method of claim 78, wherein the endogenous functional WOX gene is expressed specifically in tissues that give rise to trichomes, thorns, spines and/or prickles.

82. The method of claim 78, wherein the endogenous functional WOX gene is expressed in a tissue specific manner in the vascular tissue, the epidermal tissue and/or cells or tissues that give rise to thorns and/or prickles.

83-84. (canceled)

85. The method of claim 78, wherein the mutation is a substitution, an insertion, a deletion and/or an inversion.

86-87. (canceled)

88. The method of claim 78, wherein the mutation is an insertion or a deletion of about one base pair to about 100 base pairs.

89. (canceled)

90. The method of claim 78, wherein the mutation is an out-of-frame insertion, in-frame deletion, or an out-of-frame deletion, optionally resulting in a premature stop codon.

91-92. (canceled)

93. The method of claim 78, wherein the mutated WOX gene comprises a nucleotide sequence having at least 90% sequence identity to any one of SEQ ID NOs: 143, 144, 145, 147, 149, 151, 153 and/or 155 or encodes a polypeptide having at least 90% identity to any one of SEQ ID NOs: 146, 148, 150, 152, 154 and/or 156.

94-98. (canceled)

99. A Rosaceae plant produced by the method of claim 78.

100-124. (canceled)

125. A modified a WUSCHEL (WUS)-Related Homeobox (WOX) transcription factor polypeptide comprising a sequence having at least 90% sequence identity to the amino acid sequence of any one of SEQ ID NOs: 117, 119, 121, 123, 125, 127, 129, 131, 133, and/or 135.

126. A nucleic acid encoding the modified WUSCHEL (WUS)-Related Homeobox (WOX) transcription factor polypeptide of claim 125.

127. The nucleic acid of claim 126, wherein the nucleic acid comprises a mutated WOX gene having at least 90% sequence identity to a nucleotide sequence of any one of SEQ ID NOs: 143, 144, 145, 147, 149, 151, 153 and/or 155.

128. A Rosaceae plant or part thereof comprising the nucleic acid of claim 126.

129. A plant genome or plant genomic DNA comprising a nucleotide sequence encoding the modified WUSCHEL (WUS)-Related Homeobox (WOX) transcription factor polypeptide of claim 125, wherein the nucleotide sequence is any one of SEQ ID NOs: 143, 144, 145, 147, 149, 151, 153 and/or 155.

130. A guide nucleic acid that binds to a target site within a functional WUSCHEL (WUS)-Related Homeobox (WOX) gene, wherein the target site is in a region of the functional WOX gene having at least 80% sequence identity to any one of SEQ ID NOs: 78-82, 83-86, 87-90, 91-109, 110-125 or 131-135, optionally SEQ ID NOs: 81, 82, 84-86, 88-90, 94-97, 99-101, 103-105, 107-109, 113-116, 118-120, 122, 124, 125 or 134.


SEQUENCE LISTING (182 sequences)

SEQ ID NO: 1 - protein, 1228 aa, unidentified
  source 1..1228: note = Lachnospiraceae sp.
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 1241 aa, Butyrivibrio proteoclasticus
MLLYENYTKRNQITKSLRLELRPQGKTLRNIKELNLLEQDKAIYALLERLKPVIDEGIKDIARDTLKNCELSFEKLYEHFLSGDKKAYAKESERLKKEIVKTLIKNLPEGIGKISEINSAKYLNGVLYDFIDKTHKDSEEKQNILSDILETKGYLALFSKFLTSRITTLEQSMPKRVIENFEIYAANIPKMQDALERGAVSFAIEYESICSVDYYNQILSQEDIDSYNRLISGIMDEDGAKEKGINQTISEKNIKIKSEHLEEKPFRILKQLHKQILEEREKAFTIDHIDSDEEVVQVTKEAFEQTKEQWENIKKINGFYAKDPGDITLFIVVGPNQTHVLSQLIYGEHDRIRLLLEEYEKNTLEVLPRRTKSEDARYDKFVNAVPKKVAKESHTFDGLQKMTGDDRLFILYRDELARNYMRIKEAYGTFERDILKSRRGIKGNRDVQESLVSFYDELTKFRSALRIINSGNDEKADPIFYNTFDGIFEKANRTYKAENLCRNYVTKSPADDARIMASCLGTPARLRTHWWNGEENFAINDVAMIRRGDEYYYFVLTPDVKPVDLKTKDETDAQIFVQRKGAKSFLGLPKALFKCILEPYFESPEHKNDKNCVIEEYVSKPLTIDRRAYDIFKNGTFKKTNIGIDGLTEEKFKDDCRYLIDVYKEFIAVYTRYSCFNMSGLKRADEYNDIGEFFSDVDTRLCTMEWIPVSFERINDMVDKKEGLLFLVRSMFLYNRPRKPYERTFIQLFSDSNMEHTSMLLNSRAMIQYRAASLPRRVTHKKGSILVALRDSNGEHIPMHIREAIYKMKNNFDISSEDFIMAKAYLAEHDVAIKKANEDIIRNRRYTEDKFFLSLSYTKNADISARTLDYINDKVEEDTQDSRMAVIVTRNLKDLTYVAVVDEKNNVLEEKSLNEIDGVNYRELLKERTKIKYHDKTRLWQYDVSSKGLKEAYVELAVTQISKLATKYNAVVVVESMSSTFKDKFSFLDEQIFKAFEARLCARMSDLSFNTIKEGEAGSISNPIQVSNNNGNSYQDGVIYFLNNAYTRTLCPDTGFVDVFDKTRLITMQSKRQFFAKMKDIRIDDGEMLFTFNLEEYPTKRLLDRKEWTVKIAGDGSYFDKDKGEYVYVNDIVREQIIPALLEDKAVFDGNMAEKFLDKTAISGKSVELIYKWFANALYGIITKKDGEKIYRSPITGTEIDVSKNTTYNFGKKFMFKQEYRGDGDFLDAFLNYMQAQDIAV

SEQ ID NO: 4 - protein, 1238 aa, Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDIVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIDRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLRPVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 5 - protein, 1281 aa, Eubacterium eligens
MNGNRSIVYREFVGVIPVAKTLRNELRPVGHTQEHIIQNGLIQEDELRQEKSTELKNIMDDYYREYIDKSLSGVTDLDFTLLFELMNLVQSSPSKDNKKALEKEQSKMREQICTHLQSDSNYKNIFNAKLLKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDDHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYSDISEFYREVEMQGYRIDWTYISEADINKLDEEGKIYLFQIYNKDFAENSTGKENLHTMYFKNIFSEENLDKIIKLNGQAELFYRRASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMVVKYIAQNDDIHVIGIDRGERNLIYISVIDSHGNIVKQKSYNILNNYDYKKKLVEKEKTREYARKNWKSIGNIKELKEGYISGVVHEIAMLIVEYNAIIAMEDLNYGFKRGRFKVERQVYQKFESMLINKLNYFASKEKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDIRIDMEKMDEDKKSEFFAQLLSLYKLTVQMRNSYTEAEEQENGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 6 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKVNYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 7 - protein, 1206 aa, unidentified
  REGION 1..1206: note = Lachnospiraceae sp.
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSVIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFDTQASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGLYLQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 8 - protein, 1233 aa, unidentified
  source 1..1233: note = Lachnospiraceae sp.
MDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKKLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 9 - protein, 1227 aa, unidentified
  source 1..1227: note = Lachnospiraceae sp.
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIANINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIASNKEWLEYAQTSVKH

SEQ ID NO: 10 - protein, 1264 aa, Leptospira inadai
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYNKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNPLNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDISGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 11 - protein, 1373 aa, Moraxella bovoculi
MLFQDFTHLYPLSKTVRFELFIDRTLEHIHAKNFLSQDETMADMHQKVKVILDDYHRDFIADMMGEVKLTKLAEFYDVYLKFRKNPKDDELQKAQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFKDINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQKDFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIQASQAFFGKFDKICYNADKDYFEFHIDYAKFTDKAKNSRQIWTICSHGDKRYVYDKTANQNKGAAKGINVNDILKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 12 - protein, 1352 aa, unidentified
  source 1..1352: note = Parcubacteria bacterium
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITESKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKIKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 13 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 14 - protein, 1324 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIDALKGFYIEDGFENNVLTPLATLLSSLDKYNLNGIFIRNNEALSSLSQNVYRNFSIDEAIDAQNAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNKDDLNLSISSTEWLDFVREKPYLK

SEQ ID NO: 15 - protein, 1484 aa, unidentified
  VARIANT 1073: note = Xaa can be any naturally occurring amino acid
  source 1..1484: note = Peregrinibacteria bacterium
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAMIKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTWIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDXWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKKKRPAATKK

SEQ ID NO: 16 - protein, 1245 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSISEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQHGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 17 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYQWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 18 - DNA, 3987 bp, synthetic construct
  source 1..3987: mol_type = other DNA
atggccgggagcaagaagcgccggataaagcaggacacgcagttcgagggcttcaccaacctgtaccaagtctccaagacgctccggttcgagcttatcccgcaagggaagaccctgaaacacatccaggaacaaggtttcatcgaggaggacaaggcccgcaacgaccactacaaggagctcaagcccataatcgatcggatctacaagacgtacgccgaccagtgcctccaactggtgcagctcgactgggagaacctgagcgccgccattgacagctaccgcaaggaaaagacggaggagacgcgcaacgcccttattgaggagcaagccacctaccgcaacgccatccacgactacttcatcgggcgcaccgacaacctgacggacgcgatcaacaagcgccacgcggaaatctacaagggccttttcaaggccgagctcttcaacgggaaggtcctaaaacagctcgggactgtcacgacaaccgagcatgagaacgccctccttcgcagcttcgacaagttcaccacatacttctcgggcttctaccggaaccgcaagaacgttttcagcgccgaggacatctccaccgccatcccgcacaggatcgtccaggacaacttccccaagttcaaggagaactgccacatcttcacgcgcctgattacagccgtaccttcacttcgtgagcacttcgagaacgtcaaaaaggccatcgggatcttcgtctccacgtccatcgaggaggtattctctttcccgttctataaccagctcctgacccagacgcagatcgacctctacaaccagctactgggcggcatcagccgggaggccgggaccgagaaaataaagggcctcaacgaagttctcaacctggccatccagaagaacgacgagaccgcgcatatcatcgcatccctgccgcatcgcttcattcctttgttcaagcagatattgagcgaccggaacaccctctcgttcatcctcgaagaattcaagagcgacgaggaggtcattcagtctttctgcaagtacaagacgctcctacggaatgagaatgtgctggagaccgcggaggcactcttcaatgagctgaactccattgacctgacccacatcttcattagccacaagaaactggagacgatctccagcgccctgtgcgaccactgggacactctccgcaacgccctctacgaacgccggatctccgaacttaccggcaagataactaagtcggctaaggagaaggtgcaacggagcctcaagcacgaggacatcaaccttcaggaaatcatctcagccgcgggcaaggagctgagcgaggcgtttaagcagaaaacatcggagatactgagccacgcgcacgcggccctggatcaaccgctgccgacgactctcaagaagcaagaggagaaggaaatccttaagtcccagctcgactcgctgctcggcctctatcacttgctcgactggttcgcggttgatgagtccaacgaggtggacccggagttctccgcgcgcctcacgggtattaagctggagatggagccaagcttaagcttctacaacaaggcccgcaactacgcgaccaaaaaaccgtactcagtcgagaaattcaagctgaatttccagatgcctacattggcgagggggtgggacgtgaaccgcgagaagaacaatggagccatcctgttcgtcaaaaatgggttgtactacctgggcatcatgcccaagcagaagggccgttacaaggccctgtcattcgagcctaccgagaagacctcggagggcttcgacaagatgtactacgactatttcccggacgccgccaagatgatcccgaagtgctccacgcagctcaaagccgtcacggcccacttccagacgcataccacgccgatacttctgagcaacaacttcattgagccgctagagatcacgaaggagatatacgacctaaacaaccccgaaaaggagcccaagaagttccagacagcctacgctaagaagacaggtgatcagaagggatatagggaggcactctgcaagtggatcgacttcacgcgcgacttcctgtcgaaatatacaaagacgaccagcattgacctaagttctctccgcccatcctcccagtacaaggatctgggcgagtattatgcggagctgaacccattgctgtaccacatcagcttccagaggatcgccgagaaggagattatggacgcggtggagacggggaaactatacctgttccaaatatataacaaggacttcgctaaagggcaccacgggaagcccaacctgcacacactctactggacgggcttgttttcgccagaaaatttggccaagacttcgatcaagctcaacggccaggcggagttgttttaccgtcccaagtctcgcatgaagcgcatggcgcatcgcctcggagagaaaatgcttaacaagaagctcaaggatcagaagacgcccatacctgatacgttgtaccaggaattgtacgactacgtgaaccaccgcctatcgcacgacctctcagacgaggcccgcgccctcctcccaaacgtgattactaaggaggtttcccatgaaataatcaaggaccgacggttcaccagcgacaaattttttttccacgtgcctatcacgctcaattaccaggcggccaactccccatcgaagttcaaccagcgcgtgaacgcctaccttaaggagcacccggagaccccaatcatcgggatcgaccgtggcgagcggaacctgatctatattacggtgatcgatagcaccgggaagatcctggagcagcgctccctgaacacaatccagcagtttgactaccagaagaaactcgacaaccgggagaaggagcgcgtcgcagcccggcaagcatggagtgtggtcggcaccataaaggacctgaaacagggttacctaagtcaagttatccacgagatcgttgacctgatgatacactatcaagccgtagtcgtgctggagaacctcaacttcgggtttaagtccaagcgcaccggcatcgcggagaaggcggtgtaccagcagttcgagaagatgctgatcgacaagctgaactgcctggtgctcaaggactaccctgcggagaaggtcggcggggtcttgaacccgtaccagctaaccgaccagttcacgagcttcgccaaaatgggcacgcagtccggattcttgttttatgtcccggctccatatacaagtaagatcgacccgctgacagggtttgttgacccattcgtgtggaagaccatcaagaaccacgagagcaggaaacacttcttagagggcttcgacttcctgcattacgacgttaagacaggcgacttcatcctgcacttcaagatgaaccgcaacctgtcgttccagaggggcctgcccggcttcatgcccgcctgggatatcgtctttgagaagaatgagacgcagttcgacgcgaaggggacgccgttcatcgctggaaagcggatcgtgccggtcatcgagaaccaccgcttcacgggtcgctaccgagatttataccccgccaacgaactaattgcgctgctggaggagaaggggatcgtgttccgagatggcagcaacattctcccgaagctgctggagaacgacgactcgcacgctattgacacgatggtcgccctcatacggagcgtgcttcagatgcggaacagtaacgctgccacgggcgaggactacattaactcccccgtccgcgacctcaacggggtctgcttcgatagccgcttccagaacccggagtggcctatggatgcggacgcgaacggggcctaccacatcgccctcaagggccaactcctgctcaaccacttgaaggaaagcaaagacctcaaattgcagaatggcatcagtaaccaggactggctcgcgtacatccaggaactgagaaacgggtccaagaagcggcgtatcaagcaagattga

SEQ ID NO: 19 - DNA, 3987 bp, synthetic construct
  source 1..3987: mol_type = other DNA
atggcgggaagcaaaaagcgccggattaagcaagacacgcagttcgagggcttcacgaacctctaccaagtcagcaagaccctccggttcgagctgataccacagggaaagacgctcaagcacatccaggaacagggcttcatcgaggaggacaaggcgcgcaacgaccactacaaggagttgaaaccgatcatcgaccgcatctacaagacgtacgccgaccagtgcctccagctcgtgcagctcgactgggagaacctctccgccgccattgactcgtaccggaaggagaagactgaggagacccgcaacgccctgatcgaggagcaagcaacctaccggaacgccatccacgactacttcatcggccgcaccgacaacctcaccgacgcgatcaacaagcggcacgcggagatatacaaagggctgttcaaggcggagctgttcaacggcaaggtgctcaagcagctagggacggtgaccacgaccgagcacgagaacgcgctcctccgcagcttcgacaagttcaccacctacttcagcggcttctaccggaaccgcaagaatgtgttcagcgcggaggacatcagcacggccatcccgcaccgcatcgtccaggacaacttcccgaagttcaaggagaactgccacatcttcacccgcctgataaccgccgtcccctccctgcgggagcacttcgagaacgtcaaaaaggcaattgggatcttcgtctcgaccagcattgaggaggtgttcagcttccccttctacaaccagctcctcacccagacgcagatcgacctgtacaatcagttgctcggcgggataagccgcgaggcgggaaccgaaaaaatcaaggggctgaacgaagtgttgaacctcgccatccagaagaacgacgagaccgcgcacatcatcgcctccctgccccaccggttcatcccgctgttcaagcagatcctctctgaccggaacaccctgtccttcattcttgaggagttcaagtcggacgaggaggtcatccagagcttctgcaagtacaagacgctgctacggaacgagaacgtgctggagacggcggaggcactgttcaacgagctaaacagcatcgacctcacgcacatcttcatcagtcacaagaaactggagaccatctcctccgcgctgtgcgaccactgggacacgctcaggaacgcgctctacgagcgccgaatcagtgagctgacgggcaagatcacgaagtccgcgaaggagaaggtgcagcggtccctcaagcacgaggacatcaacctccaggagatcatctcagcggctgggaaagagctgtccgaggcgttcaagcagaaaacgagcgaaatcctgtcccacgcgcacgcggccctggatcagcctctgccgacgaccctcaagaaacaagaagaaaaggaaatcctcaagtcgcagctcgactcgctgctgggcctgtaccatctcctcgactggttcgccgtggacgagagcaacgaggtggaccccgagttctccgcgcggcttacggggatcaagctggagatggagcccagcctgtccttctacaacaaggcgcgcaactacgccaccaagaagccctacagcgtggagaagttcaagctcaacttccagatgcccactctcgcacgtgggtgggacgtcaaccgcgaaaaaaataatggggcgatcctgttcgtcaagaacggcctgtactacttgggcatcatgccgaaacagaagggccgctacaaggccctgagcttcgaaccgaccgagaaaacgagcgaggggttcgacaagatgtactacgactacttccccgacgccgcgaagatgattccaaagtgctccacgcagcttaaggccgtgacggcccacttccagacgcacacgaccccgatcctcctcagcaacaacttcatcgagcccctggagatcacgaaggagatatacgacctgaacaacccggagaaggagcccaagaaattccagaccgcctacgccaagaagacaggcgaccaaaagggttacagggaggccctctgcaagtggatcgacttcactagggacttcctgtccaagtacaccaagactacctctatcgacctgtccagcctccgcccgtcgtcccagtacaaagatttgggcgagtattacgcggagctgaacccactgctctaccacatcagcttccagcgcatcgcggagaaggagatcatggacgcagtggagacgggcaagctatacctatttcagatatacaacaaagacttcgctaagggacaccacggcaagcctaacctgcacaccctctactggacggggctcttcagcccggagaacctcgccaagacctcgatcaagctcaacggccaggccgagctgttctaccggcccaagtcccgcatgaagcggatggcccaccggctcggggagaaaatgctcaacaagaaattgaaggaccaaaaaacgccgatacccgacaccctataccaggagctgtacgactatgtgaaccaccgcctgagccacgacctcagcgacgaggcgcgggccctcctgccgaacgtcatcacaaaggaggtcagccacgagatcatcaaggaccggcgcttcacctccgacaagtttttctttcacgtgcccatcacgctcaactaccaggccgccaactcgccgtccaagttcaaccagcgcgtgaacgcctacctcaaggagcaccccgagaccccgatcatcgggattgaccgaggggagcggaacctcatctacatcaccgtcatcgacagcaccgggaagatccttgaacagcggtcgctcaacaccatccagcagttcgactaccagaagaaactcgacaaccgggagaaggagagagtggcggcccgccaggcttggtccgtcgtcgggacgattaaggacttgaaacaaggttacctgtcgcaagtgatccacgagatcgttgacctgatgatccactaccaagccgtcgtggtcctggagaacctcaacttcggcttcaagagcaaacgaaccggcatcgcggagaaggccgtgtaccagcagttcgaaaaaatgctgatcgacaagctgaactgcctcgtgctcaaggactaccccgctgagaaggtcggcggggtgctgaacccgtaccagctcactgaccagttcaccagcttcgcaaagatgggcacccagtccggcttcctgttctacgtgcctgcgccatacacctcgaagatcgacccgctcaccgggttcgtggaccccttcgtctggaagaccatcaagaaccacgagagccgcaagcacttcctggagggcttcgacttcctccactacgacgtcaagaccggggacttcatcctgcacttcaagatgaaccgcaacctcagtttccagcgcggcctgccggggttcatgcccgcttgggatatagtcttcgagaagaatgagacgcagttcgacgcgaagggcaccccgttcatcgccgggaagcgcatcgtgccggtcatcgagaaccaccggttcaccgggcgctaccgcgacctatacccggcgaacgagttgatcgccctcctggaggagaagggcatcgtgttccgcgacggctccaacatcctcccgaagctgctcgaaaacgacgactcccacgccatcgacacgatggtcgcgctgatccggtcggtgctccagatgcggaactccaacgccgcgacgggcgaggactacatcaacagtccggtccgcgatctgaacggcgtctgcttcgactcccggttccagaaccccgagtggccgatggacgcggacgcgaacggcgcataccacatcgccctaaaagggcaattgctgctcaaccacctcaaggaatccaaagacctaaagctccagaacggcatctccaaccaggactggctggcgtacatccaggaactgcggaacgggagcaaaaaacgtcggatcaagcaagattga

SEQ ID NO: 20 - DNA, 3987 bp, synthetic construct
  source 1..3987: mol_type = other DNA
atggcgggctccaagaaacgccggattaagcaagatacccagttcgaggggttcacgaacctctaccaagtgagcaagaccctccgattcgaactgattcctcaggggaagaccctcaagcacatccaggagcaagggttcatcgaggaggacaaggcgcggaacgaccactacaaggaactcaaacccatcatcgaccgcatctacaagacctacgccgatcagtgcctccagctcgtgcagttggactgggagaacctcagcgcggccattgactcctaccggaaggagaaaacggaggagacgcgcaacgcgctcatcgaggaacaggcaacctatcgcaacgccatccacgactacttcatcgggaggactgacaacctcactgacgcgattaacaagcgccacgcggagatatacaagggactcttcaaagcggagctgtttaacggcaaggttctcaagcaactcggcactgtgaccacgaccgagcatgagaacgccctgctccgctccttcgacaagttcaccacctacttctccgggttctaccgcaaccgcaagaatgtcttcagcgcggaggacatcagcacggccattccacatcgaatcgtccaagataacttcccgaagttcaaggagaactgccacatcttcacccgactcattactgctgtaccgtcgttacgcgaacacttcgagaacgtcaagaaggcaattggaatcttcgtctctacgtcaatagaggaggtgttcagcttccctttctacaaccagctccttacgcagacccagatagacctgtacaatcagctcctcggtgggatcagccgggaggcggggactgagaagattaaagggctcaacgaggtcttgaacctggccatccaaaaaaacgatgagacggcgcacatcatcgcctcgctgccccaccggttcatcccgctgttcaagcagatcctcagtgacaggaacaccttgagctttatcctagaggagttcaagagcgacgaggaggtgatccagagcttctgcaagtacaaaaccctgctgaggaacgagaacgtcctggagacggcggaggcgctgttcaacgagctgaactctatcgacttaactcacatattcatctcgcacaagaagctggagactattagctctgcactctgcgaccactgggacaccctccgcaacgcgctctacgagcgccgcatctcggagctgaccgggaagatcaccaaatccgcgaaggaaaaggtccagcgttccctcaaacacgaggatattaacttacaggagattatctcagcggctgggaaggagttgtcagaggcgttcaagcagaaaacttccgagatcctgagccacgcgcacgcagcgctcgaccagcctctgcccaccaccctcaaaaagcaggaagaaaaagagatcctcaagagccagttggactccctgctggggctctatcaccttctcgactggttcgccgtcgatgagtcgaacgaggtggaccccgagttctccgcccggctgaccggcatcaagctagagatggagccgtccctcagcttctacaataaggcccgcaactacgcgaccaaaaaaccctacagcgtggagaagttcaagctgaacttccagatgccgaccttagcacgcggttgggacgtaaacagggagaagaacaatggagccatcctgttcgtcaagaacgggctttactacctcgggataatgcccaagcagaagggccgctacaaggccctttccttcgagccgacggagaaaacctccgaggggttcgacaagatgtactacgactacttccccgacgccgccaagatgatcccgaagtgctcaacgcagctaaaagccgtgaccgcccacttccagacccacacgacgccgatcctgctgagcaacaacttcatcgagccccttgagatcactaaggagatatacgacctgaacaaccccgagaaggagcccaagaagtttcaaaccgcctacgccaaaaaaactggcgaccaaaagggctacagggaggcgctgtgtaagtggatcgacttcacacgcgacttcctttcgaagtatacgaagacaacctctattgacctgagcagcctgcgtcctagctcccagtacaaagatttgggcgagtactacgcggagcttaatccactactctaccacatctcattccagcgcatcgctgagaaggaaatcatggacgcggtggagacaggcaaactgtacctcttccagatatacaacaaagacttcgctaaggggcaccacgggaagcccaaccttcatacgctctactggacgggcctattcagccccgaaaatctggccaagacctccatcaagctgaacggccaagcggagctgttctacagacccaagagccggatgaagcggatggcccacaggctcggcgagaaaatgcttaacaaaaagttgaaggaccagaaaacccctatccccgacaccctctaccaggaactgtacgactacgtgaaccacaggctctcgcacgacctttccgacgaggcccgtgccctactcccgaacgtcattaccaaagaggtttcgcacgagatcatcaaggaccggcggttcacgagcgacaagtttttctttcacgtccccatcacccttaactaccaggcggccaactccccatccaagttcaaccagcgtgtgaatgcctacctcaaggagcacccagagaccccgatcattgggatcgaccggggcgagcggaacctgatctacatcaccgtcatcgactcgacgggcaagattcttgagcagagatcgttgaataccatacagcagttcgactaccagaagaaactcgacaaccgcgagaaggagcgcgtggcggcccgccaggcgtggtccgtcgttgggacgattaaggacttgaaacaaggttatctgtcccaagtcatccacgagatcgttgatctgatgatccactatcaggcagtggtggtgctggagaatctcaacttcggcttcaagagtaagcggacgggaatcgccgagaaggccgtgtaccagcagttcgagaagatgctgatcgacaagctcaactgccttgtgctgaaagactacccggccgagaaggtcggcggcgtcctcaacccgtaccaacttaccgaccagttcacctccttcgccaagatgggcactcagtccgggttcttgttctacgtccccgcaccttacacctctaagatcgaccctctgactggcttcgtagatccattcgtgtggaagaccattaagaaccacgagagccgcaagcacttcctggagggcttcgacttcctgcactacgacgtgaagaccggggacttcatccttcacttcaagatgaaccggaacctcagcttccagcggggcctgccggggttcatgcccgcctgggacatcgtgttcgagaagaacgagacccagttcgacgcgaagggcacgcccttcatcgccgggaagcgtatcgtgccggtgatcgagaaccatcgtttcacgggtcgctaccgtgacctctacccggcgaacgagcttatcgcactcctggaggagaagggcatcgtcttccgggacggctccaacatcctcccgaaactgctggaaaacgacgactctcacgccatcgacacgatggtggccctcatccggtccgtgctccaaatgcggaacagcaacgccgccaccggtgaggactacatcaacagcccggtccgggatctgaacggggtgtgcttcgattcgcggttccagaatcctgagtggccgatggacgcggatgcaaacggggcgtaccacatcgcgctcaagggccagttacttctgaaccaccttaaggagtctaaagatttgaaactccagaacgggatctcgaaccaggactggctggcctacatccaagagttgcggaacggcagcaagaagcggcggattaagcaagattag

SEQ ID NO: 21 - DNA, 1592 bp, Medicago truncatula
  source 1..1592: mol_type = other DNA
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacttactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagaggtgccgttggcgtaatataacagccaatcgatttttgctataaaagcaaatcaggtaaactaaacttcttcattcttttcttccccatcgctacaaaaccggttcctttggaaaagagattcattcaaacctagcacccaattccgtttcaaggtataatctactttctattcttcgattattttattattattagctactatcgtttaatcgatcttttcttttgatccgtcaaatttaaattcaattagggttttgttcttttctttcatctgattgaaatccttctgaattgaaccgtttacttgattttactgtttattgtatgatttaatcctttgtttttcaaagacagtctttagattgtgattaggggttcatataaatttttagatttggatttttgtattgtatgattcaaaaaatacgtcctttaattagattagtacatggatattttttacccgatttattgattgtcagggagaatttgatgagcaagtttttttgatgtctgttgtaaattgaattgattataattgctgatctgctgcttccagttttcataacccatattcttttaaccttgttgtacacacaatgaaaaattggtgattgattcatttgtttttctttgttttggattatacagg

SEQ ID NO: 22 - DNA, 2000 bp, Zea mays
  source 1..2000: mol_type = other DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttc

SEQ ID NO: 23 - protein, 228 aa, Rattus norvegicus
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 24 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 25 - DNA, 621 bp, Petromyzon marinus
  source 1..621: mol_type = other DNA
acagatgcagagtatgtgagaattcacgaaaagctggacatctataccttcaagaagcagttctttaacaataagaagtctgtgagccataggtgctacgtgctgttcgagctgaagagaaggggtgaaagaagggcatgtttttgggggtatgctgtgaacaagccccagtctggaactgagagaggcattcacgccgaaattttcagcatcagaaaggtggaggaatacctgagggataaccctggacagtttacaattaattggtattctagctggtctccatgcgctgactgtgccgagaagatcctggaatggtacaaccaggagctgagaggaaatggccataccctgaagatttgggcctgcaagctgtactatgaaaagaacgcaagaaatcagatcggactgtggaacctgagggataatggtgtggggctgaacgtgatggtgtccgagcactatcagtgctgtagaaagattttcattcagtcctcacataatcagctgaacgagaatagatggctggaaaagactctgaagagggctgagaagagaaggtccgaactgtcaattatgatccaggtgaagatcctgcacaccactaagtcacctgccgtg

SEQ ID NO: 26 - protein, 160 aa, synthetic construct
FERNYDPRELRKETYLLYEIKWGKSGKLWRHWCQNNRTQHAEVYFLENIFNARRFNPSTHCSITWYLSWSPCAECSQKIVDFLKEHPNVLEIYVARLYYHEDERNRQGLRDLVNSGVTIRIMDLPDYNYCWKTFVSDQGGDEDYWPGHFAPWIKQYSLKL

SEQ ID NO: 27 - protein, 207 aa, synthetic construct
TDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAV

SEQ ID NO: 28 - protein, 228 aa, synthetic construct
SSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLK

SEQ ID NO: 29 - protein, 162 aa, synthetic construct
SFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 30 - protein, 166 aa, Escherichia coli
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 31 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 32 - protein, 166 aa, synthetic construct
SEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 33 - protein, 166 aa, synthetic construct
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMRRQVFNAQKKAQSSTD

SEQ ID NO: 34 - protein, 166 aa, synthetic construct
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 35 - protein, 1763 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 36 - protein, 1565 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 37 - protein, 1565 aa, synthetic construct
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 38 - protein, 364 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 39 - protein, 167 aa, synthetic construct
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 40 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 41 - protein, 83 aa, unidentified
  source 1..83: note = Bacillus phage AR9
TNLSDIIEKETGKQLVIQESILMLPEEVEEVIGNKPESDILVHTAYDESTDENVMLLTSDAPEYKPWALVIQDSNGENKIKML

SEQ ID NO: 42 - protein, 24 aa, synthetic construct
EELLSKNYHLENEVARLKKGSGSG

SEQ ID NO: 43 - protein, 241 aa, synthetic construct
EEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSG

SEQ ID NO: 44 - protein, 277 aa, synthetic construct
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGS

SEQ ID NO: 45 - DNA, 66 bp, Saccharomyces bayanus
  source 1..66: mol_type = other DNA
ttcttgtcgtacttatagatcgctacgttatttcaattttgaaaatctgagtcctgggagtgcgga

SEQ ID NO: 46 - protein, 605 aa, Homo sapiens
MSGWESYYKTEGDEEAEEEQEENLEASGDYKYSGRDSLIFLVDASKAMFESQSEDELTPFDMSIQCIQSVYISKIISSDRDLLAWFYGTEKDKNSVNFKIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPLKEACRAYGLKSGLKKQELLEALTKHFQD

SEQ ID NO: 47 - protein, 482 aa, synthetic construct
MVRSGNKAAWLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTTWDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDIWAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 48 - DNA, 10 bp, Methanobacterium thermoautotrophicum
  source 1..10: mol_type = other DNA
aatttttgga

SEQ ID NO: 49 - protein, 83 aa, Methanobacterium thermoautotrophicum
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 50 - DNA, 25 bp, Emesvirus zinderi
  source 1..25: isolate = Escherichia phage MS2; mol_type = other DNA
gcgcacatgaggatcacccatgtgc

SEQ ID NO: 51 - protein, 116 aa, Emesvirus zinderi
  source 1..116: note = Escherichia phage MS2
MASNFTQFVLVDNGGTGDVTVAPSNFANGIAEISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 52 - DNA, 26 bp, Pepevirus rubrum
  source 1..26: isolate = Bacteriophage PP7; mol_type = other DNA
ataaggagtttatatggaaaccctta

SEQ ID NO: 53 - protein, 127 aa, Pepevirus rubrum
  source 1..127: note = Bacteriophage PP7
MSKTIVLSVGEATRTLTEIQSTADRQIFEEKVGPLVGRLRLTASLRQNGAKTAYRVNLKLDQADWDCSTSVCGELPKVRYTQVWSHDVTIVANSTEASRKSLYDLTKSLVATSQVEDLVVNLVPLGR

SEQ ID NO: 54 - DNA, 19 bp, unidentified
  misc_feature 1..19: note = Shigella phage
  source 1..19: mol_type = other DNA
ctgaatgcctgcgagcatc

SEQ ID NO: 55 - protein, 62 aa, unidentified
  REGION 1..62: note = Shigella phage
MKSIRCKNCNKLLFKADSFDHIEIRCPRCKRHIIMLNACEHPTEKHCGKREKITHSDETVRY

SEQ ID NO: 56 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 57 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 58 - DNA, 4101 bp, synthetic construct
  source 1..4101: mol_type = other DNA
gacaagaagtacagcatcgggctggcgatcgggaccaactccgtcggctgggctgtgattaccgacgagtacaaggtgccatccaagaagttcaaggtcctcggcaacactgaccggcacagcattaagaagaacctgattggggcgctgctgttcgattcgggggagactgcggaggcgaccaggctgaagcggactgcgcgccggaggtacaccaggaggaagaatcggatctgctacctccaggagattttctcgaatgagatggccaaggtggacgattccttcttccatcgcctggaggagtcgttcctcgttgaggaggacaagaagcatgagaggcatcccattttcgggaatatcgttgacgaggtggcttaccatgagaagtacccgaccatctaccatctgcggaagaagctcgtcgattcgaccgataaggccgacctgcggctgatctacctggccctcgcgcacatgattaagttccggggccatttcctcatcgagggcgacctcaacccggacaactcggacgtggataagctcttcattcagctcgtgcagacatacaaccagctcttcgaggagaatcccattaacgcctcgggggtcgacgctaaggctattctctcggctcggctgtcgaagtcgcgccggctggagaatctcattgcccagctcccaggcgagaagaagaacggcctcttcggcaacctgattgccctgtcgctggggctcacaccgaatttcaagtcgaacttcgacctcgccgaggacgctaagctccagctcagcaaggatacttacgatgatgacctcgataacctgctcgcccagattggggatcagtacgcggatctgttcctcgcggccaagaatctcagcgatgctattctcctgtcggacattctccgcgtcaacacagagattactaaggccccactgtcggcgagcatgattaagaggtacgatgagcatcatcaggacctgacactgctcaaggcgctggtccggcagcagctccccgagaagtacaaggagattttcttcgatcagtcaaagaatgggtacgcgggctacattgatggcggcgcgtcccaggaggagttctacaagttcattaagcccatcctggagaagatggacgggaccgaggagctgctggtgaagctcaatcgggaggacctgctccggaagcagcgcacattcgacaatggctcgattcctcaccagattcacctgggcgagctgcacgccattctccgcaggcaggaggacttctacccgttcctcaaggacaaccgcgagaagatcgagaagatcctgaccttccggattccatactacgtggggccgctcgcgcgggggaactcccggttcgcgtggatgactcgcaagtccgaagaaacgattacaccgtggaatttcgaggaggtcgtcgacaagggcgctagtgcgcagtcattcattgagaggatgaccaatttcgataagaacctgcctaacgagaaggtgctgccgaagcattcgctgctctacgagtacttcaccgtttacaatgagctgaccaaggtgaagtatgtgactgagggcatgaggaagccagcgttcctgagcggcgagcagaagaaggctatcgtggacctgctcttcaagactaaccggaaggtgactgtgaagcagctcaaggaggactacttcaagaagattgagtgcttcgattccgttgagattagcggggtggaggatcggttcaatgcttcgctcgggacataccacgatctcctgaagatcattaaggataaggacttcctcgacaacgaggagaacgaggacattctcgaagatattgtcctgaccctcaccctcttcgaggatcgggagatgatcgaggagaggctcaagacatacgctcatctgttcgatgataaggtcatgaagcagctgaagcgcaggcggtacacagggtgggggcggctgagccggaagctgatcaacgggattcgggataagcagtccgggaagacaattctcgacttcctcaagtccgacgggttcgctaaccggaacttcatgcagctcattcatgatgactcgctgacattcaaggaggatattcagaaggcgcaggtttcggggcagggcgactcgctccacgagcatattgcgaatctggcgggctcccccgcgattaagaagggcattctgcaaaccgtcaaggtggttgatgagctggtcaaggtcatggggcggcataagccagagaatattgtcatcgagatggcgcgggagaatcagaccacacagaaggggcagaagaactcacgggagcggatgaagcgcatcgaggagggcatcaaggagctggggtcgcagatcctgaaggagcatcccgtggagaacactcagctgcaaaatgagaagctgtacctctactacctccagaacgggagggacatgtatgtggatcaggagctggatattaataggctgagcgattacgatgtcgaccacattgtcccacagtcgttcctgaaggacgacagcattgacaacaaggtgctgacccgctcggataagaacaggggcaagagcgataatgttccaagcgaggaggttgtgaagaagatgaagaactactggcggcagctcctgaacgcgaagctcatcacacagcggaagttcgacaacctcaccaaggctgagcgcgggggcctgagcgagctggacaaggcggggttcattaagaggcagctggtcgagacacggcagattacaaagcatgttgcgcagattctcgattcccggatgaacaccaagtacgatgagaacgataagctgattcgggaggtcaaggtaattaccctgaagtccaagctggtgtccgacttcaggaaggacttccagttctacaaggttcgggagatcaacaactaccaccacgcgcatgatgcctacctcaacgcggtcgtggggaccgctctcatcaagaagtacccaaagctggagtcagagttcgtctacggggattacaaggtttacgacgtgcggaagatgatcgctaagagcgagcaggagattggcaaggctaccgctaagtacttcttctactccaacatcatgaacttcttcaagacagagattaccctcgcgaatggcgagatccggaagaggcccctcatcgagacaaatggggagacaggggagattgtctgggataaggggcgggatttcgcgaccgtccggaaggtcctgtcgatgccccaggttaatattgtcaagaagactgaggtccagactggcggcttctcaaaggagtcgattctcccaaagaggaactccgataagctcattgctcggaagaaggattgggaccccaagaagtacgggggattcgactcccccactgttgcttactctgttctggttgttgctaaggtggagaaggggaagtcgaagaagctgaagagcgtgaaggagctgctcgggattacaattatggagaggtcatccttcgagaagaatcccatcgacttcctggaggccaagggctacaaggaggtgaagaaggacctgattattaagctgcccaagtactcgctcttcgagctggagaatgggcggaagcggatgctggcgtccgcgggggagctgcaaaaggggaacgagctggcgctcccctccaagtatgtgaacttcctctacctggcgtcgcactacgagaagctgaaggggtccccagaggataatgagcagaagcagctcttcgtcgagcagcataagcactacctggacgagattatcgagcagattagcgagttctcgaagcgggtcatcctcgcggatgcgaacctggataaggtgctcagcgcctacaataagcaccgggacaagccgattcgggagcaggcggagaatattattcacctcttcacactcaccaacctcggggcaccagctgcgttcaagtacttcgacactactatcgaccggaagcggtacacctcgacgaaggaggtgctcgacgccaccctcattcaccagtcgatcacaggcctgtacgagacacggattgacctgtcccagctcgggggcgac

SEQ ID NO: 59 - DNA, 4101 bp, synthetic construct
  source 1..4101: mol_type = other DNA
gacaagaagtactccattggcctggcgattgggacaaactcggtggggtgggccgtgattacggatgagtacaaggttccaagcaagaagttcaaggtcctcgggaacacagatcggcattcgattaagaagaatctcattggggcgctcctcttcgactcgggggagacagcggaggctaccaggctcaagcggacagccaggcggcggtacacaaggcggaagaatcgcatctgctacctccaggagattttctcgaatgagatggcgaaggtggacgacagcttcttccatcggctggaggagtccttcctggtggaggaggataagaagcacgagaggcatccaattttcgggaacatcgtggacgaggttgcgtaccatgagaagtaccctacaatctaccatctgcggaagaagctggttgactccacagacaaggcggacctgaggctgatctacctcgctctggcccacatgattaagttccgcgggcatttcctgatcgagggggacctgaatcccgacaattcggatgtggacaagctcttcatccagctggtgcagacctacaaccagctgttcgaggagaatcccatcaatgcgtcgggcgttgacgctaaggccattctgtccgctaggctgtcgaagagcaggaggctggagaacctgatcgcccagctgccaggcgagaagaagaatgggctcttcgggaatctgattgcgctctccctggggctgacaccgaacttcaagagcaatttcgatctggctgaggacgcgaagctccagctctcgaaggacacttacgacgatgacctcgataacctcctcgcgcagatcggggaccagtacgctgatctcttcctcgccgctaagaacctctcggatgctatcctgctctccgacattctccgggttaataccgagattacaaaggccccactgtcggcgtccatgatcaagcggtacgatgagcatcatcaggatctcaccctgctcaaggccctcgtgcggcagcagctgcccgagaagtacaaggagattttcttcgaccagagcaagaatgggtacgctggctacattgacggcggggcctcacaggaggagttctacaagttcatcaagccaatcctggagaagatggatgggacagaggagctgctggtgaagctcaaccgggaggatctgctcaggaagcagcggacgttcgacaacgggtcgattccccatcagatccacctgggggagctgcacgcgatcctgcgccggcaggaggatttctaccctttcctgaaggataatcgggagaagatcgagaagattctcaccttccggattccctactacgtcgggccactcgcgcggggcaatagcaggttcgcctggatgacacggaagagcgaggagacaatcaccccctggaacttcgaggaggttgtcgacaagggggcgtccgcccagtcattcattgagcggatgaccaatttcgacaagaatctgccaaatgagaaggttctcccaaagcatagcctcctctacgagtacttcactgtttacaacgagctgaccaaggtgaagtatgtgaccgagggcatgcggaagcccgcgttcctgtccggcgagcagaagaaggccattgtggacctcctgttcaagaccaatcgcaaggtcacagtcaagcagctcaaggaggattacttcaagaagatcgagtgcttcgactcggttgagattagcggggtggaggatcggttcaacgcgagcctcggcacttaccacgacctcctgaagatcatcaaggataaggacttcctcgacaacgaggagaacgaggatattctggaggacatcgtgctcaccctgacgctgttcgaggatcgggagatgatcgaggagcgcctgaagacctacgctcatctcttcgatgataaggtcatgaagcagctgaagaggaggcggtacaccgggtggggccgcctgagcaggaagctcattaacgggatcagggacaagcagagcggcaagaccatcctggacttcctcaagagcgatggcttcgccaaccggaatttcatgcagctcatccacgacgactccctcaccttcaaggaggacattcagaaggctcaggtcagcggccagggcgactcgctgcatgagcacatcgctaacctggcgggcagcccagccatcaagaagggcatcctccagacagtgaaggtcgtggatgagctggtgaaggtcatgggccggcataagcccgagaatattgtgattgagatggcgcgggagaatcagaccactcagaagggccagaagaactcgcgggagcgcatgaagaggatcgaggaggggattaaggagctgggcagccagattctcaaggagcaccccgtggagaatacccagctccagaacgagaagctgtacctctactacctccagaatgggcgggacatgtatgttgatcaggagctggacatcaatcgcctctcggattacgacgtggaccacatcgtgccccagagcttcctgaaggatgatagcatcgacaataaggtcctgacccgctccgacaagaatcgcggcaagagcgacaacgtgccgagcgaggaggtcgtgaagaagatgaagaactactggcggcagctgctgaacgcgaagctcattacacagcggaagttcgataacctgacgaaggcggagaggggcggcctctccgagctggacaaggcgggcttcattaagaggcagctcgtggagactcgccagatcaccaagcacgtggctcagatcctcgatagccggatgaatacgaagtacgatgagaatgacaagctcatccgggaggtgaaggtaatcaccctgaagtcaaagctcgttagcgatttccggaaggacttccagttctacaaggtgcgggagattaacaactaccatcatgcgcacgatgcgtacctcaatgcggtggtgggcacagccctgattaagaagtaccccaagctggagagcgagttcgtctacggggactacaaggtgtacgatgttcggaagatgatcgccaagagcgagcaggagattgggaaggccaccgctaagtacttcttctactcgaatattatgaatttcttcaagaccgagatcacactcgctaatggggagattcggaagcggcccctcatcgagactaacggggagactggcgagattgtgtgggacaaggggcgcgacttcgctaccgtgcgcaaggtcctctcgatgccccaggttaatattgttaagaagacagaggtgcagacgggcgggttctccaaggagtctatcctgccgaagcggaactcggacaagctgatcgcccgcaagaaggattgggaccccaagaagtacgggggattcgatagcccaaccgtggcttacagcgtcctggtggtcgccaaggttgagaaggggaagtcgaagaagctcaagagcgttaaggagctgctgggcatcaccatcatggagcggtccagcttcgagaagaatcctatcgacttcctggaggctaaggggtacaaggaggtcaagaaggacctgatcattaagctgcccaagtactctctgttcgagctggagaacgggaggaagcggatgctggcgtctgctggcgagctacagaagggcaatgagctggcgctcccctcgaagtatgtcaacttcctctacctggcttcccattacgagaagctgaagggctcgcccgaggataatgagcagaagcagctcttcgtggagcagcacaagcactacctcgacgagatcattgagcagatttcggagttctcgaagcgggtcattctcgcggacgcgaacctcgacaaggtcctctcggcgtacaacaagcaccgggacaagcccatccgggagcaggccgagaacattatccacctcttcacactgaccaacctcggcgctcccgccgcgttcaagtacttcgacaccaccattgaccgcaagagatacacatccaccaaggaggtgctggacgcgaccctcatccaccagagcatcacaggcctctacgagacacggatcgacctctcgcagctcgggggcgat

SEQ ID NO: 60 - DNA, 4092 bp, synthetic construct
  source 1..4092: mol_type = other DNA
gacaagaagtactcgatcggcctggcgattggcacaaacagcgtggggtgggctgtgatcactgatgagtacaaggtgccatcgaagaagttcaaggtgctggggaatacagaccggcattcgatcaagaagaatctcattggcgctctcctcttcgattccggcgagactgctgaggcgacccgcctgaagcgcaccgcccggcggcgctacactcggcggaagaataggatttgctacctccaggagattttctcgaatgagatggccaaggtggatgacagcttcttccaccgcctggaggagtcgttcctggtcgaggaggacaagaagcatgagcggcaccctatcttcgggaatatcgttgatgaggtcgcctaccacgagaagtaccccactatctaccatctccgcaagaagctcgtggacagcacagataaggccgacctccgcctgatctacctcgccctcgcgcacatgattaagttccgggggcacttcctcattgagggggatctgaatcccgataactccgacgtggacaagctgttcatccagctggtgcagacatacaaccagctgttcgaggagaatcccatcaacgcgagcggcgtggacgctaaggccattctgtcggctaggctctcgaagtcgaggcggctggagaacctgattgcgcagctccccggcgagaagaagaacgggctgttcgggaatctcatcgccctctccctcggcctcacaccaaacttcaagagcaatttcgacctggctgaggacgctaagctgcaactctcaaaggatacatacgatgacgacctggacaatctcctggctcagatcggcgaccagtacgctgacctgttcctcgcggccaagaatctgtcggacgcgattctcctcagcgacatcctgcgcgtcaataccgagattacgaaggctccactgtctgcgtcaatgattaagcggtacgatgagcatcaccaggatctgaccctcctgaaggcgctcgtgcggcagcagctgcccgagaagtacaaggagattttcttcgatcagagcaagaatggctacgccggctacatcgacgggggcgcgagccaggaggagttctacaagttcatcaagcccatcctggagaagatggacggcaccgaggagctactcgtgaagctcaatcgggaggatctcctccggaagcagcggacattcgataacgggtctatcccacaccagatccacctcggcgagctgcatgcgattctgcggcggcaggaggatttctaccctttcctgaaggacaaccgggagaagatcgagaagatcctcacattccggattccatactacgtcggccccctggcgaggggcaatagccggttcgcgtggatgacaaggaagtccgaggagactattaccccgtggaatttcgaggaggtggttgacaagggcgcttccgcgcagagcttcattgagcggatgacaaacttcgacaagaatctccccaacgagaaggtcctgccgaagcatagcctcctgtacgagtacttcaccgtctacaatgagctaactaaggtcaagtatgtgacagagggcatgaggaagccagccttcctctcaggcgagcagaagaaggccattgtggacctcctgttcaagacaaaccgcaaggtgacagtgaagcagctgaaggaggattacttcaagaagattgagtgcttcgactcagtggagatttcaggcgtggaggatcggttcaacgcgagcctggggacttaccacgacctgctgaagattattaaggacaaggacttcctggataacgaggagaatgaggacatcctggaggatattgtgctcaccctcaccctgttcgaggacagggagatgattgaggagaggctcaagacctacgcgcacctgttcgatgacaaggtcatgaagcagctgaagaggcggcgctacactgggtggggccgcctgtcgcggaagctgatcaacggcattcgggataagcagtccgggaagaccattctggatttcctgaagtcggacggcttcgccaacaggaatttcatgcagctgatccacgacgactccctcaccttcaaggaggacattcagaaggcccaggttagcggccagggggactcactccacgagcatattgccaatctggccggctctccagctatcaagaagggcatcctgcaaacagttaaggttgttgacgagctggttaaggtcatggggcggcataagcccgagaacattgtcatcgagatggctcgggagaaccagacaactcagaagggccagaagaactccagggagcgcatgaagcggattgaggagggcattaaggagctggggtcccagatcctcaaggagcaccctgtcgagaacactcagctgcaaaacgagaagctctacctgtactacctccagaacgggcgggatatgtatgtggatcaggagctggacatcaacaggctctccgactacgacgtggatcacattgtcccacagtctttcctcaaggatgattccatcgacaacaaggtgctgacgcgcagcgacaagaatagggggaagtcggacaacgttccgagcgaggaggtcgtgaagaagatgaagaattactggaggcagctcctgaatgcgaagctgatcactcagaggaagttcgacaatctgacaaaggcggagaggggcgggctctcggagctggataaggcgggcttcatcaagcggcagctcgttgaaacccggcagatcaccaagcatgtcgcccagatcctcgatagccgcatgaacaccaagtacgatgagaacgacaagctcattcgggaggttaaggtcattacgctgaagtccaagctcgtcagcgacttcaggaaggatttccagttctacaaggttcgggagattaacaactaccaccacgcgcatgatgcgtacctgaacgctgttgtcggcactgctctcatcaagaagtacccaaagctggagtccgagttcgtctacggggactacaaggtctacgatgtccggaagatgatcgccaagtcggagcaggagatcgggaaggctactgcgaagtacttcttctacagcaacattatgaatttcttcaagacggagattacgctggcgaacggggagattaggaagaggcccctcattgagactaatggggagacaggcgagattgtttgggacaagggccgcgacttcgcgactgtgcggaaggtcctgtccatgccacaggtgaatattgttaagaagacagaggtgcagactgggggcttctcgaaggagagcattctcccaaagcggaacagcgataagctcatcgcgcgcaagaaggattgggaccctaagaagtacggcggcttcgattctcccactgtggcctactccgttctcgtggttgccaaggttgagaaggggaagtcgaagaagctgaagtcggtcaaggagctgctcgggattacaatcatggagcggagcagcttcgagaagaaccctattgatttcctggaggccaagggctacaaggaggttaagaaggatctcattatcaagctccctaagtactctctgttcgagctggagaatggccggaagaggatgctggcctcggctggcgagctacagaaggggaatgagctggccctcccgtcgaagtatgtgaatttcctgtacctcgcgtcgcactacgagaagctcaagggcagcccggaggataatgagcagaagcagctcttcgtggagcagcataagcactacctggacgagatcattgagcagatcagcgagttctcgaagcgggttattctggctgatgctaacctggacaaggttctgagcgcctacaataagcatcgcgacaagccgattcgcgagcaggcggagaatattatccacctgttcaccctcactaacctcggggctcccgcggccttcaagtacttcgataccacaatagataggaagcggtacacctcgacgaaggaggtcctcgacgccacactcatccatcagtcgattacaggcctgtacgagacacggattgacctctcgcagctg

SEQ ID NO: 61 - DNA, 4101 bp, synthetic construct
  source 1..4101: mol_type = other DNA
gacaagaagtattccataggcctggctatcggcaccaacagcgtgggctgggccgtcatcaccgacgagtacaaagtgccgagtaaaaagttcaaagtgctcggcaacaccgaccgccactccataaagaaaaacctgatcggggcgctcctgttcgacagcggcgagacggcggaggccacccgcttgaaacgcacggcccgacggcgctacacgcggcgcaagaaccggatctgttacctacaggagattttctctaacgagatggcgaaggtggacgactcgttctttcaccgcctcgaagagtccttcctcgtggaggaggacaagaaacacgagcgccacccgatcttcggcaacatcgtggacgaggtggcctaccacgagaagtacccgaccatctaccacctccggaagaaactcgtggacagcacggacaaggccgacctgaggctcatctacctcgccctggcgcacatgattaagttccggggccacttcctgatcgagggcgacctgaacccggacaacagcgacgtggacaagctgttcatccagctagtccagacctacaaccagcttttcgaggaaaaccccatcaacgccagcggggtggacgcgaaggcgatcctgtccgcccggctgagcaagtcccggcggctggagaacctcatcgcgcagttgcccggcgagaagaagaacgggctgttcgggaacctgatcgccctctccctggggctcaccccgaacttcaagtccaacttcgacctcgccgaggacgccaaactacagctgagcaaggacacctacgacgacgacctcgacaacctgctggcccagatcggggaccagtacgcagacctgttcctcgccgccaagaacctctccgacgccatcctgctgtcggacatcctgcgggtgaacacggagatcacgaaggccccgctctcggcctcgatgattaaacgctacgacgagcaccaccaggacttgaccctcctcaaggcgctggtccgccagcagcttcccgagaagtacaaggaaatctttttcgatcagagcaagaacgggtacgccgggtacatcgacggcggggcgtcccaggaggagttctacaagttcatcaagcccatcctggagaaaatggacgggaccgaggagctgctcgtgaagctcaaccgcgaagatttgctccgcaagcagcgcacgttcgacaacgggtcgatcccgcaccagatccacctgggcgagctgcacgcgatcctcaggcgtcaggaagacttctaccccttcctcaaggacaaccgcgagaagatagagaagattctgaccttcagaattccttattacgtgggcccgctggctcggggcaactcgcgcttcgcctggatgacgcgcaagtccgaggagaccatcaccccgtggaacttcgaggaggtggtggataagggtgcctcggcccagtccttcatcgagcggatgaccaacttcgacaagaacctgccgaacgagaaggtgctccccaagcacagcctgctctacgaatatttcacggtgtacaacgagctgacgaaggtcaagtacgtgaccgagggaatgaggaaacctgcattcctctccggggagcagaagaaagccatagtcgacctcctgttcaagaccaaccggaaggtcaccgtcaagcagctcaaggaggactacttcaagaagatcgagtgcttcgattcagtggagatcagcggcgtcgaggaccggttcaacgccagcctgggcacctaccacgacctgctcaagatcatcaaggacaaggacttcctcgacaacgaggagaacgaggacatcctggaggacatcgtgctgaccctgacgctcttcgaggaccgcgagatgatcgaggagcgcctcaagacctacgcccacctgttcgacgacaaggtgatgaagcagctcaagcggcggagatatactgggtggggccgcctctcccggaagctcattaacggtatcagggataagcagtccgggaagacgatcctcgacttcctcaagtcggacgggttcgccaaccgcaacttcatgcagctcatccacgacgactccctgacgttcaaggaggacatccagaaggcccaagtgtctggtcaaggtgactcgctccacgagcacatcgccaacctcgcgggcagcccggccatcaagaagggaatactccagaccgtcaaggtggtggacgagctggtgaaggtcatgggccgccacaagccggagaacatcgtcatcgagatggcgcgggagaaccagaccacgcagaaggggcagaaaaatagccgtgagcgcatgaagcgcatcgaggaggggattaaggagttgggcagccagatcctcaaggagcaccctgtggagaacacgcagttgcaaaacgagaagctctacctgtactacctccagaacgggagggatatgtacgtggaccaagaactggacatcaaccgcctgtccgactacgacgtggaccacatcgtgccgcagagcttcctcaaggacgacagcatcgacaacaaggtgctcacccggtccgacaagaatcggggcaagtccgacaacgtgcccagcgaggaggtcgtcaaaaagatgaaaaactactggcgacaactactgaacgccaagctcatcacccagcgcaagttcgacaacctcacaaaagccgagcgcggcgggttgagcgagctggacaaggccgggttcatcaagcgccagctcgtcgagacgcgccagatcacgaagcacgtcgcgcagatactcgacagccggatgaacaccaagtacgacgagaacgacaagctcatccgggaggtgaaggtcatcaccctcaagtcgaagctcgtgagcgacttccgcaaggacttccagttctacaaggtccgggagatcaacaactaccaccacgcccacgatgcttatcttaacgccgtggtggggacggccctcattaagaaatacccgaagctggagtcggagttcgtgtacggcgactacaaggtgtacgacgtcaggaagatgatcgccaagtccgaacaggagatcgggaaggccacggcgaaatacttcttctacagcaacatcatgaacttcttcaagaccgagatcaccctcgccaacggcgagatccgcaagcgcccgctcatcgagacgaacggggagaccggcgagatcgtctgggacaaggggcgcgacttcgccactgtgcggaaggtgctgtcgatgccccaggtcaacatcgtcaagaagacggaggtccagacgggcgggttcagcaaggagagcatcctgccgaagcgcaacagcgacaagctgatcgcccgcaaaaaggactgggatccaaaaaagtacggcggcttcgacagccccaccgtcgcctacagcgtcctcgtcgtcgctaaagtcgagaagggcaagtccaaaaagctcaagagcgtcaaggagctgctcgggatcaccatcatggagcggtccagcttcgagaagaacccaattgatttcctggaggcgaagggctacaaggaggtcaagaaagacctcatcataaagctgccgaagtactcactcttcgagctggagaacgggcgcaagcggatgctggcgtcggccggagagctccaaaagggcaacgagctggcgctgccgagcaagtacgtgaacttcctctacctggcgtcccactacgagaagctcaagggcagtccagaggataacgagcagaagcagctattcgtggagcagcacaagcactacctggacgagatcatcgagcagatcagcgagttctccaagcgcgtcatcctggcggacgccaacctggacaaggtgctgtccgcgtacaacaagcaccgcgacaagccgatccgcgagcaagccgagaacatcatccacctgttcaccctcacgaacctcggggcacccgccgccttcaaatatttcgacacgaccatcgaccgcaagcgctacaccagcacgaaggaggtgctcgacgccaccctgatccaccagagcatcaccgggctgtacgagacccgcatcgacctctcgcagctcggcggggac

SEQ ID NO: 62 - DNA, 4101 bp, synthetic construct
  source 1..4101: mol_type = other DNA
gacaagaagtacagtattggattggccatcgggacgaacagcgtgggctgggccgtcatcaccgacgagtacaaggtgccatccaagaagtttaaggttctggggaataccgaccgccactcgatcaagaaaaatctcatcggggcgctgcttttcgacagcggcgagacggcggaagcgacgcggctcaagcggacggctcgtcgccgttacacccggcgtaagaaccgcatctgttacctccaggagatattcagcaacgagatggcgaaggtggacgactcctttttccaccgtcttgaggagtccttcctggtcgaggaggacaagaagcacgagcgccacccgatcttcgggaacatcgtggacgaggtggcctaccacgagaagtaccccacgatctaccacctccgcaaaaaactcgtggactcaactgacaaggccgatttgaggcttatctacctcgccctcgcccacatgattaagttccgtgggcacttcctaatcgagggtgacctcaaccccgacaactctgacgtggacaagctgttcatccagcttgtgcagacctacaatcagctctttgaggagaatccgatcaacgcatctggtgtggacgcaaaggccatcctcagcgcgcggctgagcaagtctaggcggttggagaacctgatcgcccaactgcccggcgagaagaaaaatggcctcttcggcaacctgatcgccctgtcgctggggctcacgccgaacttcaagagtaactttgacctggcggaggacgctaagctccagctatctaaggacacatacgacgacgacctggacaacctgctggcccagatcggcgaccagtacgccgacctcttcctagccgccaagaacctgtccgacgccatcctcctcagcgacatcctgcgcgtgaacacggagatcacgaaggctccgctcagcgcctccatgattaagcggtacgacgagcaccaccaagacctaactttactcaaagccctcgtgcggcagcagcttcccgagaagtacaaagagatattttttgatcagtccaagaacggttatgcgggctacatcgacggcggcgcgagccaggaggagttctacaagttcatcaagcccatcctggagaagatggacggcacggaggagctgctcgtgaagctcaaccgtgaagacctcctgcgaaagcagcgaaccttcgacaacggttcgatcccgcaccagatccacctcggggagctgcacgccatcctgaggcgacaggaggacttctaccctttcctaaaggacaaccgcgagaagattgaaaaaatcctgacgtttcgcataccctactacgtcggcccgctggcgcgcggcaactcccggttcgcctggatgacccgtaagagcgaggagacgatcaccccgtggaacttcgaggaggtcgtggacaagggcgcgagcgcgcagagcttcatcgagcgcatgaccaacttcgacaagaacctcccgaacgagaaggtgctcccaaagcactccctcctgtacgagtatttcaccgtgtacaacgagttgacaaaggtgaagtacgtgacggagggaatgcggaagcctgcgttcctctcgggcgagcagaagaaggcaatcgtggacctgctcttcaagaccaaccggaaggtgacggtgaagcagctcaaggaggactacttcaaaaaaatcgagtgcttcgactccgtggagataagcggcgtggaggaccgattcaacgcctccctcggcacctaccacgacctccttaagatcatcaaggacaaggacttcctggacaacgaggagaacgaggacatcctggaggacatcgtgctcaccctgaccctcttcgaggaccgggagatgatcgaggagcgcctcaagacgtacgcccacttgttcgacgacaaggtgatgaagcagctcaagcggcggcgatacaccgggtggggccgcctatcccgcaaacttatcaacggcatccgcgacaagcagtccggcaagacgatcctggatttcctcaagtcggacgggttcgccaaccggaacttcatgcagctcatccacgacgacagcctcacgttcaaggaggacatccagaaggcccaagtgagcggtcaaggggacagcctccacgagcacattgcgaaccttgctgggagccctgcgatcaagaaggggatattgcaaaccgtgaaggtcgtggacgagttggtgaaggtcatggggcgacacaagcccgagaacatcgtgatcgagatggccagggaaaatcagaccacgcagaagggccaaaaaaacagccgcgagcggatgaagcggatcgaggagggcatcaaggagctggggtcgcagatcctcaaggagcacccggtggagaacacgcagctccagaacgagaagctgtacctctattacctacagaacgggcgggatatgtacgtggaccaggagctagacatcaaccgcctgtccgactacgacgtggaccatatcgtcccgcagtcgttcttgaaggacgacagcatcgacaacaaggtgctcacaagatcggataagaatcgaggcaagtccgacaacgtgccctcggaggaggtggtcaagaaaatgaaaaactactggcggcagttgctgaacgccaagctcattacgcagcggaagttcgacaacctgacgaaggctgaacgtggtgggctcagcgagctagacaaggcggggttcatcaagcggcagctcgtcgagacccggcagatcaccaagcacgtggcgcagatcctggactcgcgcatgaacaccaagtacgacgagaacgacaagctcatccgtgaggtgaaggtcatcacccttaagtctaagctggtcagtgacttccgcaaggacttccagttctacaaggtccgggagatcaacaactaccaccacgcgcacgacgcctacctcaacgcggtggtggggacggcgcttattaagaaatatcccaagctggaaagcgagttcgtttacggcgactacaaggtgtacgacgtccgcaagatgatcgcaaagtcggaacaggaaatcggaaaggcgacggccaaatatttcttttactccaacatcatgaatttttttaagacggagatcaccctggcgaacggggagatccgcaagcggcccctcatcgagaccaacggggagacgggcgagatcgtctgggacaagggccgggacttcgccaccgtgcggaaggtgctttctatgcctcaagtcaatatcgtcaaaaagacagaggtgcagaccggcgggttcagcaaggagtctatcctgccgaagcgcaactcggacaagctcatcgcgcgcaagaaagactgggaccccaaaaaatatggcgggttcgactcgccgaccgtcgcctacagcgtcctcgtggtggctaaggtcgagaagggcaagagcaaaaagctaaagtcggtgaaggagctgctgggcatcaccatcatggagcgctcgtctttcgagaagaatccaatcgacttcctagaggcgaaggggtacaaggaggtcaaaaaggatcttatcatcaaactgccgaagtacagtctgttcgagctggagaacgggcggaagcggatgctggctagtgcgggcgagttgcagaagggcaacgagttggcactgccctccaagtacgtgaacttcctgtacctggcctcccactacgagaagctcaaggggagccccgaggacaacgagcagaagcagctattcgtcgagcagcacaagcactacctggacgagatcatcgagcagatcagtgagttctccaagcgggtcatcctcgcggacgccaacctggacaaggtgctgagcgcgtacaacaagcacagggacaagccaatcagggaacaggccgagaacatcatccacctgttcaccctgaccaacctgggtgcaccggctgccttcaagtactttgacacgaccatcgaccggaagcgctacacctccacgaaggaggtgctggacgccacgctgatccaccagagcatcaccgggctctacgagacacggatcgacctgagccagcttggcggggac

SEQ ID NO: 63 - DNA, 4092 bp, synthetic construct
  source 1..4092: mol_type = other DNA
gacaaaaagtattccattggactcgctatcggcacgaacagcgtcgggtgggcggtcatcactgacgagtacaaggtgccgagcaagaagtttaaggtgctgggaaacaccgacaggcactcgatcaagaaaaatcttatcggggccctactcttcgactccggagaaaccgccgaggccacccggttgaagcgcacggcccgccgtcgctacaccaggcgcaagaaccggatctgctacctccaggagatattcagcaatgagatggcgaaggtggacgactcgttttttcacaggctagaggagtctttcctcgtggaggaggacaagaaacacgagcgccaccccatcttcggcaacatcgtggatgaggtggcatatcacgagaagtacccaaccatctaccacctccgcaaaaagctcgtggactctaccgacaaggccgacctccgtctgatctacctcgcgctggcccacatgattaagttccgaggacactttctgatcgagggcgacctgaacccagacaacagcgacgtggacaagctgttcatccaacttgtccagacctacaatcagctcttcgaggagaaccctatcaacgcctcgggcgtggacgcgaaggccatcctgtccgcccgcctgagcaagtcgcggcggctggagaacctgatcgcccagctccccggcgaaaaaaagaacggcctcttcggcaacctcatcgcgttgtcgctggggctcaccccgaacttcaagtccaacttcgacctggccgaggacgctaaactccagctctcgaaggatacctacgacgacgacctcgacaacctgctggcccagatcggcgaccagtacgcggaccttttcctggcggccaagaacctgagcgacgcgatcctccttagcgacatactccgtgtgaacaccgagatcacgaaggccccgctctccgcgtccatgattaagcgctacgacgagcaccaccaagaccttaccctgcttaaggcgctggtcaggcagcagttaccggagaagtacaaggagatcttttttgatcaatctaagaacggttacgccgggtacatcgacggcggcgcgtcccaggaggagttctacaagttcatcaagccgatcttggagaaaatggacgggaccgaggagctgctcgtgaagctcaaccgcgaagacctcctccgcaagcagcgcaccttcgacaacgggagcatcccgcaccagatccacctgggagagctgcacgcgatcctgcggagacaagaggacttctaccccttcctcaaggacaaccgggagaagattgaaaaaatacttacttttcgtatcccgtactacgtcgggccccttgcgaggggcaactccagattcgcgtggatgacccgcaagtccgaggagaccatcaccccgtggaacttcgaggaggtggtggacaagggcgcgtcggcccagtcgttcatcgagcgcatgaccaacttcgacaagaaccttccgaacgagaaggtgctcccgaagcacagcctgctctacgaatattttactgtgtacaacgagctgacgaaggtcaagtacgttacggaggggatgaggaagcccgccttcctctccggcgagcagaagaaagccattgtggatctcctgttcaagaccaaccgcaaggtgacggtgaaacagctcaaagaggactacttcaagaagatcgagtgcttcgactccgtagagatcagcggggtcgaggaccgcttcaacgcctcgctgggcacgtaccacgacctgctaaagattatcaaggacaaagacttcctagacaatgaggagaacgaggacattctggaggacatcgtgctgactctgacgctgttcgaagaccgcgagatgatcgaggagcggcttaagacgtacgcccacctgttcgacgacaaggtgatgaagcagttgaaacggcggcgctacaccgggtggggccgcctctcccgcaagctcatcaacggcatccgcgacaagcagtcggggaagacgatcctggacttcctcaagagcgacggcttcgccaaccgaaacttcatgcagctaatccacgacgacagcctgacgttcaaggaggacatccagaaggcccaagtgagcggccagggagactcgctacacgagcatatcgccaacctggctggcagcccggcgattaagaaaggaatcctccaaaccgtcaaagtggtggacgagctggtgaaggtgatgggccgccacaagcccgagaacattgtgatcgagatggcgcgggagaaccagacgacgcagaagggccaaaaaaatagcagggaaaggatgaagcgaatagaggaggggatcaaggagctggggagccagattctcaaagagcacccggtcgagaacacacagctccagaacgagaagctgtacctctactacctccaaaacggccgcgatatgtacgtggaccaggaactagacatcaaccggctgagcgactatgacgtggaccacatcgtgccgcagtccttcctcaaggacgactcgattgacaacaaagtgctcactagatccgacaagaacagaggcaagagcgataacgtcccgtcggaggaggtcgtcaagaaaatgaaaaactactggcggcagctcctaaacgccaagctcatcacgcagcgtaagttcgacaacctgacgaaggcggagcggggcgggctgagcgagctggacaaagcggggttcatcaagcggcagctcgttgagacgcggcagatcacaaagcacgtcgcgcaaatcctcgactcccgcatgaacaccaagtacgacgagaacgacaagctcatccgggaggtgaaggtcattacccttaaatcgaagctcgtcagcgactttcgtaaggacttccagttctacaaggtcagagagatcaacaactaccaccacgcccacgacgcctatctgaacgccgtggtgggcaccgcgcttattaagaagtaccccaagctggagtccgagttcgtgtacggcgactacaaggtttatgacgtcaggaagatgatcgccaagtcggaacaggagatcggaaaagctaccgccaaatatttcttctatagcaacatcatgaacttcttcaaaaccgagatcaccctcgccaacggcgagatccggaagcgcccgctcatcgagaccaacggggagaccggggagatcgtctgggacaaggggcgggacttcgctactgtccgaaaggtgctctccatgccacaagtgaatatcgtcaagaaaacagaggtgcagaccggagggttcagtaaggagtccatcctgcccaagcggaactccgacaagctaattgctcgcaaaaaggattgggatcctaaaaaatatggcggcttcgactcgcccacggtcgcctactctgtgctggtcgtggcgaaggtggagaagggcaagtccaagaagctcaagagcgtcaaggagctgctggggatcacgatcatggagcgtagttcgtttgagaagaatcccatcgacttcctggaggctaagggctacaaggaggtcaaaaaggacctcatcattaagctgccgaagtacagcctcttcgagctggagaacgggcggaagcgtatgctcgcctccgctggggagttacaaaaggggaacgagctggcgctgccgtctaagtacgtcaacttcctgtacctggcctcccactacgagaagctcaaggggtcgccggaggacaacgagcagaagcagctcttcgtagagcagcacaagcactacctggacgagatcatcgagcagatttcagagttctcaaagcgggtcatcctcgccgacgccaacctggacaaggtgctctcggcctacaacaagcaccgggacaagccgatccgcgaacaggccgaaaacatcatccacctgttcacgctcaccaacctcggtgccccggcggccttcaagtactttgacacgaccatcgaccggaagcgctatacctcgacgaaggaggtgctggacgccaccctgatccaccagtccatcaccgggctttacgagacccggatcgacctctcgcagcta

SEQ ID NO: 64 - DNA, 4101 bp, synthetic construct
  source 1..4101: mol_type = other DNA
gacaagaagtatagtattggactcgccatcggaaccaactctgtggggtgggctgttattacagatgaatataaggtgccatccaaaaagtttaaagttctgggcaatactgatagacactcaatcaagaagaatctgataggtgcacttctgtttgatagtggagagactgccgaggcaaccagacttaaaaggactgcaagaagaagatataccagaagaaagaataggatttgctatttgcaggaaatcttcagcaacgaaatggccaaggttgatgactcatttttccataggttggaggagagttttcttgtggaggaagataagaagcacgaaagacacccaattttcgggaatatagtggacgaggtggcttatcatgagaagtatcccactatctaccacctgagaaagaaacttgtggactcaaccgataaggctgatcttaggcttatatacttggcccttgcacatatgatcaaattcaggggccattttcttatcgaaggcgatcttaatcccgataactcagatgtggacaagctgtttatacaacttgtgcaaacctacaatcaactcttcgaggagaatcccattaacgcctccggcgtggatgcaaaagccatactgtcagccagactgagcaaaagtaggagactggagaatcttatagcccaactgcccggtgaaaagaagaatgggctcttcggaaatctgatcgctctttcattggggttgacacccaactttaagagtaactttgacttggcagaagatgcaaagttgcagctcagtaaagacacatatgacgatgaccttgacaatctcttggcacaaataggggatcaatacgctgaccttttcctcgctgccaagaacctcagcgacgctatactgttgtccgacattcttagggttaataccgaaattacaaaggcccctcttagtgcaagtatgatcaaaaggtatgatgagcatcaccaagaccttacactgctgaaggctctggttagacagcaactccctgaaaagtataaggaaatattcttcgaccaaagtaagaacgggtacgccggttatattgatgggggcgcaagtcaagaagaattttacaaattcatcaagccaattcttgaaaagatggacgggactgaggaattgctggtgaaactgaatagagaggaccttcttagaaaacagaggacatttgacaatgggtccatcccacaccagattcatctgggggaactccacgcaatattgaggagacaagaagacttttacccattccttaaggataatagagagaaaatcgaaaaaatcctgactttcaggattccttactatgttgggccactggccagggggaactcaagattcgcttggatgacaaggaagtcagaagaaaccataaccccttggaattttgaagaggtggttgataagggggcatcagcccagtctttcatagagaggatgaccaactttgataaaaatcttccaaatgagaaggttttgccaaaacatagtcttttgtacgagtactttactgtttataacgaattgaccaaggtgaagtatgtgaccgagggaatgaggaagccagcatttttgtccggggagcaaaagaaagcaatcgttgatcttctcttcaagaccaacagaaaagtgaccgtgaaacaactgaaggaagactacttcaaaaagatagaatgtttcgattcagtggaaattagcggtgttgaagacaggttcaatgcttcattgggtacttaccacgacctgttgaagataatcaaagacaaggactttctcgataatgaggagaacgaagacatcttggaagacattgtgcttacactcactttgtttgaggacagggaaatgattgaggaaagactcaaaacttacgctcatttgtttgatgataaggttatgaaacaactaaaaagaagaaggtacaccggctggggaagattgagtaggaaactgatcaacggtattagagataaacaatccggaaagactatcctcgatttccttaagagtgatggctttgcaaataggaattttatgcagctgattcatgacgactcacttaccttcaaagaagacatccaaaaagctcaggtgtctgggcaaggcgacagtctgcatgaacatatagctaacttggctgggagtcccgccatcaagaaggggatacttcaaacagttaaagttgtggacgaattggtgaaggtaatgggaaggcacaagcctgaaaatatagtgatagaaatggcaagggaaaatcaaacaacccagaagggacagaagaacagtagggaaaggatgaaaaggatagaagaggggatcaaagagcttggtagccagatcctcaaggaacatccagtggagaatacccaacttcaaaacgagaaactctatttgtactacttgcagaacggaagagatatgtatgtggaccaagagcttgatattaacaggctgagcgattatgacgttgaccacatagtgccccaatcattcctcaaggatgactctattgataataaggtgctgacaaggagtgacaagaatagagggaaatccgacaacgttccatccgaggaagttgtgaagaagatgaagaactactggaggcagttgctgaacgctaagctcattacccagaggaaattcgataacctgaccaaagcagagagaggcgggctgagcgaactcgataaagcaggtttcatcaagagacaactcgtggagactaggcaaattactaagcacgtggctcaaatactcgacagcaggatgaacacaaagtacgacgagaacgacaagctcattagagaggttaaggttattactctgaaaagtaaattggttagcgatttcagaaaggatttccaattctataaggttagagagatcaacaattatcatcatgcacatgatgcctatctgaatgctgtggttggtacagcccttatcaagaagtaccctaagctagagagcgagtttgtgtacggagattataaggtgtatgatgtgaggaaaatgatcgctaaaagtgagcaagagattggaaaggctaccgccaaatacttcttttattccaatattatgaatttcttcaagacagaaatcaccctggctaacggcgagataaggaagaggccgcttatcgaaactaatggggagacaggcgaaatagtgtgggacaaagggagggatttcgcaactgtgaggaaggttttgagcatgcctcaggtgaatatcgttaagaaaaccgaagttcaaactggagggttctctaaggaaagcattctccccaagaggaactccgacaagctgattgctagaaagaaagactgggaccccaagaagtatggcggattcgactcacccactgtggcatatagcgttctcgtggtggcaaaggttgaaaagggtaaatccaaaaaactcaaatccgtgaaggaactccttggcataactattatggaaaggagtagctttgaaaagaatcccatcgactttctcgaagctaagggctataaggaagttaagaaggaccttataatcaaacttccaaaatactccctttttgagttggaaaacggcagaaagagaatgttggccagtgccggggagcttcaaaagggcaacgaactggctctgcctagcaaatatgtgaactttttgtatctggcatcacactacgagaaacttaaaggctctcctgaggacaacgagcaaaaacagctctttgttgaacagcataagcactacctcgacgagattattgagcagatcagcgagttctcaaagagagttattctggctgacgctaatcttgacaaggttttgtccgcttacaacaaacacagggataagccaatcagggagcaggcagaaaacataatccatctctttaccctgacaaacctcggtgcccccgctgctttcaagtattttgatactaccattgacaggaagagatatacttccactaaggaagtgctcgacgcaaccctcatacaccaaagtatcacaggcctctatgaaactaggatagatttgtctcaacttgggggcgat

SEQ ID NO: 65 - DNA, 4101 bp, synthetic construct
  source 1..4101: mol_type = other DNA
gacaaaaagtattccatcgggcttgctatcggaaccaactctgtggggtgggcagttattaccgacgaatacaaggtgcccagcaagaagtttaaggttctggggaacacagatagacatagcataaagaaaaacctgataggcgcactgttgttcgactccggggaaacagccgaagctaccaggctgaagagaactgcaagaagaaggtacaccagaagaaaaaacagaatatgttatctccaagagattttctctaacgagatggccaaggtggacgactcattctttcacagactggaagaatctttccttgtggaagaagataagaaacacgagaggcaccctatttttggcaatatcgtggatgaggtggcttaccacgaaaaataccctacaatataccacctcaggaaaaaattggttgatagtacagacaaggccgacctcaggctcatctatttggccctggcccatatgattaaattcagggggcactttctcatcgagggagatttgaaccccgacaacagtgatgttgataagctctttattcagctcgtgcagacttacaatcagttgtttgaggaaaaccccattaatgcttccggggtggacgccaaggcaatcctttctgcaagactctcaaagtcaaggagactcgaaaatctgatagcacagcttccaggagagaagaagaacgggctctttggaaacctgatcgctctgtcactcggactcacacccaatttcaaaagcaattttgatttggcagaggacgctaagctgcaactcagtaaggatacctacgacgatgacttggataatctgctcgcacaaattggggaccagtatgcagacctgtttctcgcagctaagaacttgagtgacgccatattgctcagtgacatcctcagggttaataccgagattacaaaagctccactctctgcaagcatgatcaagaggtatgacgagcaccatcaagacctgacactccttaaggcgttggttaggcagcaacttcctgaaaagtataaggaaatcttcttcgatcaaagcaaaaacggctacgccggctatatagacgggggagcatcccaagaagaattttataagttcataaaacctatattggagaagatggacgggacagaggaattgctcgtgaaactgaacagggaggatctcctcaggaagcaaaggaccttcgacaatggctccatcccacatcagattcacctcggcgaactgcacgcaatactgagaagacaagaggacttttatcctttcctgaaggacaacagggagaaaatcgagaaaatcttgacattcagaatcccatactacgttgggcctctggccagaggtaacagtaggttcgcctggatgactaggaaatcagaggagactattacaccctggaactttgaagaagttgttgataagggagcttcagcacaatcattcatcgaaagaatgacaaactttgacaaaaatctgcctaatgagaaagtgctcccaaaacattccctgctgtatgagtattttaccgtttataacgagcttaccaaggtgaaatacgttactgaaggtatgagaaagccagcttttctttcaggggagcaaaagaaggctatcgtggatcttctctttaagaccaacagaaaggttaccgtgaagcagcttaaggaagactactttaaaaagatcgagtgttttgactcagtggaaataagcggtgttgaagatagattcaacgcatccttgggaacttatcatgatcttcttaagataatcaaggataaagactttctcgacaacgaggaaaacgaagatatactggaggacatagttctgacacttactttgttcgaggatagggagatgatcgaggaaagactgaaaacatatgctcaccttttcgacgacaaagttatgaaacaactcaagagaaggagatatacagggtgggggagattgagcaggaaactgattaatggtatcagagacaaacagtcaggaaaaacaatactcgactttttgaaatcagacgggttcgcaaataggaatttcatgcagcttatacacgacgattcacttacttttaaagaggacattcaaaaggctcaagttagtggacaaggtgactccctccacgaacacatcgcaaatctcgctggcagccctgcaattaagaagggtatactccagacagttaaggttgttgacgagctggttaaagtgatgggaagacacaaacccgagaacatagtgatagagatggccagggaaaaccaaaccactcaaaaagggcagaaaaattccagagagaggatgaaaaggattgaagaaggtatcaaggagctgggtagccaaattctgaaagaacatcctgtggaaaacactcaactccagaatgagaaactctatctgtactatctgcaaaatgggagagatatgtatgtggaccaggaactggacataaacaggctctcagattacgatgtggatcatatcgtgccacagtcctttcttaaggatgatagcatcgacaataaggtgcttaccaggtccgacaagaacaggggaaagtcagataacgtgccttctgaagaagttgttaaaaagatgaagaactactggagacagctgcttaacgctaagctcataacacagaggaagtttgacaacttgaccaaggccgagagaggcggactctcagaattggataaggcagggttcataaaaaggcagctggtggaaacaaggcagataactaaacatgtggctcagatcctcgatagtaggatgaatacaaaatacgatgagaacgacaagctcataagggaggttaaagtgataactctgaaatccaaactggttagcgattttaggaaggatttccagttttacaaagttagggagatcaacaattatcatcacgcccacgatgcctacttgaacgcagttgtgggtactgcacttatcaaaaagtaccctaagctggaatccgagtttgtttatggagactataaggtgtacgacgttagaaaaatgattgcaaagtcagagcaggagatagggaaagccactgcaaaatatttcttttatagcaatatcatgaatttctttaagacagaaatcacactggccaatggggaaataaggaagaggcccctgatcgaaactaatggcgagacaggggagattgtgtgggataaaggtagggactttgcaacagtgaggaaagtgctgagcatgccccaagttaatatcgttaaaaagaccgaggttcaaacagggggctttagtaaggaaagcattttgcccaagaggaatagtgacaaattgattgctaggaaaaaagattgggaccccaaaaagtatggcggatttgatagccccactgttgcttactccgtgctcgtggttgcaaaggtggagaagggaaagagcaagaaactgaagtcagttaaggaactccttggtatcactatcatggaaagaagctcctttgagaagaaccctattgacttcctggaggctaaagggtacaaagaggttaagaaagaccttatcattaaattgcccaaatatagtcttttcgagcttgaaaacggaagaaagaggatgcttgcatccgctggcgaattgcaaaagggcaatgagcttgctctcccttccaagtatgtgaacttcctttatcttgcctcacactatgaaaaactcaaaggttcacccgaagacaacgaacaaaagcaactatttgtggaacaacacaagcactacctggacgaaatcattgagcaaatttctgagttttcaaaaagggtaatcttggctgacgcaaatctcgacaaagttttgtcagcttacaacaaacatagagataagccaattagagagcaagctgagaatatcatccatctgtttaccctgactaaccttggagcgcctgctgcttttaaatatttcgacaccacaatcgacaggaagaggtacactagcactaaggaagttctcgacgccaccctcatccaccagagtattacaggcctgtacgagacaagaattgatctttctcaacttggtggtgac

SEQ ID NO: 66 - DNA, 4101 bp, synthetic construct
  source 1..4101: mol_type = other DNA
gataagaagtactcaatcggtctggcaatcggaaccaactctgtgggttgggcagtgattacagatgagtataaggtgccaagcaaaaaattcaaggtgctgggtaataccgacagacacagcattaagaagaatttgattggagcactcctctttgactcaggggaaacagcagaggcaacaaggctgaagaggacagcaaggcggaggtacacaaggcggaaaaacaggatatgctacctccaggaaatctttagcaacgagatggctaaagtggatgatagctttttccatagactcgaagaatcctttcttgttgaagaggacaaaaagcatgaaaggcatcccatcttcggcaatatagttgatgaggttgcataccatgagaagtaccccacaatctaccacctcagaaagaaacttgtggactccacagataaagcagacctgaggctcatatacctcgcactcgcacacatgatcaagttcagagggcactttctcatcgaaggtgacctgaatccagataattcagatgtggataaactgtttatacagctggtgcaaacatacaaccaacttttcgaggaaaacccaatcaatgcctccggtgttgatgcaaaggccatcctgtcagcaagactcagcaaaagcaggcggctcgaaaacctcatcgcccagcttcccggtgaaaagaagaacgggctctttggtaatctcatcgcattgagccttggtcttactccaaacttcaagagcaattttgatctggcagaggatgctaaactgcaactctcaaaggacacatatgacgatgaccttgacaatctgttggcccagatcggggaccaatatgcagacctcttcctggccgcaaagaatctgtcagatgcaatcctcttgtccgacatactgagagttaacactgagatcacaaaggcacctctgtccgcctccatgattaagagatacgatgagcatcaccaggatctgactttgctcaaagccctcgttagacagcagttgccagaaaagtacaaagaaatattctttgatcaatcaaaaaacggatatgcagggtacatcgacggtggggcaagccaggaagagttctacaaattcatcaaacctatcctggaaaagatggatgggacagaagagctgctggttaagctgaatagggaagacctcctcagaaagcagaggacatttgataacgggagcatccctcatcaaatccacctcggtgaactccatgctatcctgagaaggcaggaagacttttatccatttttgaaggacaatagggagaaaatcgaaaaaatcctgacattcagaatcccatactacgttggtcctctggcaagaggtaacagtaggttcgcatggatgacaaggaaaagcgaggagacaatcacaccctggaattttgaggaagttgttgacaagggtgccagcgcacaatcctttatcgaaagaatgacaaatttcgacaagaatctgcctaacgaaaaggttctcccaaagcattcactcctgtacgaatattttacagtttataacgaactgactaaagttaaatacgttaccgagggtatgaggaagccagcattcctttccggggaacagaagaaagctattgtggacctcctgttcaagacaaatagaaaagtgacagttaagcaactcaaagaggattacttcaaaaagatcgaatgttttgactctgtggagatcagcggggtggaggatagattcaacgccagcctgggtacatatcatgatctcctgaaaatcattaaagacaaggacttccttgacaacgaggagaacgaggacattctggaagacattgttctgaccctcacactctttgaggatagggagatgattgaggaaagactgaagacctacgcccacctctttgacgataaagtgatgaaacagctcaagagaagaaggtatacaggttgggggagactgagcaggaagttgatcaatgggattagggacaaacagtccgggaaaacaatcctcgattttctgaagtcagacggtttcgcaaacagaaattttatgcagctcattcacgatgacagcttgacattcaaggaagacatccaaaaggctcaagtgagcggccaaggggatagcctccacgagcatattgcaaatctggcaggttcaccagccatcaaaaagggcatacttcagacagttaaggttgtggacgaattggttaaagttatgggcaggcataagccagagaatatcgttatcgaaatggcaagggagaaccaaacaactcaaaaagggcagaaaaatagcagagagaggatgaaaagaatcgaggaagggatcaaggaacttgggtcccaaatcctcaaggagcacccagttgaaaatactcaactgcaaaacgagaagctctatctctactatctccaaaacgggagggatatgtatgttgaccaggagctggatattaacagactgtcagattatgatgttgatcatatcgtgccccagtcattcctgaaggacgattccatcgacaacaaagttctcacaaggtccgataaaaacaggggcaagtccgataacgttccaagcgaagaagtggtgaaaaagatgaaaaactattggagacaacttctgaatgcaaagttgattactcagagaaagtttgacaacctcacaaaagcagaaagaggcgggcttagcgaactcgataaggcagggtttatcaaaagacagctggttgagacaaggcagatcacaaaacatgtggcacagatccttgactcaaggatgaataccaagtatgatgagaatgataagttgatcagggaggttaaagttatcacactcaaatccaaactggtgtcagacttcaggaaagactttcaattttataaggtgagggagatcaataactaccaccatgcacatgacgcctacctgaacgcagtggtgggtacagcattgattaaaaaataccctaagctggagtctgagtttgtgtacggggactacaaggtgtacgacgtgaggaaaatgatagccaagtccgagcaggagatcgggaaagcaacagctaagtatttcttttacagtaatatcatgaatttctttaaaactgagattactctggcaaacggggagatcaggaaaagacccctcatcgagactaatggtgaaacaggtgagatcgtttgggacaaggggagggattttgctactgttagaaaagttctgagtatgccacaagtgaatattgtgaaaaagacagaagttcagacaggtgggttctccaaagaatccatcctgcccaagagaaattcagacaagctcatcgcaagaaagaaggactgggaccctaagaagtacggaggatttgacagccccaccgtggcctattccgtgcttgttgtggcaaaggtggagaaagggaagagcaaaaaactgaaatccgtgaaagaactgctgggaattaccatcatggaaagaagctcctttgagaagaacccaatcgacttcctggaagcaaaaggatataaggaagtgaaaaaggacctcattatcaagctcccaaaatactcacttttcgagttggagaacggtagaaagaggatgctggcaagcgcaggggaacttcagaaaggcaatgagctggcattgccatcaaagtatgtgaacttcctctacttggccagccattacgagaaacttaaaggtagcccagaagataacgagcaaaaacagctctttgtggaacagcataagcattatctggatgagatcatagaacaaatctcagagttttccaagagagttatcctcgcagatgcaaacctggataaggttctctcagcctataataagcatagagacaagccaattagagagcaagcagagaacattatccacttgttcactcttacaaacctgggggcaccagccgccttcaaatatttcgatacaacaatagacagaaagaggtataccagcaccaaagaagttctcgacgccacactgatccatcaatcaatcacaggcctttacgaaactaggatcgacttgtcacaactgggtggggat

SEQ ID NO: 67 - DNA, 3307 bp, synthetic construct
  source 1..3307: mol_type = other DNA
gagcaaggacacctacgacgacgacttggacaacctattggcccagataggtgaccagtatgcagacctcttccttgcggccaagaacttgagtgacgctatactgctcagtgacatcctgagggtgaacactgagatcactaaggcccctctctctgcctcaatgattaagcgttacgacgagcatcaccaggatctcaccctgcttaaggcccttgttcggcagcagctccctgagaagtacaaggagatattttttgaccagtctaagaacggctacgccggttacattgacggtggggcaagccaggaggagttctacaagttcatcaagccgatccttgagaagatggacggcaccgaggagctacttgtcaagttgaaccgggaagacctgctccggaaacagcgtacattcgacaacggcagcatccctcaccagatccacctgggcgaactacacgccatcctccgacgtcaggaggacttctatccattcttgaaagataacagggaaaaaatcgaaaaaatacttacgtttcgaataccttactacgtggggccccttgctcggggaaactccagattcgcatggatgaccaggaagtcagaggagaccatcacaccctggaactttgaggaggtggttgacaaaggtgcttctgcccagtccttcattgagcggatgactaacttcgacaagaacctgcccaacgagaaggtgctgccaaagcacagcctgctctacgaatactttactgtgtacaatgagctgacgaaggtgaagtacgtgacagaggggatgcggaagcccgctttcctgagcggcgagcaaaaaaaagcaatcgtggacctactgttcaagaccaaccgaaaggtgacagtgaagcagctcaaggaggactacttcaaaaaaatcgagtgcttcgactctgttgagataagcggcgtggaggaccgattcaacgcctcattgggaacctatcacgacctgctcaagatcattaaggacaaggacttcctggataatgaggagaatgaggacatcctggaggatattgtgctgacccttactctattcgaggacagggagatgatcgaggagcgactcaagacctacgctcacctgttcgacgacaaggttatgaagcaattgaagcgtaggcgatacacggggtggggaagactctcccgaaaactgataaacggcatcagggacaagcagtcagggaagacgatcttggacttcctgaaatccgacgggttcgccaaccgcaacttcatgcagctcattcacgacgactcactaacgttcaaagaggacattcagaaggctcaagtcagtggacaaggcgactccctgcacgagcacattgcaaaccttgcgggctccccggcgattaaaaagggcattctccaaacggttaaggtggtggacgagctggtgaaggtgatgggccgacacaagcctgagaacatcgtgatcgagatggccagggagaaccagactacccagaagggtcagaagaactctcgggaacgtatgaagcgtattgaggaggggattaaggagttgggctctcaaatcctcaaggagcaccctgtggagaacactcagctccaaaacgagaagctgtacctgtactacctgcaaaacgggcgcgatatgtacgtggatcaggagttggacatcaacaggcttagcgattacgacgtggaccacatcgtgccacagtcattcttaaaggacgacagcatcgacaacaaggttctgacgaggagcgacaagaatcgagggaaaagtgacaatgttccatccgaggaggtggtcaagaaaatgaagaactattggcgtcagcttctgaacgccaagctcatcacccagcggaaattcgacaacctgactaaggctgagcgaggcggactctccgagcttgacaaggctggcttcatcaagcggcagttggtcgaaacccgacagataacgaagcacgttgcccagatacttgactcccgtatgaacaccaagtacgacgagaacgacaagctcatcagggaggtgaaggtcattacccttaagtccaaactcgtcagcgactttcgtaaggacttccagttctacaaggtgcgcgagatcaataactaccaccacgcacacgacgcctacctgaacgcagtggttggaaccgcgttgattaaaaagtaccccaagttggagtcggagttcgtttacggggactacaaggtgtacgacgttcggaagatgatcgccaagtctgaacaggagatcgggaaagcaaccgccaagtatttcttctatagcaacatcatgaacttctttaaaaccgagatcacacttgccaatggcgagatccgtaagaggccgctgatcgagacaaatggggagactggcgagatcgtgtgggacaagggccgcgacttcgcaaccgttcggaaagtcttgtccatgcctcaagtcaacatcgtcaagaagactgaggtgcaaacaggcgggttctcgaaggagtccatactgcccaagaggaactcagacaagctcatagcacgcaaaaaagactgggatccaaagaaatacggcgggttcgactcgccgacagtcgcatactccgtgttagtggtggctaaagtggaaaaggggaagtccaagaagctcaagtccgtcaaggagttgctcgggatcaccattatggaacggtcctcattcgagaagaatcccattgacttcctagaggcgaagggctacaaagaggtcaaaaaggacctaattattaagctccccaagtattcactcttcgaacttgaaaatggtcgtaagcggatgttggcaagcgctggagagcttcagaaggggaacgagcttgcactgccttccaagtacgtgaacttcctgtacctcgcctctcattacgagaagttgaagggctcaccggaggacaacgagcagaagcagttgttcgtggagcagcacaagcactacctcgacgagatcattgagcagataagtgagttcagcaaacgggtgatccttgccgacgctaacctggacaaggtgctgagcgcctacaacaagcacagagacaagccgatccgagagcaagcggagaacatcatacacctgttcaccctcacgaacctcggggctcccgcagccttcaaatattttgacacgaccatcgaccgtaaacgctacactagcacgaaggaggtgctggacgctacccttatccaccagtccatcaccggcctgtacgagacgagaatcgacttgtcgcagctcggtggtgac

SEQ ID NO: 68 - DNA, 4101 bp, Rubus sp.
  source 1..4101: mol_type = other DNA
gacaaaaaatactcaattggtctggcaattgggaccaacagtgtcggatgggccgtgattaccgacgagtacaaggtgccgtccaaaaaattcaaggtgcttgggaacaccgaccgccactcgatcaagaaaaacctaatcggtgcgttgcttttcgacagtggggagaccgccgaggcaacacgcttaaaacgcacagctaggaggagatatacacggcgcaagaaccgaatatgctacttacaggagatattctccaatgagatggcgaaggtggacgactctttcttccatcggcttgaggaatccttcctggtcgaggaggacaagaagcacgagcgacacccgatattcgggaacatcgttgatgaggtggcgtaccacgagaagtacccaacgatataccacttacgcaagaagctcgtggactctacggacaaggccgacttgcgccttatctacttggcactggcccacatgattaagttccgaggccacttccttatcgagggtgacctgaaccccgataactccgacgtggacaagctcttcatccaactcgtccagacatacaaccagctattcgaggagaatcctatcaacgcctctggggtggacgctaaagctatcctctcagcccgcctgtcaaagtcgaggaggttggagaacctaatcgcccagcttccaggcgagaagaaaaatgggctgttcggaaaccttatcgcactctcactgggcctaaccccgaacttcaagtccaacttcgacctggcagaggacgcgaaattgcagttgtcgaaagacacctatgacgatgacctggacaacctgttggcccagataggggaccagtacgccgacctgttcctagcggccaagaacctgtccgacgccatcttgctgtcggatatactgcgggtgaacaccgagatcactaaagcacctctctccgccagcatgattaagcgttacgacgagcaccaccaagatttgaccctgctaaaggcacttgtacggcagcagcttcccgagaagtacaaggagatctttttcgaccaaagcaagaacggctacgccgggtacatcgacggaggtgccagccaggaggagttctacaagttcattaagcccatcctggagaagatggacgggactgaggaactacttgtgaagctgaaccgggaagacttactacggaagcagcgtaccttcgacaacggttctatcccacatcagatccatcttggggagttgcacgcgatcctgcgacgccaggaggacttttaccccttcctgaaagacaaccgcgagaaaatcgagaagatactgaccttcagaataccttactacgtcggaccccttgcgcgaggcaactcaagattcgcgtggatgaccaggaaatcagaggagaccatcacaccctggaatttcgaggaggtggttgacaagggtgcctccgcccagtcctttatcgaacgaatgaccaacttcgacaagaacttgcccaacgagaaggtgctccccaaacacagcctcctctacgaatatttcacagtgtacaacgagcttactaaagttaagtatgttactgagggcatgaggaaacccgccttcctgtcaggcgagcagaagaaagctattgtggacctccttttcaagaccaaccggaaggtgacagtgaagcagctcaaggaggactacttcaagaagatagagtgcttcgacagcgtggagatcagcggggtggaggacagattcaatgcctctctcggaacataccacgacttgcttaagatcatcaaggacaaggacttcctcgacaacgaggaaaacgaggatattctggaggatattgttctgactcttaccctgttcgaggaccgggagatgatcgaggagcgtctcaagacctacgcccacctgttcgacgacaaagttatgaagcagctcaagcgtcggagatataccggatggggccgtctgtctcggaagctcatcaacgggatcagggacaagcagtcagggaagacgatcttagacttccttaagtctgacggcttcgccaacaggaacttcatgcagttgatccacgacgacagccttaccttcaaggaggacatccagaaggcccaagtgagtggccagggtgacagcctccacgagcatattgctaatcttgcgggttccccagcgattaaaaagggcatacttcaaaccgttaaggtggtggacgagcttgtcaaggtgatggggcgacacaagcccgagaacatcgtgatcgagatggccagggagaaccagaccacccagaaggggcagaagaatagccgagaacgcatgaagcgcatcgaggaggggattaaggagctagggagccagatcctcaaggaacatcccgtcgagaacacccagctccagaacgagaagctatacctctactacttgcaaaacgggagggatatgtacgtggatcaggagttggacattaaccgcctaagcgactacgacgtagatcacatcgtgcctcagtcattcctcaaagacgacagcattgacaacaaagtcttgacccgatccgacaagaaccgaggaaaatccgacaatgtgccctcagaggaggtcgtcaagaaaatgaagaactattggaggcagctacttaacgccaaactcataacccagcggaagttcgacaacctgacaaaggctgagcggggtgggctcagcgagcttgacaaggctggcttcatcaagcggcagttggtggagacaagacagataacgaagcacgtggctcagatcctggactctcgcatgaacacgaagtacgacgagaacgacaaattgatccgcgaggtcaaggttattacgctcaagagcaaacttgtcagcgatttccgcaaggacttccagttctacaaggtgagggagattaacaactaccaccatgcacatgatgcctacttgaacgcagtggtggggaccgcgcttattaaaaagtaccctaagttggagtcagagttcgtttatggggactacaaggtgtacgacgtccggaagatgattgcaaagtctgaacaggaaatcgggaaggccaccgccaaatatttcttctacagtaacattatgaatttttttaagactgaaattactctcgcaaacggcgagatcaggaagcgtcccctcatcgagacaaacggggagaccggggagatagtctgggacaaggggcgggacttcgctacggtgaggaaggtgctctcgatgccacaagtgaacatcgtcaaaaagacagaggtgcagaccggtggcttctcaaaggagtcaatcctgccaaaacgtaacagcgacaagctcatcgcccgcaagaaagactgggaccctaagaagtatggtgggttcgactcaccgacggtcgcatactccgttctggtcgtggcaaaggtggaaaagggcaagtccaaaaaactgaaatccgtgaaggagttgcttggcattaccatcatggaacgcagcagcttcgagaagaaccccattgacttcctggaggctaaagggtacaaggaggtcaagaaagatttaattattaagctacctaagtacagcttgttcgagctggagaacggccgaaaacgaatgctcgcatccgccggggaacttcaaaagggcaacgagcttgcgctgccctccaagtacgtgaacttcctgtacttggcatcccactacgagaaactcaagggtagcccagaggacaacgagcagaagcagctattcgtggagcagcacaagcactacctcgacgagataatcgagcagatcagtgagttcagtaagcgggtgatactcgcggacgccaacttggacaaggtgcttagtgcctacaacaagcaccgtgacaagcccatccgagaacaggctgagaacatcatccaccttttcactctgacaaacctcggtgctcccgccgccttcaaatacttcgacactaccatcgacaggaagcgctacacatctacgaaggaagttcttgacgctacgcttattcatcagtctatcacagggctgtacgagacaaggatcgaccttagccaactcggcggggat

SEQ ID NO: 69 - DNA, 6207 bp, Rubus sp.
  source 1..6207: mol_type = genomic DNA
gctcatgcgtcataattctaggtttgggtgggacccgatcacaaagagattcatatatgatgatgaagtatgagcatactattttatttgaaggtaaatttctattgacttttattctattctatttcgttaatgttctttttttttcttctattttttggtatgttttgtttttttttttgatacatatacctttgattttattaattacattgggaggttgctctttcctttgttttctaattattttgtgtcacaacattatttaagtttttctgaaatttgtagaagttgtttataataaagtttgtagatttcataaatcaataaaaggacttaaataaatcaatagaagtctatgaaatctatgaagtctgtagacttttcaaaaagtttgtaaattaaaaaaaaaagggtccttgaccataaggtccaaagttggacaaatatttaccatcaaggacaaaggaaaaacacggtgcccacccggtccattaattgaccaatttatatttttgaattatacattttacaaaaattatgaaactgccactggaaaattttattgaccacaataattttttttttttgagaaaacgtatattcctatggtaggagagaaagaacaaaaaaaatcttttttgtgtattttattgacctcaataaaattttattgagctccataaactgttttattgaggtcaataaacttttttattgaggtcaataaactgtttcattgagatgacattggttttgaggtcaataaactgtttcattgaggtgacattggttttgaggtcattaaactattttattgaggtcaataaactgtttcattgaggtgacattggttttgaggtcaataaactattttattgaggtcaataaattatttcattgaggtgacattggtattgaggtcaataaattgtttcattgagatatcattagtattgagatcaataaatttttttattgaggtcaagaaattttttttattgaagtcaataaactgtttattgtggcatttttgtaaatattggtgaccttatttgggaaaaaatattaggtggcttttttaagaataggatcacccaaagtggctttattggtaattatttattaacttaattcatgacagattatgagaaaaaaaagagagaatagaggagagagagagagagagatagataagaagaagaagaagagaaaacaaagatgccaaaaatagacactaagggcaataatgtcacaaaaaatcactatttaatatttttgtccttactgggagtaaaacatgtttaactgaaacttagtgagaattatttaaaatttatcaaacttgaagggaaatatgtagtgtaaaagaggatctattggttatttaccttaaaaaaaatttcatacaaatccatacacaatacacattgactatttaattatatgatgtattaatttgtttatactctaaagtacacaaccttgtggctgtagtttagtggtaagaattccacgttgtggccgtggagacctgggctcgaatcccagcagccacatagttttgttttttaaatcaattcccttctttttttcatgggttacatttatggaggtctcaaacccttatattcctttttcccttgtgtaataaaaatttaaaacataattgctaattagattttccttttaataaacctctacgtatattaacgcaacatatttctcacaattccatcccctagccttgagctacgaaggctttgtatataggactaagaggtttgaataattataaacttacaaacaaaatttgtaaaataattattgcatggtaccattaccacaattgtggcaatacatattggtttaattttagaccaacccaatagcagaaggcactaatttatgaccaacaagtacttttccgaaacttagagacaaatttgtcatcacctttttgtgtatgcacaattgaaacaaaatgtgttccttgcaaagattgattgatcgatatttaagtgaaaatcaaagtatgattgattaatccttttaaataaagtaaacatgaacaatgtacgtcgaaatagataaaaagggcctgtggtggtttttgttttactacccagacaggtttttgttgaagagtaaatgcaggtgtcaggagagggtaggagctggtgcgacatgaaagtacttaattctgtcattatttccatcttttcgtttacgtaagctcaatgtcatcctcaattttaaatgtatatttccactgttcctacttgtttgaaccggaaaaggttagtatagctaccatcatgctcatgtatgtatgtatgtgtgtatatatatatcccagtctctccagtctcttctcagctttgctcaggagtccaaacattcctttgtctagttagtcttgccttcctgtttgaatcacaaatcatgtggatgatgacggggtgtcgcagcgatggttgtggagagcctaacattaacaacagggatatccatgactactcgatcaaagctgcaaatcttcagcctgcagtagtagtactcactccaaagcttagcgctacttccaacataacccaaaattctgtgttaccattgaaccaccgccagcatcagggtacgtacatacgtaaacatatatataatatagatcacacatatcgatcttagtttgatggtctgattattctgtacgtgggtaggtaatagactaatagttcatatataattatacatgtgcagtgatccctgtcgcgccgtctgagcaaggaagaagtggtagtgattatttttataatgatcaacaaccagctgctgctgctgttgctcctccgcggagttcccgatggaatccaacgccagagcagctactggccctcgaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatctatgtctcagtctcactctaatgaacaacgacaacaatcttggactaacaacctaatcagcagtatccagaacaaagaatccgcaggaggtactaattgagtcgcataattttcttattaatatatatatatatatgtacgtgcatatatatccatgcactatatatgtgcatacctaacctactgccatatttttaacagcgtaattaattaagcgtttagtgtccgtgattatctctcatggtactaattaaagttttgtttgcagaaggtcgacgtcttattagtactgtatatcaaatatgttttcggggtttttttcctccatgtgtgttactctccacctgtagcttcggggttttttataactcaattgatttcaatcaatattgttggaaaaccacctaatagaataattttagcacacacttacttggccggaactagggctcaactctaggtgaggccacctccatcctttaggtcacctacgctcaataactctacaccgtattaaatttcttgttataaggagaggagagactcagtggttgtgttaataataatttagtcttacaattaatatacctgggactagcgggcaccaagcaagatagcaaaccctagctaagagcaggacagcttgattaattgattgggcactcgatcgagtacatgtacttgtaaatttttctatatgacttaattttatgattgatctggtgcaaaaactgattaacacatatcacgtacaattgtgctcttctcccttcagacattgattaaccaataaatcacaactgtcctttgcaggattgagaaacttaagccttgaaggtgagcagcagaacaagtggactctatcaaactgtactattctttctgatcaggtatacgcgtgctatatctatcattttattaacgtacgtagttttaagacttaactatatctgaccaaaagctcaagaaaagatagaaatatattaattaattgaaagttaaattttttacagttttactctctctctctctctctctctcccccccccccccccaatagtatgtatatgtacttcatttgttaattagttactgtacgtccgcaggaatcatcttcagtgctgcaagagaggatgagcatggtggaaagcaattcctccaagcaaccggtggatcagtcctattcggctacaccgacctcacaaccaccatgcgcaaacccaactgtcaccttaataaacacaaagcccttggattgtcgtcactattatgattatggtatggtattgaccgcgccccatatatgcaaaggcgaagaaaccagagaaactcgaactctggagctctttcctctcgagagcgacaatctcaagggggttaaggattccgcattaccacaaacaaaaaaagatactactgcagacttcgtggctgaccaatattttcagtttctttgaagaatttaagggagcagcattcacatagctagaaattaatctacttaagaagggtttttcaatctatcgatctatttaataaattattatggattaattaattatgtatgtgtgtcttctctttgatggaaatgatagctagggtacatctttatcgggttctgttttggttctttccacttttatttcttctttttgtttatatgcagttcatttgtaaggacgggtaaggttgattgatgtccccctttctcgttcgttaggttctttctgtgtctctttctttctactgttgagggatatgtagttatatatcctgtcgctaggtgtacctttatctttttctgtaaataaaactccatcgatttcatggcagaccaatatatattttcaatttcattgaagaatttaagggcttatcaatttctctatctaatataaaatatataggcattttttactaatgggatccttactttttatgctacaaaattttgatttcagtcatcagatcacattatttaaaacaaatctgatagctgagatcaatactccgcaccataaaaagtaaggacctctttatataaaaattattgatatatgaagaaatttgatcacaacttaccgagagaaatgagttgcagctatatatgcttcgactagattacacataaataagccacgagtcctctagttgtaggcaactatcgtaacaaactgtaacaaaccccttggtcacgagataagcacaagctataataaacaaactatgtaaacctagctaaacttagtttattaacaaactaactagtcggaaactgatgagaaattttattaggacctacaaatttgctttacctaacttcctatattctttgcacctcttttttgcttttaaaatataagatttgcttactaaactttttttaaatttctaaaataaccataatgatataattcacattaaatatacaaaattactactataattctaaatttactcaccacacatttattaattaatttataatcaataaaaaaaatttagcatgaattattattatttttaactattcttttctcttttagctgtgcaataaaaaaaatagtaatcatttattcttacagtttatttttttctctattttttgtacctcatgattaaatattttatatatattttatgttgctagtttttttttttacaaatataatggatagacttaaatttaggtcatagtagaatttttttcgttctctatcactaatatacgttcaacaaatgatacaaatttttatgtcacataaactatcataattttaattcttattacatatatataaatattttaatgagtacgtattgaaattaaaaaatgaaaatagataagaaaaataataaaaaatacaatataatattattgaattagaaactgtagagaaaataaatataatatttttttggtataattattatccttaatagttttataggaggttataaatataatttaataattcataatggagtgaggttaactgagcaaaaatttggaggtttaagggaaactaattgatgactaattgccgacttagatgcttcatcataacatgtttgtcttctccttgttggaatggcatgcatggggtcggattctgttttggtttcccatttttctttgaatg

SEQ ID NO: 70 - DNA, 1434 bp, Rubus sp.
  source 1..1434: mol_type = other DNA
aacattcctttgtctagttagtcttgccttcctgtttgaatcacaaatcatgtggatgatgacggggtgtcgcagcgatggttgtggagagcctaacattaacaacagggatatccatgactactcgatcaaagctgcaaatcttcagcctgcagtagtagtactcactccaaagcttagcgctacttccaacataacccaaaattctgtgttaccattgaaccaccgccagcatcaggtgatccctgtcgcgccgtctgagcaaggaagaagtggtagtgattatttttataatgatcaacaaccagctgctgctgctgttgctcctccgcggagttcccgatggaatccaacgccagagcagctactggccctcgaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatctatgtctcagtctcactctaatgaacaacgacaacaatcttggactaacaacctaatcagcagtatccagaacaaagaatccgcaggaggattgagaaacttaagccttgaaggtgagcagcagaacaagtggactctatcaaactgtactattctttctgatcaggaatcatcttcagtgctgcaagagaggatgagcatggtggaaagcaattcctccaagcaaccggtggatcagtcctattcggctacaccgacctcacaaccaccatgcgcaaacccaactgtcaccttaataaacacaaagcccttggattgtcgtcactattatgattatggtatggtattgaccgcgccccatatatgcaaaggcgaagaaaccagagaaactcgaactctggagctctttcctctcgagagcgacaatctcaagggggttaaggattccgcattaccacaaacaaaaaaagatactactgcagacttcgtggctgaccaatattttcagtttctttgaagaatttaagggagcagcattcacatagctagaaattaatctacttaagaagggtttttcaatctatcgatctatttaataaattattatggattaattaattatgtatgtgtgtcttctctttgatggaaatgatagctagggtacatctttatcgggttctgttttggttctttccacttttatttcttctttttgtttatatgcagttcatttgtaaggacgggtaaggttgattgatgtccccctttctcgttcgttaggttctttctgtgtctctttctttctactgttgagggatatgtagttatatatcctgtcgctaggtgtacctttatctttttctgtaaataaaactccatcgatttcatggc

SEQ ID NO: 71 - protein, 336 aa, Rubus sp.
MWMMTGCRSDGCGEPNINNRDIHDYSIKAANLQPAVVVLTPKLSATSNITQNSVLPLNHRQHQVIPVAPSEQGRSGSDYFYNDQQPAAAAVAPPRSSRWNPTPEQLLALEELYRRGIKTPTAQQIRQVTALLRHFGKIEGKNVFYWFQNHRARERQKRRRELMSSMSQSHSNEQRQQSWTNNLISSIQNKESAGGLRNLSLEGEQQNKWTLSNCTILSDQESSSVLQERMSMVESNSSKQPVDQSYSATPTSQPPCANPTVTLINTKPLDCRHYYDYGMVLTAPHICKGEETRETRTLELFPLESDNLKGVKDSALPQTKKDTTADFVADQYFQFL

SEQ ID NO: 72 - DNA, 5563 bp, Rubus sp.
  source 1..5563: mol_type = genomic DNA
gctcatgcatcataattctaggtttgggtgggacccgatcacaaagagattcacatatgatgatgaagtatgggtagactatttgaaggtgaatttctattgccttttattctattctatttcgttaatatttttttcccttctattttttggtacgttttggtttgtttttgatacatataccttcggttctgttaattatattgagaggtttttcctttgttttctaattattttgtgtcacaacattatttaagtttctttgaaatttgtagaagttgtttataataaagtttgtagatttcataaatcaataaaaagtcaatggtaaaagtcagtaactttaaataaatcaatagaagtctatgaaatatgttgacttttcaaaaaatttgtgaattaattttttttttaatacaaatcaatatagaaacatccccctaactgacaacctcagttgctgattaattttgaaacacatacgctttgactttttaaatatatgatgtattaaaaatttaatattcttaagcacataaccttgtggctgtagtttagtggtaagaattccacgttgtggctgtggagacctgggctcgaatcccagcagccacagagtttaattttttaaatcaatccccttcttttttttcatgggttacatatatgatatatggtctaaaacccttatattcctttttcccttgtttaataaaaatttaaaacataattgctaattagattttcctttttcattttaataaacctctacgtatattaacgcaacatatttcgcacaattccctcccctagccttgagctatgaaggctttgtatataggaataagaggtttgaataattataaagttacaaacaaaatttgtaaaataattattgcatggtaccattaccacaattgtggcaatacatactggtttaattttagaccaactcaataccaaaacgcactaatttatgaccaacaagtacgtacttttccgaaacttagagacaagtttttcatcacctttttgtgtatgcacaattgaaacaaaatgtgtttcttgcaaagattgattgatcgatatttaagtgaaaatcaaagtatgattgattaatccttttaaataaagcaaacatgaacaatgttacaatgtacgtcgaaatagattaaaagggcctgtggtggtttttgttttactacccagacaggcttttgttgaagagtaaatgcatgtgtcaggagagggtaggagctggtgcgacatgaaagtacttaattctgtcattatttccatcttttcgtttacgtaagctcaatgtcatcatcaattttaaatgtatatttccactgttcatactttgtttgaaccggaaaaggttagtatagctaccatcatgctcatgtatgtatgtatgtatatatatatatatatatatatatatcccagtctctccagtctcttctcagctttgctcaggagtccaaacattcctttgtcttgttagtcttgctttcctgtttgaatcacaaatcatgtggatgatgacggggtgtcgcagcgatggttgtggagagcctaacattaacaacagggatatccatgactactcgatcaaagctgcgaatcttcagcctgcagtagtagtactcactccaaagcttagcgctacttccaacataacccaaaattctgtgttaccattgaaccaccgccatcatcagggtacgtacatacgtaaacatatatataatatagatcacacatatcgatcttagtttgatggtctgattattctgtacgtgggtagttaatagactaatagttcatatataattatacatgtgcagtgatccctgtcgcgccgactgagcaaggaagaagtggtagtgattatttttataatgatcaacaaccagctgctgctgctgttgctcctccgcggagttcccgatggaatccaacgccagagcagctactggccctggaactctggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatccatgtctcagtctcactctaatgaacaacgacaacaatcttggactaacaacctaatcagcagtatccagaacaaagaatctgcaggaggtactaatttagtcgcacaattttcttattaatatatatatatatgtacgtgcatatatatccatgcactctatatatgtgcatacctaacctactgccatatttttaacagcgtaattaattaagcgtttagtgtccgtgattatctctcatggtactaattaaagttttgttcgcagaaggtctccgtcttatttgtactgtatatcaaatatgttttcgtctctccctccatgcgtgttgctctccacctgtagcttcggggtttttttaaactcaattgatttcaatcaatattgttggaaaaccacctaatagattaattttagcacacacgtacttggcctgaactagggctcaactctaggtgaggccacctccatcctttaggtcacctacgctcaataactctacaccgtattaaatttcttgttataaggaggggagagactcaatggttgtgttaataataatttagtcttacaattaatatacctgggactagcgggcaccaagcaagatagcaaaccctagctaagagcaggacagcttgattaattgattgagcacttgatcgagtacatgaacttgtaaattttttctatatgacttaattttaagattgattaggtgcaaaaactgattaacacatatatcacgtacaactgtgctgttctcgcttcagacattgattaaccgatatatcactactgtcctttgcaggattgagaaacttaagccttgaaggtgagcagcagaacaagtggactctatcaaactgtactattctttctgatcaggtatacgcgtgctatatctatcattttcttaacgtacgtagttttaagacttaactatatctaaccaaaagctcaagaaaagatagaaatatattaattaattgaaagttaaatttttaacagttttactctctccctctctccctttctctctcataattgtacgtatatgtacttaatttgttaattagttactgtacgtccgcaggaatcatcttcagtgctgcaagagaggatgagcatggtggaaagcaattcctccaagcaaccggtggaacagtcctattcggctacaccgacctcacaaccaccatgcgcaaacccaactgtcaccttaattgacacaaagcccttggattatcatcactattatgattacggtatggtattgactgcaccccatatatgcaaaggcgaagaaaccagagaaactcgaactctggagctctttcctcttgagagcaacaatctcaagggggttaaggattccacattaccacaaacaaaaaaagatactactgcagacttcgtggctgaccaatattttcagtttctttgaagaatttaagggagcagcattcacatagctagaaattaatctctatatatctatttaataaattattatggattaattaattatgtatgtgtgtcttctctttgatggaaataatacctagggtacatctttatcaggttctgtttttggttctttccacttttatttattcttgttgtttaaattcagttcaatttgtaaggacgggtaaggttgattgatgtccccatttctcgttcgttaggttctttctgtgtctctttcgttctactgttgagggatatctagttatatatcctgtcgctaggtgtacctttatctctttctgtaaataaaactccatcgatttcatggcagaccaatatatattttccatttctttgaagaatttaagggcttgtcaatttctctatctattataaaatatgtaggcattctttgctaatgagattcttactttttatgctacgaaattttgatctcattcgtcagatcacatcatttaaaacagatctgatggctgagatcaaaactccgcaccataaaaagtaagaacctctagctttatatgagaattattgataatatatatatatatatatatatatatatataagcatgtgttgaaaactgtaagagaaacttgaagaacaaaaagggatgtatttgatatatgaagaaatctatcacaacttacagagagaaacgagttgcagctatatatacttcgactagattacacataaataagccacatgttctccgatccagtagtaggcaactattgagaaatttttcagagtgactatcctatgtgaccacttactgtgattaggccacatcattgcatagataactaatgaccaatggttgatacacaggaggaaaaaaataaaagataaggtccaattataataggtggtcacatatgccacatggcctgacggtcacacctaaaaatttctcggcaaatatcctaacaaatcataacaaaccccttggtcacgagataagcacgagctataataaacaaactatgtaaacctagctaaacttagtttttaacaaactaactagtcggaaactaatgagaaattttattaagacctacaaatttgctcacctaactttttatgtatatattctttgcacctgttttttgcttttaaaatataagattttcttactaaatctcttttaaattcataaaataatcatgatgatataattcacaaaatatacaaaattactactataattctaaatttactcaccacacatctattaattaatttataatcaacaagaaaatatcttagcatgaactgtttttttttttaactattttttttctttttgctgtgcaataaaaaaaatcatttattcttaccgtttatttattttttctacctcatgattacatattatatatatatatatatattttatgttgctagttttttttttagaaatataatggatagacttaaatttaggtcatagtagaatttatttcgttctctatcaccaatatgcgtttaacaaataatacaaatttttatgtcacatgaactatcatagttttacttcttattaaatatatataaatattttaatgggtatgtactaaaatttaaaaataaaaatagataaaaaaaataataaagaatgcaatataatattattgaattggaactctagagtaaataaatataatatctttttggtataattattgttttataggaaattataaatgttatttaataattcataataaagtgatgttaagtgagcaaatttgaaagtttcaataaaaatactcaaaactaattaatgaataaatgctgacttaattagatagttcattataacacgtttgtcttctccttgttggaatggcatgcatggggtcggattctgttttggtttcccacttttgtttgaatg

SEQ ID NO: 73 - DNA, 1423 bp, Rubus sp.
  source 1..1423: mol_type = other DNA
aacattcctttgtcttgttagtcttgctttcctgtttgaatcacaaatcatgtggatgatgacggggtgtcgcagcgatggttgtggagagcctaacattaacaacagggatatccatgactactcgatcaaagctgcgaatcttcagcctgcagtagtagtactcactccaaagcttagcgctacttccaacataacccaaaattctgtgttaccattgaaccaccgccatcatcaggtgatccctgtcgcgccgactgagcaaggaagaagtggtagtgattatttttataatgatcaacaaccagctgctgctgctgttgctcctccgcggagttcccgatggaatccaacgccagagcagctactggccctggaactctggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatccatgtctcagtctcactctaatgaacaacgacaacaatcttggactaacaacctaatcagcagtatccagaacaaagaatctgcaggaggattgagaaacttaagccttgaaggtgagcagcagaacaagtggactctatcaaactgtactattctttctgatcaggaatcatcttcagtgctgcaagagaggatgagcatggtggaaagcaattcctccaagcaaccggtggaacagtcctattcggctacaccgacctcacaaccaccatgcgcaaacccaactgtcaccttaattgacacaaagcccttggattatcatcactattatgattacggtatggtattgactgcaccccatatatgcaaaggcgaagaaaccagagaaactcgaactctggagctctttcctcttgagagcaacaatctcaagggggttaaggattccacattaccacaaacaaaaaaagatactactgcagacttcgtggctgaccaatattttcagtttctttgaagaatttaagggagcagcattcacatagctagaaattaatctctatatatctatttaataaattattatggattaattaattatgtatgtgtgtcttctctttgatggaaataatacctagggtacatctttatcaggttctgtttttggttctttccacttttatttattcttgttgtttaaattcagttcaatttgtaaggacgggtaaggttgattgatgtccccatttctcgttcgttaggttctttctgtgtctctttcgttctactgttgagggatatctagttatatatcctgtcgctaggtgtacctttatctctttctgtaaataaaactccatcgatttcatggc

SEQ ID NO: 74 - protein, 130 aa, Rubus sp.
MWMMTGCRSDGCGEPNINNRDIHDYSIKAANLQPAVVVLTPKLSATSNITQNSVLPLNHRHHQVIPVAPTEQGRSGSDYFYNDQQPAAAAVAPPRSSRWNPTPEQLLALELWKSCTDVASRHQRPSKSDK

SEQ ID NO: 75 - DNA, 6761 bp, Rubus occidentalis
  source 1..6761: mol_type = genomic DNA
gaacaagaccaagattggtcgaattcgatgcctttgtgaactttctttatcacaaagattttcttggatgctcatgaagttactttatcatagcatatattatgaatccccacgattcatataaagctctttaacttttactaaagaatgatgacattaggggtgctcatgacttacatataccgcaaacgatataaagctcgatcactcccctaatacttctcataatttttcgaagtaggtatagcttgtttgtctttttgctcatatattttcaccaagaaagtcccacgccataagaggggtcgggatgtagaagaagggaagtttttaaatccccgagaaaaaacacaaccgcatcaaaagcaggaacttttagaaaatacttacttggttttcttggaatttgaatatcttgagatccgacggtatgaacaggctgcaatttgaaccggtagcagtagacacacagaagaagcagtcgacgaaattgcagcttgatccaacggttgatgagggagaaaattgctttggaagtcggctgtctcgtgtaggcagattttagagttttgggtttttgtagctagggttagggctcgtgctgataacatgtttaagggaaacgtaaatgacgagagaaatagtagagaatgctcagaataggatcaactttattaatgaataaagggcctctatatatagaggaatacatgatacaatccatattattgtaggaaatatctaatccatattctagaaggataacctatactaagagtcctagtttgacaaggcacacaataatgttttccttcaacaagttctattttttttaagaatggtagaattaatccactaaagccagagagaagcatacatcaaatcctagaacccagcttataccctgctaggaaactaaaataaagataagcaaaactcatttagtctagaaaactcgatttatttcatcgagcctacaaaaataaaaacttatacgcatctaaaatgggaaatacaactacaaaatcgcctaagcactcaattgtggtacaacttcgtaattgttttttagataaatcaaagcttccactattgtagacacaccaccacagcaaaaacaactcacagctgcttcttacccttacgacgaagggaaggagtagcttcttggtaaaccctaaaccaaccgaacccatgaccaaaccctagcccaaagagaggccaggcccaagcatggtttgcaaagcgacttgacaccaaacaataaacccaacgcttaagcaggatgtcgtcgccgatctcatgcgatgccaacaaccaaagaagcggaccacttgctgctcctgggatcacccaaatcgaatcacctacaaaaagaaaacaaaggctacggtgataggaggccatagcttgacgacgaccagagccgcccttgaagctggagtctcccctagaaaagacgaggccagctgtattgttcgttgggaacgtgcccaaaccatgaaaggcaaggatatggcctgaaggaagccgacatcgctgctccttgacagcaaaacgatcgaaatcgccaagcttgttgattgcaatgacagagagatctgccttgcctgttgaatggatgaggcgctgcaaccctgccacaaaggagagggaggaggcggctccttggtgtttttccgagataaaacctagcatcctatttttgttgctctttatcaaggaatcaactttgtaagagatactgtagtttaatatttagatttattctctcggtaaacttgttagttcaccatcttttaataatgtaagttctattaaatataatataactgaaaaaataaataacacgtactctccttaattcctaatagtatgtgtaaatgcgaaacacaatagtaaataattatataactaccagcagctttcaatgaaaattccgaccatcatatgtattatggtgactactctagctagcctagctcgctaccccaaaacgagatggatacgcaaaaaacatgcatatagctagctagagccccgaaataaatagcactttagttagctagtactgcccaaccatatataacaataatagcacttttataaaaaatggtatatttgcgtgtatgcatacccactttatttaaggatatgggaattatgatttcattttatatcatttttttgtaccttttttatattttttataaaactatttatactattgttaagagaaaacagtcaagacactaatagtttgtagtaatcttcaaatataaaagtgtcaaatgcaattaacaattaattaaaaaaaaatcaacaacaaaactatttttaaacactcattcacttctatgggtgtcactttctagtatatttgaagaaaagaagtctaagactatcccttctcaatcacaccctaaaaaactttatttcacctgttttattagtcgatgaaggcctcaatcaatatttctagacttcatactctatgagcaattaaaagaaacaataacccactaactactagcttgctagtggggttaaaaaaaaacttactaattaattttgaaacaaatacgcttaaacttttaaatatatgatgtattaattttcatattcttaagtgcataaccttgtggctgtagtttagtggtaagaattccacgttgtggccgtggagacctgggctcgaatcccagcagccacatagtttaattttttaaatcaatccccttctttttttcatgggttacttggaggcctaaaacccttttatattcctttttccctttgtttaataaaattttaaaacataattgctaattagattttcctttttcattttaatatttcccacaattccctcccctagccttgagccacaaaggctttgtttgtatataggaataggaggtttgaataattataaacttacaaaacaaaatttatattgttgatgagaagatatataccacaaccagtgtaaaataattcttgcatggtatcattaccacaattgcggcaataaatacagctttaattttagaccaattcaaaagtaaaacgcattaattcatgaccaacaagtacttttccgaaacttcaaatttttcatcacccttttgtgtatgcacaattgaaacaaaatcaaaatgtattcttgtcaaagattgattgatcgatatttaagtgaaaatcaaagtatggtcaactacgtaaatatatataatccttttaaataaagtaaacgtacatgaacaatgtacgtcgaaatagataaaaagggcctgtggtggtttttgttttactacccagacagggttttgttgaagagtaaatgcatgtgtcaggagggggtaggagctggtgcgacatgaaagtacttaattctgtcattatttccatcttttcgtttacgtaagctcaatgtcatcctcaattttaaatgtatatttccactgttcctactttgtttgaacctgaaaaggttagtatagctaccatcatgctcatgtatgtatatatatatcccactctctccggtctcttctcagctttgctcagtagtccaaacattcctttgtctagttagtcttgctttcctgtttgaatcacaaatcatgtggatgatgacggggtgtcgcagcggtggttgtggagagcctaacattaacaatagggatatccatgactactcgatcaaagctgcaaatcttcagcctgcagtagtactcactccaaagcttagcgctacttccaacataacccaaaattctgtgttaccattcaaccaccatcaccatcaccatcagggtacgtacatacgtaaacatatatatatatataatatagatcacacatatcgatcttagtttgatggtctattctgtacgtgggtaggtaatagactaatagttcatataaaattatacatatgcagtgattcctgtcacgccgtctgagcaaggacgaagtggtagtgattatttttataatgatcaacaaccagttgctgctgctgtagctcctccgcgaagttcccgatggaatccaacgccagagcagctactggccctggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatccatgtctcagtctcactctaaagaacaacgacaacaatcttggactaacagcctaatcagcagtatccagaacaaagaatctgcaggaggtactaattgagtcgcataagtttcttattaatatatatatatatatatatatatatatccatgcactctatacatatgcatacctaacctactgccatattttttaacagcgtaattaattaagcttttagtgtccgtgattatctctcatggtactaattaaagttttgtttgcagaaggtctccgtcttattagtactgtatatcaaatatgttttcgtctctccctccatgtgtgttactctccaccagtagcttcggggtttttttaactcaattgatttcaatcaatatttttggaaaaccacctaatagattaagtttatcatacacttacttggggggaactagggctcaactctaggtgaggccacctccatcctttaggtcacctacgctcaataactctacaccgtattaaatttcttgttataaggaggggagagactcaatggttgtgttaataataatttagtcttaaaattaatatacctgggactagcgggcaccaggcaagatagcaaaccctagctaagagcaggacagcttgattaattgattgagcactcgatcgactacatgagcactcgagtacatgaacttgtaaatttgtttctatatgacttaatttcatgattgatcaggtgcaaaagctgattaacacatatatcacggccctatatcacgtacaactgtgctgttgtgtcttcagacattgattaaccatatatcacaattgtcctttgcaggattgaaaaacttaagccttgaaggtgagcagcagagcaagtggactctatcaaactgtatttctgatcaggtatacgcgtactatatttatcattttcttaacgtacgtagttttaagacttaactatatctaaccaaaagctcaagaaaagatagaaatgaattaattaattgaaagttcagaattttacagttttactctctctctctctctctctctctctctctctctctctcatatttgtatgtatatctacttaatttgttaattagttactgtacgtccgcaggaatcatcttcagtgctgcaagagaggatgagcatggtggaaagcaattcctccaagcaaccggtggatcagtcctattcggctacacagatctcacaaccaccatgcgcaaacccaacagttaccttaatagacacaaagcccttggactatcatcactattatgattatgattatggtatggtattgacagcaccccatatctgcaaaggcgaagaaaccagagaaactcgaactctggaactctttccactcgagagcgacaatctcaagggggttaaggattccacattaccacaaacaaaaaaagatactactgccgacttcgtggctgaccaatattttcagtttctttgaagaatttaagggagcagcattcacatagctagaaattaatctcctttagaagggtttttcaatctatcgatctatttaataaataattatggattaattaatatgcatgtgtgtcttctctttatggaaatgataggtagggtacatccttatcaggctctgtttttggttctttccacttttatttcttcttattgtttaaatgcagttcaatttgtaaggacgggtaaggttgattgaggtccccctttctcgttcattaggttctttctgtgtctctttctttctagtgttgagggatatctagtaatatatcctgtcgctagctaggtgtaccgtacctttatctttttctgtaaataaaactccatcgatttcatggcagaccaatatatattttcaatttctttgaagaatttaagggtttgtcaatttctctatctaatataaagtatatatgtatgtgcatgtgttgaaaactgtaagagaaacttgaagaacaagaagggatgtatttgatatatgaagaaatttgtcacaacttacagagatcgagaaacgagctgcagctatatatacttagactagattacacagaatgacagaaataagccacgtgtcctccagctgtaggcaactatcctaacagaccgtaacaaaccccttggtcatgagataagcacgagctataataaacaaactatgtaaacctagctaaacttagttaattaacaaactaactaatcaggaactaattgatgactaattgctgacttagatgcttcatcataacatgtgtgtcttcttcttggtggaatggcatgcatggggtcagattctgttttggtttcccacttttgtttgaatgcagattaatttgtttggaaaggtagggttgattgtgtctccctttctctttaataagtttttggtggtcttagaaacttgcaactttgcaagtgttgtgtgtgtgtttatataataattttcaggtgaagatatgtgaagttttaatttcaactgt

SEQ ID NO: 76 - DNA, 1452 bp, Rubus occidentalis
  source 1..1452: mol_type = other DNA
agtccaaacattcctttgtctagttagtcttgctttcctgtttgaatcacaaatcatgtggatgatgacggggtgtcgcagcggtggttgtggagagcctaacattaacaatagggatatccatgactactcgatcaaagctgcaaatcttcagcctgcagtagtactcactccaaagcttagcgctacttccaacataacccaaaattctgtgttaccattcaaccaccatcaccatcaccatcaggtgattcctgtcacgccgtctgagcaaggacgaagtggtagtgattatttttataatgatcaacaaccagttgctgctgctgtagctcctccgcgaagttcccgatggaatccaacgccagagcagctactggccctggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatccatgtctcagtctcactctaaagaacaacgacaacaatcttggactaacagcctaatcagcagtatccagaacaaagaatctgcaggaggattgaaaaacttaagccttgaaggtgagcagcagagcaagtggactctatcaaactgtatttctgatcaggaatcatcttcagtgctgcaagagaggatgagcatggtggaaagcaattcctccaagcaaccggtggatcagtcctattcggctacacagatctcacaaccaccatgcgcaaacccaacagttaccttaatagacacaaagcccttggactatcatcactattatgattatgattatggtatggtattgacagcaccccatatctgcaaaggcgaagaaaccagagaaactcgaactctggaactctttccactcgagagcgacaatctcaagggggttaaggattccacattaccacaaacaaaaaaagatactactgccgacttcgtggctgaccaatattttcagtttctttgaagaatttaagggagcagcattcacatagctagaaattaatctcctttagaagggtttttcaatctatcgatctatttaataaataattatggattaattaatatgcatgtgtgtcttctctttatggaaatgataggtagggtacatccttatcaggctctgtttttggttctttccacttttatttcttcttattgtttaaatgcagttcaatttgtaaggacgggtaaggttgattgaggtccccctttctcgttcattaggttctttctgtgtctctttctttctagtgttgagggatatctagtaatatatcctgtcgctagctaggtgtaccgtacctttatctttttctgtaaataaaactccatcgatttcatggc

SEQ ID NO: 77 - protein, 337 aa, Rubus occidentalis
MWMMTGCRSGGCGEPNINNRDIHDYSIKAANLQPAVVLTPKLSATSNITQNSVLPFNHHHHHHQVIPVTPSEQGRSGSDYFYNDQQPVAAAVAPPRSSRWNPTPEQLLALEELYRRGIKTPTAQQIRQVTALLRHFGKIEGKNVFYWFQNHRARERQKRRRELMSSMSQSHSKEQRQQSWTNSLISSIQNKESAGGLKNLSLEGEQQSKWTLSNCISDQESSSVLQERMSMVESNSSKQPVDQSYSATQISQPPCANPTVTLIDTKPLDYHHYYDYDYGMVLTAPHICKGEETRETRTLELFPLESDNLKGVKDSTLPQTKKDTTADFVADQYFQFL

SEQ ID NO: 78 - DNA, 338 bp, Rubus sp.
  source 1..338: mol_type = genomic DNA
tagttagtcttgccttcctgtttgaatcacaaatcatgtggatgatgacggggtgtcgcagcgatggttgtggagagcctaacattaacaacagggatatccatgactactcgatcaaagctgcaaatcttcagcctgcagtagtagtactcactccaaagcttagcgctacttccaacataacccaaaattctgtgttaccattgaaccaccgccagcatcagggtacgtacatacgtaaacatatatataatatagatcacacatatcgatcttagtttgatggtctgattattctgtacgtgggtaggtaatagactaatagttcatatataatt

SEQ ID NO: 79 - DNA, 258 bp, Rubus sp.
  source 1..258: mol_type = genomic DNA
gatgatgacggggtgtcgcagcgatggttgtggagagcctaacattaacaacagggatatccatgactactcgatcaaagctgcaaatcttcagcctgcagtagtagtactcactccaaagcttagcgctacttccaacataacccaaaattctgtgttaccattgaaccaccgccagcatcagggtacgtacatacgtaaacatatatataatatagatcacacatatcgatcttagtttgatggtctgattattct

SEQ ID NO: 80 - DNA, 218 bp, Rubus sp.
  source 1..218: mol_type = genomic DNA
gcgatggttgtggagagcctaacattaacaacagggatatccatgactactcgatcaaagctgcaaatcttcagcctgcagtagtagtactcactccaaagcttagcgctacttccaacataacccaaaattctgtgttaccattgaaccaccgccagcatcagggtacgtacatacgtaaacatatatataatatagatcacacatatcgatcttag

SEQ ID NO: 81 - DNA, 178 bp, Rubus sp.
  source 1..178: mol_type = genomic DNA
aacattaacaacagggatatccatgactactcgatcaaagctgcaaatcttcagcctgcagtagtagtactcactccaaagcttagcgctacttccaacataacccaaaattctgtgttaccattgaaccaccgccagcatcagggtacgtacatacgtaaacatatatataatatag

SEQ ID NO: 82 - DNA, 138 bp, Rubus sp.
  source 1..138: mol_type = genomic DNA
ccatgactactcgatcaaagctgcaaatcttcagcctgcagtagtagtactcactccaaagcttagcgctacttccaacataacccaaaattctgtgttaccattgaaccaccgccagcatcagggtacgtacatacg

SEQ ID NO: 83 - DNA, 223 bp, Rubus sp.
  source 1..223: mol_type = genomic DNA
tagttagtcttgccttcctgtttgaatcacaaatcatgtggatgatgacggggtgtcgcagcgatggttgtggagagcctaacattaacaacagggatatccatgactactcgatcaaagctgcaaatcttcagcctgcagtagtagtactcactccaaagcttagcgctacttccaacataacccaaaattctgtgttaccattgaaccaccgccagcatca

SEQ ID NO: 84 - DNA, 143 bp, Rubus sp.
  source 1..143: mol_type = genomic DNA
gatgatgacggggtgtcgcagcgatggttgtggagagcctaacattaacaacagggatatccatgactactcgatcaaagctgcaaatcttcagcctgcagtagtagtactcactccaaagcttagcgctacttccaacataa

SEQ ID NO: 85 - DNA, 103 bp, Rubus sp.
  source 1..103: mol_type = genomic DNA
gcgatggttgtggagagcctaacattaacaacagggatatccatgactactcgatcaaagctgcaaatcttcagcctgcagtagtagtactcactccaaagct

SEQ ID NO: 86 - DNA, 63 bp, Rubus sp.
  source 1..63: mol_type = genomic DNA
aacattaacaacagggatatccatgactactcgatcaaagctgcaaatcttcagcctgcagta

SEQ ID NO: 87 - DNA, 223 bp, Rubus sp.
  source 1..223: mol_type = genomic DNA
caaagctgcaaatcttcagcctgcagtagtagtactcactccaaagcttagcgctacttccaacataacccaaaattctgtgttaccattgaaccaccgccagcatcagggtacgtacatacgtaaacatatatataatatagatcacacatatcgatcttagtttgatggtctgattattctgtacgtgggtaggtaatagactaatagttcatatataatt

SEQ ID NO: 88 - DNA, 143 bp, Rubus sp.
  source 1..143: mol_type = genomic DNA
ccaaagcttagcgctacttccaacataacccaaaattctgtgttaccattgaaccaccgccagcatcagggtacgtacatacgtaaacatatatataatatagatcacacatatcgatcttagtttgatggtctgattattct

SEQ ID NO: 89 - DNA, 103 bp, Rubus sp.
  source 1..103: mol_type = genomic DNA
caacataacccaaaattctgtgttaccattgaaccaccgccagcatcagggtacgtacatacgtaaacatatatataatatagatcacacatatcgatcttag

SEQ ID NO: 90 - DNA, 63 bp, Rubus sp.
  source 1..63: mol_type = genomic DNA
tgttaccattgaaccaccgccagcatcagggtacgtacatacgtaaacatatatataatatag

SEQ ID NO: 91 - DNA, 313 bp, Rubus sp.
  source 1..313: mol_type = genomic DNA
gatcaacaaccagctgctgctgctgttgctcctccgcggagttcccgatggaatccaacgccagagcagctactggccctcgaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatctatgtctcagtctcactctaatgaacaacgacaacaatcttggactaacaacctaatcagcagta

SEQ ID NO: 92 - DNA, 233 bp, Rubus sp.
  source 1..233: mol_type = genomic DNA
gttcccgatggaatccaacgccagagcagctactggccctcgaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatctatgtctcagtctcactctaatgaa

SEQ ID NO: 93 - DNA, 193 bp, Rubus sp.
  source 1..193: mol_type = genomic DNA
ccagagcagctactggccctcgaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatctatgt

SEQ ID NO: 94 - DNA, 153 bp, Rubus sp.
  source 1..153: mol_type = genomic DNA
cgaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacg

SEQ ID NO: 95 - DNA, 113 bp, Rubus sp.
  source 1..113: mol_type = genomic DNA
gcatcaagacaccaacggcccagcaaatccgacaagtgacggcgctgcttcgacactttggaaagattgaaggcaagaatgtcttctactggtttcagaaccaccgcgcgagg

SEQ ID NO: 96 - DNA, 60 bp, Rubus sp.
  source 1..60: mol_type = genomic DNA
gcatcaagacaccaacggcccagcaaatccgacaagtgacggcgctgcttcgacactttg

SEQ ID NO: 97 - DNA, 76 bp, Rubus sp.
  source 1..76: mol_type = genomic DNA
gacggcgctgcttcgacactttggaaagattgaaggcaagaatgtcttctactggtttcagaaccaccgcgcgagg

SEQ ID NO: 98 - DNA, 223 bp, Rubus sp.
  source 1..223: mol_type = genomic DNA
gatcaacaaccagctgctgctgctgttgctcctccgcggagttcccgatggaatccaacgccagagcagctactggccctcgaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcaga

SEQ ID NO: 99 - DNA, 143 bp, Rubus sp.
  source 1..143: mol_type = genomic DNA
gttcccgatggaatccaacgccagagcagctactggccctcgaagagctgtaccgacgtggcatcaagacaccaacggcccagcaaatccgacaagtgacggcgctgcttcgacactttggaaagattgaaggcaagaatgtc

SEQ ID NO: 100 - DNA, 103 bp, Rubus sp.
  source 1..103: mol_type = genomic DNA
ccagagcagctactggccctcgaagagctgtaccgacgtggcatcaagacaccaacggcccagcaaatccgacaagtgacggcgctgcttcgacactttggaa

SEQ ID NO: 101 - DNA, 63 bp, Rubus sp.
  source 1..63: mol_type = genomic DNA
cgaagagctgtaccgacgtggcatcaagacaccaacggcccagcaaatccgacaagtgacggc

SEQ ID NO: 102 - DNA, 223 bp, Rubus sp.
  source 1..223: mol_type = genomic DNA
ggagttcccgatggaatccaacgccagagcagctactggccctcgaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatctatgtctcagtc

SEQ ID NO: 103 - DNA, 143 bp, Rubus sp.
  source 1..143: mol_type = genomic DNA
cctcgaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggc

SEQ ID NO: 104 - DNA, 103 bp, Rubus sp.
  source 1..103: mol_type = genomic DNA
gtggcatcaagacaccaacggcccagcaaatccgacaagtgacggcgctgcttcgacactttggaaagattgaaggcaagaatgtcttctactggtttcagaa

SEQ ID NO: 105 - DNA, 63 bp, Rubus sp.
  source 1..63: mol_type = genomic DNA
gcccagcaaatccgacaagtgacggcgctgcttcgacactttggaaagattgaaggcaagaat

SEQ ID NO: 106 - DNA, 223 bp, Rubus sp.
  source 1..223: mol_type = genomic DNA
taccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatctatgtctcagtctcactctaatgaacaacgacaacaatcttggactaacaacctaatcagcagta

SEQ ID NO: 107 - DNA, 143 bp, Rubus sp.
  source 1..143: mol_type = genomic DNA
gacaagtgacggcgctgcttcgacactttggaaagattgaaggcaagaatgtcttctactggtttcagaaccaccgcgcgagggaaaggcagaagagacgacgtgaactcatgtcatctatgtctcagtctcactctaatgaa

SEQ ID NO: 108 - DNA, 103 bp, Rubus sp.
  source 1..103: mol_type = genomic DNA
cgacactttggaaagattgaaggcaagaatgtcttctactggtttcagaaccaccgcgcgagggaaaggcagaagagacgacgtgaactcatgtcatctatgt

SEQ ID NO: 109 - DNA, 63 bp, Rubus sp.
  source 1..63: mol_type = genomic DNA
aggcaagaatgtcttctactggtttcagaaccaccgcgcgagggaaaggcagaagagacgacg

SEQ ID NO: 110 - DNA, 313 bp, Rubus occidentalis
  source 1..313: mol_type = genomic DNA
gatcaacaaccagttgctgctgctgtagctcctccgcgaagttcccgatggaatccaacgccagagcagctactggccctggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatccatgtctcagtctcactctaaagaacaacgacaacaatcttggactaacagcctaatcagcagta

SEQ ID NO: 111 - DNA, 233 bp, Rubus sp.
  source 1..233: mol_type = genomic DNA
gttcccgatggaatccaacgccagagcagctactggccctggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatccatgtctcagtctcactctaaagaa

SEQ ID NO: 112 - DNA, 193 bp, Rubus sp.
  source 1..193: mol_type = genomic DNA
ccagagcagctactggccctggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatccatgt

SEQ ID NO: 113 - DNA, 153 bp, Rubus sp.
  source 1..153: mol_type = genomic DNA
ggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacg

SEQ ID NO: 114 - DNA, 112 bp, Rubus occidentalis
  source 1..112: mol_type = genomic DNA
gcatcaagacaccaacggcccagcaaatccgacaagtgacggcgctgcttcgacactttggaaagattgaaggcaagaatgtcttctactggtttcagaaccaccgcgcgag

SEQ ID NO: 115 - DNA, 60 bp, Rubus occidentalis; identical to SEQ ID NO: 96
  source 1..60: mol_type = genomic DNA

SEQ ID NO: 116 - DNA, 75 bp, Rubus occidentalis
  source 1..75: mol_type = genomic DNA
gacggcgctgcttcgacactttggaaagattgaaggcaagaatgtcttctactggtttcagaaccaccgcgcgag

SEQ ID NO: 117 - DNA, 223 bp, Rubus occidentalis
  source 1..223: mol_type = genomic DNA
gatcaacaaccagttgctgctgctgtagctcctccgcgaagttcccgatggaatccaacgccagagcagctactggccctggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcaga

SEQ ID NO: 118 - DNA, 143 bp, Rubus sp.
  source 1..143: mol_type = genomic DNA
gttcccgatggaatccaacgccagagcagctactggccctggaagagctgtaccgacgtggcatcaagacaccaacggcccagcaaatccgacaagtgacggcgctgcttcgacactttggaaagattgaaggcaagaatgtc

SEQ ID NO: 119 - DNA, 103 bp, Rubus sp.
  source 1..103: mol_type = genomic DNA
ccagagcagctactggccctggaagagctgtaccgacgtggcatcaagacaccaacggcccagcaaatccgacaagtgacggcgctgcttcgacactttggaa

SEQ ID NO: 120 - DNA, 63 bp, Rubus sp.
  source 1..63: mol_type = genomic DNA
ggaagagctgtaccgacgtggcatcaagacaccaacggcccagcaaatccgacaagtgacggc

SEQ ID NO: 121 - DNA, 223 bp, Rubus occidentalis
  source 1..223: mol_type = genomic DNA
gaagttcccgatggaatccaacgccagagcagctactggccctggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatccatgtctcagtc

SEQ ID NO: 122 - DNA, 143 bp, Rubus occidentalis
  source 1..143: mol_type = genomic DNA
cctggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggc

SEQ ID NO: 123 - DNA, 223 bp, Rubus occidentalis
  source 1..223: mol_type = genomic DNA
taccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatccatgtctcagtctcactctaaagaacaacgacaacaatcttggactaacagcctaatcagcagta

SEQ ID NO: 124 - DNA, 143 bp, Rubus sp.
  source 1..143: mol_type = genomic DNA
gacaagtgacggcgctgcttcgacactttggaaagattgaaggcaagaatgtcttctactggtttcagaaccaccgcgcgagggaaaggcagaagagacgacgtgaactcatgtcatccatgtctcagtctcactctaaagaa

SEQ ID NO: 125 - DNA, 103 bp, Rubus sp.
  source 1..103: mol_type = genomic DNA
cgacactttggaaagattgaaggcaagaatgtcttctactggtttcagaaccaccgcgcgagggaaaggcagaagagacgacgtgaactcatgtcatccatgt

SEQ ID NO: 126 - protein, 41 aa, Rubus sp.
HDYSIKAANLQPAVVVLTPKLSATSNITQNSVLPLNHRQHQ

SEQ ID NO: 127 - protein, 35 aa, Rubus sp.
IKTPTAQQIRQVTALLRHFGKIEGKNVFYWFQNHR

SEQ ID NO: 128 - DNA, 6127 bp, Rubus idaeus
  source 1..6127: mol_type = genomic DNA
agtatgtgtaaatgtgaaacacaatagtaaataattacataactaccagcagtattcaatgaaaattccgaccatcatatgtattatggtgactactctagctagcctagctcgctaccccaaaacgagatggatacgcaaaaaacatgcatatagctagctagagccccaaaataaatagcactttagttagctagtactgcccaaccatatataacaataatagcacttttataaaaatggtatatttgcgtgtatgcatacccactttatttaaggatatgggaattgtgatttcattttatatcatttttttcatacgttttttctattttttataaaactatttatgctattgttaagagaaaacagtcaagacactaatagtttgtagtaatcttcaaatataaaagtgtcaatgcaattaacaattaattaaaaaaaaaactaaaaatcaacaacaaaaccatttttaaacactcattcacttctatgggtgtcgctttctagtatatttgaagaaaagaagtctaagactatcccttctcaataacaccctaaaaaactttatttcacctgttttattagtcgacgaaggcctcaatcaatatttctagacttcatactctatgagcaattaaaagaaacaataacccactaactactagcttgctagtggggttaaaaaaaacttactaattaactagcaacctcagtagttggttaattttgaaacaagtacgcttaaacttttaaatatatgatgtattacttttcatattctcaagtgcataaccttgtggctgtagtttagtggtaagaattccacgttgtggccgtggagacctgggctcgaatcccagcagccacatagttttattttttaaatcaatccccttctttttttcatgggttagatggaggcctaaaacccttttatattcctttttcccttgtttaataaaaatttaaaacataattgctaattagattttcctttttcattttaatatttcccacaattccctcccctagccttgagctacaaaggctttgtatataggaataagaggtttgaataactataataacaaattatccaaaaggccattttgggtggcctcatgcttcaaaaagccaccatatattttttacccaaataaggccaccaatatttacaaaaatgccaccgcatcaatatttacaaaaatgccactacacacgaaaacaacaataaagctattgtcaaagcacactaaagctattgtccaacgacaataaagctactgtccaacgacaataaagctactgtccaacgacaataaagcttgtcaaagcatactaaaactattgtccaacgacaataaagctactgtccaacgacaataaagctactgtccagcgacaataaagctactgtcaaagcatactaaaactaacgttaaagaacactactatccaaaaataataaagctactgtccaacgacaataaagctactgtgcaacgacaataaagctactgtccaacgacaataaaatgtcagtggcatgaatcgtaattttgaaaaaaaataatgtcagatagaaagagttaaagctactgagtgaccttatttggaaaacaaagctattgtggcattttgaagaatatggtcacccaaattggccttatgggtaattggctctaattataaacttacaaaacaaaaattatattgttgatgagaagatatataccacaacccagtcaaccagtgtaaaataattcttgcatggtatcattaccacaattgcggcaataaatacagctttaattttagaccaattcaaaagtaaaaaaaacgcattaattcatgacaaacaaatacttttccgaaacttcaaatttttcatcacccttttgtgtatgcacaattgaaacaaaatgtattcttgtcaaagattgattgatcgatatttaagtgaaaatcaaagtatggtcgactacgtaaatatatataatccttttaaataaagtaaacgttcatgaacaatgtacgtcaaaatagataaaaaagggcctgtggtggtgtttttgttttactacccagacagggttttgttgaagagtaaatgcatgtgtcaggagggggtaggagctggtgcgacatgaaagtacttaattctgtcattatttccccttccttttcgtttacgtaagctcaatgtcatcctcaattttaaatgtatatttccactgttcctactttgtttgaacctgaaaaggttagtatagctaccatcatgctcatgtatgtatatatatatatcccactctctccggtctcttctcagcgttgctcagtagtccaaacattcctttgtctagttagtcttgctttcctgtttgaataaaaaatcatgtggatgatgacggggtgtcgcagcggtggttgtggagagcctaacattaacaatagggatatccatgactactcgatcaaagctgcaaatcttcagcccgcagtagtactcactccaaagcttagcgctacttccaacataacccaaaattctgtgttaccattcaaccaccatcaccatcaccatcaccatcagggtacgtacatacgtaaacacacatatatatataatatagatcacacatatcgatcttagtttgatggtctattctgtacgtgggttggtaatagactaatagttcatatataattatacatgtgcagtgaatcctgtcacgccgtctgagcaaggaagaagtggtagtgattatttttataatgatcaacaaccagctgctgctgctgtagctcctccgcggagttcccgatggaatccaacgccagagcagctactggccctggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatccatgtctcagtctcactctaaagaacaacgacaacaatcttggactaacaacctaatcagcagtatccagaacaaagaatctgcaggaggtactaattgagtcgcataagtttcttattaatatatacatatatatatatatatatccatgcactctatacatatgcatacctaacctactgccatattttttaacagcgtaattaattaagcttttagtgtccgtgattatctctcatggtactaattaaagttttgtttgcagaaggtctccgtcttattagtactgtatatcaaatatgttttcgtctctccctccatgtgtgttactctccacctgtagcttcggggttttttaactcaattgatttcaatcaatatttttggaaaaccacctaatagattaagtttagcatacacgtacttggggggaactagggctcaactctaggtgaggccacctggtcctttaggtcacctacgctcaataactctacaccgtattaaatttcttgttataaggaggggagagactcaatggttgtgttaataataatttagtcttacaattaatatacctgggactagcgggcaccaggaaagatagcaaaccctagctaagagcaggacagcttgattaattgattgagcactcgatcgagtacatgaacttgtaaatttgtttctatatgagttaatttcatgattgatcaggtgcaaaagctgattaacacatatatcacggccctatatcacgtacaactgtgctgttgtgtcttcagacattgattaaccgatatatcacaactgtccttttcaggattgaaaaacttaagccttgaaggtgagcagcagaacaagtggactctatcaaactgtatttctgatcaggtatacgcgtactatatctatcattttcttaacgtacgtagttttaagacttaactatatctaaccaaaagctcaagaaaagatagaaatgaattaattgaaagttaagaattttacagttttactctctctctctctctctctctctctcatatttgtatgtatatgtacttaatttgttaattagttactgtacgtccgcaggaatcatcttcagtgctgcaagagaggatgagcatggtggaaagcaattcctccaagcaaccggtggatcagtcctattcggctacacagatctcacaaccaccatgcgcaaacccaacagttgccttaatagacacaaagcccttggactatcatcactattatgattatgattatggtatggtattgactgcaccccatatctgcaaaggcgaagaaaccagagaaactcgaactctggagctctttccactcgagagcgacaatctcaagggggttaaggattccacattaccacaaacaaaaaaagatactactgccgacttcgtggctgaccaatattttcagtttctttgaagaatttaagggagcagcattcacatagctagaaattaatctcctttagaagggtttttcaatctatcgatctatttaataaataattatggattaattaatatgcatgcgtgtcttctctttgatggaaatgataggtagggtacatccttatcaggctctgtttttggttctttccacttttatttcttcttattgtttaaatgcagttcaatttgtaaggacgggtaaggttgattgatgtccccctttctcgttcattaggttctttctgtgtctctttctttctagtgttgagggatatctagttatatatcctgtcgctagctaggtgtaccgtacctttatctttttctgtaaataaaactccatcgatttcatggcagaccaatatatattttcaatttctttgaagaatttaagggtttgtcaatttctctatataatataaagtatatatatatatatgtgcatgtgttgaaaactgtaagagaaacttgaagaacaagaagctagggatgtatttgatatatgaagaaatttgtcacaactaacagagatcgagaaacgagctgcagctatatatacagaaataagccacgtgtcctccagctgtaggcaactatcctaacagaccgtaacaaacccctagcttggtcatgagataagcacgagctataataaacaaactatgtaaacctagctaaacttagttaattaacaaactaactaatcaggaactaattgctgacttagatacttcatcataacatgtgtgtcttcttcttggtggaatggcatgcatggggtcagattctgttttggtttctcacttttgtttgaatgcagattaatttgtttggaaaggtagggttgattgtgtctccctttctctttataagtttttggtggtcttagaaacttgcaagtgttgtgtgtgtgtctatataatcattctcagataagtacggagtttattatttttttatggtgcagagttttgatatcaaccgtcatatatatttgttttaaatgatttgatatgacagctgatatcaaaactctgtagtataaaaaataaggatctctttaacagagaatgtttgtatatatatattgtccccgagtagtgcataaccctctcgctaggttatttttataaaattttctgttattaaacttgatccatcgcattgtccaggtccatgaaacaaagaaattacaaatgtgtagcaattaagctctcatatagtgatatagacatatagtcaaatgtctcgaaccaaaaaaaaaactctcatatcagtattcagaccagctggcctggtcctgcattttctgtttgcgtcatgcatgcgacagaagatacagttgaataactagaatatgtgattctgtggaataatattgcgtcacactgcattcgttatacatgtttgtgcgtcatactgaatttatgtatatctaggaaaattctggtaaacaccggtgagtattaaacaccgggctgaatatcagccattcattgtttgttatatccacacgattaccaatgtagcttctaaaaaataaaagtttgttctgtttttgcccttccattaccccccgtatgaagtatgaacatagccagtcttccaactctat

SEQ ID NO: 129 - DNA, 1002 bp, Rubus idaeus
  source 1..1002: mol_type = other DNA
atgatgacggggtgtcgcagcggtggttgtggagagcctaacattaacaatagggatatccatgactactcgatcaaagctgcaaatcttcagcccgcagtagtactcactccaaagcttagcgctacttccaacataacccaaaattctgtgttaccattcaaccaccatcaccatcacctgaatcctgtcacgccgtctgagcaaggaagaagtggtagtgattatttttataatgatcaacaaccagctgctgctgctgtagctcctccgcggagttcccgatggaatccaacgccagagcagctactggccctggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatccatgtctcagtctcactctaaagaacaacgacaacaatcttggactaacaacctaatcagcagtatccagaacaaagaatctgcaggaggattgaaaaacttaagccttgaaggtgagcagcagaacaagtggactctatcaaactgtatttctgatcaggaatcatcttcagtgctgcaagagaggatgagcatggtggaaagcaattcctccaagcaaccggtggatcagtcctattcggctacacagatctcacaaccaccatgcgcaaacccaacagttgccttaatagacacaaagcccttggactatcatcactattatgattatgattatggtatggtattgactgcaccccatatctgcaaaggcgaagaaaccagagaaactcgaactctggagctctttccactcgagagcgacaatctcaagggggttaaggattccacattaccacaaacaaaaaaagatactactgccgacttcgtggctgaccaatattttcagtttctttga

SEQ ID NO: 130 - protein, 333 aa, Rubus idaeus
MMTGCRSGGCGEPNINNRDIHDYSIKAANLQPAVVLTPKLSATSNITQNSVLPFNHHHHHLNPVTPSEQGRSGSDYFYNDQQPAAAAVAPPRSSRWNPTPEQLLALEELYRRGIKTPTAQQIRQVTALLRHFGKIEGKNVFYWFQNHRARERQKRRRELMSSMSQSHSKEQRQQSWTNNLISSIQNKESAGGLKNLSLEGEQQNKWTLSNCISDQESSSVLQERMSMVESNSSKQPVDQSYSATQISQPPCANPTVALIDTKPLDYHHYYDYDYGMVLTAPHICKGEETRETRTLELFPLESDNLKGVKDSTLPQTKKDTTADFVADQYFQFL

SEQ ID NO: 131 - DNA, 313 bp, Rubus idaeus
  source 1..313: mol_type = genomic DNA
gatcaacaaccagctgctgctgctgtagctcctccgcggagttcccgatggaatccaacgccagagcagctactggccctggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatccatgtctcagtctcactctaaagaacaacgacaacaatcttggactaacaacctaatcagcagta

SEQ ID NO: 132 - DNA, 223 bp, Rubus idaeus
  source 1..223: mol_type = genomic DNA
gatcaacaaccagctgctgctgctgtagctcctccgcggagttcccgatggaatccaacgccagagcagctactggccctggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcaga

SEQ ID NO: 133 - DNA, 223 bp, Rubus idaeus
  source 1..223: mol_type = genomic DNA
ggagttcccgatggaatccaacgccagagcagctactggccctggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatccatgtctcagtc

SEQ ID NO: 134 - DNA, 144 bp, Rubus idaeus
  source 1..144: mol_type = genomic DNA
ccctggaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggc

SEQ ID NO: 135 - DNA, 223 bp, Rubus idaeus
  source 1..223: mol_type = genomic DNA
taccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatccatgtctcagtctcactctaaagaacaacgacaacaatcttggactaacaacctaatcagcagta

SEQ ID NO: 136 - protein, 39 aa, Rubus sp.
HDYSIKAANLQPAVVLTPKLSATSNITQNSVLPFNHHHH

SEQ ID NO: 137 - protein, 35 aa, Rubus sp.; identical to SEQ ID NO: 127

SEQ ID NO: 138 - DNA, 23 bp, synthetic construct
  source 1..23: mol_type = other DNA
cctcgcgcggtggttctgaaacc

SEQ ID NO: 139 - DNA, 23 bp, synthetic construct
  source 1..23: mol_type = other DNA
caaagtgtcgaagcagcgccgtc

SEQ ID NO: 140 - DNA, 23 bp, synthetic construct
  source 1..23: mol_type = other DNA
ctgggccgttggtgtcttgatgc

SEQ ID NO: 141 - DNA, 23 bp, synthetic construct
  source 1..23: mol_type = other DNA
cgtatgtacgtaccctgatgctg

SEQ ID NO: 142 - DNA, 23 bp, synthetic construct
  source 1..23: mol_type = other DNA
cagctttgatcgagtagtcatgg

SEQ ID NO: 143 - DNA, 6197 bp, synthetic construct
  source 1..6197: mol_type = other DNA
gctcatgcgtcataattctaggtttgggtgggacccgatcacaaagagattcatatatgatgatgaagtatgagcatactattttatttgaaggtaaatttctattgacttttattctattctatttcgttaatgttctttttttttcttctattttttggtatgttttgtttttttttttgatacatatacctttgattttattaattacattgggaggttgctctttcctttgttttctaattattttgtgtcacaacattatttaagtttttctgaaatttgtagaagttgtttataataaagtttgtagatttcataaatcaataaaaggacttaaataaatcaatagaagtctatgaaatctatgaagtctgtagacttttcaaaaagtttgtaaattaaaaaaaaaagggtccttgaccataaggtccaaagttggacaaatatttaccatcaaggacaaaggaaaaacacggtgcccacccggtccattaattgaccaatttatatttttgaattatacattttacaaaaattatgaaactgccactggaaaattttattgaccacaataattttttttttttgagaaaacgtatattcctatggtaggagagaaagaacaaaaaaaatcttttttgtgtattttattgacctcaataaaattttattgagctccataaactgttttattgaggtcaataaacttttttattgaggtcaataaactgtttcattgagatgacattggttttgaggtcaataaactgtttcattgaggtgacattggttttgaggtcattaaactattttattgaggtcaataaactgtttcattgaggtgacattggttttgaggtcaataaactattttattgaggtcaataaattatttcattgaggtgacattggtattgaggtcaataaattgtttcattgagatatcattagtattgagatcaataaatttttttattgaggtcaagaaattttttttattgaagtcaataaactgtttattgtggcatttttgtaaatattggtgaccttatttgggaaaaaatattaggtggcttttttaagaataggatcacccaaagtggctttattggtaattatttattaacttaattcatgacagattatgagaaaaaaaagagagaatagaggagagagagagagagagatagataagaagaagaagaagagaaaacaaagatgccaaaaatagacactaagggcaataatgtcacaaaaaatcactatttaatatttttgtccttactgggagtaaaacatgtttaactgaaacttagtgagaattatttaaaatttatcaaacttgaagggaaatatgtagtgtaaaagaggatctattggttatttaccttaaaaaaaatttcatacaaatccatacacaatacacattgactatttaattatatgatgtattaatttgtttatactctaaagtacacaaccttgtggctgtagtttagtggtaagaattccacgttgtggccgtggagacctgggctcgaatcccagcagccacatagttttgttttttaaatcaattcccttctttttttcatgggttacatttatggaggtctcaaacccttatattcctttttcccttgtgtaataaaaatttaaaacataattgctaattagattttccttttaataaacctctacgtatattaacgcaacatatttctcacaattccatcccctagccttgagctacgaaggctttgtatataggactaagaggtttgaataattataaacttacaaacaaaatttgtaaaataattattgcatggtaccattaccacaattgtggcaatacatattggtttaattttagaccaacccaatagcagaaggcactaatttatgaccaacaagtacttttccgaaacttagagacaaatttgtcatcacctttttgtgtatgcacaattgaaacaaaatgtgttccttgcaaagattgattgatcgatatttaagtgaaaatcaaagtatgattgattaatccttttaaataaagtaaacatgaacaatgtacgtcgaaatagataaaaagggcctgtggtggtttttgttttactacccagacaggtttttgttgaagagtaaatgcaggtgtcaggagagggtaggagctggtgcgacatgaaagtacttaattctgtcattatttccatcttttcgtttacgtaagctcaatgtcatcctcaattttaaatgtatatttccactgttcctacttgtttgaaccggaaaaggttagtatagctaccatcatgctcatgtatgtatgtatgtgtgtatatatatatcccagtctctccagtctcttctcagctttgctcaggagtccaaacattcctttgtctagttagtcttgccttcctgtttgaatcacaaatcatgtggatgatgacggggtgtcgcagcgatggttgtggagagcctaacattaacaacagggatatccatgactactcgatcaaagctgcaaatcttcagcctgcagtagtagtactcactccaaagcttagcgctacttccaacataacccaaaattctgtgttaccattgaaccaccgcgtacgtacatacgtaaacatatatataatatagatcacacatatcgatcttagtttgatggtctgattattctgtacgtgggtaggtaatagactaatagttcatatataattatacatgtgcagtgatccctgtcgcgccgtctgagcaaggaagaagtggtagtgattatttttataatgatcaacaaccagctgctgctgctgttgctcctccgcggagttcccgatggaatccaacgccagagcagctactggccctcgaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatctatgtctcagtctcactctaatgaacaacgacaacaatcttggactaacaacctaatcagcagtatccagaacaaagaatccgcaggaggtactaattgagtcgcataattttcttattaatatatatatatatatgtacgtgcatatatatccatgcactatatatgtgcatacctaacctactgccatatttttaacagcgtaattaattaagcgtttagtgtccgtgattatctctcatggtactaattaaagttttgtttgcagaaggtcgacgtcttattagtactgtatatcaaatatgttttcggggtttttttcctccatgtgtgttactctccacctgtagcttcggggttttttataactcaattgatttcaatcaatattgttggaaaaccacctaatagaataattttagcacacacttacttggccggaactagggctcaactctaggtgaggccacctccatcctttaggtcacctacgctcaataactctacaccgtattaaatttcttgttataaggagaggagagactcagtggttgtgttaataataatttagtcttacaattaatatacctgggactagcgggcaccaagcaagatagcaaaccctagctaagagcaggacagcttgattaattgattgggcactcgatcgagtacatgtacttgtaaatttttctatatgacttaattttatgattgatctggtgcaaaaactgattaacacatatcacgtacaattgtgctcttctcccttcagacattgattaaccaataaatcacaactgtcctttgcaggattgagaaacttaagccttgaaggtgagcagcagaacaagtggactctatcaaactgtactattctttctgatcaggtatacgcgtgctatatctatcattttattaacgtacgtagttttaagacttaactatatctgaccaaaagctcaagaaaagatagaaatatattaattaattgaaagttaaattttttacagttttactctctctctctctctctctctcccccccccccccccaatagtatgtatatgtacttcatttgttaattagttactgtacgtccgcaggaatcatcttcagtgctgcaagagaggatgagcatggtggaaagcaattcctccaagcaaccggtggatcagtcctattcggctacaccgacctcacaaccaccatgcgcaaacccaactgtcaccttaataaacacaaagcccttggattgtcgtcactattatgattatggtatggtattgaccgcgccccatatatgcaaaggcgaagaaaccagagaaactcgaactctggagctctttcctctcgagagcgacaatctcaagggggttaaggattccgcattaccacaaacaaaaaaagatactactgcagacttcgtggctgaccaatattttcagtttctttgaagaatttaagggagcagcattcacatagctagaaattaatctacttaagaagggtttttcaatctatcgatctatttaataaattattatggattaattaattatgtatgtgtgtcttctctttgatggaaatgatagctagggtacatctttatcgggttctgttttggttctttccacttttatttcttctttttgtttatatgcagttcatttgtaaggacgggtaaggttgattgatgtccccctttctcgttcgttaggttctttctgtgtctctttctttctactgttgagggatatgtagttatatatcctgtcgctaggtgtacctttatctttttctgtaaataaaactccatcgatttcatggcagaccaatatatattttcaatttcattgaagaatttaagggcttatcaatttctctatctaatataaaatatataggcattttttactaatgggatccttactttttatgctacaaaattttgatttcagtcatcagatcacattatttaaaacaaatctgatagctgagatcaatactccgcaccataaaaagtaaggacctctttatataaaaattattgatatatgaagaaatttgatcacaacttaccgagagaaatgagttgcagctatatatgcttcgactagattacacataaataagccacgagtcctctagttgtaggcaactatcgtaacaaactgtaacaaaccccttggtcacgagataagcacaagctataataaacaaactatgtaaacctagctaaacttagtttattaacaaactaactagtcggaaactgatgagaaattttattaggacctacaaatttgctttacctaacttcctatattctttgcacctcttttttgcttttaaaatataagatttgcttactaaactttttttaaatttctaaaataaccataatgatataattcacattaaatatacaaaattactactataattctaaatttactcaccacacatttattaattaatttataatcaataaaaaaaatttagcatgaattattattatttttaactattcttttctcttttagctgtgcaataaaaaaaatagtaatcatttattcttacagtttatttttttctctattttttgtacctcatgattaaatattttatatatattttatgttgctagtttttttttttacaaatataatggatagacttaaatttaggtcatagtagaatttttttcgttctctatcactaatatacgttcaacaaatgatacaaatttttatgtcacataaactatcataattttaattcttattacatatatataaatattttaatgagtacgtattgaaattaaaaaatgaaaatagataagaaaaataataaaaaatacaatataatattattgaattagaaactgtagagaaaataaatataatatttttttggtataattattatccttaatagttttataggaggttataaatataatttaataattcataatggagtgaggttaactgagcaaaaatttggaggtttaagggaaactaattgatgactaattgccgacttagatgcttcatcataacatgtttgtcttctccttgttggaatggcatgcatggggtcggattctgttttggtttcccatttttctttgaatg

SEQ ID NO: 144 - DNA, 6201 bp, synthetic construct
  source 1..6201: mol_type = other DNA
gctcatgcgtcataattctaggtttgggtgggacccgatcacaaagagattcatatatgatgatgaagtatgagcatactattttatttgaaggtaaatttctattgacttttattctattctatttcgttaatgttctttttttttcttctattttttggtatgttttgtttttttttttgatacatatacctttgattttattaattacattgggaggttgctctttcctttgttttctaattattttgtgtcacaacattatttaagtttttctgaaatttgtagaagttgtttataataaagtttgtagatttcataaatcaataaaaggacttaaataaatcaatagaagtctatgaaatctatgaagtctgtagacttttcaaaaagtttgtaaattaaaaaaaaaagggtccttgaccataaggtccaaagttggacaaatatttaccatcaaggacaaaggaaaaacacggtgcccacccggtccattaattgaccaatttatatttttgaattatacattttacaaaaattatgaaactgccactggaaaattttattgaccacaataattttttttttttgagaaaacgtatattcctatggtaggagagaaagaacaaaaaaaatcttttttgtgtattttattgacctcaataaaattttattgagctccataaactgttttattgaggtcaataaacttttttattgaggtcaataaactgtttcattgagatgacattggttttgaggtcaataaactgtttcattgaggtgacattggttttgaggtcattaaactattttattgaggtcaataaactgtttcattgaggtgacattggttttgaggtcaataaactattttattgaggtcaataaattatttcattgaggtgacattggtattgaggtcaataaattgtttcattgagatatcattagtattgagatcaataaatttttttattgaggtcaagaaattttttttattgaagtcaataaactgtttattgtggcatttttgtaaatattggtgaccttatttgggaaaaaatattaggtggcttttttaagaataggatcacccaaagtggctttattggtaattatttattaacttaattcatgacagattatgagaaaaaaaagagagaatagaggagagagagagagagagatagataagaagaagaagaagagaaaacaaagatgccaaaaatagacactaagggcaataatgtcacaaaaaatcactatttaatatttttgtccttactgggagtaaaacatgtttaactgaaacttagtgagaattatttaaaatttatcaaacttgaagggaaatatgtagtgtaaaagaggatctattggttatttaccttaaaaaaaatttcatacaaatccatacacaatacacattgactatttaattatatgatgtattaatttgtttatactctaaagtacacaaccttgtggctgtagtttagtggtaagaattccacgttgtggccgtggagacctgggctcgaatcccagcagccacatagttttgttttttaaatcaattcccttctttttttcatgggttacatttatggaggtctcaaacccttatattcctttttcccttgtgtaataaaaatttaaaacataattgctaattagattttccttttaataaacctctacgtatattaacgcaacatatttctcacaattccatcccctagccttgagctacgaaggctttgtatataggactaagaggtttgaataattataaacttacaaacaaaatttgtaaaataattattgcatggtaccattaccacaattgtggcaatacatattggtttaattttagaccaacccaatagcagaaggcactaatttatgaccaacaagtacttttccgaaacttagagacaaatttgtcatcacctttttgtgtatgcacaattgaaacaaaatgtgttccttgcaaagattgattgatcgatatttaagtgaaaatcaaagtatgattgattaatccttttaaataaagtaaacatgaacaatgtacgtcgaaatagataaaaagggcctgtggtggtttttgttttactacccagacaggtttttgttgaagagtaaatgcaggtgtcaggagagggtaggagctggtgcgacatgaaagtacttaattctgtcattatttccatcttttcgtttacgtaagctcaatgtcatcctcaattttaaatgtatatttccactgttcctacttgtttgaaccggaaaaggttagtatagctaccatcatgctcatgtatgtatgtatgtgtgtatatatatatcccagtctctccagtctcttctcagctttgctcaggagtccaaacattcctttgtctagttagtcttgccttcctgtttgaatcacaaatcatgtggatgatgacggggtgtcgcagcgatggttgtggagagcctaacattaacaacagggatatccatgactactcgatcaaagctgcaaatcttcagcctgcagtagtagtactcactccaaagcttagcgctacttccaacataacccaaaattctgtgttaccattgaaccaccgccagcatacgtacatacgtaaacatatatataatatagatcacacatatcgatcttagtttgatggtctgattattctgtacgtgggtaggtaatagactaatagttcatatataattatacatgtgcagtgatccctgtcgcgccgtctgagcaaggaagaagtggtagtgattatttttataatgatcaacaaccagctgctgctgctgttgctcctccgcggagttcccgatggaatccaacgccagagcagctactggccctcgaagagctgtaccgacgtggcatcaagacaccaacggcccagcaaatccgacaagtgacggcgctgcttcgacactttggaaagattgaaggcaagaatgtcttctactggtttcagaaccaccgcgcgagggaaaggcagaagagacgacgtgaactcatgtcatctatgtctcagtctcactctaatgaacaacgacaacaatcttggactaacaacctaatcagcagtatccagaacaaagaatccgcaggaggtactaattgagtcgcataattttcttattaatatatatatatatatgtacgtgcatatatatccatgcactatatatgtgcatacctaacctactgccatatttttaacagcgtaattaattaagcgtttagtgtccgtgattatctctcatggtactaattaaagttttgtttgcagaaggtcgacgtcttattagtactgtatatcaaatatgttttcggggtttttttcctccatgtgtgttactctccacctgtagcttcggggttttttataactcaattgatttcaatcaatattgttggaaaaccacctaatagaataattttagcacacacttacttggccggaactagggctcaactctaggtgaggccacctccatcctttaggtcacctacgctcaataactctacaccgtattaaatttcttgttataaggagaggagagactcagtggttgtgttaataataatttagtcttacaattaatatacctgggactagcgggcaccaagcaagatagcaaaccctagctaagagcaggacagcttgattaattgattgggcactcgatcgagtacatgtacttgtaaatttttctatatgacttaattttatgattgatctggtgcaaaaactgattaacacatatcacgtacaattgtgctcttctcccttcagacattgattaaccaataaatcacaactgtcctttgcaggattgagaaacttaagccttgaaggtgagcagcagaacaagtggactctatcaaactgtactattctttctgatcaggtatacgcgtgctatatctatcattttattaacgtacgtagttttaagacttaactatatctgaccaaaagctcaagaaaagatagaaatatattaattaattgaaagttaaattttttacagttttactctctctctctctctctctctcccccccccccccccaatagtatgtatatgtacttcatttgttaattagttactgtacgtccgcaggaatcatcttcagtgctgcaagagaggatgagcatggtggaaagcaattcctccaagcaaccggtggatcagtcctattcggctacaccgacctcacaaccaccatgcgcaaacccaactgtcaccttaataaacacaaagcccttggattgtcgtcactattatgattatggtatggtattgaccgcgccccatatatgcaaaggcgaagaaaccagagaaactcgaactctggagctctttcctctcgagagcgacaatctcaagggggttaaggattccgcattaccacaaacaaaaaaagatactactgcagacttcgtggctgaccaatattttcagtttctttgaagaatttaagggagcagcattcacatagctagaaattaatctacttaagaagggtttttcaatctatcgatctatttaataaattattatggattaattaattatgtatgtgtgtcttctctttgatggaaatgatagctagggtacatctttatcgggttctgttttggttctttccacttttatttcttctttttgtttatatgcagttcatttgtaaggacgggtaaggttgattgatgtccccctttctcgttcgttaggttctttctgtgtctctttctttctactgttgagggatatgtagttatatatcctgtcgctaggtgtacctttatctttttctgtaaataaaactccatcgatttcatggcagaccaatatatattttcaatttcattgaagaatttaagggcttatcaatttctctatctaatataaaatatataggcattttttactaatgggatccttactttttatgctacaaaattttgatttcagtcatcagatcacattatttaaaacaaatctgatagctgagatcaatactccgcaccataaaaagtaaggacctctttatataaaaattattgatatatgaagaaatttgatcacaacttaccgagagaaatgagttgcagctatatatgcttcgactagattacacataaataagccacgagtcctctagttgtaggcaactatcgtaacaaactgtaacaaaccccttggtcacgagataagcacaagctataataaacaaactatgtaaacctagctaaacttagtttattaacaaactaactagtcggaaactgatgagaaattttattaggacctacaaatttgctttacctaacttcctatattctttgcacctcttttttgcttttaaaatataagatttgcttactaaactttttttaaatttctaaaataaccataatgatataattcacattaaatatacaaaattactactataattctaaatttactcaccacacatttattaattaatttataatcaataaaaaaaatttagcatgaattattattatttttaactattcttttctcttttagctgtgcaataaaaaaaatagtaatcatttattcttacagtttatttttttctctattttttgtacctcatgattaaatattttatatatattttatgttgctagtttttttttttacaaatataatggatagacttaaatttaggtcatagtagaatttttttcgttctctatcactaatatacgttcaacaaatgatacaaatttttatgtcacataaactatcataattttaattcttattacatatatataaatattttaatgagtacgtattgaaattaaaaaatgaaaatagataagaaaaataataaaaaatacaatataatattattgaattagaaactgtagagaaaataaatataatatttttttggtataattattatccttaatagttttataggaggttataaatataatttaataattcataatggagtgaggttaactgagcaaaaatttggaggtttaagggaaactaattgatgactaattgccgacttagatgcttcatcataacatgtttgtcttctccttgttggaatggcatgcatggggtcggattctgttttggtttcccatttttctttgaatg

SEQ ID NO: 145 - DNA, 6197 bp, synthetic construct
  source 1..6197: mol_type = other DNA
gctcatgcgtcataattctaggtttgggtgggacccgatcacaaagagattcatatatgatgatgaagtatgagcatactattttatttgaaggtaaatttctattgacttttattctattctatttcgttaatgttctttttttttcttctattttttggtatgttttgtttttttttttgatacatatacctttgattttattaattacattgggaggttgctctttcctttgttttctaattattttgtgtcacaacattatttaagtttttctgaaatttgtagaagttgtttataataaagtttgtagatttcataaatcaataaaaggacttaaataaatcaatagaagtctatgaaatctatgaagtctgtagacttttcaaaaagtttgtaaattaaaaaaaaaagggtccttgaccataaggtccaaagttggacaaatatttaccatcaaggacaaaggaaaaacacggtgcccacccggtccattaattgaccaatttatatttttgaattatacattttacaaaaattatgaaactgccactggaaaattttattgaccacaataattttttttttttgagaaaacgtatattcctatggtaggagagaaagaacaaaaaaaatcttttttgtgtattttattgacctcaataaaattttattgagctccataaactgttttattgaggtcaataaacttttttattgaggtcaataaactgtttcattgagatgacattggttttgaggtcaataaactgtttcattgaggtgacattggttttgaggtcattaaactattttattgaggtcaataaactgtttcattgaggtgacattggttttgaggtcaataaactattttattgaggtcaataaattatttcattgaggtgacattggtattgaggtcaataaattgtttcattgagatatcattagtattgagatcaataaatttttttattgaggtcaagaaattttttttattgaagtcaataaactgtttattgtggcatttttgtaaatattggtgaccttatttgggaaaaaatattaggtggcttttttaagaataggatcacccaaagtggctttattggtaattatttattaacttaattcatgacagattatgagaaaaaaaagagagaatagaggagagagagagagagagatagataagaagaagaagaagagaaaacaaagatgccaaaaatagacactaagggcaataatgtcacaaaaaatcactatttaatatttttgtccttactgggagtaaaacatgtttaactgaaacttagtgagaattatttaaaatttatcaaacttgaagggaaatatgtagtgtaaaagaggatctattggttatttaccttaaaaaaaatttcatacaaatccatacacaatacacattgactatttaattatatgatgtattaatttgtttatactctaaagtacacaaccttgtggctgtagtttagtggtaagaattccacgttgtggccgtggagacctgggctcgaatcccagcagccacatagttttgttttttaaatcaattcccttctttttttcatgggttacatttatggaggtctcaaacccttatattcctttttcccttgtgtaataaaaatttaaaacataattgctaattagattttccttttaataaacctctacgtatattaacgcaacatatttctcacaattccatcccctagccttgagctacgaaggctttgtatataggactaagaggtttgaataattataaacttacaaacaaaatttgtaaaataattattgcatggtaccattaccacaattgtggcaatacatattggtttaattttagaccaacccaatagcagaaggcactaatttatgaccaacaagtacttttccgaaacttagagacaaatttgtcatcacctttttgtgtatgcacaattgaaacaaaatgtgttccttgcaaagattgattgatcgatatttaagtgaaaatcaaagtatgattgattaatccttttaaataaagtaaacatgaacaatgtacgtcgaaatagataaaaagggcctgtggtggtttttgttttactacccagacaggtttttgttgaagagtaaatgcaggtgtcaggagagggtaggagctggtgcgacatgaaagtacttaattctgtcattatttccatcttttcgtttacgtaagctcaatgtcatcctcaattttaaatgtatatttccactgttcctacttgtttgaaccggaaaaggttagtatagctaccatcatgctcatgtatgtatgtatgtgtgtatatatatatcccagtctctccagtctcttctcagctttgctcaggagtccaaacattcctttgtctagttagtcttgccttcctgtttgaatcacaaatcatgtggatgatgacggggtgtcgcagcgatggttgtggagagcctaacattaacaacagggatatccatgactactcgatcaaagctgcaaatcttcagcctgcagtagtagtactcactccaaagcttagcgctacttccaacataacccaaaattctgtgttaccattgaaccaccgccagcatcagggtacgtacatacgtaaacatatatataatatagatcacacatatcgatcttagtttgatggtctgattattctgtacgtgggtaggtaatagactaatagttcatatataattatacatgtgcagtgatccctgtcgcgccgtctgagcaaggaagaagtggtagtgattatttttataatgatcaacaaccagctgctgctgctgttgctcctccgcggagttcccgatggaatccaacgccagagcagctactggccctcgaagagctgtaccgacgtggaccaacggcccagcaaatccgacaagtgacggcgtgcttcgacactttggaaagattgaaggcaagaatgtcttctactggtttcagaaccaccgcgcgagggaaaggcagaagagacgacgtgaactcatgtcatctatgtctcagtctcactctaatgaacaacgacaacaatcttggactaacaacctaatcagcagtatccagaacaaagaatccgcaggaggtactaattgagtcgcataattttcttattaatatatatatatatatgtacgtgcatatatatccatgcactatatatgtgcatacctaacctactgccatatttttaacagcgtaattaattaagcgtttagtgtccgtgattatctctcatggtactaattaaagttttgtttgcagaaggtcgacgtcttattagtactgtatatcaaatatgttttcggggtttttttcctccatgtgtgttactctccacctgtagcttcggggttttttataactcaattgatttcaatcaatattgttggaaaaccacctaatagaataattttagcacacacttacttggccggaactagggctcaactctaggtgaggccacctccatcctttaggtcacctacgctcaataactctacaccgtattaaatttcttgttataaggagaggagagactcagtggttgtgttaataataatttagtcttacaattaatatacctgggactagcgggcaccaagcaagatagcaaaccctagctaagagcaggacagcttgattaattgattgggcactcgatcgagtacatgtacttgtaaatttttctatatgacttaattttatgattgatctggtgcaaaaactgattaacacatatcacgtacaattgtgctcttctcccttcagacattgattaaccaataaatcacaactgtcctttgcaggattgagaaacttaagccttgaaggtgagcagcagaacaagtggactctatcaaactgtactattctttctgatcaggtatacgcgtgctatatctatcattttattaacgtacgtagttttaagacttaactatatctgaccaaaagctcaagaaaagatagaaatatattaattaattgaaagttaaattttttacagttttactctctctctctctctctctctcccccccccccccccaatagtatgtatatgtacttcatttgttaattagttactgtacgtccgcaggaatcatcttcagtgctgcaagagaggatgagcatggtggaaagcaattcctccaagcaaccggtggatcagtcctattcggctacaccgacctcacaaccaccatgcgcaaacccaactgtcaccttaataaacacaaagcccttggattgtcgtcactattatgattatggtatggtattgaccgcgccccatatatgcaaaggcgaagaaaccagagaaactcgaactctggagctctttcctctcgagagcgacaatctcaagggggttaaggattccgcattaccacaaacaaaaaaagatactactgcagacttcgtggctgaccaatattttcagtttctttgaagaatttaagggagcagcattcacatagctagaaattaatctacttaagaagggtttttcaatctatcgatctatttaataaattattatggattaattaattatgtatgtgtgtcttctctttgatggaaatgatagctagggtacatctttatcgggttctgttttggttctttccacttttatttcttctttttgtttatatgcagttcatttgtaaggacgggtaaggttgattgatgtccccctttctcgttcgttaggttctttctgtgtctctttctttctactgttgagggatatgtagttatatatcctgtcgctaggtgtacctttatctttttctgtaaataaaactccatcgatttcatggcagaccaatatatattttcaatttcattgaagaatttaagggcttatcaatttctctatctaatataaaatatataggcattttttactaatgggatccttactttttatgctacaaaattttgatttcagtcatcagatcacattatttaaaacaaatctgatagctgagatcaatactccgcaccataaaaagtaaggacctctttatataaaaattattgatatatgaagaaatttgatcacaacttaccgagagaaatgagttgcagctatatatgcttcgactagattacacataaataagccacgagtcctctagttgtaggcaactatcgtaacaaactgtaacaaaccccttggtcacgagataagcacaagctataataaacaaactatgtaaacctagctaaacttagtttattaacaaactaactagtcggaaactgatgagaaattttattaggacctacaaatttgctttacctaacttcctatattctttgcacctcttttttgcttttaaaatataagatttgcttactaaactttttttaaatttctaaaataaccataatgatataattcacattaaatatacaaaattactactataattctaaatttactcaccacacatttattaattaatttataatcaataaaaaaaatttagcatgaattattattatttttaactattcttttctcttttagctgtgcaataaaaaaaatagtaatcatttattcttacagtttatttttttctctattttttgtacctcatgattaaatattttatatatattttatgttgctagtttttttttttacaaatataatggatagacttaaatttaggtcatagtagaatttttttcgttctctatcactaatatacgttcaacaaatgatacaaatttttatgtcacataaactatcataattttaattcttattacatatatataaatattttaatgagtacgtattgaaattaaaaaatgaaaatagataagaaaaataataaaaaatacaatataatattattgaattagaaactgtagagaaaataaatataatatttttttggtataattattatccttaatagttttataggaggttataaatataatttaataattcataatggagtgaggttaactgagcaaaaatttggaggtttaagggaaactaattgatgactaattgccgacttagatgcttcatcataacatgtttgtcttctccttgttggaatggcatgcatggggtcggattctgttttggtttcccatttttctttgaatg

SEQ ID NO: 146 - protein, 179 aa, synthetic construct
MWMMTGCRSDGCGEPNINNRDIHDYSIKAANLQPAVVVLTPKLSATSNITQNSVLPLNHRQHQVIPVAPSEQGRSGSDYFYNDQQPAAAAVAPPRSSRWNPTPEQLLALEELYRRGPTAQQIRQVTACFDTLERLKARMSSTGFRTTARGKGRRDDVNSCHLCLSLTLMNNDNNLGLTT

SEQ ID NO: 147 - DNA, 6185 bp, synthetic construct
  source 1..6185: mol_type = other DNA
gctcatgcgtcataattctaggtttgggtgggacccgatcacaaagagattcatatatgatgatgaagtatgagcatactattttatttgaaggtaaatttctattgacttttattctattctatttcgttaatgttctttttttttcttctattttttggtatgttttgtttttttttttgatacatatacctttgattttattaattacattgggaggttgctctttcctttgttttctaattattttgtgtcacaacattatttaagtttttctgaaatttgtagaagttgtttataataaagtttgtagatttcataaatcaataaaaggacttaaataaatcaatagaagtctatgaaatctatgaagtctgtagacttttcaaaaagtttgtaaattaaaaaaaaaagggtccttgaccataaggtccaaagttggacaaatatttaccatcaaggacaaaggaaaaacacggtgcccacccggtccattaattgaccaatttatatttttgaattatacattttacaaaaattatgaaactgccactggaaaattttattgaccacaataattttttttttttgagaaaacgtatattcctatggtaggagagaaagaacaaaaaaaatcttttttgtgtattttattgacctcaataaaattttattgagctccataaactgttttattgaggtcaataaacttttttattgaggtcaataaactgtttcattgagatgacattggttttgaggtcaataaactgtttcattgaggtgacattggttttgaggtcattaaactattttattgaggtcaataaactgtttcattgaggtgacattggttttgaggtcaataaactattttattgaggtcaataaattatttcattgaggtgacattggtattgaggtcaataaattgtttcattgagatatcattagtattgagatcaataaatttttttattgaggtcaagaaattttttttattgaagtcaataaactgtttattgtggcatttttgtaaatattggtgaccttatttgggaaaaaatattaggtggcttttttaagaataggatcacccaaagtggctttattggtaattatttattaacttaattcatgacagattatgagaaaaaaaagagagaatagaggagagagagagagagagatagataagaagaagaagaagagaaaacaaagatgccaaaaatagacactaagggcaataatgtcacaaaaaatcactatttaatatttttgtccttactgggagtaaaacatgtttaactgaaacttagtgagaattatttaaaatttatcaaacttgaagggaaatatgtagtgtaaaagaggatctattggttatttaccttaaaaaaaatttcatacaaatccatacacaatacacattgactatttaattatatgatgtattaatttgtttatactctaaagtacacaaccttgtggctgtagtttagtggtaagaattccacgttgtggccgtggagacctgggctcgaatcccagcagccacatagttttgttttttaaatcaattcccttctttttttcatgggttacatttatggaggtctcaaacccttatattcctttttcccttgtgtaataaaaatttaaaacataattgctaattagattttccttttaataaacctctacgtatattaacgcaacatatttctcacaattccatcccctagccttgagctacgaaggctttgtatataggactaagaggtttgaataattataaacttacaaacaaaatttgtaaaataattattgcatggtaccattaccacaattgtggcaatacatattggtttaattttagaccaacccaatagcagaaggcactaatttatgaccaacaagtacttttccgaaacttagagacaaatttgtcatcacctttttgtgtatgcacaattgaaacaaaatgtgttccttgcaaagattgattgatcgatatttaagtgaaaatcaaagtatgattgattaatccttttaaataaagtaaacatgaacaatgtacgtcgaaatagataaaaagggcctgtggtggtttttgttttactacccagacaggtttttgttgaagagtaaatgcaggtgtcaggagagggtaggagctggtgcgacatgaaagtacttaattctgtcattatttccatcttttcgtttacgtaagctcaatgtcatcctcaattttaaatgtatatttccactgttcctacttgtttgaaccggaaaaggttagtatagctaccatcatgctcatgtatgtatgtatgtgtgtatatatatatcccagtctctccagtctcttctcagctttgctcaggagtccaaacattcctttgtctagttagtcttgccttcctgtttgaatcacaaatcatgtggatgatgacggggtgtcgcagcgatggttgtggagagcctaacattaacaacagggatatccatgactactcgatcaaagctgcaaatcttcagcctgcagtagtagtactcactccaaagcttagcgctacttccaacataacccaaaattctgtgttaccattgaaccaccgccagcatcagggtacgtacatacgtaaacatatatataatatagatcacacatatcgatcttagtttgatggtctgattattctgtacgtgggtaggtaatagactaatagttcatatataattatacatgtgcagtgatccctgtcgcgccgtctgagcaaggaagaagtggtagtgattatttttataatgatcaacaaccagctgctgctgctgttgctcctccgcggagttcccgatggaatccaacgccagagcagctactggccctcgaagagctgtaccgacgtggcacaccaacggcccagcaaatccgacaagtgattcgacactttggaaagattgaaggcaagaatgtcttctactgaaccaccgcgcgagggaaaggcagaagagacgacgtgaactcatgtcatctatgtctcagtctcactctaatgaacaacgacaacaatcttggactaacaacctaatcagcagtatccagaacaaagaatccgcaggaggtactaattgagtcgcataattttcttattaatatatatatatatatgtacgtgcatatatatccatgcactatatatgtgcatacctaacctactgccatatttttaacagcgtaattaattaagcgtttagtgtccgtgattatctctcatggtactaattaaagttttgtttgcagaaggtcgacgtcttattagtactgtatatcaaatatgttttcggggtttttttcctccatgtgtgttactctccacctgtagcttcggggttttttataactcaattgatttcaatcaatattgttggaaaaccacctaatagaataattttagcacacacttacttggccggaactagggctcaactctaggtgaggccacctccatcctttaggtcacctacgctcaataactctacaccgtattaaatttcttgttataaggagaggagagactcagtggttgtgttaataataatttagtcttacaattaatatacctgggactagcgggcaccaagcaagatagcaaaccctagctaagagcaggacagcttgattaattgattgggcactcgatcgagtacatgtacttgtaaatttttctatatgacttaattttatgattgatctggtgcaaaaactgattaacacatatcacgtacaattgtgctcttctcccttcagacattgattaaccaataaatcacaactgtcctttgcaggattgagaaacttaagccttgaaggtgagcagcagaacaagtggactctatcaaactgtactattctttctgatcaggtatacgcgtgctatatctatcattttattaacgtacgtagttttaagacttaactatatctgaccaaaagctcaagaaaagatagaaatatattaattaattgaaagttaaattttttacagttttactctctctctctctctctctctcccccccccccccccaatagtatgtatatgtacttcatttgttaattagttactgtacgtccgcaggaatcatcttcagtgctgcaagagaggatgagcatggtggaaagcaattcctccaagcaaccggtggatcagtcctattcggctacaccgacctcacaaccaccatgcgcaaacccaactgtcaccttaataaacacaaagcccttggattgtcgtcactattatgattatggtatggtattgaccgcgccccatatatgcaaaggcgaagaaaccagagaaactcgaactctggagctctttcctctcgagagcgacaatctcaagggggttaaggattccgcattaccacaaacaaaaaaagatactactgcagacttcgtggctgaccaatattttcagtttctttgaagaatttaagggagcagcattcacatagctagaaattaatctacttaagaagggtttttcaatctatcgatctatttaataaattattatggattaattaattatgtatgtgtgtcttctctttgatggaaatgatagctagggtacatctttatcgggttctgttttggttctttccacttttatttcttctttttgtttatatgcagttcatttgtaaggacgggtaaggttgattgatgtccccctttctcgttcgttaggttctttctgtgtctctttctttctactgttgagggatatgtagttatatatcctgtcgctaggtgtacctttatctttttctgtaaataaaactccatcgatttcatggcagaccaatatatattttcaatttcattgaagaatttaagggcttatcaatttctctatctaatataaaatatataggcattttttactaatgggatccttactttttatgctacaaaattttgatttcagtcatcagatcacattatttaaaacaaatctgatagctgagatcaatactccgcaccataaaaagtaaggacctctttatataaaaattattgatatatgaagaaatttgatcacaacttaccgagagaaatgagttgcagctatatatgcttcgactagattacacataaataagccacgagtcctctagttgtaggcaactatcgtaacaaactgtaacaaaccccttggtcacgagataagcacaagctataataaacaaactatgtaaacctagctaaacttagtttattaacaaactaactagtcggaaactgatgagaaattttattaggacctacaaatttgctttacctaacttcctatattctttgcacctcttttttgcttttaaaatataagatttgcttactaaactttttttaaatttctaaaataaccataatgatataattcacattaaatatacaaaattactactataattctaaatttactcaccacacatttattaattaatttataatcaataaaaaaaatttagcatgaattattattatttttaactattcttttctcttttagctgtgcaataaaaaaaatagtaatcatttattcttacagtttatttttttctctattttttgtacctcatgattaaatattttatatatattttatgttgctagtttttttttttacaaatataatggatagacttaaatttaggtcatagtagaatttttttcgttctctatcactaatatacgttcaacaaatgatacaaatttttatgtcacataaactatcataattttaattcttattacatatatataaatattttaatgagtacgtattgaaattaaaaaatgaaaatagataagaaaaataataaaaaatacaatataatattattgaattagaaactgtagagaaaataaatataatatttttttggtataattattatccttaatagttttataggaggttataaatataatttaataattcataatggagtgaggttaactgagcaaaaatttggaggtttaagggaaactaattgatgactaattgccgacttagatgcttcatcataacatgtttgtcttctccttgttggaatggcatgcatggggtcggattctgttttggtttcccatttttctttgaatg

SEQ ID NO: 148 - protein, 140 aa, synthetic construct
MWMMTGCRSDGCGEPNINNRDIHDYSIKAANLQPAVVVLTPKLSATSNITQNSVLPLNHRQHQVIPVAPSEQGRSGSDYFYNDQQPAAAAVAPPRSSRWNPTPEQLLALEELYRRGTPTAQQIRQVIRHFGKIEGKNVFY

SEQ ID NO: 149 - DNA, 6194 bp, synthetic construct
  source 1..6194: mol_type = other DNA
gctcatgcgtcataattctaggtttgggtgggacccgatcacaaagagattcatatatgatgatgaagtatgagcatactattttatttgaaggtaaatttctattgacttttattctattctatttcgttaatgttctttttttttcttctattttttggtatgttttgtttttttttttgatacatatacctttgattttattaattacattgggaggttgctctttcctttgttttctaattattttgtgtcacaacattatttaagtttttctgaaatttgtagaagttgtttataataaagtttgtagatttcataaatcaataaaaggacttaaataaatcaatagaagtctatgaaatctatgaagtctgtagacttttcaaaaagtttgtaaattaaaaaaaaaagggtccttgaccataaggtccaaagttggacaaatatttaccatcaaggacaaaggaaaaacacggtgcccacccggtccattaattgaccaatttatatttttgaattatacattttacaaaaattatgaaactgccactggaaaattttattgaccacaataattttttttttttgagaaaacgtatattcctatggtaggagagaaagaacaaaaaaaatcttttttgtgtattttattgacctcaataaaattttattgagctccataaactgttttattgaggtcaataaacttttttattgaggtcaataaactgtttcattgagatgacattggttttgaggtcaataaactgtttcattgaggtgacattggttttgaggtcattaaactattttattgaggtcaataaactgtttcattgaggtgacattggttttgaggtcaataaactattttattgaggtcaataaattatttcattgaggtgacattggtattgaggtcaataaattgtttcattgagatatcattagtattgagatcaataaatttttttattgaggtcaagaaattttttttattgaagtcaataaactgtttattgtggcatttttgtaaatattggtgaccttatttgggaaaaaatattaggtggcttttttaagaataggatcacccaaagtggctttattggtaattatttattaacttaattcatgacagattatgagaaaaaaaagagagaatagaggagagagagagagagagatagataagaagaagaagaagagaaaacaaagatgccaaaaatagacactaagggcaataatgtcacaaaaaatcactatttaatatttttgtccttactgggagtaaaacatgtttaactgaaacttagtgagaattatttaaaatttatcaaacttgaagggaaatatgtagtgtaaaagaggatctattggttatttaccttaaaaaaaatttcatacaaatccatacacaatacacattgactatttaattatatgatgtattaatttgtttatactctaaagtacacaaccttgtggctgtagtttagtggtaagaattccacgttgtggccgtggagacctgggctcgaatcccagcagccacatagttttgttttttaaatcaattcccttctttttttcatgggttacatttatggaggtctcaaacccttatattcctttttcccttgtgtaataaaaatttaaaacataattgctaattagattttccttttaataaacctctacgtatattaacgcaacatatttctcacaattccatcccctagccttgagctacgaaggctttgtatataggactaagaggtttgaataattataaacttacaaacaaaatttgtaaaataattattgcatggtaccattaccacaattgtggcaatacatattggtttaattttagaccaacccaatagcagaaggcactaatttatgaccaacaagtacttttccgaaacttagagacaaatttgtcatcacctttttgtgtatgcacaattgaaacaaaatgtgttccttgcaaagattgattgatcgatatttaagtgaaaatcaaagtatgattgattaatccttttaaataaagtaaacatgaacaatgtacgtcgaaatagataaaaagggcctgtggtggtttttgttttactacccagacaggtttttgttgaagagtaaatgcaggtgtcaggagagggtaggagctggtgcgacatgaaagtacttaattctgtcattatttccatcttttcgtttacgtaagctcaatgtcatcctcaattttaaatgtatatttccactgttcctacttgtttgaaccggaaaaggttagtatagctaccatcatgctcatgtatgtatgtatgtgtgtatatatatatcccagtctctccagtctcttctcagctttgctcaggagtccaaacattcctttgtctagttagtcttgccttcctgtttgaatcacaaatcatgtggatgatgacggggtgtcgcagcgatggttgtggagagcctaacattaacaacagggatatccatgactactcgatcaaagctgcaaatcttcagcctgcagtagtagtactcactccaaagcttagcgctacttccaacataacccaaaattctgtgttaccattgaaccaccgccagcatcagggtacgtacatacgtaaacatatatataatatagatcacacatatcgatcttagtttgatggtctgattattctgtacgtgggtaggtaatagactaatagttcatatataattatacatgtgcagtgatccctgtcgcgccgtctgagcaaggaagaagtggtagtgattatttttataatgatcaacaaccagctgctgctgctgttgctcctccgcggagttcccgatggaatccaacgccagagcagctactggccctcgaagagctgtaccgacgtggcatcaagacaccaacggcccagcaaatccgacaagtgacggctgcttcgacactttggaaagattgaaggcaagaatgtcttctactgaccgcgcgagggaaaggcagaagagacgacgtgaactcatgtcatctatgtctcagtctcactctaatgaacaacgacaacaatcttggactaacaacctaatcagcagtatccagaacaaagaatccgcaggaggtactaattgagtcgcataattttcttattaatatatatatatatatgtacgtgcatatatatccatgcactatatatgtgcatacctaacctactgccatatttttaacagcgtaattaattaagcgtttagtgtccgtgattatctctcatggtactaattaaagttttgtttgcagaaggtcgacgtcttattagtactgtatatcaaatatgttttcggggtttttttcctccatgtgtgttactctccacctgtagcttcggggttttttataactcaattgatttcaatcaatattgttggaaaaccacctaatagaataattttagcacacacttacttggccggaactagggctcaactctaggtgaggccacctccatcctttaggtcacctacgctcaataactctacaccgtattaaatttcttgttataaggagaggagagactcagtggttgtgttaataataatttagtcttacaattaatatacctgggactagcgggcaccaagcaagatagcaaaccctagctaagagcaggacagcttgattaattgattgggcactcgatcgagtacatgtacttgtaaatttttctatatgacttaattttatgattgatctggtgcaaaaactgattaacacatatcacgtacaattgtgctcttctcccttcagacattgattaaccaataaatcacaactgtcctttgcaggattgagaaacttaagccttgaaggtgagcagcagaacaagtggactctatcaaactgtactattctttctgatcaggtatacgcgtgctatatctatcattttattaacgtacgtagttttaagacttaactatatctgaccaaaagctcaagaaaagatagaaatatattaattaattgaaagttaaattttttacagttttactctctctctctctctctctctcccccccccccccccaatagtatgtatatgtacttcatttgttaattagttactgtacgtccgcaggaatcatcttcagtgctgcaagagaggatgagcatggtggaaagcaattcctccaagcaaccggtggatcagtcctattcggctacaccgacctcacaaccaccatgcgcaaacccaactgtcaccttaataaacacaaagcccttggattgtcgtcactattatgattatggtatggtattgaccgcgccccatatatgcaaaggcgaagaaaccagagaaactcgaactctggagctctttcctctcgagagcgacaatctcaagggggttaaggattccgcattaccacaaacaaaaaaagatactactgcagacttcgtggctgaccaatattttcagtttctttgaagaatttaagggagcagcattcacatagctagaaattaatctacttaagaagggtttttcaatctatcgatctatttaataaattattatggattaattaattatgtatgtgtgtcttctctttgatggaaatgatagctagggtacatctttatcgggttctgttttggttctttccacttttatttcttctttttgtttatatgcagttcatttgtaaggacgggtaaggttgattgatgtccccctttctcgttcgttaggttctttctgtgtctctttctttctactgttgagggatatgtagttatatatcctgtcgctaggtgtacctttatctttttctgtaaataaaactccatcgatttcatggcagaccaatatatattttcaatttcattgaagaatttaagggcttatcaatttctctatctaatataaaatatataggcattttttactaatgggatccttactttttatgctacaaaattttgatttcagtcatcagatcacattatttaaaacaaatctgatagctgagatcaatactccgcaccataaaaagtaaggacctctttatataaaaattattgatatatgaagaaatttgatcacaacttaccgagagaaatgagttgcagctatatatgcttcgactagattacacataaataagccacgagtcctctagttgtaggcaactatcgtaacaaactgtaacaaaccccttggtcacgagataagcacaagctataataaacaaactatgtaaacctagctaaacttagtttattaacaaactaactagtcggaaactgatgagaaattttattaggacctacaaatttgctttacctaacttcctatattctttgcacctcttttttgcttttaaaatataagatttgcttactaaactttttttaaatttctaaaataaccataatgatataattcacattaaatatacaaaattactactataattctaaatttactcaccacacatttattaattaatttataatcaataaaaaaaatttagcatgaattattattatttttaactattcttttctcttttagctgtgcaataaaaaaaatagtaatcatttattcttacagtttatttttttctctattttttgtacctcatgattaaatattttatatatattttatgttgctagtttttttttttacaaatataatggatagacttaaatttaggtcatagtagaatttttttcgttctctatcactaatatacgttcaacaaatgatacaaatttttatgtcacataaactatcataattttaattcttattacatatatataaatattttaatgagtacgtattgaaattaaaaaatgaaaatagataagaaaaataataaaaaatacaatataatattattgaattagaaactgtagagaaaataaatataatatttttttggtataattattatccttaatagttttataggaggttataaatataatttaataattcataatggagtgaggttaactgagcaaaaatttggaggtttaagggaaactaattgatgactaattgccgacttagatgcttcatcataacatgtttgtcttctccttgttggaatggcatgcatggggtcggattctgttttggtttcccatttttctttgaatg

SEQ ID NO: 150 - protein, 137 aa, synthetic construct
MWMMTGCRSDGCGEPNINNRDIHDYSIKAANLQPAVVVLTPKLSATSNITQNSVLPLNHRQHQVIPVAPSEQGRSGSDYFYNDQQPAAAAVAPPRSSRWNPTPEQLLALEELYRRGIKTPTAQQIRQVTAASTLWKD

SEQ ID NO: 151 - DNA, 6197 bp, synthetic construct
  source 1..6197: mol_type = other DNA
gctcatgcgtcataattctaggtttgggtgggacccgatcacaaagagattcatatatgatgatgaagtatgagcatactattttatttgaaggtaaatttctattgacttttattctattctatttcgttaatgttctttttttttcttctattttttggtatgttttgtttttttttttgatacatatacctttgattttattaattacattgggaggttgctctttcctttgttttctaattattttgtgtcacaacattatttaagtttttctgaaatttgtagaagttgtttataataaagtttgtagatttcataaatcaataaaaggacttaaataaatcaatagaagtctatgaaatctatgaagtctgtagacttttcaaaaagtttgtaaattaaaaaaaaaagggtccttgaccataaggtccaaagttggacaaatatttaccatcaaggacaaaggaaaaacacggtgcccacccggtccattaattgaccaatttatatttttgaattatacattttacaaaaattatgaaactgccactggaaaattttattgaccacaataattttttttttttgagaaaacgtatattcctatggtaggagagaaagaacaaaaaaaatcttttttgtgtattttattgacctcaataaaattttattgagctccataaactgttttattgaggtcaataaacttttttattgaggtcaataaactgtttcattgagatgacattggttttgaggtcaataaactgtttcattgaggtgacattggttttgaggtcattaaactattttattgaggtcaataaactgtttcattgaggtgacattggttttgaggtcaataaactattttattgaggtcaataaattatttcattgaggtgacattggtattgaggtcaataaattgtttcattgagatatcattagtattgagatcaataaatttttttattgaggtcaagaaattttttttattgaagtcaataaactgtttattgtggcatttttgtaaatattggtgaccttatttgggaaaaaatattaggtggcttttttaagaataggatcacccaaagtggctttattggtaattatttattaacttaattcatgacagattatgagaaaaaaaagagagaatagaggagagagagagagagagatagataagaagaagaagaagagaaaacaaagatgccaaaaatagacactaagggcaataatgtcacaaaaaatcactatttaatatttttgtccttactgggagtaaaacatgtttaactgaaacttagtgagaattatttaaaatttatcaaacttgaagggaaatatgtagtgtaaaagaggatctattggttatttaccttaaaaaaaatttcatacaaatccatacacaatacacattgactatttaattatatgatgtattaatttgtttatactctaaagtacacaaccttgtggctgtagtttagtggtaagaattccacgttgtggccgtggagacctgggctcgaatcccagcagccacatagttttgttttttaaatcaattcccttctttttttcatgggttacatttatggaggtctcaaacccttatattcctttttcccttgtgtaataaaaatttaaaacataattgctaattagattttccttttaataaacctctacgtatattaacgcaacatatttctcacaattccatcccctagccttgagctacgaaggctttgtatataggactaagaggtttgaataattataaacttacaaacaaaatttgtaaaataattattgcatggtaccattaccacaattgtggcaatacatattggtttaattttagaccaacccaatagcagaaggcactaatttatgaccaacaagtacttttccgaaacttagagacaaatttgtcatcacctttttgtgtatgcacaattgaaacaaaatgtgttccttgcaaagattgattgatcgatatttaagtgaaaatcaaagtatgattgattaatccttttaaataaagtaaacatgaacaatgtacgtcgaaatagataaaaagggcctgtggtggtttttgttttactacccagacaggtttttgttgaagagtaaatgcaggtgtcaggagagggtaggagctggtgcgacatgaaagtacttaattctgtcattatttccatcttttcgtttacgtaagctcaatgtcatcctcaattttaaatgtatatttccactgttcctacttgtttgaaccggaaaaggttagtatagctaccatcatgctcatgtatgtatgtatgtgtgtatatatatatcccagtctctccagtctcttctcagctttgctcaggagtccaaacattcctttgtctagttagtcttgccttcctgtttgaatcacaaatcatgtggatgatgacggggtgtcgcagcgatggttgtggagagcctaacattaacaacagggatatccatgactactcgatcaaagctgcaaatcttcagcctgcagtagtagtactcactccaaagcttagcgctacttccaacataacccaaaattctgtgttaccattgaaccaccgccagcatcagggtacgtacatacgtaaacatatatataatatagatcacacatatcgatcttagtttgatggtctgattattctgtacgtgggtaggtaatagactaatagttcatatataattatacatgtgcagtgatccctgtcgcgccgtctgagcaaggaagaagtggtagtgattatttttataatgatcaacaaccagctgctgctgctgttgctcctccgcggagttcccgatggaatccaacgccagagcagctactggccctcgaagagctgtaccgacgtggcatcaccaacggcccagcaaatccgacaagtgacggcttcgacactttggaaagattgaaggcaagaatgtcttctactggtttcagaaccaccgcgcgagggaaaggcagaagagacgacgtgaactcatgtcatctatgtctcagtctcactctaatgaacaacgacaacaatcttggactaacaacctaatcagcagtatccagaacaaagaatccgcaggaggtactaattgagtcgcataattttcttattaatatatatatatatatgtacgtgcatatatatccatgcactatatatgtgcatacctaacctactgccatatttttaacagcgtaattaattaagcgtttagtgtccgtgattatctctcatggtactaattaaagttttgtttgcagaaggtcgacgtcttattagtactgtatatcaaatatgttttcggggtttttttcctccatgtgtgttactctccacctgtagcttcggggttttttataactcaattgatttcaatcaatattgttggaaaaccacctaatagaataattttagcacacacttacttggccggaactagggctcaactctaggtgaggccacctccatcctttaggtcacctacgctcaataactctacaccgtattaaatttcttgttataaggagaggagagactcagtggttgtgttaataataatttagtcttacaattaatatacctgggactagcgggcaccaagcaagatagcaaaccctagctaagagcaggacagcttgattaattgattgggcactcgatcgagtacatgtacttgtaaatttttctatatgacttaattttatgattgatctggtgcaaaaactgattaacacatatcacgtacaattgtgctcttctcccttcagacattgattaaccaataaatcacaactgtcctttgcaggattgagaaacttaagccttgaaggtgagcagcagaacaagtggactctatcaaactgtactattctttctgatcaggtatacgcgtgctatatctatcattttattaacgtacgtagttttaagacttaactatatctgaccaaaagctcaagaaaagatagaaatatattaattaattgaaagttaaattttttacagttttactctctctctctctctctctctcccccccccccccccaatagtatgtatatgtacttcatttgttaattagttactgtacgtccgcaggaatcatcttcagtgctgcaagagaggatgagcatggtggaaagcaattcctccaagcaaccggtggatcagtcctattcggctacaccgacctcacaaccaccatgcgcaaacccaactgtcaccttaataaacacaaagcccttggattgtcgtcactattatgattatggtatggtattgaccgcgccccatatatgcaaaggcgaagaaaccagagaaactcgaactctggagctctttcctctcgagagcgacaatctcaagggggttaaggattccgcattaccacaaacaaaaaaagatactactgcagacttcgtggctgaccaatattttcagtttctttgaagaatttaagggagcagcattcacatagctagaaattaatctacttaagaagggtttttcaatctatcgatctatttaataaattattatggattaattaattatgtatgtgtgtcttctctttgatggaaatgatagctagggtacatctttatcgggttctgttttggttctttccacttttatttcttctttttgtttatatgcagttcatttgtaaggacgggtaaggttgattgatgtccccctttctcgttcgttaggttctttctgtgtctctttctttctactgttgagggatatgtagttatatatcctgtcgctaggtgtacctttatctttttctgtaaataaaactccatcgatttcatggcagaccaatatatattttcaatttcattgaagaatttaagggcttatcaatttctctatctaatataaaatatataggcattttttactaatgggatccttactttttatgctacaaaattttgatttcagtcatcagatcacattatttaaaacaaatctgatagctgagatcaatactccgcaccataaaaagtaaggacctctttatataaaaattattgatatatgaagaaatttgatcacaacttaccgagagaaatgagttgcagctatatatgcttcgactagattacacataaataagccacgagtcctctagttgtaggcaactatcgtaacaaactgtaacaaaccccttggtcacgagataagcacaagctataataaacaaactatgtaaacctagctaaacttagtttattaacaaactaactagtcggaaactgatgagaaattttattaggacctacaaatttgctttacctaacttcctatattctttgcacctcttttttgcttttaaaatataagatttgcttactaaactttttttaaatttctaaaataaccataatgatataattcacattaaatatacaaaattactactataattctaaatttactcaccacacatttattaattaatttataatcaataaaaaaaatttagcatgaattattattatttttaactattcttttctcttttagctgtgcaataaaaaaaatagtaatcatttattcttacagtttatttttttctctattttttgtacctcatgattaaatattttatatatattttatgttgctagtttttttttttacaaatataatggatagacttaaatttaggtcatagtagaatttttttcgttctctatcactaatatacgttcaacaaatgatacaaatttttatgtcacataaactatcataattttaattcttattacatatatataaatattttaatgagtacgtattgaaattaaaaaatgaaaatagataagaaaaataataaaaaatacaatataatattattgaattagaaactgtagagaaaataaatataatatttttttggtataattattatccttaatagttttataggaggttataaatataatttaataattcataatggagtgaggttaactgagcaaaaatttggaggtttaagggaaactaattgatgactaattgccgacttagatgcttcatcataacatgtttgtcttctccttgttggaatggcatgcatggggtcggattctgttttggtttcccatttttctttgaatg

SEQ ID NO: 152 - protein, 179 aa, synthetic construct
MWMMTGCRSDGCGEPNINNRDIHDYSIKAANLQPAVVVLTPKLSATSNITQNSVLPLNHRQHQVIPVAPSEQGRSGSDYFYNDQQPAAAAVAPPRSSRWNPTPEQLLALEELYRRGITNGPANPTSDGFDTLERLKARMSSTGFRTTARGKGRRDDVNSCHLCLSLTLMNNDNNLGLTT

SEQ ID NO: 153 - DNA, 6209 bp, synthetic construct
  source 1..6209: mol_type = other DNA
gctcatgcgtcataattctaggtttgggtgggacccgatcacaaagagattcatatatgatgatgaagtatgagcatactattttatttgaaggtaaatttctattgacttttattctattctatttcgttaatgttctttttttttcttctattttttggtatgttttgtttttttttttgatacatatacctttgattttattaattacattgggaggttgctctttcctttgttttctaattattttgtgtcacaacattatttaagtttttctgaaatttgtagaagttgtttataataaagtttgtagatttcataaatcaataaaaggacttaaataaatcaatagaagtctatgaaatctatgaagtctgtagacttttcaaaaagtttgtaaattaaaaaaaaaagggtccttgaccataaggtccaaagttggacaaatatttaccatcaaggacaaaggaaaaacacggtgcccacccggtccattaattgaccaatttatatttttgaattatacattttacaaaaattatgaaactgccactggaaaattttattgaccacaataattttttttttttgagaaaacgtatattcctatggtaggagagaaagaacaaaaaaaatcttttttgtgtattttattgacctcaataaaattttattgagctccataaactgttttattgaggtcaataaacttttttattgaggtcaataaactgtttcattgagatgacattggttttgaggtcaataaactgtttcattgaggtgacattggttttgaggtcattaaactattttattgaggtcaataaactgtttcattgaggtgacattggttttgaggtcaataaactattttattgaggtcaataaattatttcattgaggtgacattggtattgaggtcaataaattgtttcattgagatatcattagtattgagatcaataaatttttttattgaggtcaagaaattttttttattgaagtcaataaactgtttattgtggcatttttgtaaatattggtgaccttatttgggaaaaaatattaggtggcttttttaagaataggatcacccaaagtggctttattggtaattatttattaacttaattcatgacagattatgagaaaaaaaagagagaatagaggagagagagagagagagatagataagaagaagaagaagagaaaacaaagatgccaaaaatagacactaagggcaataatgtcacaaaaaatcactatttaatatttttgtccttactgggagtaaaacatgtttaactgaaacttagtgagaattatttaaaatttatcaaacttgaagggaaatatgtagtgtaaaagaggatctattggttatttaccttaaaaaaaatttcatacaaatccatacacaatacacattgactatttaattatatgatgtattaatttgtttatactctaaagtacacaaccttgtggctgtagtttagtggtaagaattccacgttgtggccgtggagacctgggctcgaatcccagcagccacatagttttgttttttaaatcaattcccttctttttttcatgggttacatttatggaggtctcaaacccttatattcctttttcccttgtgtaataaaaatttaaaacataattgctaattagattttccttttaataaacctctacgtatattaacgcaacatatttctcacaattccatcccctagccttgagctacgaaggctttgtatataggactaagaggtttgaataattataaacttacaaacaaaatttgtaaaataattattgcatggtaccattaccacaattgtggcaatacatattggtttaattttagaccaacccaatagcagaaggcactaatttatgaccaacaagtacttttccgaaacttagagacaaatttgtcatcacctttttgtgtatgcacaattgaaacaaaatgtgttccttgcaaagattgattgatcgatatttaagtgaaaatcaaagtatgattgattaatccttttaaataaagtaaacatgaacaatgtacgtcgaaatagataaaaagggcctgtggtggtttttgttttactacccagacaggtttttgttgaagagtaaatgcaggtgtcaggagagggtaggagctggtgcgacatgaaagtacttaattctgtcattatttccatcttttcgtttacgtaagctcaatgtcatcctcaattttaaatgtatatttccactgttcctacttgtttgaaccggaaaaggttagtatagctaccatcatgctcatgtatgtatgtatgtgtgtatatatatatcccagtctctccagtctcttctcagctttgctcaggagtccaaacattcctttgtctagttagtcttgccttcctgtttgaatcacaaatcatgtggatgatgacggggtgtcgcagcgatggttgtggagagcctaacattaacaacagggatatccatgactactcgatcaaagctgcaaatcttcagcctgcagtagtagtactcactccaaagcttagcgctacttccaacataacccaaaattctgtgttaccattgaaccaccgccagcatcagggtacgtacatacgtaaacatatatataatatagatcacacatatcgatcttagtttgatggtctgattattctgtacgtgggtaggtaatagactaatagttcatatataattatacatgtgcagtgatccctgtcgcgccgtctgagcaaggaagaagtggtagtgattatttttataatgatcaacaaccagctgctgctgctgttgctcctccgcggagttcccgatggaatccaacgccagagcagctactggccctcgaagagctgtaccgacgtggcatcaagacaccaacggcccagcaaatccgacaagtgacggcggcgctgcttcgacactttggaaagattgaaggcaagaatgtcttctactggttcagaaccaccgcgcgagggaaaggcagaagagacgacgtgaactcatgtcatctatgtctcagtctcactctaatgaacaacgacaacaatcttggactaacaacctaatcagcagtatccagaacaaagaatccgcaggaggtactaattgagtcgcataattttcttattaatatatatatatatatgtacgtgcatatatatccatgcactatatatgtgcatacctaacctactgccatatttttaacagcgtaattaattaagcgtttagtgtccgtgattatctctcatggtactaattaaagttttgtttgcagaaggtcgacgtcttattagtactgtatatcaaatatgttttcggggtttttttcctccatgtgtgttactctccacctgtagcttcggggttttttataactcaattgatttcaatcaatattgttggaaaaccacctaatagaataattttagcacacacttacttggccggaactagggctcaactctaggtgaggccacctccatcctttaggtcacctacgctcaataactctacaccgtattaaatttcttgttataaggagaggagagactcagtggttgtgttaataataatttagtcttacaattaatatacctgggactagcgggcaccaagcaagatagcaaaccctagctaagagcaggacagcttgattaattgattgggcactcgatcgagtacatgtacttgtaaatttttctatatgacttaattttatgattgatctggtgcaaaaactgattaacacatatcacgtacaattgtgctcttctcccttcagacattgattaaccaataaatcacaactgtcctttgcaggattgagaaacttaagccttgaaggtgagcagcagaacaagtggactctatcaaactgtactattctttctgatcaggtatacgcgtgctatatctatcattttattaacgtacgtagttttaagacttaactatatctgaccaaaagctcaagaaaagatagaaatatattaattaattgaaagttaaattttttacagttttactctctctctctctctctctctcccccccccccccccaatagtatgtatatgtacttcatttgttaattagttactgtacgtccgcaggaatcatcttcagtgctgcaagagaggatgagcatggtggaaagcaattcctccaagcaaccggtggatcagtcctattcggctacaccgacctcacaaccaccatgcgcaaacccaactgtcaccttaataaacacaaagcccttggattgtcgtcactattatgattatggtatggtattgaccgcgccccatatatgcaaaggcgaagaaaccagagaaactcgaactctggagctctttcctctcgagagcgacaatctcaagggggttaaggattccgcattaccacaaacaaaaaaagatactactgcagacttcgtggctgaccaatattttcagtttctttgaagaatttaagggagcagcattcacatagctagaaattaatctacttaagaagggtttttcaatctatcgatctatttaataaattattatggattaattaattatgtatgtgtgtcttctctttgatggaaatgatagctagggtacatctttatcgggttctgttttggttctttccacttttatttcttctttttgtttatatgcagttcatttgtaaggacgggtaaggttgattgatgtccccctttctcgttcgttaggttctttctgtgtctctttctttctactgttgagggatatgtagttatatatcctgtcgctaggtgtacctttatctttttctgtaaataaaactccatcgatttcatggcagaccaatatatattttcaatttcattgaagaatttaagggcttatcaatttctctatctaatataaaatatataggcattttttactaatgggatccttactttttatgctacaaaattttgatttcagtcatcagatcacattatttaaaacaaatctgatagctgagatcaatactccgcaccataaaaagtaaggacctctttatataaaaattattgatatatgaagaaatttgatcacaacttaccgagagaaatgagttgcagctatatatgcttcgactagattacacataaataagccacgagtcctctagttgtaggcaactatcgtaacaaactgtaacaaaccccttggtcacgagataagcacaagctataataaacaaactatgtaaacctagctaaacttagtttattaacaaactaactagtcggaaactgatgagaaattttattaggacctacaaatttgctttacctaacttcctatattctttgcacctcttttttgcttttaaaatataagatttgcttactaaactttttttaaatttctaaaataaccataatgatataattcacattaaatatacaaaattactactataattctaaatttactcaccacacatttattaattaatttataatcaataaaaaaaatttagcatgaattattattatttttaactattcttttctcttttagctgtgcaataaaaaaaatagtaatcatttattcttacagtttatttttttctctattttttgtacctcatgattaaatattttatatatattttatgttgctagtttttttttttacaaatataatggatagacttaaatttaggtcatagtagaatttttttcgttctctatcactaatatacgttcaacaaatgatacaaatttttatgtcacataaactatcataattttaattcttattacatatatataaatattttaatgagtacgtattgaaattaaaaaatgaaaatagataagaaaaataataaaaaatacaatataatattattgaattagaaactgtagagaaaataaatataatatttttttggtataattattatccttaatagttttataggaggttataaatataatttaataattcataatggagtgaggttaactgagcaaaaatttggaggtttaagggaaactaattgatgactaattgccgacttagatgcttcatcataacatgtttgtcttctccttgttggaatggcatgcatggggtcggattctgttttggtttcccatttttctttgaatg

SEQ ID NO: 154 - protein, 183 aa, synthetic construct
MWMMTGCRSDGCGEPNINNRDIHDYSIKAANLQPAVVVLTPKLSATSNITQNSVLPLNHRQHQVIPVAPSEQGRSGSDYFYNDQQPAAAAVAPPRSSRWNPTPEQLLALEELYRRGIKTPTAQQIRQVTAALLRHFGKIEGKNVFYWFRTTARGKGRRDDVNSCHLCLSLTLMNNDNNLGLTT

SEQ ID NO: 155 - DNA, 6199 bp, synthetic construct
  source 1..6199: mol_type = other DNA
gctcatgcgtcataattctaggtttgggtgggacccgatcacaaagagattcatatatgatgatgaagtatgagcatactattttatttgaaggtaaatttctattgacttttattctattctatttcgttaatgttctttttttttcttctattttttggtatgttttgtttttttttttgatacatatacctttgattttattaattacattgggaggttgctctttcctttgttttctaattattttgtgtcacaacattatttaagtttttctgaaatttgtagaagttgtttataataaagtttgtagatttcataaatcaataaaaggacttaaataaatcaatagaagtctatgaaatctatgaagtctgtagacttttcaaaaagtttgtaaattaaaaaaaaaagggtccttgaccataaggtccaaagttggacaaatatttaccatcaaggacaaaggaaaaacacggtgcccacccggtccattaattgaccaatttatatttttgaattatacattttacaaaaattatgaaactgccactggaaaattttattgaccacaataattttttttttttgagaaaacgtatattcctatggtaggagagaaagaacaaaaaaaatcttttttgtgtattttattgacctcaataaaattttattgagctccataaactgttttattgaggtcaataaacttttttattgaggtcaataaactgtttcattgagatgacattggttttgaggtcaataaactgtttcattgaggtgacattggttttgaggtcattaaactattttattgaggtcaataaactgtttcattgaggtgacattggttttgaggtcaataaactattttattgaggtcaataaattatttcattgaggtgacattggtattgaggtcaataaattgtttcattgagatatcattagtattgagatcaataaatttttttattgaggtcaagaaattttttttattgaagtcaataaactgtttattgtggcatttttgtaaatattggtgaccttatttgggaaaaaatattaggtggcttttttaagaataggatcacccaaagtggctttattggtaattatttattaacttaattcatgacagattatgagaaaaaaaagagagaatagaggagagagagagagagagatagataagaagaagaagaagagaaaacaaagatgccaaaaatagacactaagggcaataatgtcacaaaaaatcactatttaatatttttgtccttactgggagtaaaacatgtttaactgaaacttagtgagaattatttaaaatttatcaaacttgaagggaaatatgtagtgtaaaagaggatctattggttatttaccttaaaaaaaatttcatacaaatccatacacaatacacattgactatttaattatatgatgtattaatttgtttatactctaaagtacacaaccttgtggctgtagtttagtggtaagaattccacgttgtggccgtggagacctgggctcgaatcccagcagccacatagttttgttttttaaatcaattcccttctttttttcatgggttacatttatggaggtctcaaacccttatattcctttttcccttgtgtaataaaaatttaaaacataattgctaattagattttccttttaataaacctctacgtatattaacgcaacatatttctcacaattccatcccctagccttgagctacgaaggctttgtatataggactaagaggtttgaataattataaacttacaaacaaaatttgtaaaataattattgcatggtaccattaccacaattgtggcaatacatattggtttaattttagaccaacccaatagcagaaggcactaatttatgaccaacaagtacttttccgaaacttagagacaaatttgtcatcacctttttgtgtatgcacaattgaaacaaaatgtgttccttgcaaagattgattgatcgatatttaagtgaaaatcaaagtatgattgattaatccttttaaataaagtaaacatgaacaatgtacgtcgaaatagataaaaagggcctgtggtggtttttgttttactacccagacaggtttttgttgaagagtaaatgcaggtgtcaggagagggtaggagctggtgcgacatgaaagtacttaattctgtcattatttccatcttttcgtttacgtaagctcaatgtcatcctcaattttaaatgtatatttccactgttcctacttgtttgaaccggaaaaggttagtatagctaccatcatgctcatgtatgtatgtatgtgtgtatatatatatcccagtctctccagtctcttctcagctttgctcaggagtccaaacattcctttgtctagttagtcttgccttcctgtttgaatcacaaatcatgtggatgatgacggggtgtcgcagcgatggttgtggagagcctaacattaacaacagggatatccatgactactcgatcaaagctgcaaatcttcagcctgcagtagtagtactcactccaaagcttagcgctacttccaacataacccaaaattctgtgttaccattgaaccaccgccagcatcagggtacgtacatacgtaaacatatatataatatagatcacacatatcgatcttagtttgatggtctgattattctgtacgtgggtaggtaatagactaatagttcatatataattatacatgtgcagtgatccctgtcgcgccgtctgagcaaggaagaagtggtagtgattatttttataatgatcaacaaccagctgctgctgctgttgctcctccgcggagttcccgatggaatccaacgccagagcagctactggccctcgaagagctgtaccgacgtggcatcaagacaccaacggcccagcaaatccgacaagtgacttcgacactttggaaagattgaaggcaagaatgtcttctactggtttcagaaccaccgcgcgagggaaaggcagaagagacgacgtgaactcatgtcatctatgtctcagtctcactctaatgaacaacgacaacaatcttggactaacaacctaatcagcagtatccagaacaaagaatccgcaggaggtactaattgagtcgcataattttcttattaatatatatatatatatgtacgtgcatatatatccatgcactatatatgtgcatacctaacctactgccatatttttaacagcgtaattaattaagcgtttagtgtccgtgattatctctcatggtactaattaaagttttgtttgcagaaggtcgacgtcttattagtactgtatatcaaatatgttttcggggtttttttcctccatgtgtgttactctccacctgtagcttcggggttttttataactcaattgatttcaatcaatattgttggaaaaccacctaatagaataattttagcacacacttacttggccggaactagggctcaactctaggtgaggccacctccatcctttaggtcacctacgctcaataactctacaccgtattaaatttcttgttataaggagaggagagactcagtggttgtgttaataataatttagtcttacaattaatatacctgggactagcgggcaccaagcaagatagcaaaccctagctaagagcaggacagcttgattaattgattgggcactcgatcgagtacatgtacttgtaaatttttctatatgacttaattttatgattgatctggtgcaaaaactgattaacacatatcacgtacaattgtgctcttctcccttcagacattgattaaccaataaatcacaactgtcctttgcaggattgagaaacttaagccttgaaggtgagcagcagaacaagtggactctatcaaactgtactattctttctgatcaggtatacgcgtgctatatctatcattttattaacgtacgtagttttaagacttaactatatctgaccaaaagctcaagaaaagatagaaatatattaattaattgaaagttaaattttttacagttttactctctctctctctctctctctcccccccccccccccaatagtatgtatatgtacttcatttgttaattagttactgtacgtccgcaggaatcatcttcagtgctgcaagagaggatgagcatggtggaaagcaattcctccaagcaaccggtggatcagtcctattcggctacaccgacctcacaaccaccatgcgcaaacccaactgtcaccttaataaacacaaagcccttggattgtcgtcactattatgattatggtatggtattgaccgcgccccatatatgcaaaggcgaagaaaccagagaaactcgaactctggagctctttcctctcgagagcgacaatctcaagggggttaaggattccgcattaccacaaacaaaaaaagatactactgcagacttcgtggctgaccaatattttcagtttctttgaagaatttaagggagcagcattcacatagctagaaattaatctacttaagaagggtttttcaatctatcgatctatttaataaattattatggattaattaattatgtatgtgtgtcttctctttgatggaaatgatagctagggtacatctttatcgggttctgttttggttctttccacttttatttcttctttttgtttatatgcagttcatttgtaaggacgggtaaggttgattgatgtccccctttctcgttcgttaggttctttctgtgtctctttctttctactgttgagggatatgtagttatatatcctgtcgctaggtgtacctttatctttttctgtaaataaaactccatcgatttcatggcagaccaatatatattttcaatttcattgaagaatttaagggcttatcaatttctctatctaatataaaatatataggcattttttactaatgggatccttactttttatgctacaaaattttgatttcagtcatcagatcacattatttaaaacaaatctgatagctgagatcaatactccgcaccataaaaagtaaggacctctttatataaaaattattgatatatgaagaaatttgatcacaacttaccgagagaaatgagttgcagctatatatgcttcgactagattacacataaataagccacgagtcctctagttgtaggcaactatcgtaacaaactgtaacaaaccccttggtcacgagataagcacaagctataataaacaaactatgtaaacctagctaaacttagtttattaacaaactaactagtcggaaactgatgagaaattttattaggacctacaaatttgctttacctaacttcctatattctttgcacctcttttttgcttttaaaatataagatttgcttactaaactttttttaaatttctaaaataaccataatgatataattcacattaaatatacaaaattactactataattctaaatttactcaccacacatttattaattaatttataatcaataaaaaaaatttagcatgaattattattatttttaactattcttttctcttttagctgtgcaataaaaaaaatagtaatcatttattcttacagtttatttttttctctattttttgtacctcatgattaaatattttatatatattttatgttgctagtttttttttttacaaatataatggatagacttaaatttaggtcatagtagaatttttttcgttctctatcactaatatacgttcaacaaatgatacaaatttttatgtcacataaactatcataattttaattcttattacatatatataaatattttaatgagtacgtattgaaattaaaaaatgaaaatagataagaaaaataataaaaaatacaatataatattattgaattagaaactgtagagaaaataaatataatatttttttggtataattattatccttaatagttttataggaggttataaatataatttaataattcataatggagtgaggttaactgagcaaaaatttggaggtttaagggaaactaattgatgactaattgccgacttagatgcttcatcataacatgtttgtcttctccttgttggaatggcatgcatggggtcggattctgttttggtttcccatttttctttgaatg

SEQ ID NO: 156 - protein, 135 aa, synthetic construct
MWMMTGCRSDGCGEPNINNRDIHDYSIKAANLQPAVVVLTPKLSATSNITQNSVLPLNHRQHQVIPVAPSEQGRSGSDYFYNDQQPAAAAVAPPRSSRWNPTPEQLLALEELYRRGIKTPTAQQIRQVTSTLWKD

SEQ ID NO: 157 - DNA, 10 bp, Rubus sp.
  source 1..10: mol_type = genomic DNA
cagcatcagg

SEQ ID NO: 158 - DNA, 10 bp, Rubus sp.
  source 1..10: mol_type = genomic DNA
gcatcagggt

SEQ ID NO: 159 - DNA, 11 bp, Rubus sp.
  source 1..11: mol_type = genomic DNA
gtttcagaacc

SEQ ID NO: 160 - protein, 8 aa, synthetic construct
  VARIANT 3: note = Xaa denotes Asp, Glu, Gln or Pro
  VARIANT 7: note = Xaa denotes Gly, Ile, Thr, Val or Leu
  VARIANT 8: note = Xaa denotes Gly, Ser, Lys, Asn, Thr, Cys or Val
TLXLFPXX

SEQ ID NO: 161 - protein, 8 aa, synthetic construct
TLELFPLH

SEQ ID NO: 162 - DNA, 1833 bp, Rosa chinensis
  source 1..1833: mol_type = genomic DNA
acacgccaacacgcacacactcttgactctctccatccccttaccatctttgttcatgattcaaaacattcccttgtctagttagccatccacgtttgaataactatcagtgatgtacgtggatgatgatggggtgtagcaacggtgattgtagagaccccaacagaaacaataatagggatctgcctgagtactcgatcaaagctgcaaatcctcagcctgcagtacagccagtactattcactccaaagcttagctctacttccaacatgacccaaaaccctgtgttaccattcaaccaccaccatcatcgcagtgcgtacagatgagcgcgcacacacacacacacaatattatatatatacgattacatcacgcacacacatattgtactttgatggtctgattattctgtgggtaggtgataatatgacttcatatgtatgcagtggttccagtaatgtctgatgagcaaggcagaagtagcagtggttgtttttatagtgataaacaaccggcagctggagctcctcggcgaagctgccgatggaatccaaccccggagcagataaaggacctggaagagctgtatcgatctgggatcaagacaccgacggcccaacaaatccgacaggtgactgcgcggcttcgaaactatgggagggttgaaggcaagaatgtattctactggtttcagaaccaccgagccagggagaggcagaagagacgccgtgaactcatctccatgcatcagtctgacactaaacaacagcaacaaactttgattaacagcaagattaccagtctccagaaaaaagaatctgcaggaggtaccaattaattagcatcattttattattaacatcataacatgcatgcaatcgacgtgttggacacatatggctccccaggtaatagggggattagggctcaacacttggtgaggccacctcctttaggtcacctacggctcaataactgtataccatattgaatatctatgttatgaggaggggagagcgagagactcaatggttgtgttaataattaagacgattcaatacaaataatatacccgtgccttacgctcaccaggcaagtcagcaaaccctagccaagagctggagaacctaattaattgaacgggcactcgggtacaaggacttttaaatttgttggttctatatgagtttcaaagttgagccagtgcaataattattaacagattatcacaagttaaggaaattgtatacttgcagttagctctgttggtcgtcttgaagttgagcagcaaaattggccgaccctaccaaatttcactgttctttccgatcaggtaaacctatacaggtatatcaaaatctcaagaaaatatagaaattaattgaaagcgtgatatggatatttactgttttacactacatatgtgcatgagtttcatttgttactgtacatcctcaggaatcatcttcagtactgcaggagagaatgatgagcatggtgaaaagcaattcctccaggctacctatggaacaatccaattcacctccgacctcatcagacacaaacgcgtcatcagctacattaatttacacaaagctcttggactttcatcactattatgattatgactacggtatggtattgactgcacccaatgtttacaaagaagaagaaaaagaaagcagagaaactcaaactctagagctctttccacttgagagcgacaatcttaagagtgctaagcataccaaattacctatgagcagaacaagcactgcagatttcaaggaagaccaatattattagtttctttgaacaatttaagggagc

SEQ ID NO: 163 - DNA, 1569 bp, Rosa chinensis
  source 1..1569: mol_type = genomic DNA
aaatcttcaggattcgagtcatgaacttctcaatttcttcaaaattcattgaaaaagaaacttatctgaatcagagatctcacacaagagaaagaggaggatgagcaccagctaagattgtagatcaagcacgaagatcaaccacgatcgcagcctgagctcttgataccatgaaagttctagagaggaatagagaaacgaagagaaaatatctagaaaatgagcctgattcaattatgctagtacagagaggagtttatatagtactcaagaagattcccaggcttgacacgtgtcaagacagctgtggatcggagcactagtgaactaaagcatgcagctaaacgtaattaacataggctaattaccctaattgcttattacaaagcaatagaaattaactagagcaattagctaaacaacacaatcctaacaatctctccccaagtaatatggaactagggcttaacactaggctggtgaggccacctccttttggttaattgattgagtccttgagttttacaagaaattttaaaatttgtttctatatgagtttcacgtacgattgatcaggctcaaaaagtaattaacagataaatcactagttatggaaattgtctacttgcagctgcaggacagagaacgtcaagtactcttgaaattgagcaccagaataaatggaccttgtcaaactgcggtgttcttgctgatcaagtatatatcattttcttaatgtatgtggttcttagacttaactgtatctaaccaaaatctcaagaaaatatagaaatcgattgaatgctaggaatttttacttttttacattacgtatgtacgtacgtgcatgagtttcatttgttaccgtaattcagctagaattatcttcagtgatgcaaaaggggatgagcatggtgaccggcaattactctggtcaattggaggagcaaccctattcggccacacccacctcggaagcacaacgaacaaatgcatcagctacattaatatacacaaagcgcttggactatcatcatccccattatgattatgatcgattacggccgtatggtgttgagtgcacccaatatttacaaaggagcagaaagtagattagaaactcaaactctggagctctttccacttgagagcgacaatgttaagggtgttaatgattaaatcacctatgatcataaccataaacacagacaataatgaagatttcatggcagaaaaatattttcaatttctgtgaagaatctgagtacctctttatcaggttcagtaatgaagatttcatggcatgttcttcctacttatatttcgtcttttttgtttaaaggcagtttaatttgttcgcatggaaggataaggttgatgtctccctttctgtttaatttgttttttctgtattgttgatcgaggggtacgcacctgaagatccctctagttaggtgaacataccattttcttttcctgtaaataaatgaatgaatctccatcttattctcgagttccatgaaatattatgaaactaatatgcatatgtgtacgtagaagctgtcgaat

SEQ ID NO: 164 - DNA, 3363 bp, Rosa chinensis
  source 1..3363: mol_type = genomic DNA
ctatatatagacacacgtgtactcgactctctccatccccttcccatctttgctcatgattcaaaacattcccttgtctagttagccctcatagtttgaataagtactaccagtgttgtggacgatgatgggtagcagcggtggttgtggagaccctaaaactagggttctgcctgagtacttctcgatcaaagctgcatatcctcagcctctactcactccaaagcttagctgtacttccaacatgacccaaatccctttgttaccattcaaccaccaccatcagagtacgtaccgatgatttatatatacacacacaatcatatatagatgattacagtacacacacacacatatatatacatattatactatgatggtctgattattctgtgggtaggtgatagtatgatttcatgtgcatgcagtggttccagtgacgtcgaatgagcaaggcagacatagcagtgattgtttttatagtgataaacaaccggcagctgcagctcctcctcgaagttcccgatggactccaaccccggagcagataatgcatctggaagagctgtatcgatctgggatcaagacaccgacggcccaacaaatccgacaggtggctgcacggcttcgaaactatgggagggttgaaggcaagaatgtattctactggtttcagaaccgcagagccagggagaggcagaagacacgccgtgaactcatgtccatgcatcagtctgactctaaacaacaacaacaacaagctttgactaagaacacaattagcagtctccgggacaaagaatctgcaggaggtaccattgaataagcatcattttattattaacatgcagttaatctatgtgtttgacacaaggctcccccggtaatagggggattagggctcaacacttgatgaggcacctcctttaggtcacctacggctcaatacctgtatgtaccatattgaatatctatgctatgaggagaggagagtgagagacataaccaataattaagaccattcagtacaaataatatacccgtgctttacgctcaccaggcaagtcagcaaactctagccaagagctggagaacctaattaattgaacgggcactcaagtacaaggacttgtaaatttattggttctatatagttgaacgggagcaaaaattaattaacagattattacaagttatggaaattgtttccttgcagctgactttcttcgttgttttgaagttgagcagcagaataggcgaacactaccaagcttcactcttcttaccaatcaggtaagcatataccgtatatcgaaccaaaatctcaaaaaagtatagatttttattgaatgtgttatttacttttggaaaatgattattggtctcaatcccagttgtcatgtcatgtcacctacacacatcacacttattctatgtaatagttgaataaaaagactattttatcattctaaaatctaatagctctaaattatttgcttacttttaaaataaaaaaatagtattttttctatttctttttttcttttcattacactcatgcttagatttaaacaatttttttttcttcaatcatgaatagaaagtatttcatgtaattcgatcaatagagttgcataacacaagtatatgaatttagagtttgttgggttcctgcaaattatgaaaatatatgtgaaaaatacatattcaagctgatatgaattatatatatatatatatatatatttcatccaaaaaatacacacacacatatataatgaaatatatttatttaaaaaacaaataagatatttttttttaatctcagctataacctaatggttatatgatctaatggtttagaatgagagtataatcttaattataatttaggtggttattgatccgcccctaattggggtgttgtggtgtcgctagttctagcagcttgcttcaaataaaaaaatcccgaggtcgtcagggcgggcggatagcttggcacctggagatataggcggcaggatggattagatacaaattcaagagtatactttgtttgggttaggcgaagtccagaggtggagcgagatacctccatatgccgagtaaaaagttgtttgaaaataaatttaataaaattaataaaatataactaatcatccttatgctaataagttaattaggaaagtagaaaattaaattggattccgtattaaaaaaaataaattaaattagatccacataaatggaaagagaatatattgatatcgtaattttaatacataaaatctagtaatttaagtaatatttaactctcttaataagtgatggaaaaacattggaagaaagagttaagacttaagagtggatctaataataaattattttcactaaatttttctctcacaattaaatattaaattaaaacatgaaggaatacgtattaattgaagggaagaaagatatttcaaattttaattaaattctttaatgtcattgattcaccccctaaaatctaaaagaaaatataaaagggtaaaattggtgttctaagattataatttggcaatatatattatgtggaattgaaaataaaaatttgtatttgcttacttgacatgacacctaggattgaggctattgaggccctacgtgagtttcatttgttactatatgtcctcagaaatcatcttcagtactacaagagcggatgatgagcatggtgaaaagcaattcctccaggctacttatggaacacatcgattcacctggactgacctcaaacataaacaagtcagctacattagtttacagaaagctcttggactttcatcactattatgattacgactacggtatggtactattgattgcaccgaatatttacaaagaagaagaagaagaagaagaaagcagagaaactcaaactcttaagctctttccactcgagagtgacactcttaagggtgctaaggataccaaattacctatgagcagaaccagcacggacactacttcaaatttcatggcagaccaatattattagtttctttgaacatttaagggagcatatagctaggaatctatatattcatgtaataatagtatgcatgcgtgtcttctctttcatggaataatgtagcacatcttaatgatcaggccggttctgtatttgcttattcttcccacttttatttcttctatttgttcaaacacagaggaagagtaaggtttgtgtccctcttctttagtttgttagcgtacattttctgtttctatatataaaactccatcgtattgtattgtcaaagtccatcgaaaatgtaaggtcaatacatatgcataactagcaagctctcatat

SEQ ID NO: 165 - DNA, 2073 bp, Rosa chinensis
  source 1..2073: mol_type = genomic DNA
atatatgtatatatagagatacttgactctctccatccccttaccatctttgctcatgattcaaaacattcccttgtctagttggccatcatagtttgaataatagggctctgcctgaattactcgatcaaagctgcatatcctcagcctgcagtactactcactccaaagcttacctctacttccaacatgacccaaatccctttcttccgccatcagagtacgtactgatgtacacacacacacacacacacacacacacacacacacacacatatatgtatatattatactttgatcgtctgattattctgtgggtaggtgataatatgatttcaaatgtatgcagtggttccagtgacgttggatgagcaaggcagtgattgtttttatagtgataaacaaccggcagctgcagctcctccgcgaaattcccgatggaatccaaccccggagcagataatgcacctggaggagctctatcgatctgggatcaagacaccgacgacccaacaaatccgacaggtgactgcagggcttcgaaactatgggagggttgaaggcaagaatgtattctactggtttcagaaccaccgagccagggagaggcagaagagacgccgtgaactcatgtccatgtatcagtctgactctaaacaacaacaacaacaaactttgactaacaacacaattagcagtctccacgacaaagaatctgcaggaggtaccatttaattagcatcattttattattaacatgcagttagtctatgtgttggacacatggctccccggccaggtaatagggggattagggctcgacacttggtgagtccacctcctttaggtcacccacggctcaataactgtataacatattgaatatctatgttatgatcgaggaggggagagtgagagacttaatggttgtgttaataataattaagaccattcagtacaaataatgtacccgtgctttacgctcaccaggcaagtcagctaaccctagccaagagctggagaacctaattaattgaacgggcactcgtgtataaggtcttgtaaatttgttggttctatatgagtttcaaagttgagcgcgcgggtgcaaaaattaattaacagattattacaagttatgaaaatcgtttacttgcaggtgactttgttcgttgtcttgaagttgagcagcagaataggcggacactaccaaacttaactgttctttccgatcaggtaaacgtgtacccgtatatcaaaatctaaaaaaaaatatagaaattaactgaatgcgtgatatggatattaactgctttacaatacacacacacacacacacatatatatatatgcatgagtttcatttgttactgtacgtcctcaggaatcatctacagtactgcaagagaggatgatgagcatggtgaaaagcaattcctccaagctacttgtggaacaatccgattcacctggatcgacctcagacacaaacgcgtcagctacagtcatttacacaaagctcttggacttccatcactattatgattatggtatggtattgactacacccagtatttacaaagaattcaaagaagaagaagaaagcagagaaactcaaactctggagctctttccactcgagagcgacaatcttaaaggtgctaaggataccaaattacctatgagcagaaccgcaagcacagacactacttcagatttcatggcagaccaatattattagtttctttgaacaatttaagggagcacataactaggaatctatatattcaagtaataatagtatgcatgcgtgtcttctctttcatggaataatgtggcacaccattatgatcaagccggttctgttttttgtcgttcttcccacttctatttcttctatttgttcaaacacagaggaagagtaaggttggtgtcccccttctttacttaattagtttagtttgctgtaccatttctatttctatatatgaaactccatcgtattgtattgtcgatcaaaatccatcgaaaatgaaaggtcaatacatatgcataactagcaagctctcatat

SEQ ID NO: 166 - DNA, 1784 bp, Rosa chinensis
  source 1..1784: mol_type = genomic DNA
tgcctatataatatgtgcgcatgcacattctctgcatcgttctcagctctacttgctacaagatttcctagtaggacttattgtccatgctatagtatctatcactatatcacaatcccgagtcaaaagaatcccctgtcgatcgagcagcggtggttttagagagcctaacatgaacaacagtagggatatgcatgaggatgactactactcgatcaaagctgcaaatgttcatgagcccgctgcagaagtagcattcactccaaagcttagcgctacttccaacatgacccaaagttatgtgttatcatccaaccaccaccatcatcagggtacttagtaagttaattaaattttaaaattgaagatattaacatgggatatagtaaaacaattaatatatttcatacagtggttggagtgacgcagtcgcaaggcagaagtggcggtgattattgctataatggtcaccaaccctcctctgcagcttcgcagcaaagttcccgatggaatccaaccccagagcagttacgggtcctggaagagctgtatggacatggattaaagacaccaacggcagaacaaatccgacaagtgactgcgcggcttcaacactttggaaagattgaatacaagaatgtcttctactggtttcagaaccaccgagcgagggagagacagaagagacgacatgaactcctgtccatgcctcagtatcactctaaggaaaaacaactttggagcaacaaagaatctgcaggaggtattggccttgggactgtactgtattaaatatgttttcatctctccccattccccaagtaatatggaactagggcttaacactaggcccgtgtggccacctcctttaggttaattgattgagtgctagctcgagttttacaagaagttgtaaaatttgtttctatatgagtttcacgattgatcaggctcaaaaattaattaacagatcaatcacaagttatggaaattgtctacttgcagctgcaggacagagaaggtcaagtactcttgaaattgagcaccagaataaatgggcactatcaaactacggtattcttgctgatcaggtatatatcattttcttaacgtatgtggttcttggacttaactgtgtctaatcaaaatctcaataaaagatagaaatggattgaatgctaggaatttttacttttttacattacgtatgtacgtgcatgagtttcatttattactgtacgtccgcaggaattatcttcagtgctgcaaaagaggatgagcatggtgaccggcaattactccggtcaattggaggagcaatcctatttggctacaccgacctcggaagaacaacaaacaaacgcatcagctacattaatatacacaaagctcttggactatcatcatctccattatgataatgattacggtatggtgttgactacacccaatatttacaaagaagcagaaagtagattagaaactcaaactctggagctctttccacttgagagcgacaatgttaagggtgttatggataccgaatcacctatgatcagaaccacaaacacagacagtaaggaagatttcatggcataccaatattttcaatttctttgaagaatctgagaatcggagtatttagctagaaatctccttaatttggaagcgtctgccaatctatccatctaataaataataggcatgtgtgtgttcttgggggtgggggagtgtgggttgtgtaactctttatcaggttctgttcttggtcttcctacttttat

SEQ ID NO: 167 - DNA, 1532 bp, Rosa chinensis
  source 1..1532: mol_type = genomic DNA
ctgtatatatatatatatatatatatatatatatatataatcttaatgaaatatgtacacacacacacacacagatatatatatttaatgaaatatatttatttaaaaaacaaataacatatttttttaatctcaactatattaatctaaaggttatatgatctaatggtctagaatgagattataatcttaattataatttaggtggttattgttctgcccctagttggggtgttggggtgtcaaaagttgtttgtaaataaatttaataaaattaataaaaagtaactaatcatccttacactaataagttaattaggaaagtaaaaaattaaattggattacatatcaagaagaaaaaaaattaaattggattcacacaaatggaaagagaatatattgatatcgtaattttaattaataaaatctagtaatataagtaatatttaactctcttaataagtgatggaaaaacattgaaagaaagagttaagagcagatctaataataaattatttccactaaattttttctctcacaattaaatattaaattaaaacatgaaggaatacgtattaattgaagggaagaaagatatttcaaattgtaattaaattcattaatatcattgattcaccccctaaaatctaaaaggaaatataaaagggtaaaaatcggtgttccaagattataatttggcaatatatattatgtgaattgaaaataaaaatgtgtatatgcttacatggcatgacacttaggattgaagtcattgaggccatttactattttacactacatatgtgcatgagtttcatttgttactgtacgtcctcaggaatcatcttcagtactgcaagagaggatgatgagcatggtgaaaagcaattcctctgagctacttatggaacataccgattcaccaagactggcctcaaacacaaacgtgtcagctacattaatttacaaaaagctcttggactttcatcattattatgattacaattacggtatggtattgacttcaccgaatatttacaaagaagaagaagaaagcatagaaactcaaactcttaagctctttccactcaagagcgacaatcttaaaggtgctaaggataccaaattacctatgagcagagccacaagcacagacggtacttcagatttcatggcagaccaatattattagtttctttgaacacttgagggagcatatagctaggaatcaatatattcatgtaataatagtatgcatgcgtgtcttctctttcatggaataatgtggcacatctttatgatcaggccagttctgtatttgcttattcttcccacttttgtttcttctgttttttcaaacacagaggaagagtaaggttggtgtccctcttctttacttaattaggtttctttagttttctagtgcttgtaccttttttgtttctacatataaaactccatcgtattgtattgtcgaagtccatcgaaaatgtaaggtcaatacatatgccactacaagaaatctaggcttt

SEQ ID NO: 168 - DNA, 1785 bp, Rosa chinensis
  source 1..1785: mol_type = genomic DNA
atatatatacacacacacatacctgactctctccatacccttaccatctttgctcatgattcaaaacattcccttgtctagttagccatcgtagtttgaataactatcagtgatgtggacgatgatgggtagcagcagtggttgtagagaccctaatattggggctctgcctgagtactcgatcaaagctgcatatcctcagcctgcagtactactcactccaaagcttagctctacttccaacatgacccaaatccctttgttaccattcaaccaccaccatcagagtacgtaccgatgatttatacacacacacacattcatatatagatgattgcagtacacacacacacacacaatcatatatagatgattgcagtacacacacacacatacatatatatacatatcatactttgatggtctgattattctgtgggtagctgataatctgatttcatgtgtacgtatgcagtggttccagtgacgtcgaatgagcaaggcagaaatagcagtgattgttcttatagtcataatcgaagttcccgatggactccaaccccggagcagatagtgcatctggaagagctgtatcgatctgggatcaagacgccgacggcccaacaaatccgacaggtgactgcacggcttcgaaactatgggagggttgaaggcaagaatatattctactggtttcagaaccgccgagccagggagaggcagaagacacgccgtgaactcgtgtccatgcatcagtccgactctaaacaacaacaacaacaaactttgactaacaacacaattagctgtctccgagaaaaagaatctgcaggaggtatcatttaataagcatcattttattattaacatgcagttaatctatctgttggacacaaggctccccaggtaatagggggattagggttcaacacttggtgaggccagctcctttaggtcacctacgggctacggctcaattactgtataccatattgaatatctatgctatgaggaggggagagtgagagagttaatggttatgttaataataattaagaccattcagtacaaataatataaccgtgctttacactcatgactcaccaggcaagtcagcaaaccctagccaagagctggagaacctaattaattgaacgggcactcgagtacaaggacttgtaaatttattggttctatatagctgaacgggtgcaaaaattaattaacagattatttcatgttatggaaattgtttgcttgccgttgactttgttcgttgttttgaagttgagcagcagaatagatggacactacgaagcttcactcttcttaccgatcaggtaagtgtatattgtataatgtatatcgagccaaaatctcaagaaactatagaaatttattgaatgcattatttaatgttggaaaatgattattcgcctcaatctgagttgtcatgttatgtcacctactcacattacacttatgccatgtaatactttaaaaaaaaaaatcatcttgcccttctaaaatccaatagctctaaattatttgcttactttaaaaaaaatggtttttttttttctgtttcttttttcttcaatcaagaatagatttgcataacacatttatatgaattcggagtttgttgggttcctacaaattatgaaaatatatgtgaaaaatacatattcatgttcatatgaatatatatatatatatatatatatatatatatttcatccaaaaaatacacacacatataaataatgaaatatatttatttaa

SEQ ID NO: 169 - DNA, 1646 bp, Rosa chinensis
  source 1..1646: mol_type = genomic DNA
acacacacacacacacttacctgactatctccatccccttaccatctttggctctttgctcgtgattcaaaacattcccttgtctagttagccatcagtgatgtggacgatgatgggtagcagcggtggttgtggagaccccgataatagggctctgcctgagtactcgatcaaagctgcatatcctcagcctgcagtactactcactccaaagcttagctgtacttccaacatgacccaaatccctttgttaccattcaaccaccaccatcagagtacgtacggatgatgcatacacacacacaatcatatatagatgattacattacccccccccccccccacacacacacacacacacacatatatatatatatttatactttgatggaggacggaggagggtttagggactctccttgatggtctgattattctgtgggtaggtgataatatgatttgatatgtatgcagtggttccaatgacgtcgaatgagcaaggcagaaatagcagtgattgttcttatagttataaacaaccggcagctgcagctcctccgcgaagttcccgatggactccaaccccggagcagataatgcatctggaagagctgtatcaatcttggatcaagacacctacggcccaacaaatccgacaggtgactgcacggcttcgaaactatgggagggttgaaagcaagaatgtcttctactggtttcagaacaacaacaaactttgactgaccacacaattagcagtctccgggacaaagattctgcaggaggtaccatttaataagcatcattttattattaacatgcaattaatctatgtgtttgacacaaggctccccaggtaatagggggattagggctcaacacttggtgaggccacctcctttaggtcacctacggttcaataactttataccatattgaatatctatgctatgaggaggggagagtgagagacttgatggttatgttaataataattaagatgattcagtacaaataatatacccgtgctttacgctcaccgggcaagtcagcaaaccctagccaagagctggagaacctaattaattgaacgggcactcgagtacaaggacttgtaaatttattggttctatatagttgaacgggtgcaaaaattaattaacatattattacaagttatggaaattgtttacttgcagttgactttgttcgttgttttgaagttgagcagcagaataggcggacactatcaagcttcactcttcttaccgatcaggtaagcgtaaaccaaaatctcaagaaagtatagatttttattgaatgcgttattcacttttacaaaatgattattggcctcaatctcagttgtcatgttgtgtcacctacacacatcacacttatgccaagtaatacttgaaaacaaaaatcattttatccttttaaaatctaatgactttgaattatttgcttactttaaaaaaattgaattttttttcctgtttctttttttattttcattacactcatgcttagatttaaactttttttttcttcaattaagaatagaaagtatttcatgtaattcgatcaatagatttgcataacacatgtatatgaattcggagtttgttgggttcctgcaaatt

SEQ ID NO: 170 - DNA, 2029 bp, Rosa chinensis
  source 1..2029: mol_type = genomic DNA
tcctttactaataaaacggcctatataatatgcgcgtgtgcatctccatcgatcctcagctctacttcttcttttttttggtcaatcctcagctctactagctaaatgattctcccttatgtagtcggacttattgttcatgctatttgaatatcaatcattttatcacaatcccgagtcaaaagatttccttgtcgagcagcggtggttttagaaagcctaacatgaacaatagtagggatatccatcaggatgaccactactcgatcaaagctgcaaatgttcatgagcccgctgcagaagtagcattcactccaaagcttagcggtacttccaacatgacccaaagtcatgtgttatcattcaaccaccaccatcatcagggtacctagtagattttctcagttaaattttaaatttgaagatgttgacatgggatatagtaacacaattaatatatttcatacagtggttggagtgacatcgtctgaccaaggcagaagtgccagtgttggtcaacaacccccctctgcagcttcccagcgaagttccagatggaatccaaccccggagcagttacggttcctggaagagctgtacggacatggtttaaagacaccgacggcagagaaaattcgacaagtgactgcgcggcttcgacactttggaaagattgaaggcaagaatgtcttctactggtttcagaaccaccgagcgagggagagacagaagagacgaagagaactcatgtccgtgcctcagtatcactcaaaggaacaacaactttggagtaacaaagaatctgcaggaggtattgttctcgggactgtactgtattaaatattttcatcttttcccaagtaatatggaactagggcttaacactaggctggtgaggctacctcctttaggttaattgattgagtcctcgagttttacaagaagttttaaaatttgtttctatatgagtttcacgtaagattgataggctcaagaagtaattaacagataaatcactagtatggaaattgtctacttgcagctgcaggactgagaaggtcaagtactcttgaaattgagcgccagaataaatggacactgtcaaactgccgtgttcttgttgatcaggtatatatcattttcttaacgtatgtggttcttagacttaacttcatctaaccaaaatctcaagaaaatatagaaatggattgaatgctaggaatttttactattttacattacgtatgtacgtacgtgcatgagtttcatttgttacagtaactctgcaggaattatctccagggcttcgaaagaggatgagcatggtaacgggcaattactctggtcaattggaggagcaatcctattcggctacactgacctcggaagcacaacgaacaaactcatcagctacattaatatacccaaagctcttcgactatcatcatccccattatgattatgattacggtatggtgttgagtgcacccaatatttacaaaggagcagaaagtagattagaaactcaaactctggagctctttccacttgagagcggcaatgttaagggtgttaatgatactaaatcaccaatgattagaaccacaaacacagacagtaatgaagatttcattgcagaccaatattttcaatttctttgaagaatcggaatgtctagctagaaagagagggagggagagaggaggggggttgtgttgtttaagtacctctttatcaggttgtgttctttgttcttcctacttttatttcatcttttttgtttaaaggcagttttatttgtttggaagggtaacgttgaccgttgatgtctccctttctgtttaattttttttttttttttctgtattgttgatcgatcggggggtacgtaccagaaagttcgtctagttagttgaacgtacctttttctttttctgtaaataaatgaataagaatctccatcgtattctcgggttccatgaaatattatgaaactaatatgcttatgtgtacgtagaagctgtcgaat

SEQ ID NO: 171 - DNA, 1608 bp, Rosa chinensis
  source 1..1608: mol_type = genomic DNA
ttttcgcacggagcttccaaaaaatttgagatggctttgatatagtgttataattattttgaataagttatagtaaaaacaatcggtatatatatattcttacagtacgtagttggtgtggcgtggtctgagtatggcagaaatggcggtgattactgctatattgatcaccaaccagcctcttcccgatggaatccaaccctagagcagctacgggtcctggaagagctatatggacatggattaaagacactgacagcacaacaaatccagggagtgactgcatgtgcggctttgacactttggaaaggtcgaaggcaaaatgtcttttctactggtttcaaaaccaccgagcgagagagaggaagaagtgacgacggtgaactcacgtctatgcctctgtctcactctaaggaacaacaactactttggagtagcaaataatctgcaagagccaagaggtattggccggttttgggactgtacgtactgtatcaaatatattttcatctctccccaagtaatatatggaactagggctcaacactggggccggtgaggctacctcttttagatcacttgcgctgatcaataattccacacaccatactaaattaatattacatatatagattgtgtgaggaggagagactctatggttgtgttcataataattaacagataaatcacaggttatggaaattgtctacttgtagctgcgcaactgagaagttgagcaccagaatacttaacatgctgttttcttgctgatcaggtatgcatcattttctacatatgtggttcttagacttaattgtatctaaccgaaatctcaatatcgaaaagatagaaagggattgaatgctagaagtttttacttttttacattatgtatgtgcatatgtttcaatcttaccgtaaatccgcaggaatcatcttcagtgttgcaaaagtggaagagcatggggaccggcaattactccggtcaattggtggagcaattcttctcggctacaccgacctcgaaagcacaatgaacaaacgcatcagctacactaatatacacaaagctcttggactatcatcaccaccattacgattacgattatggtacctctcattatcaggttctgtaatttcttcttttttgttcaaaggcagtttaatttttggaagggtaaataatttattaatttagcaaagtatttcttcttttttcttaagtattgttacccttttttttacctatttacgtccctttttttcacctatttacgtaccgtttcttttccctacttatgtaccctttctttttctgtaaataaattattctccatcacctacctacttatgtaccctttctttttctgtaaataatttattaatttatcaaagtatttttattttcttaaatattgttatattttcaatgtgctatgctgactgatgagcatataattgagagtgttttgggaaatgatgatgaagtttaagatgatagttctactatggagcctgtttcgcgaaagaaagctctcagagcagcgatgactgatgacactgaataagtttttgttgcaacataagaaaaccacaccagaacttttt

SEQ ID NO: 172 - DNA, 1967 bp, Rosa chinensis
  source 1..1967: mol_type = genomic DNA
tccacataaagaaatagaactgctatataatatgtgcgcatgcacactctccacatccttatcagctctactagctaaaagattcccccttatctagttggacttatcgttcatgctatttgaataatcccgagtcaaaagaatcccttgtcgagcagcggtggttttagaaagcctaacatgaacaataatcaggatatgcatgaggatgactactactcgatcaaagctgcaaatcttcatgagcccgctgcagaagtagtattcactcccaagcttaccgctacttccaacatgacccaaagtcatgtgttatcattcaaccaccagcagcatgatcagggtacttagtaaaaattcttagttaaattttaattttgaagatgttgacatgggatatagtaaaacaattaatatatttcatacagaggttggagtgaagtcgttgcaaggccggagtggcggtgatgattgctataatggtcaccaaccttcctctgcagcttcgcagccaagttcccgatggaatccaaccccagagcagttacgggttctggatgagctgttcggacatggattaaagacaccgacggcacagcaaatcctacaagtgactgcacggcttcgacactttggaaagattgaaggcaagaatgtcttctactggtttcagaaccaccgagcgagggagagacagaagagacgaagagaactcatgtccatgcgtcagtatcactctaaggaacaacaactttggagtaacaaagaatctgcaggaggtattggtcttgggactgtactgtattaaatatgttttcatctctccccaagtagtatggaactagggctcaacactaggccggtgaggccacctcctttaggttaaatgattgagtactcgagtttcaaaataggttgtaaaatttgtttctataaatttcacgattgctcaggctcaaaaattaattaacagataaatcacaagttatcgaaattgtctacttgcagctgcaggactgagaaggtcaggtactcttgaaattgagcaccagaataaatggacactatcaaactgcggtgttcttgctgatcaggtatatatcattttcttacgaatacggttcttagacttaactgtatctaaccaaaatctcaagaaaagataggaatggattgaatgctaggaatttttacttttttacattacatgtacgttcatgcatgagtttcatttgttactgtaagtccacaggaattatctccagggttgcggaagaggatgagcatggtgacgggtaattactccggtcaattggaggagcaatcctattcagctacacagaccctggaagcacgcgcagctacattaatatacccaaagctcttggactatcatcatccccattatgattatgattatgattatgattatgattatgattacggtactgtgttgagtgcacccaatatttacaaaggagcagaaagtagattagaagctcaaactctggagctctttccacttgagagcgacaatgttaagggtgttaatgaatcacctatgatcagaactacaaacatagacagtaataaagacttcatggcagacctatattttcaatttctttgaagaatctgagaatcagagtatttagctagaaatctccttgatttggaagcgtttgtcaatctatccatgtaataataaactaaaaaagacccactgcactcttgcatcctttgattcttctcccgttttggggggggggggggggggtaggggtcctctttatcaggttctattcttgttccttcatacttttatttcttctttttgttaaaggccgtagggtaaggttgatgcctccctttatgtttaatttgtttgttttgtattgttggtggggtacgtgaagcggccacacatattgtttcctactaaatgcgaagctctttacattgtttac

SEQ ID NO: 173 - DNA, 2065 bp, Rosa chinensis
  source 1..2065: mol_type = genomic DNA
acacacacacatgcatgcatactcgactctatccatccccttaccatctttgctcatgattcaaaacattcccttgtctagttagccatcatagtttgaataatagggctctgcctgaatactcgatcaaagctgcatatcctcagcctgcagtactactcactccaaagcttagctctacttccaacatgacccaaatccctttgttcctccatcagagtacgtacagatgttagatgtacacacacacacacacacatatgtatatattatactttgatggtctgattattctgtgggtaggtgataatatgatttcaaatgtatgcagtggttccagtgacgtctgatgagcaaggcagaaatagcagtgattgtttttatagtgataaacaaccggcagctgcagcccctccgcgaagttcccgatggaatccaatcccggagcagataatgcacctggaggagctctatcgatctgggatcaagacaccgacggcccaacaaatccgacaggtgactgcacggcttcgaaactatgggagggttgaaggcaagaatgtattctactggtttcagaaccaccgtgccagggagaggcagaagagacgccgtgaactcatgtccatgtatcagtctgactcacaacaacaacaacaaactttgactaacaccacaattagcggtctcccggacaaagaatctgcaggaggtaccaatgaattagcatcattttcttattaacatccgtgcactctttgtgttgcacacacggctccccaagaaataggcggactagggctcaacactaattgaggccacctcctttaggtcacctacggctcaataataactgtataccatatttaatatctatgttttgaggaggggagagcgagagactcaatgattttgttaataataattaagaccattcagtacaaataatataactgtgccttgtactcaccaggctagtcagcaaacacctagctaagagcaggctaacctaattaattgaacaggcactcgagcacaaggacttgtaaatttattggttctatatgaggttcaaagttgagcaggtgttaaaattaattaacagatcagattatcacaagttatggaaattgtctacttgcagttgactttgttggtcgtcttgaagttgagcagcaaaataagcggacactaccaaagtttactttaccttctgatcaggtaaacgtataccagtatatcgaaccaaaatctcaagaaaatatagaaatgaattgaaatgtgtgatatggatatttactgttttaaactacatatgtgcatgagtttcatttgttactgtacgtcctcaggaatcatcttcagtaccgcgagagaggatgatgagcatggtgaaaagaaattcctccaggcaacttgtggaacaatccggttcacctggaccgacctcagacacaaactggtcagatacattaatttacaaaaagcccttggactttcatcactattacgattatgattacggtatgatattgactacacccaatatttacaaagacgagcaagaagaagaaagtagagaaactcaaactctggagctctttccacttgagagtgacaatctcaagggtgctaaggatacgaaattacacctatgagcacaagcccacatgggcttgtacaaacagagacaccactgcagatttcatggcataccaatattatcagttgctttgaacaaattaaggatgcatataactaggaatctatatattcatgtaataaaagtatgcatgcgtgtcttctctttgatggcataatgcatacggcacatctttttgatcgggccggttctgtatttgcttcatcccgcttttgtttcttctatttgttcagacgcagaggaagagtaaggttggtgtccctcttctttaattaattaggtttctttagtttgctagtgtaccctttcgatttttgtaaataaaactccgatccatcatattctggaggtccatcgaaaatgaaaggtcaatacatatgcataactagcaagctctcatat

SEQ ID NO: 174 - protein, 133 aa, Rosa chinensis
MYAVVPVMSDEQGRSSSGCFYSDKQPAAGAPRRSCRWNPTPEQIKDLEELYRSGIKTPTAQQIRQVTARLRNYGRVEGKNVFYWFQNHRARERQKRRRELISMHQSDTKQQQQTLINSKITSLQKKESAGGTN

SEQ ID NO: 175 - protein, 320 aa, Rosa chinensis
MMGSSGGCGDPKTRVLPEYFSIKAAYPQPLLTPKLSCTSNMTQIPLLPFNHHHQMVPVTSNEQGRHSSDCFYSDKQPAAAAPPRSSRWTPTPEQIMHLEELYRSGIKTPTAQQIRQVAARLRNYGRVEGKNVFYWFQNRRARERQKTRRELMSMHQSDSKQQQQQALTKNTISSLRDKESAGVEQQNRRTLPSFTLLTNQKSSSVLQERMMSMVKSNSSRLLMEHIDSPGLTSNINKSATLVYRKLLDFHHYYDYDYGMVLLIAPNIYKEEEEEEESRETQTLKLFPLESDTLKGAKDTKLPMSRTSTDTTSNFMADQYY

SEQ ID NO: 176 - protein, 279 aa, Rosa chinensis
MTQIPFFRHQMVPVTLDEQGSDCFYSDKQPAAAAPPRNSRWNPTPEQIMHLEELYRSGIKTPTTQQIRQVTAGLRNYGRVEGKNVFYWFQNHRARERQKRRRELMSMYQSDSKQQQQQTLTNNTISSLHDKESAGGDFVRCLEVEQQNRRTLPNLTVLSDQESSTVLQERMMSMVKSNSSKLLVEQSDSPGSTSDTNASATVIYTKLLDFHHYYDYGMVLTTPSIYKEFKEEEESRETQTLELFPLESDNLKGAKDTKLPMSRTASTDTTSDFMADQYY

SEQ ID NO: 177 - protein, 138 aa, Rosa chinensis
MGYSKTINIFHTVVGVTQSQGRSGGDYCYNGHQPSSAASQQSSRWNPTPEQLRVLEELYGHGLKTPTAEQIRQVTARLQHFGKIEYKNVFYWFQNHRARERQKRRHELLSMPQYHSKEKQLWSNKESAGGIGLGTVLY

SEQ ID NO: 178 - protein, 128 aa, Rosa chinensis
MCTYAVVPVTSNEQGRNSSDCSYSHNRSSRWTPTPEQIVHLEELYRSGIKTPTAQQIRQVTARLRNYGRVEGKNIFYWFQNRRARERQKTRRELVSMHQSDSKQQQQQTLTNNTISCLREKESAGGII

SEQ ID NO: 179 - protein, 319 aa, Rosa chinensis
MNNSRDIHQDDHYSIKAANVHEPAAEVAFTPKLSGTSNMTQSHVLSFNHHHHQVVGVTSSDQGRSASVGQQPPSAASQRSSRWNPTPEQLRFLEELYGHGLKTPTAEKIRQVTARLRHFGKIEGKNVFYWFQNHRARERQKRRRELMSVPQYHSKEQQLWSNKESAGAAGLRRSSTLEIERQNKWTLSNCRVLVDQELSPGLRKRMSMVTGNYSGQLEEQSYSATLTSEAQRTNSSATLIYPKLFDYHHPHYDYDYGMVLSAPNIYKGAESRLETQTLELFPLESGNVKGVNDTKSPMIRTTNTDSNEDFIADQYFQFL

SEQ ID NO: 180 - protein, 102 aa, Rosa chinensis
MYVHMFQSYRKSAGIIFSVAKVEEHGDRQLLRSIGGAILLGYTDLESTMNKRISYTNIHKALGLSSPPLRLRLWYLSLSGSVISSFLFKGSLIFGRVNNLLI

SEQ ID NO: 181 - protein, 138 aa, Rosa chinensis
MGYSKTINIFHTEVGVKSLQGRSGGDDCYNGHQPSSAASQPSSRWNPTPEQLRVLDELFGHGLKTPTAQQILQVTARLRHFGKIEGKNVFYWFQNHRARERQKRRRELMSMRQYHSKEQQLWSNKESAGGIGLGTVLY

SEQ ID NO: 182 - protein, 257 aa, Rosa chinensis
MYAVVPVTSDEQGRNSSDCFYSDKQPAAAAPPRSSRWNPIPEQIMHLEELYRSGIKTPTAQQIRQVTARLRNYGRVEGKNVFYWFQNHRARERQKRRRELMSMYQSDSQQQQQTLTNTTISGLPDKESAGVDFVGRLEVEQQNKRTLPKFTLPSDQESSSVPRERMMSMVKRNSSRQLVEQSGSPGPTSDTNWSDTLIYKKPLDFHHYYDYDYGMILTTPNIYKDEQEEESRETQTLELFPLESDNLKGAKDTKLHL